(19) United States
(12) Patent Application Publication
(10) Pub. No.: US 2026/0306646 A1
(43) Pub. Date: Oct. 8, 2026

(54) NOVEL CONJUGATE OF IMMUNE-STIMULATING IL-2 ANALOG AND PREPARATION METHOD THEREOF

(71) Applicant: HANMI PHARM. CO., LTD., Hwaseong-si (KR)

(72) Inventors: Yong Ho HEO, Hwaseong-si (KR); Euh Lim OH, Hwaseong-si (KR); Da Hyeon PARK, Hwaseong-si (KR); Jin Young KIM, Hwaseong-si (KR); Jun Sub PARK, Hwaseong-si (KR); Yu Yon KIM, Hwaseong-si (KR); A Ram LEE, Hwaseong-si (KR); Sang Yun KIM, Hwaseong-si (KR)

(73) Assignee: HANMI PHARM. CO., LTD., Hwaseong-si (KR)

(21) Appl. No.: 19/541,949

(22) Filed: Feb. 17, 2026

Related U.S. Application Data
(62) Division of application No. 18/000,096, filed on Mar. 31, 2022, now Pat. No. 12,599,675.

(30) Foreign Application Priority Data
Mar. 31, 2021 (KR) 10-2021-0042305

(51) Int. Cl. A61K 47/68; A61K 38/20; C07K 16/46

(52) U.S. Cl. CPC A61K 47/68; A61K 38/2013; C07K 16/46

(57) ABSTRACT

The present invention relates to a long-acting conjugate of an interleukin-2 analog with altered binding affinity for interleukin-2 receptors.

### CROSS REFERENCE TO RELATED APPLICATIONS

This application is a Divisional of U.S. application Ser. No. 18/000,096 filed Nov. 28, 2022, which is a National Stage of International Application No. PCT/KR2022/004620 filed Mar. 31, 2022, claiming priority based on Korean Patent Application No. 10-2021-0042305 filed Mar. 31, 2021.

### INCORPORATION BY REFERENCE OF SEQUENCE LISTING

The content of the electronically submitted sequence listing, file name: Q317452SEQLIST-ST26.xxxml; size: 624,573 bytes; and date of creation: Feb. 15, 2026, filed herewith, is incorporated herein by reference in its entirety.

### TECHNICAL FIELD

The present invention relates to a novel conjugate of an interleukin-2 analog, a preparation method thereof, and use thereof.

### BACKGROUND ART

Interleukin-2 is an important immunostimulant with a molecular weight of about 15 kDa, which consists of a total of 133 amino acid residues, and activates various cells of the immune system including T cells and B cells. The high efficacy of interleukin-2 as an immunostimulant can be used for the treatment of various immune-related conditions including cancer and AIDS (Korean Patent Application Publication No. 10-2017-0070091). Currently, interleukin-2 (trademark name: Proleukin) is an FDA-approved drug for the treatment of metastatic renal cell carcinoma and metastatic melanoma. However, due to the severe toxicity associated with high-dose interleukin-2 therapy, the applicable patients are limited. In fact, this therapy is applied to only a small number of eligible patients. The toxicity associated with interleukin-2 includes severe fever, nausea, vomiting, vascular leak, severe hypotension, pulmonary edema, and vascular leak syndrome, which causes liver damage.

The interleukin-2 receptor has three kinds of subunit receptors. The subunit consists of an alpha chain (IL-2Rα, CD25), a beta chain (IL-2Rβ or CD122), and a gamma chain (IL-2Rγ or CD132). Interleukin-2 can exhibit various functions by binding to receptor subunits of various combinations. A single interleukin-2 alpha receptor is called a low-affinity interleukin-2 receptor, and it is not involved in signaling. A complex of interleukin-2 beta and gamma receptors binds to interleukin-2 with intermediate affinity. A complex of interleukin-2 alpha, beta, and gamma receptors binds to interleukin-2 with high affinity. The complex of interleukin-2 beta and gamma receptors is required for effective signal conversion through kinase activation in multiple signaling pathways. In particular, interleukin-2 beta- and gamma-binding receptors are prominent in CD8⁺ cells and natural killer (NK) cells. In addition, complexes of high-affinity interleukin-2 alpha, beta, and gamma receptors are usually found in CD4⁺ T regulatory cells (Treg), and recently they were also found in activated T cells. Since interleukin-2 beta receptors are distributed in CD8⁺ T cells or natural killer cells (NK cells) and are involved in the immune response in the body, studies have been conducted to develop therapeutic agents by increasing the activity of beta receptors for immune activation.

Meanwhile, despite the potential of interleukin-2 as a therapeutic agent for various immune-related conditions, there are still not many drugs which can reduce their doses while reducing toxicity and side effects, and thus there is an increasing demand for studies on new and improved drugs.

### DISCLOSURE

### Technical Problem

As part of the development of a therapeutic agent for immune-related diseases, it is required to develop a drug that includes an interleukin-2 analog and has excellent durability and pharmacological effect.

### Technical Solution

An object of the present invention is to provide a long-acting conjugate including an interleukin-2 analog.

Another object of the present invention is to provide a method for preparing the long-acting conjugate.

Still another object of the present invention is to provide a pharmaceutical composition for preventing or treating cancer, including the interleukin-2 analog or a long-acting conjugate thereof.

Yet another object of the present invention is to provide a method for preventing or treating cancer, including administering the interleukin-2 analog, a long-acting conjugate thereof, or a composition including the same to an individual in need thereof.

Even another object of the present invention is to provide the use of the interleukin-2 analog, a long-acting conjugate thereof, or a composition including the same for the prevention or treatment of cancer.

Further another object of the present invention is to provide the use for a providing medicament for the prevention or treatment of cancer, including the interleukin-2 analog or a long-acting conjugate thereof, or a composition including the same.

### Advantageous Effects

The interleukin-2 analog or a long-acting conjugate including the same is an analog which has an increased binding affinity for interleukin-2 beta receptors in vivo and has the effect of improving administration convenience and side effects for anticancer treatment purposes.

### BRIEF DESCRIPTION OF DRAWINGS

FIG. 1 is the SDS-PAGE result of the long-acting conjugate of interleukin-2 analogs (analogs 21, 41, and 52).

FIGS. 2A-2C are the result of analyzing purity of the long-acting conjugate of interleukin-2 analogs (analogs 21, 41, and 52).

FIG. 3A and FIG. 3B show the results of analyzing the pharmacokinetics of the long-acting conjugate of interleukin-2 analogs in ICR and C57BL/6 mice. A is the results of analyzing the pharmacokinetics of an interleukin-2 analog conjugate 41 and interleukin-2 analog conjugate 52 in ICR mice, and B is the results of analyzing the pharmacokinetics of an interleukin-2 analog conjugate 21, interleukin-2 analog conjugate 86, interleukin-2 analog conjugate 104, and interleukin-2 analog conjugate 105 in C57BL/6 mice. In FIG. 3, the dose and serum concentration of the long-acting conjugate are shown based only on the weight occupied by the interleukin-2 analog region in the entire conjugate.

FIG. 4 shows the results of confirming the anticancer effect in an animal model of an interleukin-2 analog conjugate 21, interleukin-2 analog conjugate 41, and interleukin-2 analog conjugate 52 in mice. In FIG. 4, the dose of the long-acting conjugate is a value expressed based only on the weight occupied by the interleukin-2 analog region in the entire conjugate.

FIGS. 5A and 5B are the result confirming the memory T cell change in an animal model of the long-acting conjugate of interleukin-2 analogs. A shows the tumor growth inhibitory effect of an interleukin-2 analog conjugate 21, interleukin-2 analog conjugate 41, and interleukin-2 analog conjugate 52, and B is the result of analyzing the number of memory T cells of an interleukin-2 analog conjugate 21, interleukin-2 analog conjugate 41, and interleukin-2 analog conjugate 52. In FIGS. 5A and 5B are, the dose of the long-acting conjugate is a value expressed based only on the weight occupied by the interleukin-2 analog region in the entire conjugate.

FIGS. 6A and 6B are the result of evaluating the anti-tumor efficacy of the long-acting conjugate of interleukin-2 analogs in an animal model of malignant melanoma. A shows the tumor size of the aldesleukin and interleukin-2 analog conjugate 86, and B is the result of confirming the survival rate of the aldesleukin and interleukin-2 analog conjugate 86. In FIGS. 6A and 6B are, the dose of the long-acting conjugate is a value expressed based only on the weight occupied by the interleukin-2 analog region in the entire conjugate.

### BEST MODE

An aspect of the present invention provides a novel interleukin-2 analog (or IL-2 analog). The interleukin-2 analog is an interleukin-2 analog which has an increased binding affinity for interleukin-2 beta receptors compared to native interleukin-2 or aldesleukin (i.e., an interleukin-2 analog). The interleukin-2 analog may include a sequence in which one or more amino acids in native interleukin-2 are modified.

In a specific embodiment, the long-acting conjugate is characterized in that it is a long-acting conjugate represented by Chemical Formula 1 below:

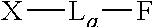

wherein, X is an interleukin-2 analog that when X does not form a part of the long-acting conjugate and is present alone, it is an interleukin-2 analog having an increased binding affinity for interleukin-2 beta receptors compared to aldesleukin;
L is a polyethylene glycol linker;
a is 0 or a natural number, with the proviso that when a is 2 or more, each L is independent of each other;
F is an immunoglobulin Fc region in the form of a dimer; and
- indicates a covalent linkage between X and L and between L and F;
wherein the interleukin-2 analog comprises a sequence in which one or more amino acids corresponding to positions 1, 12, 18, 19, 20, 22, 32, 35, 38, 42, 43, 45, 48, 49, 61, 68, 69, 74, 76, 80, 81, 82, 84, 85, 86, 87, 88, 89, 91, 92, 94, 95, 96, 125, 126, and 133 in native interleukin-2 are modified.

In another specific embodiment, the long-acting conjugate includes an interleukin-2 analog in which one or more amino acids in native interleukin-2 are mutated by one of the methods selected from the group consisting of substitution, addition, deletion, modification, and combinations thereof.

The long-acting conjugate according to any one of the previous specific embodiments is characterized in that the interleukin-2 alpha receptor binding affinity is changed compared to the native interleukin-2 or aldesleukin, and it includes the interleukin-2 analog having an increased binding affinity for interleukin-2 beta receptors as a part of the conjugate.

The long-acting conjugate according to any one of the previous specific embodiments is characterized in that it includes an interleukin-2 analog in which one or more amino acids are added to the amino acid corresponding to position 133 as a part of the conjugate.

The long-acting conjugate according to any one of the previous specific embodiments is characterized in that it includes an interleukin-2, as a part of the conjugate, in which the amino acid at position 1 is removed and the amino acid at position 125 is substituted with a different amino acid in the native interleukin-2.

The long-acting conjugate according to any one of the previous specific embodiments is characterized in that it includes an interleukin-2 analog further including 1 to 10 amino acid substitutions as a part of the conjugate.

The long-acting conjugate according to any one of the previous specific embodiments is characterized in that it includes an interleukin-2 analog, as a part of the conjugate, in which one or more amino acids at positions 18, 19, 20, 22, 38, 42, 43, 45, 61, 68, 69, 74, 80, 81, 84, 85, 86, 88, 89, 91, 92, 94, and 96 of the interleukin-2 analog are further substituted with different amino acids

The long-acting conjugate according to any one of the previous specific embodiments is characterized in that it includes an interleukin-2 analog, as a part of the conjugate, in which one or more amino acids at positions 18, 22, 38, 42, 61, 68, 80, 81, 85, 86 and 92 are further substituted with different amino acids.

The long-acting conjugate according to any one of the previous specific embodiments includes any one of the following analogs as a part of an interleukin-2 analog:
(a) an interleukin-2 analog in which the amino acid at position 1 is removed and the amino acids at positions 125 and 32 are substituted with different amino acids in native interleukin-2;
(b) an interleukin-2 analog in which the amino acid at position 1 is removed and the amino acids at positions 125 and 35 are substituted with different amino acids in native interleukin-2;
(c) an interleukin-2 analog in which the amino acid at position 1 is removed and the amino acids at positions 125 and 38 are substituted with different amino acids in native interleukin-2;
(d) an interleukin-2 analog in which the amino acid at position 1 is removed and the amino acids at positions 125 and 42 are substituted with different amino acids in native interleukin-2;
(e) an interleukin-2 analog in which the amino acid at position 1 is removed and the amino acids at positions 125 and 43 are substituted with different amino acids in native interleukin-2;
(f) an interleukin-2 analog in which the amino acid at position 1 is removed and the amino acids at positions 125 and 48 are substituted with different amino acids in native interleukin-2;
(g) an interleukin-2 analog in which the amino acid at position 1 is removed and the amino acids at positions 125 and 49 are substituted with different amino acids in native interleukin-2;
(h) an interleukin-2 analog in which the amino acid at position 1 is removed and the amino acids at positions 125 and 76 are substituted with different amino acids in native interleukin-2;
(i) an interleukin-2 analog in which the amino acid at position 1 is removed and the amino acids at positions 125, 38, 42, 81, 92, 94, and 96 are substituted with different amino acids in native interleukin-2;
(j) an interleukin-2 analog in which the amino acid at position 1 is removed and the amino acids at positions 125 and 87 are substituted with different amino acids in native interleukin-2;
(k) an interleukin-2 analog in which the amino acid at position 1 is removed and the amino acids at positions 125, 38, and 42 are substituted with different amino acids in native interleukin-2;
(l) an interleukin-2 analog in which the amino acid at position 1 is removed and the amino acids at positions 125, 38, and 80 are substituted with different amino acids in native interleukin-2;
(m) an interleukin-2 analog in which the amino acid at position 1 is removed and the amino acids at positions 125, 38, and 84 are substituted with different amino acids in native interleukin-2;
(n) an interleukin-2 analog in which the amino acid at position 1 is removed and the amino acids at positions 125, 19, 38, and 42 are substituted with different amino acids in native interleukin-2;
(o) an interleukin-2 analog in which the amino acid at position 1 is removed and the amino acids at positions 125, 12, 38, and 42 are substituted with different amino acids in native interleukin-2;
(p) an interleukin-2 analog in which the amino acid at position 1 is removed and the amino acids at positions 125, 38, 42, and 61 are substituted with different amino acids in native interleukin-2;
(q) an interleukin-2 analog in which the amino acid at position 1 is removed and the amino acids at positions 125, 38, 42, and 84 are substituted with different amino acids in native interleukin-2;
(r) an interleukin-2 analog in which the amino acid at position 1 is removed and the amino acids at positions 125, 38, 42, and 88 are substituted with different amino acids in native interleukin-2;
(s) an interleukin-2 analog in which the amino acid at position 1 is removed and the amino acids at positions 125, 38, 42, and 89 are substituted with different amino acids in native interleukin-2;
(t) an interleukin-2 analog in which the amino acid at position 1 is removed and the amino acids at positions 125, 38, 42, and 91 are substituted with different amino acids in native interleukin-2;
(u) an interleukin-2 analog in which the amino acid at position 1 is removed and the amino acids at positions 125, 38, 42, and 94 are substituted with different amino acids in native interleukin-2;
(v) an interleukin-2 analog in which the amino acid at position 1 is removed and the amino acids at positions 125, 38, 42, and 126 are substituted with different amino acids in native interleukin-2;
(w) an interleukin-2 analog in which the amino acid at position 1 is removed and the amino acids at positions 125, 38, 80, and 84 are substituted with different amino acids in native interleukin-2;
(x) an interleukin-2 analog in which the amino acid at position 1 is removed and the amino acids at positions 125, 38, 94, and 96 are substituted with different amino acids in native interleukin-2;
(y) an interleukin-2 analog in which the amino acid at position 1 is removed and the amino acids at positions 125, 38, 81, and 92 are substituted with different amino acids in native interleukin-2;
(z) an interleukin-2 analog in which the amino acid at position 1 is removed and the amino acids at positions 125, 61, 81, and 92 are substituted with different amino acids in native interleukin-2;
(aa) an interleukin-2 analog in which the amino acid at position 1 is removed and the amino acids at positions 125, 38, 42, 81, and 92 are substituted with different amino acids in native interleukin-2;
(ab) an interleukin-2 analog in which the amino acid at position 1 is removed and the amino acids at positions 125, 38, 80, 81, and 92 are substituted with different amino acids in native interleukin-2;
(ac) an interleukin-2 analog in which the amino acid at position 1 is removed and the amino acids at positions 125, 38, 81, 84, and 92 are substituted with different amino acids in native interleukin-2;
(ad) an interleukin-2 analog in which the amino acid at position 1 is removed and the amino acids at positions 125, 20, 38, 42, 81, and 92 are substituted with different amino acids in native interleukin-2;
(ae) an interleukin-2 analog in which the amino acid at position 1 is removed and the amino acids at positions 125, 38, 42, 80, 81, and 92 are substituted with different amino acids in native interleukin-2;
(af) an interleukin-2 analog in which the amino acid at position 1 is removed and the amino acids at positions 125, 38, 42, 74, 81, and 92 are substituted with different amino acids in native interleukin-2;
(ag) an interleukin-2 analog in which the amino acid at position 1 is removed and the amino acids at positions 125, 38, 42, 81, 84, and 92 are substituted with different amino acids in native interleukin-2;
(ah) an interleukin-2 analog in which the amino acid at position 1 is removed and the amino acids at positions 125, 38, 42, 81, 88, and 92 are substituted with different amino acids in native interleukin-2;
(ai) an interleukin-2 analog in which the amino acid at position 1 is removed and the amino acids at positions 125, 38, 42, 85, 86, and 92 are substituted with different amino acids in native interleukin-2;
(aj) an interleukin-2 analog in which the amino acid at position 1 is removed and the amino acids at positions 125, 38, 80, 81, 85, and 92 are substituted with different amino acids in native interleukin-2;
(ak) an interleukin-2 analog in which the amino acid at position 1 is removed and the amino acids at positions 125, 38, 80, 81, 86, and 92 are substituted with different amino acids in native interleukin-2;
(al) an interleukin-2 analog in which the amino acid at position 1 is removed and the amino acids at positions 125, 80, 81, 85, 86, and 92 are substituted with different amino acids in native interleukin-2;
(am) an interleukin-2 analog in which the amino acid at position 1 is removed and the amino acids at positions 125, 38, 42, 45, 74, 81, and 92 are substituted with different amino acids in native interleukin-2;
(an) an interleukin-2 analog in which the amino acid at position 1 is removed and the amino acids at positions 125, 38, 42, 74, 80, 81, and 92 are substituted with different amino acids in native interleukin-2;
(ao) an interleukin-2 analog in which the amino acid at position 1 is removed and the amino acids at positions 125, 38, 42, 80, 81, 84, and 92 are substituted with different amino acids in native interleukin-2;
(ap) an interleukin-2 analog in which the amino acid at position 1 is removed and the amino acids at positions 125, 38, 42, 45, 80, 85, 86, and 92 are substituted with different amino acids in native interleukin-2;
(aq) an interleukin-2 analog in which the amino acid at position 1 is removed and the amino acids at positions 125, 38, 80, 81, 85, 86, and 92 are substituted with different amino acids in native interleukin-2;
(ar) an interleukin-2 analog in which the amino acid at position 1 is removed and the amino acids at positions 125, 42, 80, 81, 85, 86, and 92 are substituted with different amino acids in native interleukin-2;
(as) an interleukin-2 analog in which the amino acid at position 1 is removed and the amino acids at positions 125, 61, 80, 81, 85, 86, and 92 are substituted with different amino acids in native interleukin-2;
(at) an interleukin-2 analog in which the amino acid at position 1 is removed and the amino acids at positions 125, 69, 80, 81, 85, 86, and 92 are substituted with different amino acids in native interleukin-2;
(au) an interleukin-2 analog in which the amino acid at position 1 is removed and the amino acids at positions 125, 80, 81, 85, 86, 91, and 92 are substituted with different amino acids in native interleukin-2;
(av) an interleukin-2 analog in which the amino acid at position 1 is removed and the amino acids at positions 125, 18, 22, 80, 81, 85, 86, and 92 are substituted with different amino acids in native interleukin-2;
(aw) an interleukin-2 analog in which the amino acid at position 1 is removed and the amino acids at positions 125, 38, 42, 80, 81, 85, 86, and 92 are substituted with different amino acids in native interleukin-2;
(ax) an interleukin-2 analog in which the amino acid at position 1 is removed and the amino acids at positions 125, 38, 42, 45, 74, 80, 81, and 92 are substituted with different amino acids in native interleukin-2;
(ay) an interleukin-2 analog in which the amino acid at position 1 is removed and the amino acids at positions 125, 38, 68, 80, 81, 85, 86, and 92 are substituted with different amino acids in native interleukin-2;
(az) an interleukin-2 analog in which the amino acid at position 1 is removed and the amino acids at positions 125, 69, 74, 80, 81, 85, 86, and 92 are substituted with different amino acids in native interleukin-2;
(ba) an interleukin-2 analog in which the amino acid at position 1 is removed and the amino acids at positions 125, 80, 81, 84, 85, 86, 91, and 92 are substituted with different amino acids in native interleukin-2;
(bb) an interleukin-2 analog in which the amino acid at position 1 is removed and the amino acids at positions 125, 80, 81, 85, 86, 92, 94, and 96 are substituted with different amino acids in native interleukin-2;
(bc) an interleukin-2 analog in which the amino acid at position 1 is removed and the amino acids at positions 125, 18, 19, 22, 80, 81, 85, 86, and 92 are substituted with different amino acids in native interleukin-2;
(bd) an interleukin-2 analog in which the amino acid at position 1 is removed and the amino acids at positions 125, 18, 22, 38, 80, 81, 85, 86, and 92 are substituted with different amino acids in native interleukin-2;
(be) an interleukin-2 analog in which the amino acid at position 1 is removed and the amino acids at positions 125, 18, 22, 61, 80, 81, 85, 86, and 92 are substituted with different amino acids in native interleukin-2;
(bf) an interleukin-2 analog in which the amino acid at position 1 is removed and the amino acids at positions 125, 18, 22, 68, 80, 81, 85, 86, and 92 are substituted with different amino acids in native interleukin-2;
(bg) an interleukin-2 analog in which the amino acid at position 1 is removed and the amino acids at positions 125, 35, 38, 42, 80, 81, 85, 86, and 92 are substituted with different amino acids in native interleukin-2;
(bh) an interleukin-2 analog in which the amino acid at position 1 is removed and the amino acids at positions 125, 38, 42, 45, 80, 81, 85, 86, and 92 are substituted with different amino acids in native interleukin-2;
(bi) an interleukin-2 analog in which the amino acid at position 1 is removed and the amino acids at positions 125, 38, 42, 80, 81, 85, 86, 91, and 92 are substituted with different amino acids in native interleukin-2;
(bj) an interleukin-2 analog in which the amino acid at position 1 is removed and the amino acids at positions 125, 38, 42, 80, 81, 85, 86, 92, and 95 are substituted with different amino acids in native interleukin-2;
(bk) an interleukin-2 analog in which the amino acid at position 1 is removed and the amino acids at positions 125, 35, 38, 42, 74, 80, 81, 85, 86, and 92 are substituted with different amino acids in native interleukin-2;
(bl) an interleukin-2 analog in which the amino acid at position 1 is removed and the amino acids at positions 125, 38, 42, 43, 61, 80, 81, 85, 86, and 92 are substituted with different amino acids in native interleukin-2;
(bm) an interleukin-2 analog in which the amino acid at position 1 is removed and the amino acids at positions 125, 38, 42, 80, 81, 85, 86, 91, 92, and 95 are substituted with different amino acids in native interleukin-2;
(bn) an interleukin-2 analog in which the amino acid at position 1 is removed and the amino acids at positions 125, 35, 38, 42, 74, 80, 81, 82, 85, 86, and 92 are substituted with different amino acids in native interleukin-2;
(bo) an interleukin-2 analog in which the amino acid at position 1 is removed and the amino acids at positions 125, 80, 81, 86, and 92 are substituted with different amino acids in native interleukin-2; and (bp) an interleukin-2 analog in which the amino acid at position 1 is removed and the amino acids at positions 125, 80, 81, 85, and 86 are substituted with different amino acids in native interleukin-2.

The long-acting conjugate according to any one of the previous specific embodiments is characterized in that it includes an interleukin-2 analog including any one or more substitutions selected from the group consisting of the following amino acid substitutions:
(a) a substitution in which the amino acid at position 12 is substituted with valine or phenylalanine;
(b) a substitution in which the amino acid at position 18 is substituted with arginine;
(c) a substitution in which the amino acid at position 19 is substituted with tyrosine, valine, phenylalanine, or arginine;
(d) a substitution in which the amino acid at position 20 is substituted with valine or phenylalanine;
(e) a substitution in which the amino acid at position 22 is substituted with glutamic acid;
(f) a substitution in which the amino acid at position 32 is substituted with cysteine;
(g) a substitution in which the amino acid at position 35 is substituted with cysteine or glutamic acid;
(h) a substitution in which the amino acid at position 38 is substituted with alanine or aspartic acid;
(i) a substitution in which the amino acid at position 42 is substituted with lysine, alanine, or tryptophan;
(j) a substitution in which the amino acid at position 43 is substituted with cysteine, glutamic acid, or glutamine;
(k) a substitution in which the amino acid at position 45 is substituted with alanine;
(l) a substitution in which the amino acid at position 48 is substituted with cysteine;
(m) a substitution in which the amino acid at position 49 is substituted with cysteine;
(n) a substitution in which the amino acid at position 61 is substituted with glutamine, arginine, or aspartic acid;
(o) a substitution in which the amino acid at position 68 is substituted with aspartic acid or glutamine;
(p) a substitution in which the amino acid at position 69 is substituted with glycine;
(q) a substitution in which the amino acid at position 74 is substituted with histidine or alanine;
(r) a substitution in which the amino acid at position 76 is substituted with cysteine;
(s) a substitution in which the amino acid at position 80 is substituted with phenylalanine, tyrosine, valine, aspartic acid, or tryptophan;
(t) a substitution in which the amino acid at position 81 is substituted with aspartic acid, glutamic acid, or asparagine;
(u) a substitution in which the amino acid at position 82 is substituted with glycine or valine;
(v) a substitution in which the amino acid at position 84 is substituted with glutamic acid, valine, or phenylalanine;
(w) a substitution in which the amino acid at position 85 is substituted with valine, alanine, glycine, tryptophan, tyrosine, threonine, isoleucine, glutamic acid, or phenylalanine;
(x) a substitution in which the amino acid at position 86 is substituted with valine, alanine, glycine, or leucine;
(y) a substitution in which the amino acid at position 87 is substituted with cysteine;
(z) a substitution in which the amino acid at position 88 is substituted with glutamine, valine, or phenylalanine;
(aa) a substitution in which the amino acid at position 89 is substituted with phenylalanine;
(ab) a substitution in which the amino acid at position 91 is substituted with threonine, phenylalanine, or glutamic acid;
(ac) a substitution in which the amino acid at position 92 is substituted with phenylalanine, leucine, tyrosine, or tryptophan;
(ad) a substitution in which the amino acid at position 94 is substituted with phenyl alanine or valine;
(ae) a substitution in which the amino acid at position 95 is substituted with aspartic acid;
(af) a substitution in which the amino acid at position 96 is substituted with phenylalanine, valine, or isoleucine;
(ag) a substitution in which the amino acid at position 125 is substituted with serine; and
(ah) a substitution in which the amino acid at position 126 is substituted with threonine.

The long-acting conjugate according to any one of the previous specific embodiments is characterized in that it includes an interleukin-2 analog including an amino acid sequence selected from the group consisting of SEQ ID NOS: 3 to 106.

The long-acting conjugate according to any one of the previous specific embodiments is characterized in that it includes an interleukin-2 analog of any one of the following analogs:
(a) an interleukin-2 analog, wherein the interleukin-2 analog is native interleukin-2 in which the amino acid at position 1 is removed and the amino acids at positions 125, 38, 42, 80, 81 and 92 are substituted with different amino acids;
(b) an interleukin-2 analog, wherein the interleukin-2 analog is native interleukin-2 in which the amino acid at position 1 is removed and the amino acids at positions 125, 80, 81, 85, 86, and 92 are substituted with different amino acids;
(c) an interleukin-2 analog, wherein the interleukin-2 analog is native interleukin-2 in which the amino acid at position 1 is removed and the amino acids at positions 125, 18, 22, 80, 81, 85, 86, and 92 are substituted with different amino acids;
(d) an interleukin-2 analog, wherein the interleukin-2 analog is native interleukin-2 in which the amino acid at position 1 is removed and the amino acids at positions 125, 38, 42, 80, 81, 85, 86, and 92 are substituted with different amino acids;
(e) an interleukin-2 analog, wherein the interleukin-2 analog is native interleukin-2 in which the amino acid at position 1 is removed and the amino acids at positions 125, 18, 22, 61, 80, 81, 85, 86, and 92 are substituted with different amino acids; and
(f) an interleukin-2 analog, wherein the interleukin-2 analog is native interleukin-2 in which the amino acid at position 1 is removed and the amino acids at positions 125, 18, 22, 68, 80, 81, 85, 86, and 92 are substituted with different amino acids.

The long-acting conjugate according to any one of the previous specific embodiments is characterized in that it includes an interleukin-2 analog including any one or more amino acid substitutions selected from the group consisting of the following amino acid substitutions:
(a) a substitution in which the amino acid at position 18 is substituted with arginine;
(b) a substitution in which the amino acid at position 22 is substituted with glutamic acid;
(c) a substitution in which the amino acid at position 38 is substituted with alanine;
(d) a substitution in which the amino acid at position 42 is substituted with lysine;
(e) a substitution in which the amino acid at position 61 is substituted with aspartic acid;
(f) a substitution in which the amino acid at position 68 is substituted with aspartic acid;
(g) a substitution in which the amino acid at position 80 is substituted with phenylalanine;
(h) a substitution in which the amino acid at position 81 is substituted with glutamic acid;
(i) a substitution in which the amino acid at position 85 is substituted with valine;
(j) a substitution in which the amino acid at position 86 is substituted with valine;
(k) a substitution in which the amino acid at position 92 is substituted with phenylalanine; and
(l) a substitution in which the amino acid at position 125 is substituted with serine.

The long-acting conjugate according to any one of the previous specific embodiments is characterized in that it includes an immunoglobulin Fc region which is derived from IgG, IgA, IgD, IgE, IgM, or a combination thereof, or a hybrid thereof.

The long-acting conjugate according to any one of the previous specific embodiments is characterized in that it includes an IgG4 Fc region.

The long-acting conjugate according to any one of the previous specific embodiments is characterized in that it includes an immunoglobulin Fc region which is non-glycosylated.

The long-acting conjugate according to any one of the preceding embodiments is characterized in that the immunoglobulin Fc region is derived from a human IgG4-derived non-glycosylated Fc region.

In the long-acting conjugate according to any one of the previous specific embodiments, the immunoglobulin Fc region has a structure in which two polypeptide chains are linked by a disulfide bond, and is linked only through a nitrogen atom in one of the two chains.

The long-acting conjugate according to any one of the previous specific embodiments is characterized in that the immunoglobulin Fc region includes a monomer having an amino acid sequence of SEQ ID NO: 438.

The long-acting conjugate according to any one of the previous specific embodiments is characterized in that the immunoglobulin Fc region is a homodimer of the monomers of the amino acid sequence of SEQ ID NO: 438.

The long-acting conjugate according to any one of the previous specific embodiments is characterized in that the immunoglobulin Fc region is linked through a nitrogen atom of the N-terminal proline thereof.

The long-acting conjugate according to any one of the previous specific embodiments is characterized in that the L contains ethylene glycol repeating units, and the formula weight of the ethylene glycol repeating unit moiety is in the range of 1 kDa to 100 kDa.

The long-acting conjugate according to any one of the previous specific embodiments is characterized in that one molecule of X is covalently linked to one of the Fc regions of the dimeric immunoglobulin Fc region through the polyethylene glycol linker.

The long-acting conjugate according to any one of the previous specific embodiments is characterized in that the ethylene glycol repeating unit has a formula of [OCH₂CH₂]ₙ, wherein n is a natural number, and the average molecular weight of the [OCH₂CH₂]ₙ region in the interleukin-2 analog conjugate, for example, the number average molecular weight is determined to be 1 kDa to 100 kDa.

The long-acting conjugate according to any one of the previous specific embodiments is characterized in that the value of n is determined such that the average molecular weight of the [OCH₂CH₂]ₙ region in the interleukin-2 analog conjugate, for example, the number average molecular weight is 3.4 kDa.

The long-acting conjugate according to any one of the previous specific embodiments is characterized in that a is 1, and one molecule of X is covalently linked to one of the Fc region chains of the dimeric immunoglobulin Fc region through a linker containing the ethylene glycol repeating units.

The long-acting conjugate according to any one of the previous specific embodiments is characterized in that one end of the linker is linked only to one of the two Fc region chains of the dimeric immunoglobulin Fc region.

As the long-acting conjugate according to any one of the previous specific embodiments, the conjugate is characterized in that one end of L is linked to F and the other end of L is linked to X by a covalent bond formed by reacting with an amine or thiol group of F and with an amine or thiol group of X, respectively.

Another aspect of the present invention provides a long-acting conjugate of an interleukin-2 analog, represented by Chemical Formula 2 below:

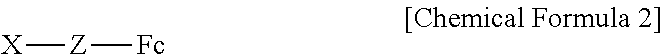

wherein X is an interleukin-2 analog including any one of polypeptide sequences selected from the amino acid sequence of SEQ ID NOS: 3 to 106;
Z is a polyethylene glycol linker having a molecular weight of 2 kDa to 30 kDa;
Fc is an immunoglobulin Fc region in the form of a dimer; and
- indicates a covalent linkage between X and Z and between Z and Fc;
wherein the long-acting conjugate is a long-acting conjugate in which one end of Z is covalently linked to only one polypeptide chain in the dimeric Fc region, and one molecule of X is covalently linked to the opposite end of Z.

In a specific embodiment, the interleukin-2 analog is characterized in that it includes any one of sequences selected from the group consisting of amino acid sequences of SEQ ID NOS: 10, 13, 14, 15, 16, 17, 20, 21, 22, 32, 35, 36, 42, 53, 54, 56, 58, 59, 60, 62, 71, 72, 74, 75, 76, 77, 78, 85, 87, 89, 91, 92, 93, 94, 95, 98, 99, 100, 101, 103, 104, 105, and 106.

In another specific embodiment, the interleukin-2 analog is characterized in that it includes any one of sequences selected from the group consisting of amino acid sequences of SEQ ID NOS: 10, 13, 14, 16, 17, 20, 21, 22, 32, 35, 36, 42, 53, 54, 87, 89, 91, 92, 93, 94, 98, 99, 100, 101, 103, 104, and 105.

The long-acting conjugate according to any one of the previous specific embodiments is characterized in that it includes an interleukin-2 analog including any one of sequences selected from the group consisting of the amino acid sequences of SEQ ID NOS: 22, 42, 53, 87, 105 and 106.

The interleukin-2 analog of the long-acting conjugate according to any one of the previous specific embodiments is characterized in that it further includes one or more amino acids at the C-terminus thereof.

Still another aspect of the present invention provides a long-acting conjugate of an interleukin-2 analog, represented by Chemical Formula 3 below:

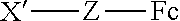

wherein X′ is an interleukin-2 analog including an amino acid sequence represented by General Formula 1 below:

[General Formula 1]
(General Formula 1, SEQ ID NO: 212)
X1-P-T-S-S-S-T-K-K-T-Q-L-Q-L-E-H-L-X18-X19-D-L-
X22-M-I-L-N-G-I-N-N-Y-K-N-P-K-L-T-X38-M-L-T-X42-
X43-F-X45-M-P-K-K-A-T-E-L-K-H-L-Q-C-L-E-X61-E-L-K-
P-L-E-X68-V-L-N-L-A-X74-S-K-N-F-H-X80-X81-P-R-X84-
X85-X86-S-N-I-N-X91-X92-V-X94-E-X96-K-G-S-E-T-T-F-
M-C-E-Y-A-D-E-T-A-T-I-V-E-F-L-N-R-W-I-T-F-S-Q-S-I-
I-S-T-L-T

wherein in General Formula 1 above,
X1 is a deletion;
X18 is leucine (L) or arginine (R);
X19 is leucine (L) or tyrosine (Y);
X22 is glutamic acid (E) or glutamine (Q);
X38 is alanine (A), aspartic acid (D), or arginine (R);
X42 is alanine (A), phenylalanine (F), lysine (K), or tryptophan (W);
X43 is glutamic acid (E), lysine (K), or glutamine (Q);
X45 is alanine (A) or tyrosine (Y);
X61 is aspartic acid (D), glutamic acid (E), glutamine (Q), or arginine (R);
X68 is aspartic acid (D) or glutamic acid (E);
X74 is histidine (H) or glutamine (Q);
X80 is phenylalanine (F), leucine (L), valine (V), or tyrosine (Y);
X81 is aspartic acid (D), glutamic acid (E), or arginine (R);
X84 is aspartic acid (D) or glutamic acid (E);
X85 is alanine (A), glutamic acid (E), glycine (G), leucine (L), valine (V), tryptophan
(W), or tyrosine (Y);
X86 is alanine (A), glycine (G), isoleucine (I), or valine (V);
X91 is threonine (T) or valine (V);
X92 is phenylalanine (F), isoleucine (I), or tyrosine (Y);
X94 is phenylalanine (F) or leucine (L);
X96 is phenylalanine (F) or leucine (L);
Z is a polyethylene glycol linker having a molecular weight of 2 kDa to 30 kDa;
Fc is an immunoglobulin Fc region in the form of a dimer; and
- indicates a covalent linkage between X′ and Z and between Z and Fc;
wherein the long-acting conjugate is a long-acting conjugate in which one end of Z is covalently linked only to one polypeptide chain in the dimeric Fc region, and one molecule of X′ is covalently linked to the opposite end of Z.

In a specific embodiment, in General Formula 1 above,
X43 is lysine (K);
X45 is tyrosine (Y);
X61 is aspartic acid (D), glutamic acid (E), or glutamine (Q);
X68 is glutamic acid (E);
X74 is glutamine (Q);
X80 is phenylalanine (F) or leucine (L);
X85 is leucine (L), valine (V), or tyrosine (Y);
X86 is isoleucine (I) or valine (V); and
X92 is phenylalanine (F) or isoleucine (I).

In another embodiment, the interleukin-2 analog further includes one or more amino acids at the C-terminus thereof.

Yet another aspect of the present invention provides a long-acting conjugate of an interleukin-2 analog, represented by Chemical Formula 4 below:

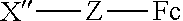

wherein X″ is an interleukin-2 analog including an amino acid sequence represented by General Formula 2 below:

[General Formula 2]
(General Formula 2, SEQ ID NO: 213)
X1-P-T-S-S-S-T-K-K-T-Q-L-Q-L-E-H-L-X18-L-D-L-X22-
M-I-L-N-G-I-N-N-Y-K-N-P-K-L-T-X38-M-L-T-X42-K-F-Y-
M-P-K-K-A-T-E-L-K-H-L-Q-C-L-E-X61-E-L-K-P-L-E-X68-
V-L-N-L-A-Q-S-K-N-F-H-F-X81-P-R-D-X85-X86-S-N-I-N-
V-F-V-L-E-L-K-G-S-E-T-T-F-M-C-E-Y-A-D-E-T-A-T-I-V-
E-F-L-N-R-W-I-T-F-S-Q-S-I-I-S-T-L-T

X1 is a deletion;
X18 is leucine (L) or arginine (R);
X22 is glutamic acid (E) or glutamine (Q);
X38 is alanine (A) or arginine (R);
X42 is phenylalanine (F) or lysine (K);
X61 is aspartic acid (D) or glutamic acid (E);
X68 is aspartic acid (D) or glutamic acid (E);
X81 is aspartic acid (D) or glutamic acid (E);
X85 is leucine (L) or valine (V);
X86 is isoleucine (I) or valine (V);
Z is a polyethylene glycol linker having a molecular weight of 2 kDa to 30 kDa;
Fc is an immunoglobulin Fc region in the form of a dimer; and
- indicates a covalent linkage between X″ and Z and between Z and Fc;
wherein the long-acting conjugate is a long-acting conjugate in which one end of Z is covalently linked only to one polypeptide chain in the dimeric Fc region, and one molecule of X″ is covalently linked to the opposite end of Z.

In a specific embodiment, the interleukin-2 analog is characterized in that it includes any one of sequences selected from the group consisting of amino acid sequences of SEQ ID NOS: 22, 42, 53, 87, 105, and 106.

Even another aspect of the present invention provides a method for preparing a long-acting conjugate including an interleukin-2 analog, including: linking the interleukin-2 analog with an immunoglobulin Fc region through a non-peptidyl polymer.

In a specific embodiment, the interleukin-2 analog is characterized in that one or more amino acids selected from the group consisting of amino acids corresponding to positions 1, 12, 18, 19, 20, 22, 32, 35, 38, 42, 43, 45, 48, 49, 61, 68, 69, 74, 76, 80, 81, 82, 84, 85, 86, 87, 88, 89, 91, 92, 94, 95, 96, 125, 126, and 133 in native interleukin-2 are modified.

Further another aspect of the present invention provides a pharmaceutical composition for preventing or treating cancer, including an interleukin-2 analog or a long-acting conjugate thereof.

In a specific embodiment, the pharmaceutical composition is characterized in that it includes a pharmaceutically effective amount of a long-acting conjugate of an interleukin-2 analog and a pharmaceutically acceptable excipient.

In another specific embodiment, the interleukin-2 analog of the pharmaceutical composition is characterized in that it includes a sequence in which one or more amino acids corresponding to positions 1, 12, 18, 19, 20, 22, 32, 35, 38, 42, 43, 45, 48, 49, 61, 68, 69, 74, 76, 80, 81, 82, 84, 85, 86, 87, 88, 89, 91, 92, 94, 95, 96, 125, 126, and 133 in native interleukin-2 are modified.

The pharmaceutical composition according to any one of the previous specific embodiments is characterized in that it includes an interleukin-2 analog having an increased binding affinity for interleukin-2 beta receptors compared to aldesleukin.

The pharmaceutical composition according to any one of the previous specific embodiments is characterized in that the interleukin-2 analog is selected from the group consisting of SEQ ID NOS: 3 to 106.

The pharmaceutical composition according to any one of the previous specific embodiments is characterized in that the interleukin-2 analog is selected from the group consisting of SEQ ID NOS: 22, 42, 53, 87, 105, and 106.

The pharmaceutical composition according to any one of the previous specific embodiments is characterized in that the long-acting conjugate is represented by Chemical Formula 1 below:

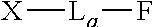

wherein, X is an interleukin-2 analog having an increased binding affinity for interleukin-2 beta receptors compared to aldesleukin;
L is a polyethylene glycol linker;
a is 0 or a natural number, with the proviso that when a is 2 or more, each L is independent of each other;
F is an immunoglobulin Fc region in the form of a dimer; and
- indicates a covalent linkage between X and L and between L and F.

The pharmaceutical composition according to any one of the previous specific embodiments is characterized in that the cancer is any one selected from the group consisting of metastatic renal cell cancer, metastatic melanoma, colorectal cancer, liver cancer, ovarian cancer, pancreatic cancer, gallbladder cancer, kidney cancer, colorectal cancer, lung cancer, skin cancer, melanoma, breast cancer, bladder cancer, and stomach cancer.

The pharmaceutical composition according to any one of the previous specific embodiments is characterized in that the composition exhibits high blood exposure, tumor growth inhibition and/or memory T-cell response relative to aldesleukin.

The pharmaceutical composition according to any one of the previous specific embodiments is characterized in that the pharmaceutical composition is administered via an intraperitoneal, intravenous, intramuscular, subcutaneous, intradermal, oral, topical, intranasal, intrapulmonary, or intrarectal route.

The pharmaceutical composition according to any one of the previous specific embodiments is characterized in that the pharmaceutical composition is administered at a time interval ranging from 1 week to 1 month.

Still further another aspect of the present invention provides a method for preventing or treating cancer, including: administering the interleukin-2 analog, a long-acting conjugate thereof, or a composition including the same to an individual in need thereof.

Still further another aspect of the present invention provides the use of the interleukin-2 analog, a long-acting conjugate thereof, or a composition including the same for the prevention or treatment of cancer.

Still further another aspect of the present invention provides the use for providing a medicament for the prevention or treatment of cancer, including the interleukin-2 analog or a long-acting conjugate thereof, or a composition including the same.

### MODE FOR INVENTION

The details for carrying out the present invention will be described as follows. Meanwhile, respective descriptions and embodiments disclosed in the present invention may also be applied to other descriptions and embodiments. That is, all combinations of various elements disclosed in the present invention fall within the scope of the present invention. Further, the scope of the present invention cannot be considered to be limited by the specific description below. Furthermore, a number of papers and patent documents are referenced and cited throughout this specification. The disclosures of the cited papers and patent documents are incorporated herein by reference in their entirety to further clarify the level and scope of the subject matter to which the present invention pertains.

Over the entire specification of the present invention, the conventional one-letter and three-letter codes for amino acids are used. Additionally, the amino acids mentioned herein are abbreviated according to the nomenclature rules of the IUPAC-IUB as follows:

| | |
|---|---|
| alanine | A |
| arginine | R |
| asparagine | N |
| aspartic acid | D |
| cysteine | C |
| glutamic acid | E |
| glutamine | Q |
| glycine | G |
| histidine | H |
| isoleucine | 1 |
| leucine | L |
| lysine | K |
| methionine | M |
| phenylalanine | F |
| proline | P |
| serine | S |
| threonine | T |
| tryptophan | W |
| tyrosine | Y |
| valine | V |

As used herein, “Aib” may be used interchangeably with “2-aminoisobutyric acid” or “aminoisobutyric acid”, and 2-aminoisobutyric acid and aminoisobutyric acid may be used interchangeably with each other.

One aspect of the present invention provides a long-acting conjugate including interleukin-2 analog.

Specifically, the long-acting conjugate may be represented by Chemical Formula 1 below:

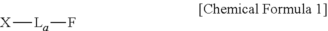

wherein, X is an interleukin-2 analog having an increased binding affinity for interleukin-2 beta receptors compared to aldesleukin;
L is a polyethylene glycol linker;
a is 0 or a natural number, with the proviso that when a is 2 or more, each L is independent of each other;
F is an immunoglobulin Fc region in the form of a dimer; and
- indicates a covalent linkage between X and L and between L and F.

The interleukin-2 analog of the long-acting conjugate of the present invention is characterized in that its binding affinity for interleukin-2 receptors is altered when it is present alone without forming a part of the conjugate, and in particular in that it has increased binding affinity for interleukin-2 beta receptors. Specifically, the interleukin-2 analog of the present invention may be one which has increased binding affinity for interleukin-2 beta receptors compared to native interleukin-2 or known aldesleukin, when it is present alone without forming a part of the conjugate, and more specifically one which also has altered (increased or decreased) binding affinity for interleukin-2 alpha receptors, and may include a sequence in which one or more amino acids in native interleukin-2 are modified.

As used herein, the term “interleukin-2 (IL-2)” refers to a type of cytokine which transmits signals in the immune system in vivo. The interleukin-2 is generally known as an important immunostimulator with a size of about 15 kDa.

As used herein, the term “interleukin-2 analog” refers to native interleukin-2 in which one or more amino acids in the sequence thereof are modified. Particularly in the present invention, the interleukin-2 analog may be an interleukin-2 analog which has reduced or increased binding affinity for interleukin-2 receptors compared to native interleukin-2, in which amino acids in native interleukin-2 are modified. Specifically, the interleukin-2 analog of the present invention may be one which is not naturally occurring.

The native interleukin-2 may be a human interleukin-2, and its sequence may be obtained from known databases, etc. Specifically, the native interleukin-2 may have an amino acid sequence of SEQ ID NO: 1, but is not limited thereto.

In the present invention, what is meant by “native interleukin-2 may have an amino acid sequence of SEQ ID NO: 1” is that not only the sequence which is the same as SEQ ID NO: 1, but also sequences which have a homology of 80%, 85%, 90%, 91%, 92%, 93%, 94%, 95%, 96%, 97%, 98%, or 99% or higher to SEQ ID NO: 1 belong to the scope of native interleukin-2 of the present invention; and that the corresponding position(s) of amino acid modification is (are) altered on the amino acid sequence of SEQ ID NO: 1 when sequences having a homology of 80%, 85%, 90%, 91%, 92%, 93%, 94%, 95%, 96%, 97%, 98%, or 99% or higher are aligned based on SEQ ID NO: 1.

In the present invention, what is meant by “one or more amino acids in the native sequence are altered” may be that a modification selected from the group consisting of substitution, addition, deletion, modification, and a combination thereof has occurred in at least one amino acid in the native sequence.

Specifically, the interleukin-2 analog of the present invention may include a sequence in which one or more amino acids corresponding to positions 1, 12, 18, 19, 20, 22, 32, 35, 38, 42, 43, 45, 48, 49, 61, 68, 69, 74, 76, 80, 81, 82, 84, 85, 86, 87, 88, 89, 91, 92, 94, 95, 96, 125, 126, and 133 in native interleukin-2 are modified. Specifically, the interleukin-2 analog of the present invention may be one in which the amino acid at position 1 is removed and the amino acid at position 125 is substituted with a different amino acid in native interleukin-2; and which further includes 1, 2, 3, 4, 5, 6, 7, 8, 9, 10, or more amino acid substitutions. Although not limited thereto, the amino acid at position 125 (i.e., cysteine) may be substituted with serine, and the amino acid(s) at which a further substitution occurs may be amino acids corresponding to positions 12, 18, 19, 20, 22, 32, 35, 38, 42, 43, 45, 48, 49, 61, 68, 69, 74, 76, 80, 81, 82, 84, 85, 86, 87, 88, 89, 91, 92, 94, 95, 96, 126, and 133.

Additionally, interleukin-2 analogs which include substitution, addition, deletion, modification, etc. of amino acid residues in addition to the positions for modification above to the extent that can be performed for the stability and increase of half-life of a peptide known in the art are also included within the scope of the present invention.

As used herein, the term “aldesleukin” or “interleukin-2 analog (aldesleukin)”, which is a commercially available interleukin-2 analog, may be aldesleukin (trademark name: Proleukin®), and specifically may be one which has the amino acid sequence of SEQ ID NO: 2. In the present invention, these terms are used interchangeably with “interleukin-2 analog 1”. The interleukin analog according to the present invention may have altered binding affinity for interleukin-2 alpha receptors and/or increased binding affinity for interleukin-2 beta receptors compared to the interleukin-2 analog 1.

Although interleukin-2 alpha receptors are not known to be involved in the signaling system of interleukin-2, they increase the binding affinity of interleukin-2 for other interleukin-2 receptors (beta or gamma receptors) by 10 to 100 times and are expressed in CD4+ regulatory T cells, etc.

Since interleukin-2 beta receptors are mainly distributed in CD8+ T cells or natural killer cells (NK cells) and have an important role of activating immune responses and macrophages, it is expected that tumor cell death and activation of the body's immune responses can be promoted through the activation of interleukin-2 beta receptors.

Accordingly, the interleukin-2 analog of the present invention which has increased binding affinity for interleukin-2 beta receptors can have a therapeutic effect where the suppression and death of tumors is increased while side effects are reduced.

In the present invention, the interleukin-2 analog may include a sequence in which the amino acid at position 1 is removed and the amino acid at position 125 is substituted with a different amino acid in native interleukin-2, and which further includes 1 to 10 amino acid modifications. For example, the interleukin-2 analog may include a sequence in which the amino acid at position 125 is substituted with serine and one or more amino acids at positions 12, 18, 19, 20, 22, 32, 35, 38, 42, 43, 45, 48, 49, 61, 68, 69, 74, 76, 80, 81, 82, 84, 85, 86, 87, 88, 89, 91, 92, 94, 95, 96, and 126 are substituted with different amino acids and/or one or more amino acids are added on the amino acid at position 133 in native interleukin-2, but the sequence is not limited thereto, and any interleukin-2 analog which has altered binding affinity for interleukin-2 alpha receptors and increased binding affinity for interleukin-2 beta receptors compared to native interleukin-2 and/or aldesleukin is included without limitation.

As an example of the interleukin-2 analog of the present invention, the interleukin-2 analog may be one in which one or more amino acids are added to the amino acid corresponding to position 133, but is not limited thereto. For the purpose of the present invention, the amino acids to be added are not limited with regard to the type or length thereof as long as the interleukin-2 analog has altered binding affinity for interleukin-2 alpha receptors and increased binding affinity for interleukin-2 beta receptors compared to native interleukin-2 or aldesleukin, and amino acids which are not naturally occurring and amino acids with a chemical modification can also be added in addition to natural amino acids.

In another embodiment, the interleukin-2 analog may be one in which the amino acid at position 1 is removed; the amino acid at position 125 is substituted with a different amino acid; and 1, 2, 3, 4, 5, 6, 7, 8, 9, or more amino acids among the amino acids at positions 18, 19, 20, 22, 38, 42, 43, 45, 61, 68, 69, 74, 80, 81, 84, 85, 86, 88, 89, 91, 92, 94, and 96 are substituted with different amino acids in native interleukin-2, but is not limited thereto.

In still another embodiment, the interleukin-2 analog may be one in which the amino acid at position 1 is removed; the amino acid at position 125 is substituted with a different amino acid; and 1, 2, 3, 4, 5, 6, 7, 8, 9, or more amino acids among the amino acids at positions 18, 19, 22, 38, 42, 43, 45, 61, 68, 74, 80, 81, 84, 85, 86, 88, 91, 92, 94, and 96 are substituted with different amino acids in native interleukin-2, but is not limited thereto.

In still another embodiment, the interleukin-2 analog may be one in which the amino acid at position 1 is removed; the amino acid at position 125 is substituted with a different amino acid; and one or more amino acids among the amino acids at positions 18, 22, 38, 42, 61, 68, 80, 81, 85, 86, and 92 are substituted with different amino acids in native interleukin-2, but is not limited thereto.

In still another embodiment, the interleukin-2 analog may be any one selected from the group consisting of the following analogs:
(a) an interleukin-2 analog in which the amino acid at position 1 is removed and the amino acids at positions 125 and 32 are substituted with different amino acids in native interleukin-2;
(b) an interleukin-2 analog in which the amino acid at position 1 is removed and the amino acids at positions 125 and 35 are substituted with different amino acids in native interleukin-2;
(c) an interleukin-2 analog in which the amino acid at position 1 is removed and the amino acids at positions 125 and 38 are substituted with different amino acids in native interleukin-2;
(d) an interleukin-2 analog in which the amino acid at position 1 is removed and the amino acids at positions 125 and 42 are substituted with different amino acids in native interleukin-2;
(e) an interleukin-2 analog in which the amino acid at position 1 is removed and the amino acids at positions 125 and 43 are substituted with different amino acids in native interleukin-2;
(f) an interleukin-2 analog in which the amino acid at position 1 is removed and the amino acids at positions 125 and 48 are substituted with different amino acids in native interleukin-2;
(g) an interleukin-2 analog in which the amino acid at position 1 is removed and the amino acids at positions 125 and 49 are substituted with different amino acids in native interleukin-2;
(h) an interleukin-2 analog in which the amino acid at position 1 is removed and the amino acids at positions 125 and 76 are substituted with different amino acids in native interleukin-2;
(i) an interleukin-2 analog in which the amino acid at position 1 is removed and the amino acids at positions 125, 38, 42, 81, 92, 94, and 96 are substituted with different amino acids in native interleukin-2;
(j) an interleukin-2 analog in which the amino acid at position 1 is removed and the amino acids at positions 125 and 87 are substituted with different amino acids in native interleukin-2;
(k) an interleukin-2 analog in which the amino acid at position 1 is removed and the amino acids at positions 125, 38, and 42 are substituted with different amino acids in native interleukin-2;
(l) an interleukin-2 analog in which the amino acid at position 1 is removed and the amino acids at positions 125, 38, and 80 are substituted with different amino acids in native interleukin-2;
(m) an interleukin-2 analog in which the amino acid at position 1 is removed and the amino acids at positions 125, 38, and 84 are substituted with different amino acids in native interleukin-2;
(n) an interleukin-2 analog in which the amino acid at position 1 is removed and the amino acids at positions 125, 19, 38, and 42 are substituted with different amino acids in native interleukin-2;
(o) an interleukin-2 analog in which the amino acid at position 1 is removed and the amino acids at positions 125, 12, 38, and 42 are substituted with different amino acids in native interleukin-2;
(p) an interleukin-2 analog in which the amino acid at position 1 is removed and the amino acids at positions 125, 38, 42, and 61 are substituted with different amino acids in native interleukin-2;
(q) an interleukin-2 analog in which the amino acid at position 1 is removed and the amino acids at positions 125, 38, 42, and 84 are substituted with different amino acids in native interleukin-2;
(r) an interleukin-2 analog in which the amino acid at position 1 is removed and the amino acids at positions 125, 38, 42, and 88 are substituted with different amino acids in native interleukin-2;
(s) an interleukin-2 analog in which the amino acid at position 1 is removed and the amino acids at positions 125, 38, 42, and 89 are substituted with different amino acids in native interleukin-2;
(t) an interleukin-2 analog in which the amino acid at position 1 is removed and the amino acids at positions 125, 38, 42, and 91 are substituted with different amino acids in native interleukin-2;
(u) an interleukin-2 analog in which the amino acid at position 1 is removed and the amino acids at positions 125, 38, 42, and 94 are substituted with different amino acids in native interleukin-2;
(v) an interleukin-2 analog in which the amino acid at position 1 is removed and the amino acids at positions 125, 38, 42, and 126 are substituted with different amino acids in native interleukin-2;
(w) an interleukin-2 analog in which the amino acid at position 1 is removed and the amino acids at positions 125, 38, 80, and 84 are substituted with different amino acids in native interleukin-2;
(x) an interleukin-2 analog in which the amino acid at position 1 is removed and the amino acids at positions 125, 38, 94, and 96 are substituted with different amino acids in native interleukin-2;
(y) an interleukin-2 analog in which the amino acid at position 1 is removed and the amino acids at positions 125, 38, 81, and 92 are substituted with different amino acids in native interleukin-2;
(z) an interleukin-2 analog in which the amino acid at position 1 is removed and the amino acids at positions 125, 61, 81, and 92 are substituted with different amino acids in native interleukin-2;
(aa) an interleukin-2 analog in which the amino acid at position 1 is removed and the amino acids at positions 125, 38, 42, 81, and 92 are substituted with different amino acids in native interleukin-2;
(ab) an interleukin-2 analog in which the amino acid at position 1 is removed and the amino acids at positions 125, 38, 80, 81, and 92 are substituted with different amino acids in native interleukin-2;
(ac) an interleukin-2 analog in which the amino acid at position 1 is removed and the amino acids at positions 125, 38, 81, 84, and 92 are substituted with different amino acids in native interleukin-2;
(ad) an interleukin-2 analog in which the amino acid at position 1 is removed and the amino acids at positions 125, 20, 38, 42, 81, and 92 are substituted with different amino acids in native interleukin-2;
(ae) an interleukin-2 analog in which the amino acid at position 1 is removed and the amino acids at positions 125, 38, 42, 80, 81, and 92 are substituted with different amino acids in native interleukin-2;
(af) an interleukin-2 analog in which the amino acid at position 1 is removed and the amino acids at positions 125, 38, 42, 74, 81, and 92 are substituted with different amino acids in native interleukin-2;
(ag) an interleukin-2 analog in which the amino acid at position 1 is removed and the amino acids at positions 125, 38, 42, 81, 84, and 92 are substituted with different amino acids in native interleukin-2;
(ah) an interleukin-2 analog in which the amino acid at position 1 is removed and the amino acids at positions 125, 38, 42, 81, 88, and 92 are substituted with different amino acids in native interleukin-2;
(ai) an interleukin-2 analog in which the amino acid at position 1 is removed and the amino acids at positions 125, 38, 42, 85, 86, and 92 are substituted with different amino acids in native interleukin-2;
(aj) an interleukin-2 analog in which the amino acid at position 1 is removed and the amino acids at positions 125, 38, 80, 81, 85, and 92 are substituted with different amino acids in native interleukin-2;
(ak) an interleukin-2 analog in which the amino acid at position 1 is removed and the amino acids at positions 125, 38, 80, 81, 86, and 92 are substituted with different amino acids in native interleukin-2;
(al) an interleukin-2 analog in which the amino acid at position 1 is removed and the amino acids at positions 125, 80, 81, 85, 86, and 92 are substituted with different amino acids in native interleukin-2;
(am) an interleukin-2 analog in which the amino acid at position 1 is removed and the amino acids at positions 125, 38, 42, 45, 74, 81, and 92 are substituted with different amino acids in native interleukin-2;
(an) an interleukin-2 analog in which the amino acid at position 1 is removed and the amino acids at positions 125, 38, 42, 74, 80, 81, and 92 are substituted with different amino acids in native interleukin-2;
(ao) an interleukin-2 analog in which the amino acid at position 1 is removed and the amino acids at positions 125, 38, 42, 80, 81, 84, and 92 are substituted with different amino acids in native interleukin-2;
(ap) an interleukin-2 analog in which the amino acid at position 1 is removed and the amino acids at positions 125, 38, 42, 45, 80, 85, 86, and 92 are substituted with different amino acids in native interleukin-2;
(aq) an interleukin-2 analog in which the amino acid at position 1 is removed and the amino acids at positions 125, 38, 80, 81, 85, 86, and 92 are substituted with different amino acids in native interleukin-2;
(ar) an interleukin-2 analog in which the amino acid at position 1 is removed and the amino acids at positions 125, 42, 80, 81, 85, 86, and 92 are substituted with different amino acids in native interleukin-2;
(as) an interleukin-2 analog in which the amino acid at position 1 is removed and the amino acids at positions 125, 61, 80, 81, 85, 86, and 92 are substituted with different amino acids in native interleukin-2;
(at) an interleukin-2 analog in which the amino acid at position 1 is removed and the amino acids at positions 125, 69, 80, 81, 85, 86, and 92 are substituted with different amino acids in native interleukin-2;
(au) an interleukin-2 analog in which the amino acid at position 1 is removed and the amino acids at positions 125, 80, 81, 85, 86, 91, and 92 are substituted with different amino acids in native interleukin-2;
(av) an interleukin-2 analog in which the amino acid at position 1 is removed and the amino acids at positions 125, 18, 22, 80, 81, 85, 86, and 92 are substituted with different amino acids in native interleukin-2;
(aw) an interleukin-2 analog in which the amino acid at position 1 is removed and the amino acids at positions 125, 38, 42, 80, 81, 85, 86, and 92 are substituted with different amino acids in native interleukin-2;
(ax) an interleukin-2 analog in which the amino acid at position 1 is removed and the amino acids at positions 125, 38, 42, 45, 74, 80, 81, and 92 are substituted with different amino acids in native interleukin-2;
(ay) an interleukin-2 analog in which the amino acid at position 1 is removed and the amino acids at positions 125, 38, 68, 80, 81, 85, 86, and 92 are substituted with different amino acids in native interleukin-2;
(az) an interleukin-2 analog in which the amino acid at position 1 is removed and the amino acids at positions 125, 69, 74, 80, 81, 85, 86, and 92 are substituted with different amino acids in native interleukin-2;
(ba) an interleukin-2 analog in which the amino acid at position 1 is removed and the amino acids at positions 125, 80, 81, 84, 85, 86, 91, and 92 are substituted with different amino acids in native interleukin-2;
(bb) an interleukin-2 analog in which the amino acid at position 1 is removed and the amino acids at positions 125, 80, 81, 85, 86, 92, 94, and 96 are substituted with different amino acids in native interleukin-2;
(bc) an interleukin-2 analog in which the amino acid at position 1 is removed and the amino acids at positions 125, 18, 19, 22, 80, 81, 85, 86, and 92 are substituted with different amino acids in native interleukin-2;
(bd) an interleukin-2 analog in which the amino acid at position 1 is removed and the amino acids at positions 125, 18, 22, 38, 80, 81, 85, 86, and 92 are substituted with different amino acids in native interleukin-2;
(be) an interleukin-2 analog in which the amino acid at position 1 is removed and the amino acids at positions 125, 18, 22, 61, 80, 81, 85, 86, and 92 are substituted with different amino acids in native interleukin-2;
(bf) an interleukin-2 analog in which the amino acid at position 1 is removed and the amino acids at positions 125, 18, 22, 68, 80, 81, 85, 86, and 92 are substituted with different amino acids in native interleukin-2;
(bg) an interleukin-2 analog in which the amino acid at position 1 is removed and the amino acids at positions 125, 35, 38, 42, 80, 81, 85, 86, and 92 are substituted with different amino acids in native interleukin-2;
(bh) an interleukin-2 analog in which the amino acid at position 1 is removed and the amino acids at positions 125, 38, 42, 45, 80, 81, 85, 86, and 92 are substituted with different amino acids in native interleukin-2;
(bi) an interleukin-2 analog in which the amino acid at position 1 is removed and the amino acids at positions 125, 38, 42, 80, 81, 85, 86, 91, and 92 are substituted with different amino acids in native interleukin-2;
(bj) an interleukin-2 analog in which the amino acid at position 1 is removed and the amino acids at positions 125, 38, 42, 80, 81, 85, 86, 92, and 95 are substituted with different amino acids in native interleukin-2;
(bk) an interleukin-2 analog in which the amino acid at position 1 is removed and the amino acids at positions 125, 35, 38, 42, 74, 80, 81, 85, 86, and 92 are substituted with different amino acids in native interleukin-2;
(bl) an interleukin-2 analog in which the amino acid at position 1 is removed and the amino acids at positions 125, 38, 42, 43, 61, 80, 81, 85, 86, and 92 are substituted with different amino acids in native interleukin-2;
(bm) an interleukin-2 analog in which the amino acid at position 1 is removed and the amino acids at positions 125, 38, 42, 80, 81, 85, 86, 91, 92, and 95 are substituted with different amino acids in native interleukin-2;
(bn) an interleukin-2 analog in which the amino acid at position 1 is removed and the amino acids at positions 125, 35, 38, 42, 74, 80, 81, 82, 85, 86, and 92 are substituted with different amino acids in native interleukin-2;
(bo) an interleukin-2 analog in which the amino acid at position 1 is removed and the amino acids at positions 125, 80, 81, 86, and 92 are substituted with different amino acids in native interleukin-2; and
(bp) an interleukin-2 analog in which the amino acid at position 1 is removed and the amino acids at positions 125, 80, 81, 85, and 86 are substituted with different amino acids in native interleukin-2.

In particular, the amino acid substitutions included in the interleukin-2 analog may be any one or more selected from the group consisting of the following amino acid substitutions:
(a) a substitution in which the amino acid at position 12 is substituted with valine or phenylalanine;
(b) a substitution in which the amino acid at position 18 is substituted with arginine;
(c) a substitution in which the amino acid at position 19 is substituted with tyrosine, valine, phenylalanine, or arginine;
(d) a substitution in which the amino acid at position 20 is substituted with valine or phenylalanine;
(e) a substitution in which the amino acid at position 22 is substituted with glutamic acid;
(f) a substitution in which the amino acid at position 32 is substituted with cysteine;
(g) a substitution in which the amino acid at position 35 is substituted with cysteine or glutamic acid;
(h) a substitution in which the amino acid at position 38 is substituted with alanine or aspartic acid;
(i) a substitution in which the amino acid at position 42 is substituted with lysine, alanine, or tryptophan;
(j) a substitution in which the amino acid at position 43 is substituted with cysteine, glutamic acid, or glutamine;
(k) a substitution in which the amino acid at position 45 is substituted with alanine;
(l) a substitution in which the amino acid at position 48 is substituted with cysteine;
(m) a substitution in which the amino acid at position 49 is substituted with cysteine;
(n) a substitution in which the amino acid at position 61 is substituted with glutamine, arginine, or aspartic acid;
(o) a substitution in which the amino acid at position 68 is substituted with aspartic acid or glutamine;
(p) a substitution in which the amino acid at position 69 is substituted with glycine;
(q) a substitution in which the amino acid at position 74 is substituted with histidine or alanine;
(r) a substitution in which the amino acid at position 76 is substituted with cysteine;
(s) a substitution in which the amino acid at position 80 is substituted with phenylalanine, tyrosine, valine, aspartic acid, or tryptophan;
(t) a substitution in which the amino acid at position 81 is substituted with aspartic acid, glutamic acid, or asparagine;
(u) a substitution in which the amino acid at position 82 is substituted with glycine or valine;
(v) a substitution in which the amino acid at position 84 is substituted with glutamic acid, valine, or phenylalanine;
(w) a substitution in which the amino acid at position 85 is substituted with valine, alanine, glycine, tryptophan, tyrosine, threonine, isoleucine, glutamic acid, or phenylalanine;
(x) a substitution in which the amino acid at position 86 is substituted with valine, alanine, glycine, or leucine;
(y) a substitution in which the amino acid at position 87 is substituted with cysteine;
(z) a substitution in which the amino acid at position 88 is substituted with glutamine, valine, or phenylalanine;
(aa) a substitution in which the amino acid at position 89 is substituted with phenylalanine;
(ab) a substitution in which the amino acid at position 91 is substituted with threonine, phenylalanine, or glutamic acid;
(ac) a substitution in which the amino acid at position 92 is substituted with phenylalanine, leucine, tyrosine, or tryptophan;
(ad) a substitution in which the amino acid at position 94 is substituted with phenyl alanine or valine;
(ae) a substitution in which the amino acid at position 95 is substituted with aspartic acid;
(af) a substitution in which the amino acid at position 96 is substituted with phenylalanine, valine, or isoleucine;
(ag) a substitution in which the amino acid at position 125 is substituted with serine; and
(ah) a substitution in which the amino acid at position 126 is substituted with threonine.

In still another embodiment, the interleukin-2 analog may be any one selected from the following interleukin-2 analogs:
(a) an interleukin-2 analog in which the amino acid at position 1 is removed and the amino acids at positions 125, 38, 42, 80, 81 and 92 are substituted with different amino acids in native interleukin-2 analog;
(b) an interleukin-2 analog in which the amino acid at position 1 is removed and the amino acids at positions 125, 80, 81, 85, 86, and 92 are substituted with different amino acids in native interleukin-2 analog;
(c) an interleukin-2 analog in which the amino acid at position 1 is removed and the amino acids at positions 125, 18, 22, 80, 81, 85, 86, and 92 are substituted with different amino acids in native interleukin-2 analog;
(d) an interleukin-2 analog in which the amino acid at position 1 is removed and the amino acids at positions 125, 38, 42, 80, 81, 85, 86, and 92 are substituted with different amino acids in native interleukin-2 analog;
(e) an interleukin-2 analog in which the amino acid at position 1 is removed and the amino acids at positions 125, 18, 22, 61, 80, 81, 85, 86, and 92 are substituted with different amino acids in native interleukin-2 analog; and
(f) an interleukin-2 analog in which the amino acid at position 1 is removed and the amino acids at positions 125, 18, 22, 68, 80, 81, 85, 86, and 92 are substituted with different amino acids in native interleukin-2 analog.

In particular, the amino acid substitutions included in the interleukin-2 analog may be any one or more selected from the group consisting of the following amino acid substitutions:
(a) a substitution in which the amino acid at position 18 is substituted with arginine;
(b) a substitution in which the amino acid at position 22 is substituted with glutamic acid;
(c) a substitution in which the amino acid at position 38 is substituted with alanine;
(d) a substitution in which the amino acid at position 42 is substituted with lysine;
(e) a substitution in which the amino acid at position 61 is substituted with aspartic acid;
(f) a substitution in which the amino acid at position 68 is substituted with aspartic acid;
(g) a substitution in which the amino acid at position 80 is substituted with phenylalanine;
(h) a substitution in which the amino acid at position 81 is substituted with glutamic acid;
(i) a substitution in which the amino acid at position 85 is substituted with valine;
(j) a substitution in which the amino acid at position 86 is substituted with valine;
(k) a substitution in which the amino acid at position 92 is substituted with phenylalanine; and
(l) a substitution in which the amino acid at position 125 is substituted with serine.

As used herein, the term “corresponding to” refers to an amino acid residue at a position listed in a peptide, or an amino acid residue which is similar, identical, or homologous to a residue listed in a peptide. Confirmation of the amino acid at the corresponding position may be determining a specific amino acid in a sequence that refers to a specific sequence.

For example, each amino acid residue in the amino acid sequence can be numbered by aligning any amino acid sequence with SEQ ID NO: 1, and based on the same, referring to the numerical position of the amino acid residue corresponding to the amino acid residue of SEQ ID NO: 1.

As such an alignment, for example, the Needleman-Wunsch algorithm (Needleman and Wunsch, 1970, J. Mol. Biol. 48:443-453), the Needle program of the EMBOSS package (EMBOSS: The European Molecular Biology Open Software Suite, Rice et al., 2000), (Trends Genet. 16:276-277), etc. may be used, but the available programs are not limited thereto, and a sequence alignment program known in the art, a pairwise sequence comparison algorithm, etc. may be used appropriately.

In the present invention, even if expressed as a specific position of an amino acid in a peptide, such expression may refer to a corresponding position in a reference sequence.

In another embodiment, the interleukin-2 analog may include, essentially consist of, or consist of an amino acid sequence which is selected from the group consisting of SEQ ID NOS: 3 to 106, but is not limited thereto.

Additionally, even if the interleukin-2 analog is expressed as “an interleukin-2 analog consisting of a particular SEQ ID NO” in the present invention, it does not exclude a mutation that may occur by the addition of a meaningless sequence upstream or downstream of the amino acid sequence of the corresponding SEQ ID NO, or a mutation that may occur naturally, or a silent mutation thereof, as long as the interleukin-2 analog has an activity identical or equivalent to the interleukin-2 analog consisting of the amino acid sequence of the corresponding SEQ ID NO, and even if the sequence addition or mutation is present, the interleukin-2 analog apparently belongs to the scope of the present invention.

The interleukin-2 analog of the present invention may include an amino acid sequence which has a homology or identity of 40%, 45%, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% or higher to the amino acid sequences of SEQ ID NOS: 3 to 106, but is not limited thereto.

In a specific embodiment, the interleukin-2 analog of the present invention may include, essentially consist of, or consist of an amino acid sequence which is selected from the group consisting of SEQ ID NOS: 21, 41, 52, 86, 104, and 105, but is not limited thereto.

As used herein, the terms “homology” and “identity” refer to a degree of relatedness between two given amino acid sequences or nucleotide sequences and may be expressed as a percentage.

Sequence homology or identity of a conserved polynucleotide or polypeptide may be determined by a standard alignment algorithm, and default gap penalties established by a program to be used may be used in combination. Substantially, homologous or identical sequences may generally hybridize with all or part of the sequences under moderately or highly stringent conditions. It is apparent that hybridization also includes hybridization of a polynucleotide with a polynucleotide, which includes a general codon or a codon where codon degeneracy is considered.

The terms homology and identity can frequently be used interchangeably.

Whether any two nucleotide or peptide sequences have homology, similarity, or identity may be determined by, for example, a known computer algorithm (e.g., the “FASTA” program) using default parameters as in Pearson et al. (1988) Proc. Natl. Acad. Sci. USA 85:2444). Alternatively, they may be determined using the Needleman-Wunsch algorithm (Needleman and Wunsch, 1970, J. Mol. Biol. 48:443-453) as performed in the Needleman program of the EMBOSS package (EMBOSS: The European Molecular Biology Open Software Suite, Rice et al., 2000, Trends Genet. 16:276-277) (version 5.0.0 or later) (including GCG program package (Devereux, J. et al., Nucleic Acids Research 12:387 (1984)), BLASTP, BLASTN, FASTA (Atschul, S. F. et al., J Molec Biol 215:403 (1990); Guide to Huge Computers, Martin J. Bishop, ed., Academic Press, San Diego, 1994, and CARILLO et al. (1988) SIAM J Applied Math 48:1073). For example, homology, similarity, or identity may be determined using BLAST or ClustalW of the National Center for Biotechnology Information.

Homology, similarity, or identity of nucleotide or peptide sequences may be determined by comparing sequence information using the GAP computer program (e.g., Needleman et al. (1970), J Mol Biol 48:443) as disclosed in Smith and Waterman, Adv. Appl. Math (1981) 2:482. Briefly, the GAP program defines homology, similarity, or identity as the number of similar aligned symbols (i.e., nucleotides or amino acids), divided by the total number of symbols in the shorter of the two sequences. The default parameters for the GAP program may include: (1) a unary comparison matrix (containing a value of 1 for identities and 0 for non-identities) and the weighted comparison matrix (or EDNAFULL (EMBOSS version of NCBI NUC4.4) substitution matrix) of Gribskov et al. (1986) Nucl. Acids Res. 14:6745, as disclosed by Schwartz and Dayhoff, eds., Atlas Of Protein Sequence And Structure, National Biomedical Research Foundation, pp. 353-358 (1979); (2) a penalty of 3.0 for each gap and an additional 0.10 penalty for each symbol in each gap (or gap open penalty 10, gap extension penalty 0.5); and (3) no penalty for end gaps. Therefore, the terms “homology” and “identity”, as used herein, represent relatedness between sequences.

The above may be applied to other embodiments or other aspects of the present invention, but is not limited thereto.

The interleukin-2 analog of the present invention may be used as a novel interleukin-2 substitute that alters its in vitro activity by weakening or increasing the binding affinity of the interleukin-2 analog for interleukin-2 alpha and/or beta receptors. In particular, the interleukin-2 analog of the present invention can be used as an effective therapeutic agent due to its activities for the two types of receptors because it not only has an increased binding affinity for beta receptors but also has altered (i.e., increased or decreased) binding affinity for alpha receptors.

In the present invention, such modification for preparing analogs of interleukin-2 includes all of the modifications using L-type or D-type amino acids and/or non-natural amino acids; and/or a modification of native sequence, for example, a modification of a side chain functional group, an intramolecular covalent bonding (e.g., a ring formation between side chains), methylation, acylation, ubiquitination, phosphorylation, aminohexanation, biotinylation, etc.

Additionally, the modification includes all of those where one or more amino acids are added to the amino and/or carboxy terminus of native interleukin-2.

As the amino acids to be substituted or added, not only the 20 amino acids commonly observed in human proteins, but also atypical amino acids or those which are not naturally occurring can be used. Commercial sources of atypical amino acids include Sigma-Aldrich, ChemPep Inc., and Genzyme Pharmaceuticals. The peptides including these amino acids and typical peptide sequences may be synthesized and purchased from commercial suppliers for peptide synthesis, e.g., American Peptide Company, Bachem (USA), or Anygen (Korea).

Amino acid derivatives may be obtained in the same manner, and as one such example, 4-imidazoacetic acid, etc. may be used.

Additionally, the interleukin-2 analog according to the present invention may be in a modified form where the N-terminus and/or C-terminus, etc. of the interleukin-2 is chemically modified or protected by organic groups, or amino acids may be added to the terminus of the peptide, etc. for its protection from proteases in vivo while increasing its stability.

In particular, in the case of a chemically synthesized peptide, its N- and C-termini are electrically charged, and thus the N-terminus of the peptide may be acetylated and/or C-terminus of the peptide may be amidated to remove the charge, but the peptide is not particularly limited thereto.

The interleukin-2 analog of the present invention may be synthesized by a solid-phase synthesis method, and may also be produced by a recombinant method, or may be prepared commercially, but is not limited thereto.

Further, the interleukin-2 analog of the present invention may be synthesized by a method well-known in the art, according to its length, e.g., by an automatic peptide synthesizer, and may also be produced by genetic engineering technology.

Specifically, the interleukin-2 analog of the present invention may be prepared by a standard synthesis method, a recombinant expression system, or any other method known in the art. Accordingly, the interleukin-2 analog of the present invention may be synthesized by many methods including, for example, the following methods:
(a) a method of synthesizing a peptide by a solid-phase or liquid-phase method stepwise or by fragment assembly, followed by isolation and purification of the final peptide product;
(b) a method of expressing a nucleic acid construct encoding a peptide in a host cell and recovering the expression product from the host cell culture; or
(c) a method of performing an in vitro cell-free expression of a nucleic acid construct encoding a peptide and recovering the expression product therefrom; or
a method of obtaining peptide fragments by any combination of the methods (a), (b), and (c), obtaining a peptide by linking the peptide fragments, and then recovering the peptide.

In the present invention, the binding affinity of any interleukin-2 analog (or a long-acting conjugate including the same) for native interleukin-2 receptors can be measured using surface plasmon resonance (SPR), which is a method for measuring the affinity for the receptors.

Specifically, in the SPR analysis, a method, using the protein-ligand binding principle, in which the interleukin-2 receptor is immobilized to the sensor chip and the interleukin-2 analog diluted in the experimental buffer using a serial dilution method is flowed to induce binding to the immobilized receptor, and then, only the experimental buffer was flowed at the same flow rate to induce the dissociation of the interleukin-2 analog with the receptor, thereby measuring the binding affinity; or a method in which an antibody for a human immunoglobulin Fc region is immobilized to a sensor chip, then the interleukin-2 receptor to which the Fc region is bound is immobilized, and the interleukin-2 analog is flowed to measure binding affinity may be used, but the method is not limited thereto.

More specifically, biotin-labeled human interleukin-2 receptors were immobilized to a streptavidin biosensor chip, and the long-acting conjugate of the interleukin-2 analog diluted to HBS-P+ buffer using a two-fold serial dilution method was flowed at a flow rate of 20 μL/min for 3 minutes, and only the HBS-P+ buffer was flowed at the same flow rate to induce the dissociation between the interleukin-2 receptors and the long-acting conjugate of the interleukin-2 analog, and the obtained association constant and dissociation constant were used to measure the binding affinity according to the 1:1 binding fitting model using the Biaevaluation program, but is not limited thereto.

More specifically, the interleukin-2 analog of the present invention may have a reduced or increased binding affinity for interleukin-2 alpha receptors compared to the native interleukin-2 or interleukin-2 analog (aldesleukin; or interleukin-2 analog 1).

Specifically, the interleukin-2 analog of the present invention may have binding affinity for interleukin-2 alpha receptors of about 0.001-fold or greater, about 0.005-fold or greater, about 0.01-fold or greater, about 0.05-fold or greater, about 0.1-fold or greater, about 0.3-fold or greater, about 0.5-fold or greater, about 0.7-fold or greater, about 0.9-fold or greater, about 1.1-fold or greater, about 1.3-fold or greater, about 1.5-fold or greater, or about 1.7-fold or greater compared to the binding affinity of native interleukin-2 or aldesleukin for interleukin-2 alpha receptors, but the numerical value of the binding affinity is not limited, and the value will belong to the scope of the present invention as long as there is a change in the binding affinity compared to that of native interleukin-2 or aldesleukin.

Alternatively, based on the binding affinity of aldesleukin for interleukin-2 alpha receptors (set at 100%), the interleukin-2 analog of the present invention may have no binding affinity for interleukin-2 alpha receptors or have binding affinity for interleukin-2 alpha receptors of about 1% or greater, about 5% or greater, about 7% or greater, about 10% or greater, about 15% or greater, about 20% or greater, about 30% or greater, about 50% or greater, about 70% or greater, about 90% or greater, about 100% or greater, about 150% or greater, or about 200% or greater, but the numerical value of the binding affinity is not limited, and the value will belong to the scope of the present invention as long as there is a change in the binding affinity compared to that of native interleukin-2 or aldesleukin.

Additionally, the interleukin-2 analog of the present invention may specifically have binding affinity for interleukin-2 beta receptors of about 0.1-fold or greater, about 0.3-fold or greater, about 0.5-fold or greater, about 0.7-fold or greater, about 1.0-fold or greater, about 10-fold or greater, about 20-fold or greater, about 30-fold or greater, about 40-fold or greater, about 50-fold or greater, about 60-fold or greater, about 70-fold or greater, about 80-fold or greater, about 90-fold or greater, or about 100-fold or greater compared to the binding affinity of native interleukin-2 or aldesleukin for interleukin-2 beta receptors, but the numerical value of the binding affinity is not limited, and the value will belong to the scope of the present invention as long as there is a change or increase in the binding affinity compared to that of native interleukin-2 or aldesleukin.

Alternatively, based on the binding affinity of aldesleukin for interleukin-2 beta receptors (set at 100%), the interleukin-2 analog of the present invention may have binding affinity for interleukin-2 beta receptors of about 5% or greater, about 9% or greater, about 10% or greater, about 20% or greater, about 30% or greater, about 50% or greater, about 100% or greater, about 200% or greater, about 500% or greater, about 700% or greater, about 1,000% or greater, about 1,500% or greater, about 3,000% or greater, about 5,000% or greater, about 7,000% or greater, about 10,000% or greater, about 12,000% or greater, about 15,000% or greater, about 20,000% or greater, or about 25,000%, but the numerical value of the binding affinity is not limited, and the value will belong to the scope of the present invention as long as there is an increase in the binding affinity compared to that of native interleukin-2 or aldesleukin.

As used herein, the term “about” refers to a range which includes all of ±0.5, ±0.4, ±0.3, ±0.2, ±0.1, etc. and includes all of the values that are equivalent or similar to those following the values, but the range is not limited thereto.

The interleukin-2 analog of the present invention is characterized in that it has altered binding affinity for interleukin-2 alpha receptors and increased binding affinity for interleukin-2 beta receptors compared to native interleukin-2 or aldesleukin.

In a specific embodiment of the present invention, for the preparation of the interleukin-2 analog of the present invention, an interleukin-2 analog, into which a modification was introduced based on native interleukin-2 (SEQ ID NO: 1), was prepared. The interleukin-2 analog prepared in the present invention may be one which includes any one amino acid sequence among SEQ ID NOS: 3 to 106, or may be one which is encoded by any one nucleotide sequence among SEQ ID NOS: 108 to 211.

The nucleic acid encoding the interleukin-2 analog of the present invention may be one which is modified so that a modification (deletion, substitution, and/or addition of an amino acid) can be introduced into an amino acid at a particular position in a nucleotide sequence encoding the native interleukin-2 of SEQ ID NO: 1, and specifically, the interleukin-2 analog of the present invention may include a nucleotide sequence encoding any one amino acid sequence among SEQ ID NOS: 3 to 106. For example, the nucleic acid of the present invention may have or include a nucleotide sequence of any one among SEQ ID NOS: 108 to 211.

The nucleotide sequence of the present invention may be modified variously in the coding region within a range not altering the amino acid sequence of the interleukin-2 analog of the present invention, considering codon degeneracy or the codons preferred in the organism where the nucleic acid of the present invention is to be expressed. Specifically, the nucleic acid of the present invention may have or include a nucleotide sequence which has a homology or identity of 70% or higher, 75% or higher, 80% or higher, 85% or higher, 90% or higher, 95% or higher, 96% or higher, 97% or higher, 98% or higher, and less than 100% to any one of the sequences of SEQ ID NOS: 108 to 211; or may consist of or essentially consist of a nucleotide sequence which has a homology or identity of 70% or higher, 75% or higher, 80% or higher, 85% or higher, 90% or higher, 95% or higher, 96% or higher, 97% or higher, 98% or higher, and less than 100% to any one of the sequences of SEQ ID NOS: 108 to 211, but the nucleic acid is not limited thereto.

Additionally, the nucleic acid of the present invention can include, without limitation, a probe which can be prepared from a known gene sequence (e.g., a sequence that can hybridize with a sequence complementary to all or part of the nucleic acid sequence of the present invention under stringent conditions). The “stringent conditions” refer to conditions that enable specific hybridization between polynucleotides. Such conditions are described in detail in the literature (see J. Sambrook et al., Molecular Cloning, A Laboratory Manual, 2nd Edition, Cold Spring Harbor Laboratory press, Cold Spring Harbor, New York, 1989; F. M. Ausubel et al., Current Protocols in Molecular Biology, John Wiley & Sons, Inc., New York, 9.50-9.51, 11.7-11.8).

Hybridization requires that two nucleic acids have complementary sequences, although mismatches between bases may be possible depending on hybridization stringency. The term “complementary” is used to describe the relationship between nucleotide bases that can hybridize to each other. For example, with respect to DNA, adenine is complementary to thymine, and cytosine is complementary to guanine. Accordingly, the nucleic acid of the present invention can include isolated nucleic acid fragments complementary to the entire sequence as well as substantially similar nucleic acid sequences.

The appropriate stringency for hybridizing polynucleotides depends on the length of the polynucleotides and the degree of complementarity, and variables are well known in the art (e.g., Sambrook et al., supra).

The homology and identity are as described above.

Further, the interleukin-2 analog of the present invention may have an increased in vivo half-life, compared to native interleukin-2 or aldesleukin, but is not particularly limited thereto. For example, the interleukin-2 analog of the present invention may be in the form of a long-acting conjugate having an increased half-life by linking a biocompatible material (e.g., an immunoglobulin Fc region) for increasing the half-life to the interleukin-2 analog directly or via a linker, but is not limited thereto.

The long-acting conjugate according to the present invention not only includes an interleukin-2 analog with increased binding affinity for the interleukin-2 beta receptors, but also binds to an immunoglobulin Fc region as a representative carrier for increasing its half-life, thereby increasing the half-life of the interleukin-2 analog, increasing blood exposure, and increasing in vivo immune response, and thus, growth inhibition and reduction of cancer cells can be effectively achieved.

Another aspect for implementing the present invention provides a long-acting conjugate of an interleukin-2 analog.

In the present invention, the long-acting conjugate of an interleukin-2 analog may be in the form, in which a biocompatible material capable of increasing the in vivo half-life thereof is linked to the interleukin-2 analog. In the present invention, the biocompatible material may be used interchangeably with a carrier.

In the present invention, the long-acting conjugate may exhibit increased duration of the efficacy as compared with the interleukin-2 analog to which the carrier is not linked, and in the present invention, such a conjugate is referred to as a “long-acting conjugate” or “conjugate”

Meanwhile, the conjugate may be one which does not occur naturally.

In one specific embodiment of the present invention, the long-acting conjugate may be a long-acting conjugate represented by Chemical Formula 1 below:

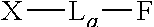

wherein, X is an interleukin-2 analog having an increased binding affinity for interleukin-2 beta receptors compared to aldesleukin;
L is a polyethylene glycol linker;
a is 0 or a natural number, with the proviso that when a is 2 or more, each L is independent of each other;
F is an immunoglobulin Fc region in the form of a dimer; and
- indicates a covalent linkage.

More specifically, X and L, and L and F may be linked to each other via a covalent bond. In particular, the conjugate is a conjugate, in which X, L, and F are linked via covalent bonds, respectively, as in the order of Chemical Formula 1.

Further, F may be directly linked to X (i.e., a is 0 in Chemical Formula 1), or may be linked through a linker (L).

In the present invention, the interleukin-2 analog may correspond to an element of one moiety constituting the conjugate. Specifically, it corresponds to X in Chemical Formula 1 above, and the interleukin-2 analog is the same as described above.

In the conjugate, F is a material capable of increasing the half-life of X, i.e., the interleukin-2 analog, and it corresponds to an element of one moiety constituting the conjugate of the present invention.

F and X may be linked to each other by a covalent chemical bond, and F and X may be linked to each other via L by a covalent chemical bond.

Specifically, F is an immunoglobulin Fc region, and the immunoglobulin Fc region may be an IgG Fc region or a non-glycosylated IgG4 Fc region, but is not particularly limited thereto.

As a specific embodiment of the present invention, F (immunoglobulin Fc region) is a dimer consisting of two polypeptide chains, and may have a structure in which one end of L is linked only to one of the two polypeptide chains, but not limited thereto.

One or more amino acid side chains within the peptide of the present invention may be attached to the biocompatible material in order to increase in vivo solubility and/or half-life, and/or to increase bioavailability thereof. These modifications may reduce the clearance of therapeutic proteins and peptides.

The above-described biocompatible material may be soluble (amphipathic or hydrophilic) and/or non-toxic and/or pharmaceutically acceptable.

Further, F may be directly linked to X (i.e., a is 0 in Chemical Formula 1), or may be linked through a polyethylene glycol linker (L).

In one specific embodiment, the long-acting conjugate of the present invention may be one in which an interleukin-2 analog and an immunoglobulin Fc region are linked, but is not limited thereto.

In the present invention, the “immunoglobulin Fc region” refers to a region including a heavy chain constant region 2(CH2) and/or a heavy chain constant region 3(CH3), excluding heavy chain and light chain variable regions of the immunoglobulin. The immunoglobulin Fc region may be an element constituting the moiety of the conjugate of the present invention. Specifically, the immunoglobulin Fc region corresponds to F in Chemical Formula 1 above.

In the present specification, the Fc region encompasses not only a native sequence obtained from papain digestion of an immunoglobulin, but also derivatives thereof, for example, variants, in which one or more amino acid residues in the native sequence are converted by deletion, insertion, non-conservative or conservative substitution, or a combination thereof, and thus become different from the native sequence, etc.

The above derivatives, substituents, and variants are required to retain FcRn-binding ability. In the present invention, F may be a human immunoglobulin region, but is not limited thereto. In the present specification, “biocompatible material” or “carrier” may refer to the Fc region.

F (e.g., an immunoglobulin Fc region) has a structure, in which two polypeptide chains are linked to each other via a disulfide bond, only via a nitrogen atom of one chain of the two chains, but is not limited thereto. The linkage via the nitrogen atom may be linked via reductive amination to an epsilon amino group or the N-terminal amino group of lysine.

The reductive amination reaction refers to a reaction in which an amine group or an amino group of a reactant reacts with an aldehyde (i.e., a functional group capable of reductive amination) of another reactant to produce an amine, and then forms an amine bond by a reduction reaction. It is an organic synthesis reaction well known in the art.

In a specific embodiment of the long-acting conjugate of the present invention, the immunoglobulin Fc region is linked to the linker via a nitrogen atom at the N-terminus thereof.

Such an immunoglobulin Fc region may include a hinge region in the heavy chain constant region, but is not limited thereto.

In the present invention, the immunoglobulin Fc region may include a specific hinge sequence at the N-terminus.

As used herein, the term “hinge sequence” refers to a region that is located in the heavy chain and forms a dimer of the immunoglobulin Fc region through an inter-disulfide bond.

In the present invention, the hinge sequence may be altered to have only one cysteine residue by deleting a part in a hinge sequence having the following amino acid sequence, but is not limited thereto:

(SEQ ID NO: 418)
Glu-Ser-Lys-Tyr-Gly-Pro-Pro-Cys-Pro-Ser-Cys-Pro.

The hinge sequence may include only one cysteine residue by deleting a cysteine residue at position 8 or 11 in the hinge sequence of SEQ ID NO: 418. The hinge sequence of the present invention may include only one cysteine residue and may consist of 3 to 12 amino acids, but is not limited thereto. More specifically, the hinge sequence of the present invention may have the following sequence:

(SEQ ID NO: 419)
Glu-Ser-Lys-Tyr-Gly-Pro-Pro-Pro-Ser-Cys-Pro,
(SEQ ID NO: 420)
Glu-Ser-Lys-Tyr-Gly-Pro-Pro-Cys-Pro-Ser-Pro,
(SEQ ID NO: 421)
Glu-Ser-Lys-Tyr-Gly-Pro-Pro-Cys-Pro-Ser,
(SEQ ID NO: 422)
Glu-Ser-Lys-Tyr-Gly-Pro-Pro-Cys-Pro-Pro,
(SEQ ID NO: 423)
Lys-Tyr-Gly-Pro-Pro-Cys-Pro-Ser,
(SEQ ID NO: 424)
Glu-Ser-Lys-Tyr-Gly-Pro-Pro-Cys,
(SEQ ID NO: 425)
Glu-Lys-Tyr-Gly-Pro-Pro-Cys,
(SEQ ID NO: 426)
Glu-Ser-Pro-Ser-Cys-Pro,
(SEQ ID NO: 427)
Glu-Pro-Ser-Cys-Pro,
(SEQ ID NO: 428)
Pro-Ser-Cys-Pro,
(SEQ ID NO: 429)
Glu-Ser-Lys-Tyr-Gly-Pro-Pro-Ser-Cys-Pro,
(SEQ ID NO: 430)
Lys-Tyr-Gly-Pro-Pro-Pro-Ser-Cys-Pro,
(SEQ ID NO: 431)
Glu-Ser-Lys-Tyr-Gly-Pro-Ser-Cys-Pro,
(SEQ ID NO: 432)
Glu-Ser-Lys-Tyr-Gly-Pro-Pro-Cys,
(SEQ ID NO: 433)
Lys-Tyr-Gly-Pro-Pro-Cys-Pro,
(SEQ ID NO: 434)
Glu-Ser-Lys-Pro-Ser-Cys-Pro,
(SEQ ID NO: 435)
Glu-Ser-Pro-Ser-Cys-Pro,
(SEQ ID NO: 436)
Glu-Pro-Ser-Cys,
(SEQ ID NO: 437)
Ser-Cys-Pro.

More specifically, the hinge sequence may include an amino acid sequence of SEQ ID NO: 428 (Pro-Ser-Cys-Pro) or SEQ ID NO: 437 (Ser-Cys-Pro), but is not limited thereto.

In a more specific embodiment of the long-acting conjugate of the present invention, in the conjugate, the N-terminus of the immunoglobulin Fc region is proline. In this conjugate, the Fc region is linked to the linker via a nitrogen atom of the proline.

In an embodiment of the long-acting conjugate of the present invention, the immunoglobulin Fc region may have a dimer form in which two chains of the immunoglobulin Fc region form a homodimer or a heterodimer due to the presence of a hinge sequence. The conjugate of Chemical Formula 1 of the present invention may be in the form in which one end of the linker is linked to one chain of the immunoglobulin Fc region of the dimer, but is not limited thereto.

As used herein, the term “N-terminus” refers to the amino terminus of a protein or polypeptide, and includes the extreme end of the amino terminus or includes up to 1, 2, 3, 4, 5, 6, 7, 8, 9, or 10 or more amino acids from the extreme end. The immunoglobulin Fc region of the present invention may include a hinge sequence at the N-terminus, but is not limited thereto.

Further, the immunoglobulin Fc region of the present invention may be an extended Fc region including a part or the entirety of the heavy chain constant region 1 (CH1) and/or light chain constant region 1 (CL1), excluding the heavy chain and light chain variable regions of an immunoglobulin, as long as it has an effect substantially equivalent or improved, as compared to its native form. Further, the immunoglobulin Fc region may be a region in which a part of a significantly long amino acid sequence corresponding to CH₂ and/or CH₃ is removed.

For example, the immunoglobulin Fc region of the present invention may be 1) CH1 domain, CH2 domain, CH3 domain, and CH4 domain, 2) CH1 domain and CH2 domain, 3) CH1 domain and CH3 domain, 4) CH2 domain and CH3 domain, 5) a combination between one or two or more domains among CH1 domain, CH2 domain, CH3 domain and CH4 domain, and an immunoglobulin hinge region (or a part of the hinge region), and 6) a dimer between each domain of the heavy chain constant region and the light chain constant region, but is not limited thereto.

In the present invention, the immunoglobulin Fc region may be a dimer or multimer consisting of single-chain immunoglobulins consisting of domains of the same origin, but is not limited thereto.

Further, in an embodiment of the long-acting conjugate of the present invention, the immunoglobulin Fc region F is a dimer consisting of two polypeptide chains, wherein the dimeric Fc region F and X may be covalently linked to each other via one identical linker L containing ethylene glycol repeating units. In a specific embodiment, X is covalently linked only to one polypeptide chain of the two polypeptide chains of the dimeric Fc region F via the linker L. In a more specific embodiment, only one X molecule is covalently linked via L to one polypeptide chain, to which X is linked, of the two polypeptide chains of the dimeric Fc region F. In the most specific embodiment, F is a homodimer.

In another specific embodiment, the immunoglobulin Fc region F is a dimer consisting of two polypeptide chains, and one end of L is linked only to one polypeptide chain of the two polypeptide chains, but is not limited thereto.

In another embodiment of the long-acting conjugate of the present invention, it is also possible for two molecules of X to bind symmetrically to one Fc region in a dimeric form. In particular, the immunoglobulin Fc region and X may be linked to each other via the linker (L), but are not limited to the above-described examples.

Further, the immunoglobulin Fc region of the present invention includes the native amino acid sequence as well as sequence derivatives thereof. The amino acid sequence derivative means that one or more amino acid residues in the natural amino acid sequence have a different sequence due to deletion, insertion, non-conservative or conservative substitution, or a combination thereof.

For example, amino acid residues at positions 214 to 238, 297 to 299, 318 to 322, or 327 to 331 in IgG Fc, which are known to be important for linkage, may be used as the sites suitable for variation.

Further, various types of derivatives are possible, for example, those where the site capable of forming a disulfide bond is removed, those where several N-terminal amino acids are removed from native Fc, those where a methionine residue is added to the N-terminus of native Fc, etc. Further, complement binding sites, e.g., C1q binding sites, or antibody-dependent cell-mediated cytotoxicity (ADCC) sites may be removed to remove the effector function. The techniques for preparing the sequence derivatives of the immunoglobulin Fc region are disclosed in International Publication Nos. WO 97/34631, WO 96/32478, etc.

Amino acid exchanges in a protein or peptide that do not alter the entire activity of a molecule are well known in the art (H. Neurath, R. L. Hill, The Proteins, Academic Press, New York, 1979). The most common exchanges occur between amino acid residues of Ala/Ser, Val/Ile, Asp/Glu, Thr/Ser, Ala/Gly, Ala/Thr, Ser/Asn, Ala/Val, Ser/Gly, Thy/Phe, Ala/Pro, Lys/Arg, Asp/Asn, Leu/Ile, Leu/Val, Ala/Glu, Asp/Gly. In some cases, amino acids may be modified by phosphorylation, sulfation, acrylation, glycosylation, methylation, farnesylation, acetylation, amidation, etc.

The Fc derivatives described above may be those which exhibit the same biological activity as that of the Fc region of the present invention, and have increased structural stability of the Fc region against heat, pH, etc.

Further, such an Fc region may be obtained from a native type isolated from humans or animals such as cows, goats, pigs, mice, rabbits, hamsters, rats, guinea pigs, etc., or may be their recombinants or derivatives obtained from transformed animal cells or microorganisms. In particular, the method of obtaining from a native form is a method of isolating whole immunoglobulins from human or animal organisms and then treating them with a protease. When treated with papain, the native form is digested into Fab and Fc, and when treated with pepsin, it is cleaved into pF′ c and F(ab)₂. Fc or pF′ c may be isolated using size exclusion chromatography, etc. In a more specific embodiment, the Fc region may be a recombinant immunoglobulin Fc region, in which a human-derived Fc region is obtained from a microorganism.

In addition, the immunoglobulin Fc region may have natural glycans or increased or decreased glycans compared to the natural type, or be in a deglycosylated form. The increase, decrease, or removal of glycans of the immunoglobulin Fc may be achieved by any methods commonly used in the art such as a chemical method, an enzymatic method, and a genetic engineering method using a microorganism. In particular, the immunoglobulin Fc region obtained by removing glycans shows a significant decrease in binding affinity to a complement c1q and a decrease in or loss of antibody-dependent cytotoxicity or complement-dependent cytotoxicity, and thus unnecessary immune responses are not induced thereby in living organisms. Based thereon, a deglycosylated or aglycosylated immunoglobulin Fc region may be more suitable as a drug carrier in view of the objects of the present invention.

As used herein, the term “deglycosylation” refers to a Fc region from which glycan is removed using an enzyme and the term “aglycosylation” refers to a Fc region that is not glycosylated and produced in prokaryotes, more specifically E. coli.

Meanwhile, the immunoglobulin Fc region may be derived from humans or animals including cows, goats, pigs, mice, rabbits, hamsters, rats, guinea pigs, etc., and in a more specific embodiment, it may be derived from humans.

Further, the immunoglobulin Fc region may be an Fc region derived from IgG, IgA, IgD, IgE, IgM, or a combination or hybrid thereof. In a more specific embodiment, it may be derived from IgG or IgM, which are the most abundant proteins in human blood, and in an even more specific embodiment, it may be derived from IgG, which is known to enhance the half-lives of ligand binding proteins. In a more specific embodiment, the immunoglobulin Fc region may be an IgG4 Fc region, and in a most specific embodiment, the immunoglobulin Fc region may be an aglycosylated Fc region derived from a human IgG4, but is not limited thereto.

In addition, in a specific embodiment, the immunoglobulin Fc region may be a human IgG4 Fc fragment in the form of a homodimer in which two monomers are linked to each other via a disulfide bond (inter-chain) formed between cysteines that are amino acids located at position 3 of each monomer. In particular, each monomer of the homodimer has or may have two disulfide bonds (intra-chain), i.e., a disulfide bond formed between cysteines at positions 35 and 95 and a disulfide bond formed between cysteines at positions 141 and 199.

Each monomer may consist of 221 amino acids and the number of amino acids constituting the homodimer may be 442 in total, without being limited thereto. Specifically, the immunoglobulin Fc fragment may be in the form of a homodimer in which two monomers each having an amino acid sequence of SEQ ID NO: 438 (consisting of 221 amino acids) are linked to each other via a disulfide bond between cysteines at position 3 of each monomer, wherein the monomers of the homodimer each independently have an intra-chain disulfide bond formed between cysteines at positions 35 and 95 and an intra-chain disulfide bond formed between cysteines at positions 141 and 199, without being limited thereto.

F of Chemical Formula 1 may include a monomer of the amino acid sequence of SEQ ID NO: 438, and F may be a homodimer of the monomer of the amino acid sequence of SEQ ID NO: 438, but is not limited thereto.

In an embodiment, the immunoglobulin Fc region may be a homodimer including an amino acid sequence of SEQ ID NO: 439 (consisting of 442 amino acids), but is not limited thereto.

In a specific embodiment, the immunoglobulin Fc region and X may not be glycosylated, but are not limited thereto.

Meanwhile, as used herein, the term “combination” means that polypeptides encoding single-chain immunoglobulin Fc regions of the same origin are linked to a single-chain polypeptide of a different origin to form a dimer or multimer. In other words, a dimer or a multimer may be prepared from two or more fragments selected from the group consisting of Fc regions of IgG Fc, IgA Fc, IgM Fc, IgD Fc, and IgE.

As used herein, the term “hybrid” means that sequences corresponding to two or more immunoglobulin Fc regions of different origins are present in a single-chain immunoglobulin constant region. In the present invention, various hybrid forms are possible. In other words, the hybrid domain may be composed of one to four domains selected from the group consisting of CH1, CH2, CH3, and CH4 of IgG Fc, IgM Fc, IgA Fc, IgE Fc, and IgD Fc, and may include a hinge region.

Meanwhile, IgG may be divided into the IgG1, IgG2, IgG3, and IgG4 subclasses, and the present invention may include combinations or hybrids thereof, specifically the IgG2 and IgG4 subclasses, and most specifically the Fc fragment of IgG4, which rarely has effector functions such as complement-dependent cytotoxicity (CDC).

Further, the above-described conjugate may have increased duration of efficacy, as compared with the native interleukin-2 or aldesleukin, or as compared with X not modified with F, and such a conjugate may be not only in the above-described form but also in the form, in which it is encapsulated in biodegradable nanoparticles, but is not limited thereto.

In Chemical Formula 1, L is a polyethylene glycol linker.

As used herein, the term “polyethylene glycol linker” includes a biocompatible polymer in which two or more ethylene glycol repeating units are conjugated. The repeating units are linked to each other through any covalent bond, not a peptide bond. The polyethylene glycol linker may be an element constituting the moiety of the conjugate of the present invention, and corresponds to L in Chemical Formula 1. In the present specification, the linker may be used interchangeably with “non-peptidyl linker” or “non-peptidyl polymer”.

In La, a may be 1 or more, and when a is 2 or more, L may be independent of each other.

In a specific embodiment, the conjugate may be one in which F and X are covalently linked to each other via a non-peptidyl linker including a reactive group capable of binding to F (specifically an immunoglobulin Fc region) and X (specifically an interleukin-2 analog) at both ends.

Specifically, in the present invention, the non-peptidyl linker may include reactive groups at ends thereof to form a conjugate through a reaction with other components constituting the conjugate. When the non-peptidyl linker having reactive functional groups at both ends bind to X and F of Chemical Formula 1 via respective reactive groups to form the conjugate, the non-peptidyl linker or non-peptidyl polymer may be referred to as a non-peptidyl polymer linker moiety or a non-peptidyl linker moiety.

In a specific embodiment, L (polyethylene glycol linker) may be a linker containing ethylene glycol repeating units, for example, polyethylene glycol, but is not limited thereto. As used herein, the polyethylene glycol is a term including all of an ethylene glycol homopolymer, a PEG copolymer, or a monomethyl-substituted PEG polymer (mPEG), but is not particularly limited thereto. Further, derivatives thereof that are known in the art and derivatives that may be easily prepared by ordinary skill in the art are also included in the scope of the present invention.

The polyethylene glycol linker may include the ethylene glycol repeating units while including, at the ends thereof, functional groups used in the preparation of a conjugate before being formed into the conjugate. In the long-acting conjugate according to the present invention, X and F may be linked to each other though the functional groups, but it is not limited thereto. In the present invention, the non-peptidyl linker may include two or three or more functional groups, wherein the respective functional groups are the same as or different from each other, but is not limited thereto.

Specifically, the linker may be polyethylene glycol (PEG) represented by Chemical Formula 5 below, but is not limited thereto:

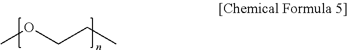

Here, n=10 to 2400, n=10 to 480, or n=50 to 250, but is not limited thereto.

In the long-acting conjugate, the PEG moiety may include a structure of —(CH₂CH₂O)ₙ— and an oxygen atom interposed between the linking element and —(CH₂CH₂O)ₙ—, but is not limited thereto.

In a specific embodiment, the ethylene glycol repeating unit may be represented by, for example, [OCH₂CH₂]ₙ, wherein the value of n is a natural number and may be determined such that an average molecular weight, for example, a number average molecular weight of the [OCH₂CH₂]ₙ site in the conjugate of an interleukin-2 analog is more than 0 kDa to about 100 kDa, but is not limited thereto. In another embodiment, the value of n is a natural number and may be determined such that an average molecular weight, for example, a number average molecular weight of the [OCH₂CH₂]ₙ site in the peptide conjugate may be about 1 kDa to about 100 kDa, about 1 kDa to about 80 kDa, about 1 kDa to about 50 kDa, about 1 kDa to about 30 kDa, about 1 kDa to about 25 kDa, about 1 kDa to about 20 kDa, about 1 kDa to about 15 kDa, about 1 kDa to about 13 kDa, about 1 kDa to about 11 kDa, about 1 kDa to about 10 kDa, about 1 kDa to about 8 kDa, about 1 kDa to about 5 kDa, about 1 kDa to about 3.4 kDa, about 3 kDa to about 30 kDa, about 3 kDa to about 27 kDa, about 3 kDa to about 25 kDa, about 3 kDa to about 22 kDa, about 3 kDa to about 20 kDa, about 3 kDa to about 18 kDa, about 3 kDa to about 16 kDa, about 3 kDa to about 15 kDa, about 3 kDa to about 13 kDa, about 3 kDa to about 11 kDa, about 3 kDa to about 10 kDa, about 3 kDa to about 8 kDa, about 3 kDa to about 5 kDa, about 3 kDa to about 3.4 kDa, about 8 kDa to about 30 kDa, about 8 kDa to about 27 kDa, about 8 kDa to about 25 kDa, about 8 kDa to about 22 kDa, about 8 kDa to about 20 kDa, about 8 kDa to about 18 kDa, about 8 kDa to about 16 kDa, about 8 kDa to about 15 kDa, about 8 kDa to about 13 kDa, about 8 kDa to about 11 kDa, about 8 kDa to about 10 kDa, about 9 kDa to about 15 kDa, about 9 kDa to about 14 kDa, about 9 kDa to about 13 kDa, about 9 kDa to about 12 kDa, about 9 kDa to about 11 kDa, about 9.5 kDa to about 10.5 kDa, or about 10 kDa, but is not limited thereto.

Further, in a specific embodiment, the conjugate may have a structure in which the interleukin-2 analog and the immunoglobulin Fc region (F) are covalently linked to each other via the linker (L) including ethylene glycol repeating units, but is not limited thereto.

In another specific embodiment, in the long-acting conjugate, L may be a linker including ethylene glycol repeating units, and F may be a dimeric immunoglobulin Fc region. More specifically, one X molecule is covalently linked to one Fc region of the dimeric immunoglobulin Fc region via the linker containing ethylene glycol repeating units, but is not limited thereto. In still another specific embodiment, one end of the linker containing ethylene glycol repeating units may be linked only to one Fc region chain of two Fc region chains of the dimeric immunoglobulin Fc region, but is not limited thereto.

The molecular weight of the polyethylene glycol linker that can be used in the present invention may be in the range of more than 0 kDa to 200 kDa, specifically in the range of about 1 kDa to 100 kDa, about 1 kDa to 50 kDa, about 1 kDa to 30 kDa, about 2 kDa to 30 kDa, about 1 kDa to 20 kDa, more specifically in the range of about 3.4 kDa to 10 kDa, and much more specifically about 3.4 kDa, but is not limited thereto.

In addition, as the non-peptidyl linker of the present invention conjugated to the polypeptide corresponding to F, not only one type of polymer but also a combination of different types of polymers may be used.

As used herein, the term “about” includes all of the ranges including ±0.5, ±0.4, ±0.3, ±0.2, ±0.1, etc., and includes all values in a range equal to or similar to the value following the term “about”, but is not limited thereto.

Specifically, the non-peptidyl linker may have reactive groups at both ends thereof in a state where F and X are not bound thereto, and may bind with F and X via the reactive groups.

In a specific embodiment, both ends of the linker may bind to a thiol group, an amino group, or a hydroxyl group of the immunoglobulin Fc region, and a thiol group, an amino group, an azide group, or a hydroxyl group of the interleukin-2 analog (X), but are not limited thereto.

Specifically, the linker may include, at both ends thereof, reactive groups capable of binding to the immunoglobulin Fc region and the interleukin-2 analog (X), respectively, specifically reactive groups capable of binding to a thiol group of cysteine; an amino group located at the N-terminus, lysine, arginine, glutamine, and/or histidine; and/or a hydroxyl group at the C-terminus of the immunoglobulin Fc region; and a thiol group of cysteine; an amino group of lysine, arginine, glutamine, and/or histidine; an azide group of azido-lysine; and/or a hydroxyl group of the interleukin-2 analog (X), but is not limited thereto.

More specifically, the reactive group of the linker may be one or more selected from the group consisting of an aldehyde group, a maleimide group, and a succinimide derivative, but is not limited thereto.

In the above, examples of the aldehyde group may include a propionaldehyde group, or a butyraldehyde group, but are not limited thereto.

In the above, examples of the succinimide derivative may include succinimidyl valerate, succinimidyl methylbutanoate, succinimidyl methylpropionate, succinimidyl butanoate, succinimidyl propionate, N-hydroxysuccinimide, hydroxy succinimidyl, succinimidyl carboxymethyl, or succinimidyl carbonate, but are not limited thereto.

The linker may be linked to the immunoglobulin Fc region F and the interleukin-2 analog X via the reactive groups to be converted into a linker moiety.

Further, a final product produced by reductive alkylation via aldehyde bonds is more stable than a linkage formed by an amide bond. The aldehyde reactive group selectively reacts with the N-terminus at low pH while forming a covalent bond with a lysine residue at high pH, e.g., at a pH of 9.0.

In addition, the reactive groups of both ends of the linker may be the same as or different from each other, for example, aldehyde groups may be provided at both ends, and a maleimide group may be provided at one end and an aldehyde group, a propionaldehyde group, or a butyraldehyde group may be provided at the other end. However, the reactive groups are not particularly limited as long as F, specifically the immunoglobulin Fc region, and X may be linked to the respective ends of the linker.

For example, the linker may include a maleimide group at one end and an aldehyde group, a propionaldehyde group, or a butyraldehyde group at the other end, as reactive groups.

When polyethylene glycol having hydroxyl reactive groups at both ends is used as a linker, the long-acting conjugate of an interleukin-2 analog of the present invention may be prepared by activating the hydroxyl groups to various reactive groups by known chemical reactions, or using commercially available polyethylene glycol having modified reactive groups.

In a specific embodiment, the polymer may be linked to a cysteine residue of X, more specifically a —SH group of cysteine, but is not limited thereto.

Specifically, the reactive group of the linker may be linked to the —SH group of the cysteine residue, and all of those described above will be applied to the reactive group. When maleimide-PEG-aldehyde is used, the maleimide group may be linked to the —SH group of X via a thioether bond, and the aldehyde group may be linked to the —NH₂ group of F, specifically the immunoglobulin Fc, via a reductive amination, but is not limited thereto.

In another specific embodiment, the linker may be linked to the lysine residue of X, more specifically the amino group of the lysine, but is not limited thereto.

In addition, in the conjugate, the reactive group of the linker may be linked to —NH₂ located at the N-terminus of the immunoglobulin Fc region, but is not limited thereto.

Further, in the conjugate, the interleukin-analog according to the present invention may be linked to the linker having reactive groups via the C-terminus, but this is merely an example.

As used herein, the term “C-terminus” refers to a carboxy terminus of a peptide, and with respect to the objects of the present invention, it refers to a site capable of binding with the linker. For example, the C-terminus may include all of an amino acid residue at the extreme end of the C-terminus and amino acid residues near the C-terminus, and specifically include the 1^{st} to 20^{th} amino acid residues from the extreme end, but is not limited thereto.

Further, the above-described conjugate may have increased duration of efficacy, as compared with X not modified with F, and such a conjugate may be not only in the above-described form but also in a form, in which it is encapsulated in biodegradable nanoparticles.

Still another aspect for implementing the present invention provides a long-acting conjugate, in which an interleukin-2 analog including any one amino acid sequence selected from the group consisting of amino acid sequences of SEQ ID NOS: 3 to 106 and an immunoglobulin Fc region are linked through a linker.

The interleukin-2 analog, the linker, and the long-acting conjugate including the same are as described above.

Yet another aspect for implementing the present invention provides a long-acting conjugate of an interleukin-2 analog represented by Chemical Formula 2 below:

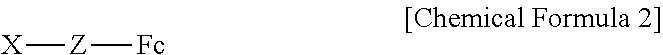

wherein X is an interleukin-2 analog including any one of polypeptide sequences selected from the amino acid sequence of SEQ ID NOS: 3 to 106;
Z is a polyethylene glycol linker having a molecular weight of 2 kDa to 30 kDa;
Fc is an immunoglobulin Fc region in the form of a dimer; and
- indicates a covalent linkage between X and Z and between Z and Fc;
wherein the long-acting conjugate is a long-acting conjugate in which one end of Z is covalently linked only to one polypeptide chain in the dimeric Fc region, and one molecule of X is covalently linked to the opposite end of Z.

The interleukin-2 analog, the linker, the Fc region, and the long-acting conjugate including the same are as described above.

In a specific embodiment, the interleukin-2 analog may include, essentially consist of, or consist of any one of sequences selected from the group consisting of amino acid sequences of SEQ ID NOS: 10, 13, 14, 15, 16, 17, 20, 21, 22, 32, 35, 36, 42, 53, 54, 56, 58, 59, 60, 62, 71, 72, 74, 75, 76, 77, 78, 85, 87, 89, 91, 92, 93, 94, 95, 98, 99, 100, 101, 103, 104, 105, and 106, but is not limited thereto.

In another specific embodiment, the interleukin-2 analog may include, essentially consist of, or consist of any one of sequences selected from the group consisting of amino acid sequences of SEQ ID NOS: 10, 13, 14, 16, 17, 20, 21, 22, 32, 35, 36, 42, 53, 54, 87, 89, 91, 92, 93, 94, 98, 99, 100, 101, 103, 104, and 105, but is not limited thereto.

In still another specific embodiment, the interleukin-2 analog may include, essentially consist of, or consist of any one of sequences selected from the group consisting of amino acid sequences of SEQ ID NOS: 22, 42, 53, 87, 105 and 106, but is not limited thereto.

Further, the interleukin-2 analog may be one further including one or more amino acids at the C-terminus thereof.

Even another aspect for implementing the present invention provides a long-acting conjugate of an interleukin-2 analog represented by Chemical Formula 3 below:

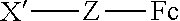

wherein X′ is an interleukin-2 analog including an amino acid sequence represented by General Formula 1 below:

[General Formula 1]
(General Formula 1, SEQ ID NO: 212)
X1-P-T-S-S-S-T-K-K-T-Q-L-Q-L-E-H-L-X18-X19-D-L-
X22-M-I-L-N-G-I-N-N-Y-K-N-P-K-L-T-X38-M-L-T-X42-
X43-F-X45-M-P-K-K-A-T-E-L-K-H-L-Q-C-L-E-X61-E-L-K-
P-L-E-X68-V-L-N-L-A-X74-S-K-N-F-H-X80-X81-P-R-X84-
X85-X86-S-N-I-N-X91-X92-V-X94-E-X96-K-G-S-E-T-T-F-
M-C-E-Y-A-D-E-T-A-T-I-V-E-F-L-N-R-W-I-T-F-S-Q-S-I-
I-S-T-L-T

in General Formula 1 above,
X1 is a deletion;
X18 is leucine (L) or arginine (R);
X19 is leucine (L) or tyrosine (Y);
X22 is glutamic acid (E) or glutamine (Q);
X38 is alanine (A), aspartic acid (D), or arginine (R);
X42 is alanine (A), phenylalanine (F), lysine (K), or tryptophan (W);
X43 is glutamic acid (E), lysine (K), or glutamine (Q);
X45 is alanine (A) or tyrosine (Y);
X61 is aspartic acid (D), glutamic acid (E), glutamine (Q), or arginine (R);
X68 is aspartic acid (D) or glutamic acid (E);
X74 is histidine (H) or glutamine (Q);
X80 is phenylalanine (F), leucine (L), valine (V), or tyrosine (Y);
X81 is aspartic acid (D), glutamic acid (E), or arginine (R);
X84 is aspartic acid (D) or glutamic acid (E);
X85 is alanine (A), glutamic acid (E), glycine (G), leucine (L), valine (V), tryptophan (W), or tyrosine (Y);
X86 is alanine (A), glycine (G), isoleucine (I), or valine (V);
X91 is threonine (T) or valine (V);
X92 is phenylalanine (F), isoleucine (I), or tyrosine (Y);
X94 is phenylalanine (F) or leucine (L);
X96 is phenylalanine (F) or leucine (L);
Z is a polyethylene glycol linker having a molecular weight of 2 kDa to 30 kDa;
Fc is an immunoglobulin Fc region in the form of a dimer; and
- indicates a covalent linkage between X′ and Z and between Z and Fc;
wherein the long-acting conjugate is a long-acting conjugate in which one end of Z is covalently linked only to one polypeptide chain in the dimeric Fc region, and one molecule of X′ is covalently linked to the opposite end of Z.

The interleukin-2 analog, the linker, the Fc region, and the long-acting conjugate including the same are as described above.

In General Formula 1 above, one or more amino acids may be added to threonine (T) corresponding to X133, but is not limited thereto.

Specifically, the interleukin-2 analog may include, essentially consist of, or consist of any one of sequences selected from the group consisting of amino acid sequences of SEQ ID NOS: 10, 13, 14, 15, 16, 17, 20, 21, 22, 32, 35, 36, 42, 53, 54, 56, 58, 59, 60, 62, 71, 72, 74, 75, 76, 77, 78, 85, 87, 89, 91, 92, 93, 94, 95, 98, 99, 100, 101, 103, 104, 105, and 106, but is not limited thereto.

Such an interleukin-2 analog may have an increased binding affinity for beta receptors compared to aldesleukin or native interleukin-2, but is not limited thereto.

In a specific embodiment, in General Formula 1,
X43 is lysine (K);
X45 is tyrosine (Y);
X61 is aspartic acid (D), glutamic acid (E), or glutamine (Q);
X68 is glutamic acid (E);
X74 is glutamine (Q);
X80 is phenylalanine (F) or leucine (L);
X85 is leucine (L), valine (V), or tyrosine (Y);
X86 is isoleucine (I) or valine (V); and
X92 is phenylalanine (F) or isoleucine (I), but is not limited thereto.

Specifically, the interleukin-2 analog is characterized in that it includes any one of sequences selected from the group consisting of amino acid sequences of SEQ ID NOS: 10, 13, 14, 16, 17, 20, 21, 22, 32, 35, 36, 42, 53, 54, 87, 89, 91, 92, 93, 94, 98, 99, 100, 101, 103, 104, and 105, but is not limited thereto.

The interleukin-2 analog of the present invention may further include one or more amino acids at the C-terminus thereof, but is not limited thereto.

Further another aspect for implementing the present invention provides a long-acting conjugate of an interleukin-2 analog represented by Chemical Formula 4 below:

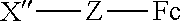

wherein X″ is an interleukin-2 analog including an amino acid sequence represented by General Formula 2 below:

[General Formula 2]
(General Formula 2, SEQ ID NO: 213)
X1-P-T-S-S-S-T-K-K-T-Q-L-Q-L-E-H-L-X18-L-D-L-X22-
M-I-L-N-G-I-N-N-Y-K-N-P-K-L-T-X38-M-L-T-X42-K-F-Y-
M-P-K-K-A-T-E-L-K-H-L-Q-C-L-E-X61-E-L-K-P-L-E-X68-
V-L-N-L-A-Q-S-K-N-F-H-F-X81-P-R-D-X85-X86-S-N-I-N-
V-F-V-L-E-L-K-G-S-E-T-T-F-M-C-E-Y-A-D-E-T-A-T-I-V-
E-F-L-N-R-W-I-T-F-S-Q-S-I-I-S-T-L-T

X1 is a deletion;
X18 is leucine (L) or arginine (R);
X22 is glutamic acid (E) or glutamine (Q);
X38 is alanine (A) or arginine (R);
X42 is phenylalanine (F) or lysine (K);
X61 is aspartic acid (D) or glutamic acid (E);
X68 is aspartic acid (D) or glutamic acid (E);
X81 is aspartic acid (D) or glutamic acid (E);
X85 is leucine (L) or valine (V);
X86 is isoleucine (I) or valine (V);
Z is a polyethylene glycol linker having a molecular weight of 2 kDa to 30 kDa;
Fc is an immunoglobulin Fc region in the form of a dimer; and
- indicates a covalent linkage between X″ and Z and between Z and Fc;
wherein the long-acting conjugate is a long-acting conjugate in which one end of Z is covalently linked only to one polypeptide chain in the dimeric Fc region, and one molecule of X″ is covalently linked to the opposite end of Z.

The interleukin-2 analog, the linker, the Fc region, and the long-acting conjugate including the same are as described above.

Specifically, the interleukin-2 analog may include any one of sequences selected from the group consisting of amino acid sequences of SEQ ID NOS: 22, 42, 53, 87, 105 and 106, but is not limited thereto.

In addition, in General Formula 2, one or more amino acids may be added to threonine (T) corresponding to X133, or one or more amino acids may be further included at the C-terminus of the interleukin-2 analog, but is not limited thereto.

Still further another aspect for implementing the present invention provides a long-acting conjugate including an interleukin-2 analog.

The interleukin-2 analog and the long-acting conjugate including the same are as described above.

Specifically, the method may include a step of linking the interleukin-2 analog, into which a modification was introduced into one or more amino acids selected from the group consisting of amino acids corresponding to positions 1, 12, 18, 19, 20, 22, 32, 35, 38, 42, 43, 45, 48, 49, 61, 68, 69, 74, 76, 80, 81, 82, 84, 85, 86, 87, 88, 89, 91, 92, 94, 95, 96, 125, 126, and 133 in native interleukin-2, with a biocompatible material (e.g., an immunoglobulin Fc region) via a linker (e.g., a non-peptidyl polymer).

The method of preparing a long-acting conjugate of the present invention may further include a step of introducing a modification into one or more amino acids selected from the group consisting of amino acids corresponding to positions 1, 12, 18, 19, 20, 22, 32, 35, 38, 42, 43, 45, 48, 49, 61, 68, 69, 74, 76, 80, 81, 82, 84, 85, 86, 87, 88, 89, 91, 92, 94, 95, 96, 125, 126, and 133 in native interleukin-2, but the method is not limited by specific steps or orders thereof as long as a long-acting conjugate, in which the interleukin-2 analog according to the present invention is linked to a biocompatible substance (e.g., immunoglobulin Fc region), is prepared.

In addition, the method of preparing a long-acting conjugate of the present invention may further include the steps of: a) culturing a transformant containing a nucleic acid encoding the interleukin-2 analog to express the interleukin-2 analog; and b) isolating and purifying the expressed interleukin-2 analog, but is not limited thereto.

The nucleic acid encoding the interleukin-2 analog may include or (essentially) consist of a nucleotide sequence of any one of SEQ ID NOS: 109 to 212, but is not limited thereto.

The medium used for culturing a transformant in the present invention must meet the requirements for culturing host cells in an appropriate manner. The carbon sources that can be included in the medium for the growth of host cells can be appropriately selected as a decision by those skilled in the art according to the type of transformants being produced, and appropriate culture conditions can be adopted to control the time and amount of culture.

Sugar sources that can be used may include sugars and carbohydrates (e.g., glucose, saccharose, lactose, fructose, maltose, starch, and cellulose); oils and fats (e.g., soybean oil, sunflower oil, castor oil, coconut oil, etc.); fatty acids (e.g., palmitic acid, stearic acid, and linoleic acid); alcohols (e.g., glycerol and ethanol); and organic acids (e.g., acetic acid). These materials can be used individually or as a mixture.

Nitrogen sources that can be used may include peptone, yeast extract, gravy, malt extract, corn steep liquor, soybean meal, and urea, or inorganic compounds (e.g., ammonium sulfate, ammonium chloride, ammonium phosphate, ammonium carbonate, and ammonium nitrate). Nitrogen sources can also be used individually or as a mixture.

Phosphorous sources that can be used may include potassium dihydrogen phosphate or dipotassium hydrogen phosphate, or corresponding sodium-containing salts thereof. In addition, the culture medium may contain a metal salt (e.g., magnesium sulfate and iron sulfate) required for growth.

Finally, in addition to these materials, essential growth materials (e.g., amino acids and vitamins) may be used. In addition, suitable precursors for culture media may be used. The above-mentioned raw materials can be added in a batch or continuous mode in a manner appropriate to the culture during the cultivation. Basic compounds (e.g., sodium hydroxide, potassium hydroxide, and ammonia) or acidic compounds (e.g., phosphoric acid and sulfuric acid) can be used in an appropriate manner to adjust the pH of the culture. In addition, antifoaming agents (e.g., fatty acid polyglycol esters) may be used to inhibit bubble generation. In order to maintain aerobic conditions, oxygen or oxygen-containing gas (e.g., air) is injected into the culture.

Culturing of the transformant according to the present invention is usually performed at a temperature of 20° C. to 45° C., specifically 25° C. to 40° C. In addition, the culture is continued until the maximum amount of the desired interleukin-2 analog is obtained, and for this purpose, the culture can usually last for 10 to 160 hours.

As described above, if the appropriate culture conditions are established depending on the host cell, the transformant according to the present invention will produce an interleukin-2 analog, and depending on the composition of the vector and the characteristics of the host cell, the interleukin-2 analog produced can be secreted into the cytoplasm of the host cell, into the periplasmic space, or extracellularly.

Proteins expressed in the host cell or outside thereof can be purified in a conventional manner. Examples of purification methods include salting out (e.g.: ammonium sulfate precipitation, sodium phosphate precipitation, etc.), solvent precipitation (e.g., protein fractionation precipitation using acetone, ethanol, etc.), dialysis, gel filtration, ion exchange, chromatography (e.g., reverse-phase column chromatography), ultrafiltration, etc. and can be used alone or in combination.

Further, the interleukin-2 analog prepared by a peptide synthesis method may be linked to a biocompatible substance (e.g., an immunoglobulin Fc region) via a non-peptidyl polymer. Since the sequences of the interleukin-2 analogs of the present invention are already provided, the synthesis of peptides can be performed using a known peptide synthesis method.

The interleukin-2 analog is as described above.

Still further another aspect for implementing the present invention provides a long-acting conjugate prepared by the method above.

Unless specified otherwise herein, the description in the detailed description or claims with respect to “interleukin-2 analog” according to the present invention or a “conjugate”, in which an interleukin-2 analog is covalently linked to a biocompatible material, may be applied to the forms, which include not only the corresponding interleukin-2 analog or conjugate but also the salts of the corresponding interleukin-2 analog or conjugate (e.g., pharmaceutically acceptable salts of the interleukin-2 analog), or solvates thereof. Accordingly, even in a case where an “interleukin-2 analog” or “conjugate” is merely described herein, the description may also be equally applied to a particular salt thereof, a particular solvate thereof, and a particular solvate of the particular salt thereof. These salts may be, for example, in a form where any pharmaceutically acceptable salts are used. The kind of the salt is not particularly limited. However, the salt is preferably one that is safe and effective to a subject, e.g., a mammal, but is not particularly limited thereto.

The term “pharmaceutically acceptable” refers to a material which can be effectively used for a desired purpose without causing excessive toxicity, irritation, allergic reactions, etc. within the scope of medical judgment.

As used herein, the term “pharmaceutically acceptable salt” includes salts which are derived from pharmaceutically acceptable inorganic acids, organic acids, or bases. Examples of suitable acids include hydrochloric acid, bromic acid, sulfuric acid, nitric acid, perchloric acid, fumaric acid, maleic acid, phosphoric acid, glycolic acid, lactic acid, salicylic acid, succinic acid, toluene-p-sulfonic acid, tartaric acid, acetic acid, citric acid, methanesulfonic acid, formic acid, benzoic acid, malonic acid, naphthalene-2-sulfonic acid, benzenesulfonic acid, etc. Salts derived from suitable bases may include alkali metals (e.g., sodium, potassium, etc.), alkaline earth metals (e.g., magnesium, etc.), ammonium, etc.

Additionally, as used herein, the term “solvate” refers to a complex formed between the interleukin-2 analog according to the present invention or a salt thereof and a solvent molecule.

Still further another aspect for implementing the present invention provides a composition including an interleukin-2 analog or a long-acting conjugate thereof. The interleukin-2 analog and the long-acting conjugate thereof are as described above. The composition according to the present invention may be a pharmaceutical composition, and in a specific embodiment, it may be a pharmaceutical composition with the use for preventing or treating cancer.

The composition according to the present invention may include an interleukin-2 analog or a long-acting conjugate thereof, and specifically may include a pharmacologically effective amount of an interleukin-2 analog or a long-acting conjugate thereof. In addition, it may further include a pharmaceutically acceptable carrier.

In a specific embodiment of the composition according to the present invention, the composition may include an interleukin-2 analog including any one amino acid sequence selected from the group consisting of SEQ ID NOS: 3 to 106; or a long-acting conjugate including the same, and more specifically an interleukin-2 analog including any one amino acid sequence selected from the group consisting of SEQ ID NOS: 22, 42, 53, 87, 105, and 106; or a long-acting conjugate including the interleukin-2 analog, but is not limited thereto.

As used herein, the term “pharmaceutically effective amount” may refer to a safe administration dose in which the interleukin-2 or a conjugate thereof exhibits cancer prevention or treatment effects, but does not show toxicity or side effects to patients, and may refer to an administration dose capable of exhibiting significant activities on interleukin-2 receptors, specifically beta and/or alpha receptors.

The composition according to the present invention may exhibit one or more of the following properties, but is not limited as long as it exhibits an increased immune response and anticancer effect, etc. based on increased binding affinity for interleukin-2 beta receptors:
(i) high blood exposure relative to aldesleukin;
(ii) high tumor growth inhibition compared to aldesleukin,
(iii) high memory T cell-generating response compared to aldesleukin.

Interleukin-2, known as a T cell growth factor, is a protein involved in immunomodulation, and has the activity of proliferating T cells, stimulating B cells, and secreting y-interferon by acting on T cells. Based on the immunomodulatory activity of interleukin-2, cancer prevention or treatment effects can be obtained by removing cancer cells using the body's immune system.

In particular, the interleukin-2 analog of the present invention has an increased binding affinity for interleukin-2 beta receptors, which play a major role in signal transduction, leading to a more effective anticancer effect in the immune system of an individual. In addition, the long-acting conjugate including the interleukin-2 analog exhibits excellent bioavailability due to increased blood exposure as the duration of efficacy is increased while having increased binding affinity for interleukin-2 beta receptors, and consequently exhibiting excellent tumor growth inhibition and memory T cell-producing ability, thereby showing effective cancer prevention or treatment effects.

Specifically, the long-acting conjugate of an interleukin-2 analog of the present invention may have no binding affinity or may have binding affinity for interleukin-2 alpha receptors of about 0.001-fold or greater, about 0.005-fold or greater, about 0.01-fold or greater, about 0.05-fold or greater, about 0.1-fold or greater, about 0.3-fold or greater, about 0.5-fold or greater, about 0.6-fold or greater, about 0.7-fold or greater, about 0.8-fold or greater, about 0.9-fold or greater, about 1.1-fold or greater, about 1.3-fold or greater, about 1.5-fold or greater, or about 1.7-fold or greater compared to the binding affinity of native interleukin-2, aldesleukin, or the long-acting conjugate including the same for interleukin-2 alpha receptors, but the numerical value of the binding affinity is not limited, and the value will belong to the scope of the present invention as long as there is a change in the binding affinity compared to that of native interleukin-2 or aldesleukin.

Additionally, the long-acting conjugate of an interleukin-2 analog of the present invention may specifically have binding affinity for interleukin-2 beta receptors of about 0.1-fold or greater, about 0.3-fold or greater, about 0.5-fold or greater, about 0.7-fold or greater, about 1.0-fold or greater, about 10-fold or greater, about 20-fold or greater, about 30-fold or greater, about 40-fold or greater, about 50-fold or greater, about 60-fold or greater, about 70-fold or greater, about 80-fold or greater, about 90-fold or greater, about 100-fold or greater, about 130-fold or greater, about 150-fold or greater, and about 200-fold or greater compared to the binding affinity of native interleukin-2, aldesleukin, or the long-acting conjugate including the same for interleukin-2 beta receptors, but the numerical value of the binding affinity is not limited, and the value will belong to the scope of the present invention as long as there is a change or increase in the binding affinity compared to that of native interleukin-2 or aldesleukin.

In the present invention, the cancer may be any one selected from the group consisting of colorectal cancer, liver cancer, ovarian cancer, pancreatic cancer, gallbladder cancer, kidney cancer, lung cancer, skin cancer, melanoma, breast cancer, bladder cancer, and stomach cancer, or may be metastatic cancer, but is not limited thereto. It may also include metastatic renal cell carcinoma or metastatic melanoma.

As used herein, the term “prevention” refers to all kinds of actions associated with inhibition or delay of cancer or tumor by administering the interleukin-2 analog (e.g., the interleukin-2 analog itself or a long-acting conjugate form in which a biocompatible material is bound thereto), or a composition including the same.

As used herein, the term “treatment” refers to all kinds of actions associated with the improvement or advantageous changes in symptoms of cancer by administering the interleukin-2 analog (e.g., the interleukin-2 analog itself or a long-acting conjugate form in which a biocompatible material is bound thereto), or a composition including the same.

The use of the interleukin-2 analog of the present invention or a long-acting conjugate thereof has great advantages of improving the quality of life of patients by reducing the number of administrations to chronic patients who are subjected to daily administration due to a dramatic increase in blood exposure, blood half-life, and in vivo duration of efficacy.

The pharmaceutical composition of the present invention may further include a pharmaceutically acceptable carrier or diluent. Such a pharmaceutically acceptable carrier or diluent may be one which does not occur naturally.

As used herein, the term “pharmaceutically acceptable” refers to an amount sufficient to exhibit therapeutic effects without causing side-effects, and may be easily determined by those of ordinary skill in the art, based on factors well known in the medical field such as the type of disease, a patient's age, body weight, health status, gender, and sensitivity to drug, administration route, administration method, frequency of administration, duration of treatment, and a drug used in combination or concurrently.

The pharmaceutical composition including the interleukin-2 analog of the present invention or a long-acting conjugate thereof may further include a pharmaceutically acceptable excipient. Although the excipient is not particularly limited, a binder, a lubricant, a disintegrator, a solubilizer, a dispersant, a stabilizer, a suspending agent, a coloring agent, and a flavoring agent may be used for oral administration, a buffer, a preservative, an analgesic, a solubilizer, an isotonic agent, and a stabilizer may be used in combination for injectable preparations, and a base, an excipient, a lubricant, a preservative, etc. may be used for topical administration.

The composition of the present invention may be formulated into various forms in combination with the above-mentioned pharmaceutically acceptable excipient. For example, for oral administration, the composition may be formulated into tablets, troches, capsules, elixirs, suspensions, syrups, wafers, etc. For injectable preparations, the composition may be formulated into a single-dose ampoule or multidose form. The composition may also be formulated into solutions, suspensions, tablets, pills, capsules, sustained-release preparations, etc.

Meanwhile, examples of the carrier, excipient, and diluent suitable for formulation may include lactose, dextrose, sucrose, sorbitol, mannitol, xylitol, erythritol, maltitol, starch, acacia, alginate, gelatin, calcium phosphate, calcium silicate, cellulose, methyl cellulose, microcrystalline cellulose, polyvinyl pyrrolidone, water, methylhydroxy benzoate, propylhydroxy benzoate, talc, magnesium stearate, or mineral oils. Also, the composition may further include a filler, an anti-coagulant, a lubricant, a humectant, a flavoring agent, a preservative, etc.

In addition, the pharmaceutical composition of the present invention may have any one formulation selected from the group consisting of tablets, pills, powders, granules, capsules, suspensions, formulations for internal use, emulsions, syrups, sterilized aqueous solutions, non-aqueous solvents, lyophilized preparations, and suppositories.

Also, the composition may be formulated in a unit dosage form suitable for administration into a patient's body, specifically in a form useful for administration of protein medicines, according to a method commonly used in the art, and administered via an oral administration route or a parenteral administration route such as an intradermal, intravenous, intramuscular, intraarterial, intramedullary, intrathecal, intraventricular, intrapulmonary, transdermal, subcutaneous, intraperitoneal, intranasal, intestinal, topical, sublingual, vaginal, or rectal route using an administration method commonly used in the art, but is not limited thereto.

In addition, the conjugate may be used in combination with various carriers permitted as medicaments such as a saline solution or an organic solvent. As the medicaments, carbohydrates such as glucose, sucrose, or dextran, antioxidants such as ascorbic acid or glutathione, chelating agents, low-molecular-weight proteins, or other stabilizers may be used to improve stability or absorbability.

Still further another aspect for implementing the present invention provides a method for preventing or treating cancer, including administering an interleukin-2 analog, a long-acting conjugate thereof, or a pharmaceutical composition including the same to an individual in need thereof.

The interleukin-2 analog and/or long-acting conjugate of the interleukin-2 analog, composition including the same, cancer, prevention and treatment are as described above.

As used herein, the individual refers to an individual having cancer or suspected of having cancer, and may refer to mammals such as humans, rats, livestock, etc., but any individual that may be treated with the interleukin-2 analog and/or conjugate, or a composition including the same may be included without limitation.

As used herein, the term “administration” refers to an introduction of a particular material (e.g., interleukin-2 analog or long-acting conjugate thereof) to a patient by an appropriate manner. The composition may be, but is not particularly limited to, administered by a general route that enables the delivery of the composition to a target tissue in vivo, for example, intraperitoneal, intravenous, intramuscular, subcutaneous, intradermal, oral, topical, intranasal, intrapulmonary, and intrarectal administration.

The method of the present invention may include administering the pharmaceutical composition including the interleukin-2 analog or a long-acting conjugate thereof at a pharmaceutically effective amount. The suitable total daily dose should be determined within appropriate medical judgment by a physician, and administered once or several times in divided doses. For the purpose of the present invention, the specific therapeutically effective dose for any particular patient may be preferably applied differently, depending on various factors well known in the medical art, including the kind and degree of the response to be achieved, specific compositions including whether other agents are occasionally used therewith or not, the patient's age, body weight, general health conditions, gender and diet, the time and route of administration, secretion rate of the composition, duration of treatment, other drugs used in combination or concurrently with the specific composition, and like factors well-known in the medical arts.

In the method of the present invention, the dosage and frequency during administration are determined according to the type of drug, an active ingredient, along with several related factors such as the disease to be treated, the route of administration, patient's age, gender and weight, and disease severity. Specifically, the composition of the present invention may include the interleukin-2 analog or the long-acting conjugate including the same in a pharmaceutically effective amount, but is not limited thereto.

The inclusion of the interleukin-2 analog or the long-acting conjugate in a pharmaceutically effective amount means the degree to which the desired pharmacological activity (e.g., prevention, improvement or treatment of cancer) can be obtained due to the interleukin-2 analog or the long-acting conjugate, or may additionally mean a pharmaceutically acceptable level as a level that does not cause toxicity or side effects in the administered individual, but is not limited thereto. Such a pharmaceutically effective amount may be comprehensively determined in consideration of administration frequency, patient, formulation, etc.

The pharmaceutical composition of the present invention may contain the component (active ingredient) in an amount of 0.01% to 99% by weight to volume, but is not particularly limited thereto.

The total effective dose of the composition of the present invention may be administered to a patient in a single dose or may be administered for a long period of time in multiple doses according to a fractionated treatment protocol. In the pharmaceutical composition of the present invention, the content of the active ingredient may vary depending on the disease severity. Specifically, a preferred total daily dose of the interleukin-2 analog of the present invention or a long-acting conjugate thereof may be about 0.0001 mg to 500 mg per 1 kg of body weight of a patient. However, the effective dose of the interleukin-2 analog or a conjugate thereof is determined considering various factors including a patient's age, body weight, health conditions, gender, disease severity, diet, and excretion rate, in addition to administration route of the pharmaceutical composition and treatment frequency. In this regard, those skilled in the art may easily determine the effective dose suitable for the particular use of the composition of the present invention. The pharmaceutical composition according to the present invention is not particularly limited to the formulation, and administration route and mode, as long as it shows the effects of the present invention.

The pharmaceutical composition of the present invention may have excellent in vivo duration and titer, thereby remarkably reducing the number and frequency of administration of the pharmaceutical preparation of the present invention. The pharmaceutical composition may be administered by intraperitoneal, intravenous, intramuscular, subcutaneous, intradermal, oral, topical, intranasal, intrapulmonary, or intrarectal route, but the administration is not limited to a specific route of administration as long as the desired pharmacological effect can be obtained.

In an example, the pharmaceutical composition of the present invention may be administered once a week, once every two weeks, once every three weeks, once every four weeks, or once a month, or may be administered once or multiple times at a time interval ranging from 1 week to 1 month, but the frequency is not limited thereto.

Still further aspect for implementing the present invention provides the use of the interleukin-2 analog, a long-acting conjugate thereof, or a composition including the same in the preparation of medicaments for prevention or treatment of cancer.

The interleukin-2 analog and/or long-acting conjugate thereof, or composition including the same, cancer, prevention, treatment, route of administration, and frequency of administration are the same as described above.

Still further aspect for implementing the present invention provides the use of the interleukin-2 analog, a long-acting conjugate thereof, or a composition including the same in the prevention or treatment of cancer.

The interleukin-2 analog and/or long-acting conjugate thereof, or composition including the same, cancer, prevention, treatment, route of administration, and frequency of administration are the same as described above.

Meanwhile, unless otherwise required by context in the present specification, expressions such as “include”, “including”, “containing”, etc. mean that they include a specified integer or group of integers, but it should be understood that these expressions do not exclude other integers or a set of integers.

Hereinafter, the present invention will be described in more detail with reference to the following Examples. However, these Examples are only for describing the present invention in more detail only, and the scope of the present invention is not intended to be limited by these Examples.

### Example 1: Preparation of Expression Vectors for Native Interleukin-2 and Interleukin-2 Analogs

For the preparation of expression vectors for native interleukin-2 encoding 133 amino acids, an interleukin-2 that was synthesized based on the reported interleukin-2 sequence (NM_000586.3; SEQ ID NO: 1) was cloned into the pET-22b vector (Novagen). Additionally, a novel interleukin-2 analog was prepared in which an amino acid(s) of interleukin-2 were modified using the interleukin-2 as a template.

The PCR conditions for the amplification of the interleukin-2 analog were 16 cycles of a process consisting of 95° C. for 30 seconds, 55° C. for 60 seconds, and 65° C. for 6.5 minutes. In order to confirm whether the amino acid(s) at the desired position had been correctly substituted, sequence analysis was performed on the mutagenesis product obtained under the conditions above. As a result, it was confirmed that the modifications shown in Table 1 below were found based on the native type at the desired mutation positions for each interleukin-2 analog. The thus-obtained expression vectors were named pET22b-interleukin-2 analogs 1 to 105.

Table 1 below shows the altered sequences of amino acids and analog names for each. In order to prepare these interleukin-2 analogs, forward (F) and reverse (R) primers were synthesized, and then PCR was performed to amplify each analog gene.

In Table 1 below, analog 1 represents aldesleukin, and primer nos. 1 to 204 correspond to SEQ ID NOS: 214 to 417, respectively.

TABLE 1
Types of Interleukin-2 Analogs, Modification Positions and Altered Sequences thereof

| Analog | Modification Positions and Altered Sequences Thereof | Primer # |
|---|---|---|
| 1 | desA1, C125S | 197, 198, 201, 202 |
| 2 | desA1, C125S, S87C | 197, 198, 201, 202, 149, 150 |
| 3 | desA1, C125S, K32C | 197, 198, 201, 202, 19, 20 |
| 4 | desA1, C125S, K35C | 197, 198, 201, 202, 21, 22 |
| 5 | desA1, C125S, K43C | 197, 198, 201, 202, 47, 48 |
| 6 | desA1, C125S, K48C | 197, 198, 201, 202, 53, 54 |
| 7 | desA1, C125S, K49C | 197, 198, 201, 202, 55, 56 |
| 8 | desA1, C125S, K76C | 197, 198, 201, 202, 73, 74 |
| 9 | desA1, C125S, R38A | 197, 198, 201, 202, 25, 26 |
| 10 | desA1, C125S, F42K | 197, 198, 201, 202, 35, 36 |
| 11 | desA1, C125S, F42A | 197, 198, 201, 202, 33, 34 |
| 12 | desA1, C125S, R38A, F42K | 197, 198, 201, 202, 25, 26, 35, 36 |
| 13 | desA1, C125S, R38A, F42A | 197, 198, 201, 202, 25, 26, 33, 34 |
| 14 | desA1, C125S, L19Y, R38A, F42K | 197, 198, 201, 202, 13, 14, 25, 26, |
| | | 35, 36 |
| 15 | desA1, C125S, R38A, F42K, D84E | 197, 198, 201, 202, 25, 26, 35, 36, |
| | | 109, 110 |
| 16 | desA1, C125S, R38A, F42K, N88Q | 197, 198, 201, 202, 25, 26, 35, 36, |
| | | 153, 154 |
| 17 | desA1, C125S, R38A, F42K, V91T | 197, 198, 201, 202, 25, 26, 35, 36, |
| | | 165, 166 |
| 18 | desA1, C125S, R38A, F42K, E61Q | 197, 198, 201, 202, 25, 26, 35, 36, |
| | | 59, 60 |
| 19 | desA1, C125S, R38A, F42K, R81D, I92F | 197, 198, 201, 202, 25, 26, 35, 36, |
| | | 97, 98, 167, 168 |
| 20 | desA1, C125S, R38A, F42K, L85V, I86V, I92F | 197, 198, 201, 202, 25, 26, 35, 36, |
| | | 139, 140, 167, 168 |
| 21 | desA1, C125S, R38A, F42K, L80F, R81D, L85V, I86V, I92F | 197, 198, 201, 202, 25, 26, 35, 36, |
| | | 79, 80, 167, 168 |
| 22 | desA1, C125S, L12V, R38A, F42K | 197, 198, 201, 202, 3, 4, 25, 26, |
| | | 35, 36 |
| 23 | desA1, C125S, L12F, R38A, F42K | 197, 198, 201, 202, 1, 2, 25, 26, |
| | | 35, 36 |
| 24 | desA1, C125S, L19V, R38A, F42K | 197, 198, 201, 202, 11, 12, 25, 26, |
| | | 35, 36 |
| 25 | desA1, C125S, L19F, R38A, F42K | 197, 198, 201, 202, 9, 10, 25, 26, |
| | | 35, 36 |
| 26 | desA1, C125S, R38A, F42K, I89F | 197, 198, 201, 202, 25, 26, 35, 36, |
| | | 157, 158 |
| 27 | desA1, C125S, R38A, F42K, V91F | 197, 198, 201, 202, 25, 26, 35, 36, |
| | | 163, 164 |
| 28 | desA1, C125S, R38A, F42K, L94V | 197, 198, 201, 202, 25, 26, 35, 36 |
| 29 | desA1, C125S, R38A, F42K, Q126T | 197, 198, 201, 202, 25, 26, 35, 36, |
| | | 199, 200 |
| 30 | desA1, C125S, R38A, R81D, I92F | 197, 198, 201, 202, 27, 28, 97, 98, |
| | | 169, 170 |
| 31 | desA1, C125S, R38A, D84E | 197, 198, 201, 202, 27, 28, 109, |
| | | 110 |
| 32 | desA1, C125S, R38A, R81D, D84E, I92F | 197, 198, 201, 202, 27, 28, 95, 96, |
| | | 169, 170 |
| 33 | desA1, C125S, R38A, L80F | 197, 198, 201, 202, 27, 28, 77, 78 |
| 34 | desA1, C125S, R38A, L80F, D84E | 197, 198, 201, 202, 27, 28, 77, 78, |
| | | 109, 110 |
| 35 | desA1, C125S, R38A, L94F, L96F | 197, 198, 201, 202, 27, 28, 189, |
| | | 190 |
| 36 | desA1, C125S, R38A, L94F, L96V | 197, 198, 201, 202, 27, 28, 193, |
| | | 194 |
| 37 | desA1, C125S, R38A, L94F, L96I | 197, 198, 201, 202, 27, 28, 191, |
| | | 192 |
| 38 | desA1, C125S, R38A, F42K, R81D, I92F, L94F, L96F | 197, 198, 201, 202, 25, 26, 35, 36, |
| | | 97, 98, 175, 176 |
| 39 | desA1, C125S, R38A, F42K, R81D, I92F, L94F, L96V | 197, 198, 201, 202, 25, 26, 35, 36, |
| | | 97, 98, 179, 180 |
| 40 | desA1, C125S, R38A, F42K, R81D, I92F, L94F, L96I | 197, 198, 201, 202, 25, 26, 35, 36, |
| | | 97, 98, 177, 178 |
| 41 | desA1, C125S, L80F, R81D, L85V, I86V, I92F | 197, 198, 201, 202, 25, 26, 29, 30, |
| | | 35, 36, 41, 42, 79, 80, 167, 168 |
| 42 | desA1, C125S, R38A, F42K, R81E, I92F | 197, 198, 201, 202, 25, 26, 35, 36, |
| | | 103, 104, 169, 170 |
| 43 | desA1, C125S, R38A, F42K, R81D, I92L | 197, 198, 201, 202, 25, 26, 35, 36, |
| | | 99, 100, 183, 184 |
| 44 | desA1, C125S, R38A, F42K, R81D, D84V, I92F | 197, 198, 201, 202, 25, 26, 35, 36, |
| | | 99, 100, 113, 114, 169, 170 |
| 45 | desA1, C125S, R38A, F42K, R81D, D84F, I92F | 197, 198, 201, 202, 25, 26, 35, 36, |
| | | 99, 100, 111, 112, 169, 170 |
| 46 | desA1, C125S, D20V, R38A, F42K, R81D, I92F | 197, 198, 201, 202, 17, 18, 25, 26, |
| | | 35, 36, 99, 100, 169, 170 |
| 47 | desA1, C125S, D20F, R38A, F42K, R81D, I92F | 197, 198, 201, 202, 15, 16, 25, 26, |
| | | 35, 36, 99, 100, 169, 170 |
| 48 | desA1, C125S, R38A, F42K, R81D, N88V, I92F | 197, 198, 201, 202, 25, 26, 35, 36, |
| | | 99, 100, 155, 156, 169, 170 |
| 49 | desA1, C125S, R38A, F42K, R81D, N88F, I92F | 197, 198, 201, 202, 25, 26, 35, 36, |
| | | 99, 100, 151, 152, 169, 170 |
| 50 | desA1, C125S, F42K, L80F, R81D, L85V, I86V, I92F | 197, 198, 201, 202, 25, 26, 29, 30, |
| | | 35, 36, 79, 80, 139, 140, 169, 170 |
| 51 | desA1, C125S, E61Q, R81D, I92F | 197, 198, 201, 202, 59, 60, 99, |
| | | 100, 169, 170 |
| 52 | desA1, C125S, R38A, F42K, L80F, R81D, I92F | 197, 198, 201, 202, 25, 26, 35, 36, |
| | | 79, 80, 169, 170 |
| 53 | desA1, C125S, R38A, F42K, L80F, R81D, D84E, I92F | 197, 198, 201, 202, 25, 26, 35, 36, |
| | | 79, 80, 95, 96, 169, 170 |
| 54 | desA1, C125S, R38A, F42K, Q74H, R81D, I92F | 197, 198, 201, 202, 25, 26, 35, 36, |
| | | 71, 72, 99, 100, 169, 170 |
| 55 | desA1, C125S, R38A, F42K, Q74H, L80F, R81D, I92F | 197, 198, 201, 202, 25, 26, 35, 36, |
| | | 71, 72, 79, 80, 169, 170 |
| 56 | desA1, C125S, R38A, F42K, Y45A, Q74H, R81D, I92F | 197, 198, 201, 202, 25, 26, 35, 36, |
| | | 49, 50, 71, 72, 99, 100, 169, 170 |
| 57 | desA1, C125S, R38A, F42K, Y45A, Q74H, L80F, R81D, I92F | 197, 198, 201, 202, 25, 26, 35, 36, |
| | | 49, 50, 71, 72, 79, 80, 169, 170 |
| 58 | desA1, C125S, R38A, F42K, L80F, R81D, L85A, I86A, I92F | 197, 198, 201, 202, 25, 26, 35, 36, |
| | | 79, 80, 115, 116, 167, 168 |
| 59 | desA1, C125S, R38A, F42K, L80F, R81D, L85A, I86A, I92Y | 197, 198, 201, 202, 25, 26, 35, 36, |
| | | 79, 80, 115, 116, 187, 188 |
| 60 | desA1, C125S, R38A, Y45A, L80Y, L85A, I86A, I92Y | 197, 198, 201, 202, 27, 28, 35, 36, |
| | | 41, 42, 51, 52, 93, 94, 115, 116, |
| | | 187, 188 |
| 61 | desA1, C125S, R38A, F42K, L80Y, R81D, L85G, I86V, | 197, 198, 201, 202, 25, 26, 35, 36, |
| | I92Y | 87, 88, 125, 126, 187, 188 |
| 62 | desA1, C125S, R38A, L80W, R81E, L85G, I86A, I92F | 197, 198, 201, 202, 27, 28, 35, 36, |
| | | 41, 42, 85, 86, 123, 124, 171, 172 |
| 63 | desA1, C125S, R38A, F42K, L80D, R81E, L85T, I86G, I92F | 197, 198, 201, 202, 25, 26, 35, 36, |
| | | 75, 76, 133, 134, 167, 168 |
| 64 | desA1, C125S, R38A, F42K, L80Y, R81N, L85V, I86V, I92F | 197, 198, 201, 202, 25, 26, 35, 36, |
| | | 91, 92, 139, 140, 167, 168 |
| 65 | desA1, C125S, R38A, F42K, L80Y, R81E, L85V, I86V, I92F | 197, 198, 201, 202, 25, 26, 35, 36, |
| | | 89, 90, 139, 140, 167, 168 |
| 66 | desA1, C125S, R38A, F42K, L80F, R81E, L85F, I86V, I92F | 197, 198, 201, 202, 25, 26, 35, 36, |
| | | 81, 82, 121, 122, 167, 168 |
| 67 | desA1, C125S, R38A, F42K, L80Y, R81D, L85F, I86V, | 197, 198, 201, 202, 25, 26, 35, 36, |
| | I92W, E95D | 87, 88, 121, 122, 185, 186, 195, |
| | | 196 |
| 68 | desA1, C125S, R38A, F42K, L80F, R81E, L85I, I86V, V91E, | 197, 198, 201, 202, 25, 26, 35, 36, |
| | I92F | 81, 82, 127, 128, 159, 160, 167, |
| | | 168 |
| 69 | desA1, C125S, R38A, F42K, L80Y, R81E, L85F, I86L, | 197, 198, 201, 202, 25, 26, 35, 36, |
| | V91E, I92W, E95D | 89, 90, 119, 120, 161, 162, 185, |
| | | 186, 195, 196 |
| 70 | desA1, C125S, R38A, F42K, L80Y, R81D, L85V, I86V, I92F | 197, 198, 201, 202, 25, 26, 35, 36, |
| | | 87, 88, 139, 140, 167, 168 |
| 71 | desA1, C125S, R38A, F42K, L80F, R81E, L85V, I86V, I92F | 197, 198, 201, 202, 25, 26, 35, 36, |
| | | 81, 82, 139, 140, 167, 168 |
| 72 | desA1, C125S, R38A, F42K, L80F, R81D, L85V, I86G, I92F | 197, 198, 201, 202, 25, 26, 35, 36, |
| | | 79, 80, 135, 136, 167, 168 |
| 73 | desA1, C125S, R38A, F42K, L80F, R81D, L85W, I86V, I92F | 197, 198, 201, 202, 25, 26, 35, 36, |
| | | 79, 80, 141, 142, 167, 168 |
| 74 | desA1, C125S, R38D, F42K, L80Y, R81D, L85V, I86V, I92F | 197, 198, 201, 202, 31, 32, 35, 36, |
| | | 87, 88, 139, 140, 167, 168 |
| 75 | desA1, C125S, R38A, F42K, Y45A, L80F, R81E, L85V, | 197, 198, 201, 202, 25, 26, 35, 36, |
| | I86V, I92F | 51, 52, 81, 82, 139, 140, 167, 168 |
| 76 | desA1, C125S, R38A, F42K, K43Q, E61R, L80F, R81D, | 197, 198, 201, 202, 25, 26, 39, 40, |
| | L85V, I86G, I92F | 61, 62, 79, 80, 135, 136, 167, 168 |
| 77 | desA1, C125S, R38A, F42K, K43E, E61R, L80F, R81D, | 197, 198, 201, 202, 25, 26, 37, 38, |
| | L85W, I86V, I92F | 61, 62, 79, 80, 143, 144, 167, 168 |
| 78 | desA1, C125S, K35E, R38A, F42K, L80F, R81E, L85V, | 197, 198, 201, 202, 23, 24, 25, 26, |
| | I86V, I92F | 35, 36, 81, 82, 139, 140, 167, 168 |
| 79 | desA1, C125S, K35E, R38A, F42K, Q74H, L80F, R81E, | 197, 198, 201, 202, 23, 24, 25, 26, |
| | L85V, I86V, I92F | 35, 36, 71, 72, 81, 82, 139, 140, |
| | | 167, 168 |
| 80 | desA1, C125S, K35E, R38A, F42K, Q74H, L80F, R81E, | 197, 198, 201, 202, 23, 24, 25, 26, |
| | P82G, L85V, I86V, I92F | 35, 36, 71, 72, 81, 82, 105, 106, |
| | | 139, 140, 167, 168 |
| 81 | desA1, C125S, K35E, R38A, F42K, Q74H, L80F, R81E, | 197, 198, 201, 202, 23, 24, 25, 26, |
| | P82V, L85V, I86V, I92F | 35, 36, 71, 72, 81, 82, 107, 108, |
| | | 139, 140, 167, 168 |
| 82 | desA1, C125S, L18R, Q22E, L80F, R81D, L85E, I86V, I92F | 197, 198, 201, 202, 7, 8, 25, 26, |
| | | 29, 30, 35, 36, 43, 44, 79, 80, 117, |
| | | 118, 167, 168 |
| 83 | desA1, C125S, L18R, L19R, Q22E, L80F, R81D, L85E, | 197, 198, 201, 202, 5, 6, 25, 26, |
| | I86V, I92F | 29, 30, 35, 36, 43, 44, 79, 80, 117, |
| | | 118, 167, 168 |
| 84 | desA1, C125S, L18R, Q22E, L80V, R81D, L85E, I86V, I92F | 197, 198, 201, 202, 7, 8, 25, 26, |
| | | 29, 30, 35, 36, 43, 44, 83, 84, 117, |
| | | 118, 167, 168 |
| 85 | desA1, C125S, L80F, R81E, L85V, I86V, I92F | 197, 198, 201, 202, 25, 26, 29, 30, |
| | | 35, 36, 43, 44, 81, 82, 139, 140, |
| | | 167, 168 |
| 86 | desA1, C125S, L18R, Q22E, L80F, R81E, L85V, I86V, I92F | 197, 198, 201, 202, 7, 8, 25, 26, |
| | | 29, 30, 35, 36, 43, 44, 81, 82, 139, |
| | | 140, 167, 168 |
| 87 | desA1, C125S, L18R, L19R, Q22E, L80F, R81E, L85V, | 197, 198, 201, 202, 5, 6, 25, 26, |
| | I86V, I92F | 29, 30, 35, 36, 43, 44, 81, 82, 139, |
| | | 140, 167, 168 |
| 88 | desA1, C125S, E61D, L80F, R81E, L85V, I86V, I92F | 197, 198, 201, 202, 25, 26, 29, 30, |
| | | 35, 36, 43, 44, 57, 58, 81, 82, 139, |
| | | 140, 167, 168 |
| 89 | desA1, C125S, R38A, E68Q, L80F, R81E, L85V, I86V, I92F | 197, 198, 201, 202, 27, 28, 35, 36, |
| | | 43, 44, 65, 66, 81, 82, 139, 140, |
| | | 167, 168 |
| 90 | desA1, C125S, F42W, L80F, R81E, L85V, I86V, I92F | 197, 198, 201, 202, 25, 26, 29, 30, |
| | | 45, 46, 81, 82, 139, 140, 167, 168 |
| 91 | desA1, C125S, E61Q, L80F, R81E, L85V, I86V, I92F | 197, 198, 201, 202, 25, 26, 29, 30, |
| | | 35, 36, 43, 44, 59, 60, 81, 82, 139, |
| | | 140, 167, 168 |
| 92 | desA1, C125S, L80F, R81E, L85V, I86V, V91T, I92F | 197, 198, 201, 202, 25, 26, 29, 30, |
| | | 35, 36, 43, 44, 81, 82, 139, 140, |
| | | 147, 148, 167, 168 |
| 93 | desA1, C125S, L80F, R81E, D84E, L85V, I86V, V91T, I92F | 197, 198, 201, 202, 25, 26, 29, 30, |
| | | 35, 36, 43, 44, 81, 82, 101, 102, |
| | | 139, 140, 167, 168 |
| 94 | desA1, C125S, L80F, R81E, L85V, I86V, I92F, L94F, L96F | 197, 198, 201, 202, 25, 26, 29, 30, |
| | | 35, 36, 43, 44, 81, 82, 139, 140, |
| | | 173, 174 |
| 95 | desA1, C125S, V69G, L80F, R81E, L85V, I86V, I92F | 197, 198, 201, 202, 25, 26, 29, 30, |
| | | 35, 36, 43, 44, 67, 68, 81, 82, 139, |
| | | 140, 167, 168 |
| 96 | desA1, C125S, V69G, Q74A, L80F, R81E, L85V, I86V, I92F | 197, 198, 201, 202, 25, 26, 29, 30, |
| | | 35, 36, 43, 44, 69, 70, 81, 82, 139, |
| | | 140, 167, 168 |
| 97 | desA1, C125S, R38A, L80F, R81D, I92F | 197, 198, 201, 202, 27, 28, 35, 36, |
| | | 41, 42, 79, 80, 167, 168 |
| 98 | desA1, C125S, R38A, L80F, R81E, L85V, I92F | 197, 198, 201, 202, 27, 28, 35, 36, |
| | | 41, 42, 81, 82, 137, 138, 167, 168 |
| 99 | desA1, C125S, R38A, L80F, R81E, I86V, I92F | 197, 198, 201, 202, 27, 28, 35, 36, |
| | | 41, 42, 81, 82, 131, 132, 167, 168 |
| 100 | desA1, C125S, L80F, R81E, L85Y, I86V, I92F | 197, 198, 201, 202, 25, 26, 29, 30, |
| | | 35, 36, 41, 42, 81, 82, 145, 146, |
| | | 167, 168 |
| 101 | desA1, C125S, L80F, R81E, I86A, I92F | 197, 198, 201, 202, 25, 26, 29, 30, |
| | | 35, 36, 41, 42, 81, 82, 129, 130, |
| | | 167, 168 |
| 102 | desA1, C125S, L80F, R81E, L85V, I86V | 197, 198, 201, 202, 25, 26, 29, 30, |
| | | 35, 36, 41, 42, 81, 82, 139, 140, |
| | | 181, 182 |
| 103 | desA1, C125S, L18R, Q22E, R38A, L80F, R81E, L85V, | 197, 198, 201, 202, 7, 8, 27, 28, |
| | I86V, I92F | 35, 36, 41, 42, 81, 82, 139, 140, |
| | | 167, 168 |
| 104 | desA1, C125S, L18R, Q22E, E61D, L80F, R81E, L85V, | 197, 198, 201, 202, 7, 8, 25, 26, |
| | I86V, I92F | 29, 30, 35, 36, 41, 42, 57, 58, 81, |
| | | 82, 139, 140, 167, 168 |
| 105 | desA1, C125S, L18R, Q22E, E68D, L80F, R81E, L85V, | 197, 198, 201, 202, 7, 8, 25, 26, |
| | I86V, I92F | 29, 30, 35, 36, 41, 42, 63, 64, 81, |
| | | 82, 139, 140, 167, 168 |

In Table 1 above, desA1 represents a deletion of alanine, which is the first amino acid in interleukin-2.

Table 2 below shows full-length protein sequences of interleukin-2 analogs. The letters shown in bold in Table 2 represent the positions for modification.

TABLE 2
Sequences of Interleukin-2 Analogs

| | | SEQ ID | SEQ ID |
|---|---|---|---|
| | | NO: of | NO: of |
| Analog | Protein Sequence | Proteins | Nucleotides |
| 1 | PTSSSTKKT QLQLEHLLLD LQMILNGINN YKNPKLTRML TFKFYMPKKA | 2 | 107 |
| | TELKHLQCLE EELKPLEEVL NLAQSKNFHL RPRDLISNIN VIVLELKGSE | | |
| | TTFMCEYADE TATIVEFLNR WITFSQSIIS TLT | | |
| 2 | PTSSSTKKT QLQLEHLLLD LQMILNGINN YKNPKLTRML TFKFYMPKKA | 3 | 108 |
| | TELKHLQCLE EELKPLEEVL NLAQSKNFHL RPRDLICNIN VIVLELKGSE | | |
| | TTFMCEYADE TATIVEFLNR WITFSQSIIS TLT | | |
| 3 | PTSSSTKKT QLQLEHLLLD LQMILNGINN YCNPKLTRML TFKFYMPKKA | 4 | 109 |
| | TELKHLQCLE EELKPLEEVL NLAQSKNFHL RPRDLISNIN VIVLELKGSE | | |
| | TTFMCEYADE TATIVEFLNR WITFSQSIIS TLT | | |
| 4 | PTSSSTKKT QLQLEHLLLD LQMILNGINN YKNPCLTRML TFKFYMPKKA | 5 | 110 |
| | TELKHLQCLE EELKPLEEVL NLAQSKNFHL RPRDLISNIN VIVLELKGSE | | |
| | TTFMCEYADE TATIVEFLNR WITFSQSIIS TLT | | |
| 5 | PTSSSTKKT QLQLEHLLLD LQMILNGINN YKNPKLTRML TFCFYMPKKA | 6 | 111 |
| | TELKHLQCLE EELKPLEEVL NLAQSKNFHL RPRDLISNIN VIVLELKGSE | | |
| | TTFMCEYADE TATIVEFLNR WITFSQSIIS TLT | | |
| 6 | PTSSSTKKT QLQLEHLLLD LQMILNGINN YKNPKLTRML TFKFYMPCKA | 7 | 112 |
| | TELKHLQCLE EELKPLEEVL NLAQSKNFHL RPRDLISNIN VIVLELKGSE | | |
| | TTFMCEYADE TATIVEFLNR WITFSQSIIS TLT | | |
| 7 | PTSSSTKKT QLQLEHLLLD LQMILNGINN YKNPKLTRML TFKFYMPKCA | 8 | 113 |
| | TELKHLQCLE EELKPLEEVL NLAQSKNFHL RPRDLISNIN VIVLELKGSE | | |
| | TTFMCEYADE TATIVEFLNR WITFSQSIIS TLT | | |
| 8 | PTSSSTKKT QLQLEHLLLD LQMILNGINN YKNPKLTRML TFKFYMPKKA | 9 | 114 |
| | TELKHLQCLE EELKPLEEVL NLAQSCNFHL RPRDLISNIN VIVLELKGSE | | |
| | TTFMCEYADE TATIVEFLNR WITFSQSIIS TLT | | |
| 9 | PTSSSTKKT QLQLEHLLLD LQMILNGINN YKNPKLTAML TFKFYMPKKA | 10 | 115 |
| | TELKHLQCLE EELKPLEEVL NLAQSKNFHL RPRDLISNIN VIVLELKGSE | | |
| | TTFMCEYADE TATIVEFLNR WITFSQSIIS TLT | | |
| 10 | PTSSSTKKT QLQLEHLLLD LQMILNGINN YKNPKLTRML TKKFYMPKKA | 11 | 116 |
| | TELKHLQCLE EELKPLEEVL NLAQSKNFHL RPRDLISNIN VIVLELKGSE | | |
| | TTFMCEYADE TATIVEFLNR WITFSQSIIS TLT | | |
| 11 | PTSSSTKKT QLQLEHLLLD LQMILNGINN YKNPKLTRML TAKFYMPKKA | 12 | 117 |
| | TELKHLQCLE EELKPLEEVL NLAQSKNFHL RPRDLISNIN VIVLELKGSE | | |
| | TTFMCEYADE TATIVEFLNR WITFSQSIIS TLT | | |
| 12 | PTSSSTKKT QLQLEHLLLD LQMILNGINN YKNPKLTAML TKKFYMPKKA | 13 | 118 |
| | TELKHLQCLE EELKPLEEVL NLAQSKNFHL RPRDLISNIN VIVLELKGSE | | |
| | TTFMCEYADE TATIVEFLNR WITFSQSIIS TLT | | |
| 13 | PTSSSTKKT QLQLEHLLLD LQMILNGINN YKNPKLTAML TAKFYMPKKA | 14 | 119 |
| | TELKHLQCLE EELKPLEEVL NLAQSKNFHL RPRDLISNIN VIVLELKGSE | | |
| | TTFMCEYADE TATIVEFLNR WITFSQSIIS TLT | | |
| 14 | PTSSSTKKT QLQLEHLLYD LQMILNGINN YKNPKLTAML TKKFYMPKKA | 15 | 120 |
| | TELKHLQCLE EELKPLEEVL NLAQSKNFHL RPRDLISNIN VIVLELKGSE | | |
| | TTFMCEYADE TATIVEFLNR WITFSQSIIS TLT | | |
| 15 | PTSSSTKKT QLQLEHLLLD LQMILNGINN YKNPKLTAML TKKFYMPKKA | 16 | 121 |
| | TELKHLQCLE EELKPLEEVL NLAQSKNFHL RPRELISNIN VIVLELKGSE | | |
| | TTFMCEYADE TATIVEFLNR WITFSQSIIS TLT | | |
| 16 | PTSSSTKKT QLQLEHLLLD LQMILNGINN YKNPKLTAML TKKFYMPKKA | 17 | 122 |
| | TELKHLQCLE EELKPLEEVL NLAQSKNFHL RPRDLISQIN VIVLELKGSE | | |
| | TTFMCEYADE TATIVEFLNR WITFSQSIIS TLT | | |
| 17 | PTSSSTKKT QLQLEHLLLD LQMILNGINN YKNPKLTAML TKKFYMPKKA | 18 | 123 |
| | TELKHLQCLE EELKPLEEVL NLAQSKNFHL RPRDLISNIN TIVLELKGSE | | |
| | TTFMCEYADE TATIVEFLNR WITFSQSIIS TLT | | |
| 18 | PTSSSTKKT QLQLEHLLLD LQMILNGINN YKNPKLTAML TKKFYMPKKA | 19 | 124 |
| | TELKHLQCLE QELKPLEEVL NLAQSKNFHL RPRDLISNIN VIVLELKGSE | | |
| | TTFMCEYADE TATIVEFLNR WITFSQSIIS TLT | | |
| 19 | PTSSSTKKT QLQLEHLLLD LQMILNGINN YKNPKLTAML TKKFYMPKKA | 20 | 125 |
| | TELKHLQCLE EELKPLEEVL NLAQSKNFHL DPRDLISNIN VFVLELKGSE | | |
| | TTFMCEYADE TATIVEFLNR WITFSQSIIS TLT | | |
| 20 | PTSSSTKKT QLQLEHLLLD LQMILNGINN YKNPKLTAML TKKFYMPKKA | 21 | 126 |
| | TELKHLQCLE EELKPLEEVL NLAQSKNFHL RPRDVVSNIN VFVLELKGSE | | |
| | TTFMCEYADE TATIVEFLNR WITFSQSIIS TLT | | |
| 21 | PTSSSTKKT QLQLEHLLLD LQMILNGINN YKNPKLTAML TKKFYMPKKA | 22 | 127 |
| | TELKHLQCLE EELKPLEEVL NLAQSKNFHF DPRDVVSNIN VFVLELKGSE | | |
| | TTFMCEYADE TATIVEFLNR WITFSQSIIS TLT | | |
| 22 | PTSSSTKKT QVQLEHLLLD LQMILNGINN YKNPKLTAML TKKFYMPKKA | 23 | 128 |
| | TELKHLQCLE EELKPLEEVL NLAQSKNFHL RPRDLISNIN VIVLELKGSE | | |
| | TTFMCEYADE TATIVEFLNR WITFSQSIIS TLT | | |
| 23 | PTSSSTKKT QFQLEHLLLD LQMILNGINN YKNPKLTAML TKKFYMPKKA | 24 | 129 |
| | TELKHLQCLE EELKPLEEVL NLAQSKNFHL RPRDLISNIN VIVLELKGSE | | |
| | TTFMCEYADE TATIVEFLNR WITFSQSIIS TLT | | |
| 24 | PTSSSTKKT QLQLEHLLVD LQMILNGINN YKNPKLTAML TKKFYMPKKA | 25 | 130 |
| | TELKHLQCLE EELKPLEEVL NLAQSKNFHL RPRDLISNIN VIVLELKGSE | | |
| | TTFMCEYADE TATIVEFLNR WITFSQSIIS TLT | | |
| 25 | PTSSSTKKT QLQLEHLLFD LQMILNGINN YKNPKLTAML TKKFYMPKKA | 26 | 131 |
| | TELKHLQCLE EELKPLEEVL NLAQSKNFHL RPRDLISNIN VIVLELKGSE | | |
| | TTFMCEYADE TATIVEFLNR WITFSQSIIS TLT | | |
| 26 | PTSSSTKKT QLQLEHLLLD LQMILNGINN YKNPKLTAML TKKFYMPKKA | 27 | 132 |
| | TELKHLQCLE EELKPLEEVL NLAQSKNFHL RPRDLISNFN VIVLELKGSE | | |
| | TTFMCEYADE TATIVEFLNR WITFSQSIIS TLT | | |
| 27 | PTSSSTKKT QLQLEHLLLD LQMILNGINN YKNPKLTAML TKKFYMPKKA | 28 | 133 |
| | TELKHLQCLE EELKPLEEVL NLAQSKNFHL RPRDLISNIN FIVLELKGSE | | |
| | TTFMCEYADE TATIVEFLNR WITFSQSIIS TLT | | |
| 28 | PTSSSTKKT QLQLEHLLLD LQMILNGINN YKNPKLTAML TKKFYMPKKA | 29 | 134 |
| | TELKHLQCLE EELKPLEEVL NLAQSKNFHL RPRDLISNIN VIVVELKGSE | | |
| | TTFMCEYADE TATIVEFLNR WITFSQSIIS TLT | | |
| 29 | PTSSSTKKT QLQLEHLLLD LQMILNGINN YKNPKLTAML TKKFYMPKKA | 30 | 135 |
| | TELKHLQCLE EELKPLEEVL NLAQSKNFHL RPRDLISNIN VIVLELKGSE | | |
| | TTFMCEYADE TATIVEFLNR WITFSTSIIS TLT | | |
| 30 | PTSSSTKKT QLQLEHLLLD LQMILNGINN YKNPKLTAML TFKFYMPKKA | 31 | 136 |
| | TELKHLQCLE EELKPLEEVL NLAQSKNFHL DPRDLISNIN VFVLELKGSE | | |
| | TTFMCEYADE TATIVEFLNR WITFSQSIIS TLT | | |
| 31 | PTSSSTKKT QLQLEHLLLD LQMILNGINN YKNPKLTAML TFKFYMPKKA | 32 | 137 |
| | TELKHLQCLE EELKPLEEVL NLAQSKNFHL RPRELISNIN VIVLELKGSE | | |
| | TTFMCEYADE TATIVEFLNR WITFSQSIIS TLT | | |
| 32 | PTSSSTKKT QLQLEHLLLD LQMILNGINN YKNPKLTAML TFKFYMPKKA | 33 | 138 |
| | TELKHLQCLE EELKPLEEVL NLAQSKNFHL DPRELISNIN VFVLELKGSE | | |
| | TTFMCEYADE TATIVEFLNR WITFSQSIIS TLT | | |
| 33 | PTSSSTKKT QLQLEHLLLD LQMILNGINN YKNPKLTAML TFKFYMPKKA | 34 | 139 |
| | TELKHLQCLE EELKPLEEVL NLAQSKNFHF RPRDLISNIN VIVLELKGSE | | |
| | TTFMCEYADE TATIVEFLNR WITFSQSIIS TLT | | |
| 34 | PTSSSTKKT QLQLEHLLLD LQMILNGINN YKNPKLTAML TFKFYMPKKA | 35 | 140 |
| | TELKHLQCLE EELKPLEEVL NLAQSKNFHF RPRELISNIN VIVLELKGSE | | |
| | TTFMCEYADE TATIVEFLNR WITFSQSIIS TLT | | |
| 35 | PTSSSTKKT QLQLEHLLLD LQMILNGINN YKNPKLTAML TFKFYMPKKA | 36 | 141 |
| | TELKHLQCLE EELKPLEEVL NLAQSKNFHL RPRDLISNIN VIVFEFKGSE | | |
| | TTFMCEYADE TATIVEFLNR WITFSQSIIS TLT | | |
| 36 | PTSSSTKKT QLQLEHLLLD LQMILNGINN YKNPKLTAML TFKFYMPKKA | 37 | 142 |
| | TELKHLQCLE EELKPLEEVL NLAQSKNFHL RPRDLISNIN VIVFEVKGSE | | |
| | TTFMCEYADE TATIVEFLNR WITFSQSIIS TLT | | |
| 37 | PTSSSTKKT QLQLEHLLLD LQMILNGINN YKNPKLTAML TFKFYMPKKA | 38 | 143 |
| | TELKHLQCLE EELKPLEEVL NLAQSKNFHL RPRDLISNIN VIVFEIKGSE | | |
| | TTFMCEYADE TATIVEFLNR WITFSQSIIS TLT | | |
| 38 | PTSSSTKKT QLQLEHLLLD LQMILNGINN YKNPKLTAML TKKFYMPKKA | 39 | 144 |
| | TELKHLQCLE EELKPLEEVL NLAQSKNFHL DPRDLISNIN VFVFEFKGSE | | |
| | TTFMCEYADE TATIVEFLNR WITFSQSIIS TLT | | |
| 39 | PTSSSTKKT QLQLEHLLLD LQMILNGINN YKNPKLTAML TKKFYMPKKA | 40 | 145 |
| | TELKHLQCLE EELKPLEEVL NLAQSKNFHL DPRDLISNIN VFVFEVKGSE | | |
| | TTFMCEYADE TATIVEFLNR WITFSQSIIS TLT | | |
| 40 | PTSSSTKKT QLQLEHLLLD LQMILNGINN YKNPKLTAML TKKFYMPKKA | 41 | 146 |
| | TELKHLQCLE EELKPLEEVL NLAQSKNFHL DPRDLISNIN VFVFEIKGSE | | |
| | TTFMCEYADE TATIVEFLNR WITFSQSIIS TLT | | |
| 41 | PTSSSTKKT QLQLEHLLLD LQMILNGINN YKNPKLTRML TFKFYMPKKA | 42 | 147 |
| | TELKHLQCLE EELKPLEEVL NLAQSKNFHF DPRDVVSNIN VFVLELKGSE | | |
| | TTFMCEYADE TATIVEFLNR WITFSQSIIS TLT | | |
| 42 | PTSSSTKKT QLQLEHLLLD LQMILNGINN YKNPKLTAML TKKFYMPKKA | 43 | 148 |
| | TELKHLQCLE EELKPLEEVL NLAQSKNFHL EPRDLISNIN VFVLELKGSE | | |
| | TTFMCEYADE TATIVEFLNR WITFSQSIIS TLT | | |
| 43 | PTSSSTKKT QLQLEHLLLD LQMILNGINN YKNPKLTAML TKKFYMPKKA | 44 | 149 |
| | TELKHLQCLE EELKPLEEVL NLAQSKNFHL DPRDLISNIN VLVLELKGSE | | |
| | TTFMCEYADE TATIVEFLNR WITFSQSIIS TLT | | |
| 44 | PTSSSTKKT QLQLEHLLLD LQMILNGINN YKNPKLTAML TKKFYMPKKA | 45 | 150 |
| | TELKHLQCLE EELKPLEEVL NLAQSKNFHL DPRVLISNIN VFVLELKGSE | | |
| | TTFMCEYADE TATIVEFLNR WITFSQSIIS TLT | | |
| 45 | PTSSSTKKT QLQLEHLLLD LQMILNGINN YKNPKLTAML TKKFYMPKKA | 46 | 151 |
| | TELKHLQCLE EELKPLEEVL NLAQSKNFHL DPRFLISNIN VFVLELKGSE | | |
| | TTFMCEYADE TATIVEFLNR WITFSQSIIS TLT | | |
| 46 | PTSSSTKKT QLQLEHLLLV LQMILNGINN YKNPKLTAML TKKFYMPKKA | 47 | 152 |
| | TELKHLQCLE EELKPLEEVL NLAQSKNFHL DPRDLISNIN VFVLELKGSE | | |
| | TTFMCEYADE TATIVEFLNR WITFSQSIIS TLT | | |
| 47 | PTSSSTKKT QLQLEHLLLF LQMILNGINN YKNPKLTAML TKKFYMPKKA | 48 | 153 |
| | TELKHLQCLE EELKPLEEVL NLAQSKNFHL DPRDLISNIN VFVLELKGSE | | |
| | TTFMCEYADE TATIVEFLNR WITFSQSIIS TLT | | |
| 48 | PTSSSTKKT QLQLEHLLLD LQMILNGINN YKNPKLTAML TKKFYMPKKA | 49 | 154 |
| | TELKHLQCLE EELKPLEEVL NLAQSKNFHL DPRDLISVIN VFVLELKGSE | | |
| | TTFMCEYADE TATIVEFLNR WITFSQSIIS TLT | | |
| 49 | PTSSSTKKT QLQLEHLLLD LQMILNGINN YKNPKLTAML TKKFYMPKKA | 50 | 155 |
| | TELKHLQCLE EELKPLEEVL NLAQSKNFHL DPRDLISFIN VFVLELKGSE | | |
| | TTFMCEYADE TATIVEFLNR WITFSQSIIS TLT | | |
| 50 | PTSSSTKKT QLQLEHLLLD LQMILNGINN YKNPKLTRML TKKFYMPKKA | 51 | 156 |
| | TELKHLQCLE EELKPLEEVL NLAQSKNFHF DPRDVVSNIN VFVLELKGSE | | |
| | TTFMCEYADE TATIVEFLNR WITFSQSIIS TLT | | |
| 51 | PTSSSTKKT QLQLEHLLLD LQMILNGINN YKNPKLTRML TFKFYMPKKA | 52 | 157 |
| | TELKHLQCLE QELKPLEEVL NLAQSKNFHL DPRDLISNIN VFVLELKGSE | | |
| | TTFMCEYADE TATIVEFLNR WITFSQSIIS TLT | | |
| 52 | PTSSSTKKT QLQLEHLLLD LQMILNGINN YKNPKLTAML TKKFYMPKKA | 53 | 158 |
| | TELKHLQCLE EELKPLEEVL NLAQSKNFHF DPRDLISNIN VFVLELKGSE | | |
| | TTFMCEYADE TATIVEFLNR WITFSQSIIS TLT | | |
| 53 | PTSSSTKKT QLQLEHLLLD LQMILNGINN YKNPKLTAML TKKFYMPKKA | 54 | 159 |
| | TELKHLQCLE EELKPLEEVL NLAQSKNFHF DPRELISNIN VFVLELKGSE | | |
| | TTFMCEYADE TATIVEFLNR WITFSQSIIS TLT | | |
| 54 | PTSSSTKKT QLQLEHLLLD LQMILNGINN YKNPKLTAML TKKFYMPKKA | 55 | 160 |
| | TELKHLQCLE EELKPLEEVL NLAHSKNFHL DPRDLISNIN VFVLELKGSE | | |
| | TTFMCEYADE TATIVEFLNR WITFSQSIIS TLT | | |
| 55 | PTSSSTKKT QLQLEHLLLD LQMILNGINN YKNPKLTAML TKKFYMPKKA | 56 | 161 |
| | TELKHLQCLE EELKPLEEVL NLAHSKNFHF DPRDLISNIN VFVLELKGSE | | |
| | TTFMCEYADE TATIVEFLNR WITFSQSIIS TLT | | |
| 56 | PTSSSTKKT QLQLEHLLLD LQMILNGINN YKNPKLTAML TKKFAMPKKA | 57 | 162 |
| | TELKHLQCLE EELKPLEEVL NLAHSKNFHL DPRDLISNIN VFVLELKGSE | | |
| | TTFMCEYADE TATIVEFLNR WITFSQSIIS TLT | | |
| 57 | PTSSSTKKT QLQLEHLLLD LQMILNGINN YKNPKLTAML TKKFAMPKKA | 58 | 163 |
| | TELKHLQCLE EELKPLEEVL NLAHSKNFHF DPRDLISNIN VFVLELKGSE | | |
| | TTFMCEYADE TATIVEFLNR WITFSQSIIS TLT | | |
| 58 | PTSSSTKKT QLQLEHLLLD LQMILNGINN YKNPKLTAML TKKFYMPKKA | 59 | 164 |
| | TELKHLQCLE EELKPLEEVL NLAQSKNFHF DPRDAASNIN VFVLELKGSE | | |
| | TTFMCEYADE TATIVEFLNR WITFSQSIIS TLT | | |
| 59 | PTSSSTKKT QLQLEHLLLD LQMILNGINN YKNPKLTAML TKKFYMPKKA | 60 | 165 |
| | TELKHLQCLE EELKPLEEVL NLAQSKNFHF DPRDAASNIN VYVLELKGSE | | |
| | TTFMCEYADE TATIVEFLNR WITFSQSIIS TLT | | |
| 60 | PTSSSTKKT QLQLEHLLLD LQMILNGINN YKNPKLTAML TKKFAMPKKA | 61 | 166 |
| | TELKHLQCLE EELKPLEEVL NLAQSKNFHY RPRDAASNIN VYVLELKGSE | | |
| | TTFMCEYADE TATIVEFLNR WITFSQSIIS TLT | | |
| 61 | PTSSSTKKT QLQLEHLLLD LQMILNGINN YKNPKLTAML TKKFYMPKKA | 62 | 167 |
| | TELKHLQCLE EELKPLEEVL NLAQSKNFHY DPRDGVSNIN VYVLELKGSE | | |
| | TTFMCEYADE TATIVEFLNR WITFSQSIIS TLT | | |
| 62 | PTSSSTKKT QLQLEHLLLD LQMILNGINN YKNPKLTAML TKKFYMPKKA | 63 | 168 |
| | TELKHLQCLE EELKPLEEVL NLAQSKNFHW EPRDGASNIN VFVLELKGSE | | |
| | TTFMCEYADE TATIVEFLNR WITFSQSIIS TLT | | |
| 63 | PTSSSTKKT QLQLEHLLLD LQMILNGINN YKNPKLTAML TKKFYMPKKA | 64 | 169 |
| | TELKHLQCLE EELKPLEEVL NLAQSKNFHD EPRDTGSNIN VFVLELKGSE | | |
| | TTFMCEYADE TATIVEFLNR WITFSQSIIS TLT | | |
| 64 | PTSSSTKKT QLQLEHLLLD LQMILNGINN YKNPKLTAML TKKFYMPKKA | 65 | 170 |
| | TELKHLQCLE EELKPLEEVL NLAQSKNFHY NPRDVVSNIN VYVLELKGSE | | |
| | TTFMCEYADE TATIVEFLNR WITFSQSIIS TLT | | |
| 65 | PTSSSTKKT QLQLEHLLLD LQMILNGINN YKNPKLTAML TKKFYMPKKA | 66 | 171 |
| | TELKHLQCLE EELKPLEEVL NLAQSKNFHY EPRDVVSNIN VFVLELKGSE | | |
| | TTFMCEYADE TATIVEFLNR WITFSQSIIS TLT | | |
| 66 | PTSSSTKKT QLQLEHLLLD LQMILNGINN YKNPKLTAML TKKFYMPKKA | 67 | 172 |
| | TELKHLQCLE EELKPLEEVL NLAQSKNFHF EPRDFVSNIN VFVLELKGSE | | |
| | TTFMCEYADE TATIVEFLNR WITFSQSIIS TLT | | |
| 67 | PTSSSTKKT QLQLEHLLLD LQMILNGINN YKNPKLTAML TKKFYMPKKA | 68 | 173 |
| | TELKHLQCLE EELKPLEEVL NLAQSKNFHY DPRDFVSNIN VWVLDLKGSE | | |
| | TTFMCEYADE TATIVEFLNR WITFSQSIIS TLT | | |
| 68 | PTSSSTKKT QLQLEHLLLD LQMILNGINN YKNPKLTAML TKKFYMPKKA | 69 | 174 |
| | TELKHLQCLE EELKPLEEVL NLAQSKNFHF EPRDIVSNIN EFVLELKGSE | | |
| | TTFMCEYADE TATIVEFLNR WITFSQSIIS TLT | | |
| 69 | PTSSSTKKT QLQLEHLLLD LQMILNGINN YKNPKLTAML TKKFYMPKKA | 70 | 175 |
| | TELKHLQCLE EELKPLEEVL NLAQSKNFHY EPRDFLSNIN EWVLDLKGSE | | |
| | TTFMCEYADE TATIVEFLNR WITFSQSIIS TLT | | |
| 70 | PTSSSTKKT QLQLEHLLLD LQMILNGINN YKNPKLTAML TKKFYMPKKA | 71 | 176 |
| | TELKHLQCLE EELKPLEEVL NLAQSKNFHY DPRDVVSNIN VFVLELKGSE | | |
| | TTFMCEYADE TATIVEFLNR WITFSQSIIS TLT | | |
| 71 | PTSSSTKKT QLQLEHLLLD LQMILNGINN YKNPKLTAML TKKFYMPKKA | 72 | 177 |
| | TELKHLQCLE EELKPLEEVL NLAQSKNFHF EPRDVVSNIN VFVLELKGSE | | |
| | TTFMCEYADE TATIVEFLNR WITFSQSIIS TLT | | |
| 72 | PTSSSTKKT QLQLEHLLLD LQMILNGINN YKNPKLTAML TKKFYMPKKA | 73 | 178 |
| | TELKHLQCLE EELKPLEEVL NLAQSKNFHF DPRDVGSNIN VFVLELKGSE | | |
| | TTFMCEYADE TATIVEFLNR WITFSQSIIS TLT | | |
| 73 | PTSSSTKKT QLQLEHLLLD LQMILNGINN YKNPKLTAML TKKFYMPKKA | 74 | 179 |
| | TELKHLQCLE EELKPLEEVL NLAQSKNFHF DPRDWVSNIN VFVLELKGSE | | |
| | TTFMCEYADE TATIVEFLNR WITFSQSIIS TLT | | |
| 74 | PTSSSTKKT QLQLEHLLLD LQMILNGINN YKNPKLTDML TKKFYMPKKA | 75 | 180 |
| | TELKHLQCLE EELKPLEEVL NLAQSKNFHY DPRDVVSNIN VFVLELKGSE | | |
| | TTFMCEYADE TATIVEFLNR WITFSQSIIS TLT | | |
| 75 | PTSSSTKKT QLQLEHLLLD LQMILNGINN YKNPKLTAML TKKFAMPKKA | 76 | 181 |
| | TELKHLQCLE EELKPLEEVL NLAQSKNFHF EPRDVVSNIN VFVLELKGSE | | |
| | TTFMCEYADE TATIVEFLNR WITFSQSIIS TLT | | |
| 76 | PTSSSTKKT QLQLEHLLLD LQMILNGINN YKNPKLTAML TKQFYMPKKA | 77 | 182 |
| | TELKHLQCLE RELKPLEEVL NLAQSKNFHF DPRDVGSNIN VFVLELKGSE | | |
| | TTFMCEYADE TATIVEFLNR WITFSQSIIS TLT | | |
| 77 | PTSSSTKKT QLQLEHLLLD LQMILNGINN YKNPKLTAML TKEFYMPKKA | 78 | 183 |
| | TELKHLQCLE RELKPLEEVL NLAQSKNFHF DPRDWVSNIN VFVLELKGSE | | |
| | TTFMCEYADE TATIVEFLNR WITFSQSIIS TLT | | |
| 78 | PTSSSTKKT QLQLEHLLLD LQMILNGINN YKNPELTAML TKKFYMPKKA | 79 | 184 |
| | TELKHLQCLE EELKPLEEVL NLAQSKNFHF EPRDVVSNIN VFVLELKGSE | | |
| | TTFMCEYADE TATIVEFLNR WITFSQSIIS TLT | | |
| 79 | PTSSSTKKT QLQLEHLLLD LQMILNGINN YKNPELTAML TKKFYMPKKA | 80 | 185 |
| | TELKHLQCLE EELKPLEEVL NLAHSKNFHF EPRDVVSNIN VFVLELKGSE | | |
| | TTFMCEYADE TATIVEFLNR WITFSQSIIS TLT | | |
| 80 | PTSSSTKKT QLQLEHLLLD LQMILNGINN YKNPELTAML TKKFYMPKKA | 81 | 186 |
| | TELKHLQCLE EELKPLEEVL NLAHSKNFHF EGRDVVSNIN VFVLELKGSE | | |
| | TTFMCEYADE TATIVEFLNR WITFSQSIIS TLT | | |
| 81 | PTSSSTKKT QLQLEHLLLD LQMILNGINN YKNPELTAML TKKFYMPKKA | 82 | 187 |
| | TELKHLQCLE EELKPLEEVL NLAHSKNFHF EVRDVVSNIN VFVLELKGSE | | |
| | TTFMCEYADE TATIVEFLNR WITFSQSIIS TLT | | |
| 82 | PTSSSTKKT QLQLEHLRLD LEMILNGINN YKNPKLTRML TFKFYMPKKA | 83 | 188 |
| | TELKHLQCLE EELKPLEEVL NLAQSKNFHF DPRDEVSNIN VFVLELKGSE | | |
| | TTFMCEYADE TATIVEFLNR WITFSQSIIS TLT | | |
| 83 | PTSSSTKKT QLQLEHLRRD LEMILNGINN YKNPKLTRML TFKFYMPKKA | 84 | 189 |
| | TELKHLQCLE EELKPLEEVL NLAQSKNFHF DPRDEVSNIN VFVLELKGSE | | |
| | TTFMCEYADE TATIVEFLNR WITFSQSIIS TLT | | |
| 84 | PTSSSTKKT QLQLEHLRLD LEMILNGINN YKNPKLTRML TFKFYMPKKA | 85 | 190 |
| | TELKHLQCLE EELKPLEEVL NLAQSKNFHV DPRDEVSNIN VFVLELKGSE | | |
| | TTFMCEYADE TATIVEFLNR WITFSQSIIS TLT | | |
| 85 | PTSSSTKKT QLQLEHLLLD LQMILNGINN YKNPKLTRML TFKFYMPKKA | 86 | 191 |
| | TELKHLQCLE EELKPLEEVL NLAQSKNFHF EPRDVVSNIN VFVLELKGSE | | |
| | TTFMCEYADE TATIVEFLNR WITFSQSIIS TLT | | |
| 86 | PTSSSTKKT QLQLEHLRLD LEMILNGINN YKNPKLTRML TFKFYMPKKA | 87 | 192 |
| | TELKHLQCLE EELKPLEEVL NLAQSKNFHF EPRDVVSNIN VFVLELKGSE | | |
| | TTFMCEYADE TATIVEFLNR WITFSQSIIS TLT | | |
| 87 | PTSSSTKKT QLQLEHLRRD LEMILNGINN YKNPKLTRML TFKFYMPKKA | 88 | 193 |
| | TELKHLQCLE EELKPLEEVL NLAQSKNFHF EPRDVVSNIN VFVLELKGSE | | |
| | TTFMCEYADE TATIVEFLNR WITFSQSIIS TLT | | |
| 88 | PTSSSTKKT QLQLEHLLLD LQMILNGINN YKNPKLTRML TFKFYMPKKA | 89 | 194 |
| | TELKHLQCLE DELKPLEEVL NLAQSKNFHF EPRDVVSNIN VFVLELKGSE | | |
| | TTFMCEYADE TATIVEFLNR WITFSQSIIS TLT | | |
| 89 | PTSSSTKKT QLQLEHLLLD LQMILNGINN YKNPKLTAML TFKFYMPKKA | 90 | 195 |
| | TELKHLQCLE EELKPLEQVL NLAQSKNFHF EPRDVVSNIN VFVLELKGSE | | |
| | TTFMCEYADE TATIVEFLNR WITFSQSIIS TLT | | |
| 90 | PTSSSTKKT QLQLEHLLLD LQMILNGINN YKNPKLTRML TWKFYMPKKA | 91 | 196 |
| | TELKHLQCLE EELKPLEEVL NLAQSKNFHF EPRDVVSNIN VFVLELKGSE | | |
| | TTFMCEYADE TATIVEFLNR WITFSQSIIS TLT | | |
| 91 | PTSSSTKKT QLQLEHLLLD LQMILNGINN YKNPKLTRML TFKFYMPKKA | 92 | 197 |
| | TELKHLQCLE QELKPLEEVL NLAQSKNFHF EPRDVVSNIN VFVLELKGSE | | |
| | TTFMCEYADE TATIVEFLNR WITFSQSIIS TLT | | |
| 92 | PTSSSTKKT QLQLEHLLLD LQMILNGINN YKNPKLTRML TFKFYMPKKA | 93 | 198 |
| | TELKHLQCLE EELKPLEEVL NLAQSKNFHF EPRDVVSNIN TFVLELKGSE | | |
| | TTFMCEYADE TATIVEFLNR WITFSQSIIS TLT | | |
| 93 | PTSSSTKKT QLQLEHLLLD LQMILNGINN YKNPKLTRML TFKFYMPKKA | 94 | 199 |
| | TELKHLQCLE EELKPLEEVL NLAQSKNFHF EPREVVSNIN TFVLELKGSE | | |
| | TTFMCEYADE TATIVEFLNR WITFSQSIIS TLT | | |
| 94 | PTSSSTKKT QLQLEHLLLD LQMILNGINN YKNPKLTRML TFKFYMPKKA | 95 | \|200 |
| | TELKHLQCLE EELKPLEEVL NLAQSKNFHF EPRDVVSNIN VFVFEFKGSE | | |
| | TTFMCEYADE TATIVEFLNR WITFSQSIIS TLT | | |
| 95 | PTSSSTKKT QLQLEHLLLD LQMILNGINN YKNPKLTRML TFKFYMPKKA | 96 | 201 |
| | TELKHLQCLE EELKPLEEGL NLAQSKNFHF EPRDVVSNIN VFVLELKGSE | | |
| | TTFMCEYADE TATIVEFLNR WITFSQSIIS TLT | | |
| 96 | PTSSSTKKT QLQLEHLLLD LQMILNGINN YKNPKLTRML TFKFYMPKKA | 97 | 202 |
| | TELKHLQCLE EELKPLEEGL NLAASKNFHF EPRDVVSNIN VFVLELKGSE | | |
| | TTFMCEYADE TATIVEFLNR WITFSQSIIS TLT | | |
| 97 | PTSSSTKKT QLQLEHLLLD LQMILNGINN YKNPKLTAML TFKFYMPKKA | 98 | 203 |
| | TELKHLQCLE EELKPLEEVL NLAQSKNFHF DPRDLISNIN VFVLELKGSE | | |
| | TTFMCEYADE TATIVEFLNR WITFSQSIIS TLT | | |
| 98 | PTSSSTKKT QLQLEHLLLD LQMILNGINN YKNPKLTAML TFKFYMPKKA | 99 | 204 |
| | TELKHLQCLE EELKPLEEVL NLAQSKNFHF EPRDVISNIN VFVLELKGSE | | |
| | TTFMCEYADE TATIVEFLNR WITFSQSIIS TLT | | |
| 99 | PTSSSTKKT QLQLEHLLLD LQMILNGINN YKNPKLTAML TFKFYMPKKA | 100 | 205 |
| | TELKHLQCLE EELKPLEEVL NLAQSKNFHF EPRDLVSNIN VFVLELKGSE | | |
| | TTFMCEYADE TATIVEFLNR WITFSQSIIS TLT | | |
| 100 | PTSSSTKKT QLQLEHLLLD LQMILNGINN YKNPKLTRML TFKFYMPKKA | 101 | 206 |
| | TELKHLQCLE EELKPLEEVL NLAQSKNFHF EPRDYVSNIN VFVLELKGSE | | |
| | TTFMCEYADE TATIVEFLNR WITFSQSIIS TLT | | |
| 101 | PTSSSTKKT QLQLEHLLLD LQMILNGINN YKNPKLTRML TFKFYMPKKA | 102 | 207 |
| | TELKHLQCLE EELKPLEEVL NLAQSKNFHF EPRDLASNIN VFVLELKGSE | | |
| | TTFMCEYADE TATIVEFLNR WITFSQSIIS TLT | | |
| 102 | PTSSSTKKT QLQLEHLLLD LQMILNGINN YKNPKLTRML TFKFYMPKKA | 103 | 208 |
| | TELKHLQCLE EELKPLEEVL NLAQSKNFHF EPRDVVSNIN VIVLELKGSE | | |
| | TTFMCEYADE TATIVEFLNR WITFSQSIIS TLT | | |
| 103 | PTSSSTKKT QLQLEHLRLD LEMILNGINN YKNPKLTAML TFKFYMPKKA | 104 | 209 |
| | TELKHLQCLE EELKPLEEVL NLAQSKNFHF EPRDVVSNIN VFVLELKGSE | | |
| | TTFMCEYADE TATIVEFLNR WITFSQSIIS TLT | | |
| 104 | PTSSSTKKT QLQLEHLRLD LEMILNGINN YKNPKLTRML TFKFYMPKKA | 105 | 210 |
| | TELKHLQCLE DELKPLEEVL NLAQSKNFHF EPRDVVSNIN VFVLELKGSE | | |
| | TTFMCEYADE TATIVEFLNR WITFSQSIIS TLT | | |
| 105 | PTSSSTKKT QLQLEHLRLD LEMILNGINN YKNPKLTRML TFKFYMPKKA | 106 | 211 |
| | TELKHLQCLE EELKPLEDVL NLAQSKNFHF EPRDVVSNIN VFVLELKGSE | | |
| | TTFMCEYADE TATIVEFLNR WITFSQSIIS TLT | | |

### Example 2: Expression of Interleukin-2 Analogs

A recombinant interleukin-2 analog under the control of T7 promoter was expressed using the expression vectors prepared in Example 1. An expression E. coli strain, E. coli BL21DE3 (E. coli B F⁻dcm ompT hsdS (r_{B}⁻m_{B}⁻) gal λ(DE3); Novagen), was transformed with each recombinant interleukin-2 analog expression vector. As for the transformation method, a method recommended by Novagen was used. Each single colony, in which each recombinant expression vector was transformed, was collected, inoculated into a 2× Luria Broth medium containing ampicillin (50 μg/mL), and cultured at 37° C. for 15 hours. Each recombinant strain culture solution and the 2× LB medium containing 30% glycerol were mixed at a 1:1 (v/v) ratio, and each 1 mL of the mixture was dispensed into a cryo-tube, and stored at −150° C. This was used as a cell stock for the production of a recombinant protein.

For the expression of each recombinant interleukin-2 analog, one vial of each cell stock was dissolved, inoculated into 500 mL of 2× LB, and cultured with shaking at 37° C. for 14 to 16 hours. When the absorbance value at 600 nm reached 4.0 or higher, the culture was terminated, and this was used as a seed culture solution. The seed culture was inoculated into 1.6 L of a fermentation medium, and initial fermentation was started Using a 5 L fermentor (Bioflo-320, NBS, USA). Culture conditions were maintained at a pH of 6.70 using a temperature of 37° C., an air volume of 2.0 L/min (1 vvm), a stirring speed of 650 rpm, and 30% aqueous ammonia. As for the fermentation process, when nutrients in the culture medium were limited, fed-batch culture was performed by adding an additional medium (feeding solution). The growth of the strain was observed by absorbance, and a final concentration of 500 μM IPTG was introduced at an absorbance value of 70 or higher. The culture was performed further until for about 23 to 25 hours after the introduction of IPTG, and after termination of the culture, and the recombinant strain was recovered using a centrifuge and stored at −80° C. until use.

### Example 3: Extraction and Refolding of Interleukin-2 Analogs

In order to convert the interleukin-2 analogs from the interleukin-2 analog expressing E. coli obtained in Example 2 in a soluble form, cells were disrupted and refolded. Cell pellets corresponding to 100 mL of the culture were suspended in 1-200 mL of a lysis buffer solution (20 mM Tris-HCl (pH 9.0), 1 mM EDTA (pH 9.0), 0.2 M NaCl, 0.5% Triton X-100), and the recombinant E. coli cells were disrupted at 15,000 psi using a microfluidizer. After centrifugation at 13,900 g for 30 minutes, the supernatant was discarded, and the pellet was washed with 400 mL of a first washing buffer solution (50 mM Tris-HCl (pH 8.0), 5 mM EDTA (pH 9.0)). After centrifugation under the same conditions as above, the supernatant was discarded, and the pellet was washed with 400 ml of a second washing buffer solution (50 mM Tris-HCl (pH 8.0), 5 mM EDTA (pH 9.0), 2% Triton X-100). After centrifugation under the same conditions as above, the supernatant was discarded, and the pellet was washed with 400 mL of a third washing buffer solution (50 mM Tris-HCl (pH 8.0), 5 mM EDTA (pH 9.0), 1% sodium deoxycholorate). After centrifugation under the same conditions as above, the supernatant was discarded, and the pellet was washed with 400 mL of a fourth washing buffer solution (50 mM Tris-HCl (pH 8.0), 5 mM EDTA (pH 9.0), 1 M NaCl). The resultant was subjected to centrifugation under the same conditions as above and washing, and E. coli inclusion bodies were obtained therefrom. The pellet of the washed inclusion bodies was resuspended in 400 mL of soluble/reducing buffer (6 M guanidine, 100 mM Tris (pH 8.0), 2 mM EDTA (pH 9.0), 50 mM DTT) and stirred at 50° C. for 30 minutes. To the soluble/reduced interleukin-2 analogs, 100 mL of distilled water was added to dilute the 6 M guanidine to 4.8 M guanidine, and then the resultant was centrifuged at 13,900 g for 30 minutes and the pellet discarded to obtain only the solution therein. To the diluted solution was additionally added 185.7 mL of distilled water, and the 4.8 M guanidine was diluted to 3.5 M guanidine, and the pH was adjusted to 5.0 using 100% acetic acid. The pH-adjusted solution was stirred at room temperature for one hour. The solution with precipitated impurities was centrifuged at 13,900 g for 30 minutes, and the supernatant was discarded and the pellet washed with a final washing buffer solution (3.5 M guanidine, 20 mM sodium acetate (pH 5.0), 5 mM DTT). The resultant was centrifuged under the same conditions as above to obtain a pellet. The washed interleukin-2 analogs were dissolved in 400 mL of a refolding buffer solution (6 mM guanidine, 100 mM Tris (pH 8.0), 0.1 mM CuCl₂). The refolding process was performed by stirring the mixed solution at 4° C. for 15 to 24 hours.

### Example 4: Size-Exclusion Column Chromatography

The interleukin-2 analog refolding solution obtained in Example 3 was concentrated to less than 1 mL to be applied to a size-exclusion column for purification. The column was equilibrated with a buffer solution (2 M guanidine, 100 mM Tris (pH 8.0)) before introducing with the refolding solution and was eluted by flowing a buffer solution thereto after the introduction of the refolding solution. Since the eluted sample contained guanidine, it was replaced with a stabilized solution (10 mM sodium acetate (pH 4.5), 5% trehalose), and the purity was measured through RP-HPLC and peptide mapping analysis. The sample was used in the experiment when its measured purity reached 80% or higher.

### Example 5: Evaluation of Binding Affinity of Interleukin-2 Analogs for Receptors

In order to measure the binding affinity of the interleukin-2 analogs obtained in Example 4 for each of interleukin-2 alpha receptors and beta receptors, surface plasmon resonance measurement (BIACORE T200, GE Healthcare) was used. The binding affinity of the prepared analogs for the alpha receptors and beta receptors was measured, and the binding affinity of each of the prepared analogs was compared with that of an interleukin-2 analog 1 (aldesleukin).

First, an anti-human immunoglobulin antibody (Abcam, #ab97221) was immobilized to CM5 chips (GE Healthcare) by as much as about 5,000 RU (resonance unit) through amine coupling, and then, the immunoglobulin antibody was finally immobilized by allowing the interleukin-2 alpha receptors (SYMANSIS, #4102H) or interleukin-2 beta receptors (SYMANSIS, #4122H), to each of which a human immunoglobulin Fc region was bound, to bind to each immunoglobulin antibody using an antigen-antibody binding reaction. Thereafter, the recombinant interleukin-2 analog prepared above was diluted to HBS-P+ buffer (Cytiva, BR100671) using a two-fold serial dilution method at various concentrations and was flowed onto the CM5 chips, to which the interleukin-2 receptors were finally immobilized, to measure the binding affinity of each interleukin-2 receptor. The measurement of binding affinity consisted of measurements of an association rate constant (ka) and a dissociation rate constant (k_{d}), in which the binding rate was measured by flowing each interleukin-2 analog at a flow rate of 10 μL/min for 3 minutes while the dissociation rate was measured from each interleukin-2 receptor by flowing only the HBS-P+ buffer for the same period of time and at the same flow rate. After the measurement was completed, the binding affinity for the receptors was evaluated according to the 1:1 binding fitting model in the Biaevaluation program.

$$Relative Binding Affinity ( {K}_{D} ) ( \% ) = Binding Affinity of Analog 1 ( aldesleukin ) ( {K}_{D} ) / Binding Affinity of Analog ( {K}_{D} ) \times 100$$

In Table 3 below, the expression “cannot be defined” indicates that the corresponding physical quantity cannot be defined for the corresponding receptor because no binding to the receptor was observed in the surface plasmon resonance measurement.

TABLE 3
Relative Binding Affinity of Interleukin-2 Analogs for Interleukin-2
Alpha or Beta Receptors Compared to Analog 1 (Aldesleukin)

| Interleukin-2 Receptor | Test Material | Relative Binding Affinity (%) |
|---|---|---|
| Alpha Receptor | analog 1 | 100.0 |
| | analog 9 | 74.5 |
| | analog 12 | cannot be defined |
| | analog 13 | 1.1 |
| | analog 15 | cannot be defined |
| | analog 16 | 0.2 |
| | analog 17 | 29.6 |
| | analog 19 | cannot be defined |
| | analog 20 | cannot be defined |
| | analog 21 | cannot be defined |
| | analog 31 | 5.0 |
| | analog 34 | 9.4 |
| | analog 35 | 31.7 |
| | analog 41 | 121.3 |
| | analog 52 | cannot be defined |
| | analog 53 | cannot be defined |
| | analog 86 | 71.1 |
| | analog 88 | 101.5 |
| | analog 90 | 98.4 |
| | analog 91 | 7.9 |
| | analog 92 | 97.3 |
| | analog 93 | 92.8 |
| | analog 95 | 10.7 |
| | analog 96 | 14.9 |
| | analog 97 | 18.8 |
| | analog 98 | 7.7 |
| | analog 99 | 19.9 |
| | analog 100 | 29.1 |
| | analog 101 | 24.7 |
| | analog 102 | 151.4 |
| | analog 103 | 6.1 |
| | analog 104 | 122.4 |
| | analog 105 | 246.8 |
| Beta Receptor | analog 01 | 100.0 |
| | analog 09 | 337.4 |
| | analog 12 | 166.2 |
| | analog 13 | 148.6 |
| | analog 14 | 129.7 |
| | analog 15 | 98.1 |
| | analog 16 | 1261.8 |
| | analog 17 | 9.4 |
| | analog 18 | 35.3 |
| | analog 19 | 455.0 |
| | analog 20 | 156.5 |
| | analog 21 | 14,084.2 |
| | analog 24 | 37.9 |
| | analog 25 | 21.7 |
| | analog 31 | 235.7 |
| | analog 34 | 321.8 |
| | analog 35 | 232.7 |
| | analog 41 | 22,776.2 |
| | analog 52 | 3,821.1 |
| | analog 53 | 690.7 |
| | analog 55 | 3,025.7 |
| | analog 57 | 2,569.7 |
| | analog 58 | 7,771.2 |
| | analog 59 | 1,533.5 |
| | analog 61 | 1,039.1 |
| | analog 70 | 10,199.2 |
| | analog 71 | 17,083.8 |
| | analog 73 | 1,591.8 |
| | analog 74 | 8,153.4 |
| | analog 75 | 9,571.2 |
| | analog 76 | 1,040.4 |
| | analog 77 | 644.4 |
| | analog 84 | 710.7 |
| | analog 86 | 18,745.8 |
| | analog 88 | 13,856.6 |
| | analog 90 | 12,776.2 |
| | analog 91 | 7,361.9 |
| | analog 92 | 1,510.3 |
| | analog 93 | 696.8 |
| | analog 94 | 35.5 |
| | analog 95 | 17.1 |
| | analog 96 | 229.3 |
| | analog 97 | 3,019.4 |
| | analog 98 | 11,084.5 |
| | analog 99 | 1,509.1 |
| | analog 100 | 2,534.1 |
| | analog 101 | 113.1 |
| | analog 102 | 4,452.0 |
| | analog 103 | 13,100.0 |
| | analog 104 | 25,439.8 |
| | analog 105 | 26,837.8 |

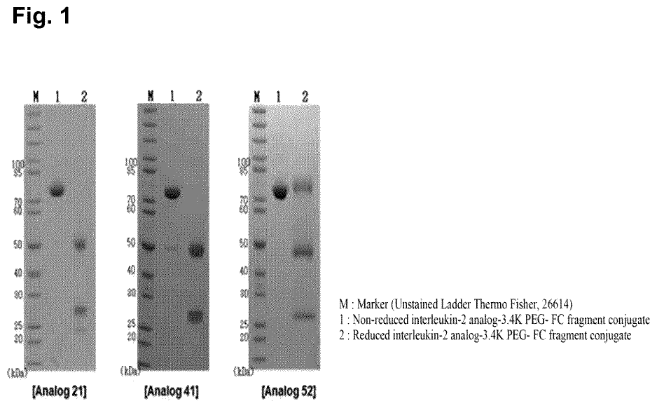

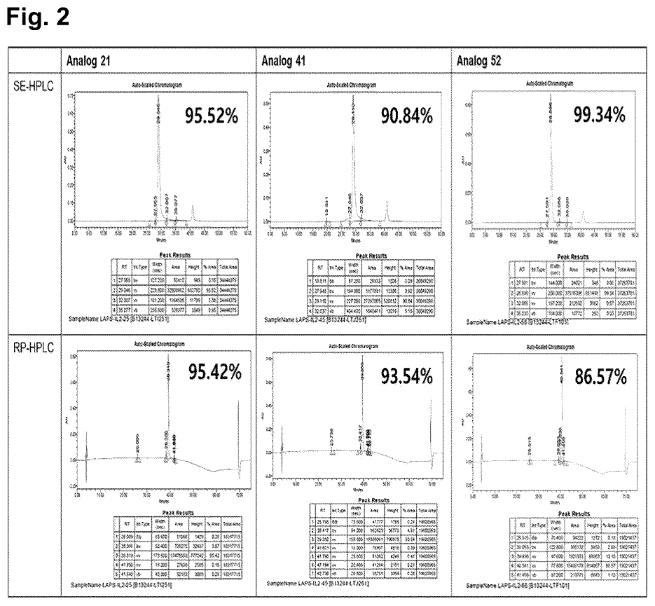

As explicitly shown in the test results (FIGS. 1 and 2A-2C and Table 3), it was confirmed that the interleukin-2 analogs of the present invention had no binding affinity, reduced/increased binding affinity for interleukin-2 alpha receptors compared to the interleukin-2 analog 1, etc., thus showing an altered binding affinity for interleukin-2 alpha receptors compared to native interleukin-2 or aldesleukin. In contrast, as for the interleukin-2 beta receptors, the interleukin-2 analogs of the present invention showed a stronger binding affinity of up to 100-fold compared to native interleukin-2 or aldesleukin. From the above results, it was confirmed that the amino acid sequence of the interleukin-2 analog has an effect on its binding to the interleukin-2 alpha or beta receptors. These results suggest that the binding affinity for interleukin-2 receptors can be altered by substituting an amino acid at a specific position.

These experimental results suggest that the interleukin-2 analogs according to the present invention have altered binding affinity for interleukin-2 alpha receptors and interleukin-2 beta receptors and thus can be used in the development of various drugs based on the same.

### Example 6: Coupling Reaction of Interleukin-2 Analog and Polyethylene Glycol (3.4K PEG) Linker and Purification of Interleukin-2 Analog Linked Material

In order to prepare a long-acting conjugate in which the interleukin-2 analog obtained in Example 4 was bound to the immunoglobulin Fc region, a linked material in which the interleukin-2 analog was linked to one end of a polyethylene glycol (PEG) linker was first prepared. Interleukin-2 analogs 21, 41, 52, 86, 104 and 105 were used for the preparation of the linked material, and as the PEG linker, polyethylene glycol (ALD(2)3.4K PEG manufactured by NOF, Japan) with a molecular weight of 3.4 kDa and in which hydroxy hydrogens at both ends were modified with propylaldehyde groups were conjugated to the N-terminus of the interleukin-2 analogs. The molar ratio of an interleukin-2 analog: PEG linker was 1:15 to 1:20, and they were reacted at 2° C. to 10° C. for 1 hour while the concentration of the interleukin-2 analogs was 1 mg/mL or less. Herein, the reaction was carried out under 100 mM potassium phosphate (pH 5.5), and 20 mM sodium cyanoborohydride (SCB) was added as a reducing agent. After changing the reaction solution to 20 mM triethylamine (pH 8.0) buffer using a desalting column, an interleukin-2 analog-3.4K PEG linked material was obtained by purification via Fractogel® EMD TMAE (S) (Merck Millipore) or Source 15Q (Cytiva) column using the concentration gradients of triethylamine (pH 8.0) and sodium chloride.

### Example 7: Preparation of Long-Acting Conjugate of Interleukin-2 Analog-3.4K PEG-Immunoglobulin Fc Region

In order to prepare a long-acting conjugate of an interleukin-2 analog-3.4K PEG-immunoglobulin Fc region, the mole ratio of the interleukin-2 analog-3.4K PEG linked material obtained using the method of Example 6 and the immunoglobulin Fc region (SEQ ID NO: 438) was set to 1:10, and the total protein concentration was set to 30 mg/ml to carry out the reaction at 2° C. to 10° C. for 15 to 16 hours. Herein, 100 mM potassium phosphate (pH 6.0) was used as a reaction solution, and 20 mM sodium cyanoborohydride was added as a reducing agent. In the immunoglobulin region used at this time, two monomers having the amino acid sequence of SEQ ID NO: 423 (consisting of 221 amino acids) form a homodimer through a disulfide bond between cysteine, which is the 3rd amino acid of each monomer, and the monomers of the homodimer each independently form an internal disulfide bond between cysteines at positions 35 and 95 and an internal disulfide bond between cysteines at positions 141 and 199, respectively.

TABLE 4
Protein Sequences of Immunoglobulin Fc

| | | SEQ |
|---|---|---|
| | | ID |
| Name | Protein Sequence | NO: |
| Immuno- | PSCPAPEFLG GPSVFLFPPK PKDTLMISRT | 438 |
| globulin | PEVTCVVVDV SQEDPEVQFN WYVDGVEVHN | |
| Fc | AKTKPREEQF NSTYRVVSVL TVLHQDWLNG | |
| | KEYKCKVSNK GLPSSIEKTI SKAKGQPREP | |
| | QVYTLPPSQE EMTKNQVSLT CLVKGFYPSD | |
| | IAVEWESNGQ PENNYKTTPP VLDSDGSFFL | |
| | YSRLTVDKSR WQEGNVFSCS VMHEALHNHY | |
| | TQKSLSLSLG K | |

After the reaction was completed, the unreacted immunoglobulin Fc region was removed from the reaction solution by Butyl FF (Cytiva) using Bis-Tris (pH 6.5) and sodium chloride, and the reaction solution was purified using sodium citrate buffer (pH 5.5) and ammonium sulfate by Source 15ISO (Cytiva) to obtain an interleukin-2 analog-3.4K PEG-immunoglobulin Fc region conjugate (long-acting conjugate) in which the N-terminus of the interleukin-2 analog is linked to one end of a 3.4 kDa PEG linker, and the opposite end of the 3.4 kDa PEG linker is linked to the nitrogen of the N-terminal proline of the Fc region. The long-acting conjugate was analyzed by SDS-PAGE, RP-HPLC, SE-HPLC (FIGS. 1 and 2A-2C).

### Example 8: Evaluation of Interleukin-2 Receptor Binding Affinity of Interleukin-2 Analog Conjugates

Surface plasma resonance (SPR, BIACORE T200, GE Healthcare) was used to measure the respective receptor binding affinity of the interleukin-2 analog long-acting conjugate obtained in Example 7 and the interleukin-2 alpha and beta receptors.

Specifically, biotin-labeled human interleukin-2 receptor alpha and beta subunits (ACROBiosystems) were each immobilized to a streptavidin biosensor chip (SA chip, Cytiva) at about 100 RU and 500 RU, and interleukin-2 analog long-acting conjugates diluted in HBS-EP+ buffer (Cytiva, BR100669) using a two-fold dilution method or aldesleukin were flowed at a flow rate of 20 μL/min. After the binding process for 3 minutes, only HBS-EP+ buffer was flowed for 3 minutes at the same flow rate to induce dissociation of the interleukin-2 analog long-acting conjugate or aldesleukin from the interleukin-2 receptors, and the binding affinity was calculated through the obtained association constant and dissociation constant. The binding affinity for the receptors was evaluated according to the 1:1 binding fitting model in the Biaevaluation program. Intrinsic interleukin-2 receptor binding affinity of the evaluated interleukin-2 analog conjugates was confirmed, and in particular, a clear difference in binding affinity between each candidate substance was confirmed in the interleukin-2 receptor alpha subunit. With reference to the results in detail, the interleukin-2 analog conjugates 21 and 52 did not bind to the interleukin-2 receptor alpha subunit, and the interleukin analog conjugates 41, 86, 104, and 105 showed a relative binding affinity of 50.8%, 65.2%, 81.0%, and 112.9% compared to aldesleukin. In the case of the interleukin-2 receptor beta subunit, higher binding affinity was confirmed compared to aldesleukin in all interleukin-2 analog conjugates including interleukin-2 analog conjugates 21 and 52.

Table 5 below summarizes the relative binding affinity (%) of the interleukin-2 analog long-acting conjugates prepared for each alpha and beta receptor compared with the binding affinity of aldesleukin. In Table 5, the corresponding long-acting conjugate is indicated by the number of the interleukin-2 analog constituting the same (e.g., the long-acting conjugate of an interleukin-2 analog 21 is indicated as “interleukin-2 analog conjugate 21”).

TABLE 5
Interleukin-2 Receptor Binding Affinity
of Interleukin-2 Analog Conjugates

| | Interleukin-2 | Interleukin-2 |
|---|---|---|
| | Receptor | Receptor |
| | Alpha Subunit | Beta Subunit |
| | Relative Binding | Relative Binding |
| Material for Evaluation | Affinity (%) | Affinity (%) |
| Aldesleukin | 100.0 | 100.0 |
| Interleukin-2 analog conjugate 21 | — | 5,426.6 |
| Interleukin-2 analog conjugate 41 | 50.8 | 5,483.0 |
| Interleukin-2 analog conjugate 52 | — | 2,271.8 |
| Interleukin-2 analog conjugate 86 | 65.2 | 15,393.1 |
| Interleukin-2 analog conjugate 104 | 81.0 | 13,423.5 |
| Interleukin-2 analog conjugate 105 | 112.9 | 13,293.8 |

### Example 9: Pharmacokinetic Evaluation of Interleukin-2 Analog Long-Acting Conjugates

It was confirmed whether medicaments using the interleukin-2 analogs and the long-acting conjugate had excellent blood exposure and bioavailability based on the interleukin-2 analogs confirmed in Examples 5 and 7, and the binding ability of the long-acting conjugates including the same for the interleukin-2 receptors.

Specifically, the present inventors compared and analyzed the pharmacokinetics of an interleukin-2 analog long-acting conjugates and aldesleukin in ICR and C57BL/6 lineage normal mouse models.

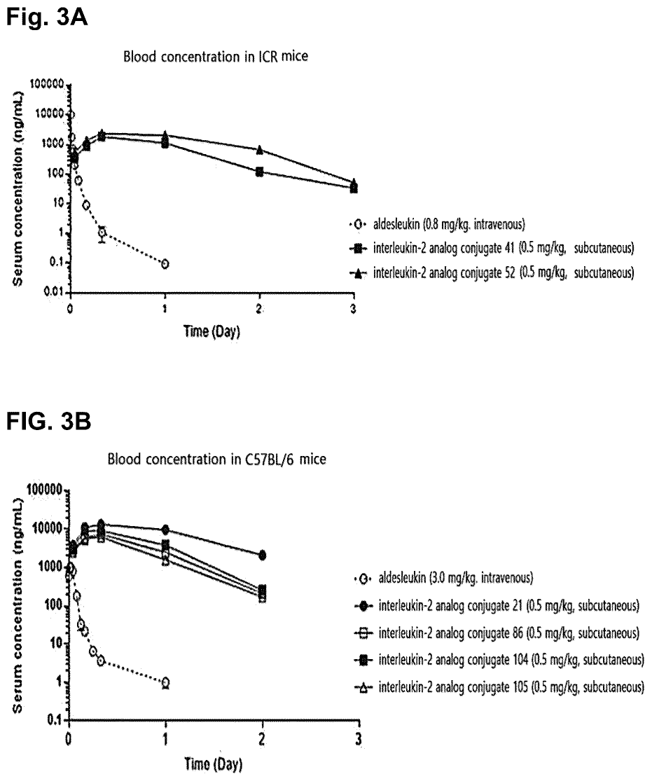

More specifically, the control group administered with aldesleukin and the experimental group administered with the interleukin-2 analog conjugates (interleukin-2 analog conjugate 21, interleukin-2 analog conjugate 41, interleukin-2 analog conjugate 52, interleukin-2 analog conjugate 86, interleukin-2 analog conjugate 104, and interleukin-2 analog conjugate 105) were subcutaneously, intravenously, or intraperitoneally injected into normal mice. In the group in which the interleukin-2 analog conjugates were subcutaneously injected, blood was drawn from 3 animals via retro-orbital blood collection after 1, 4, 8, 24, 48, 72, and 96 hours, and in the group to which aldesleukin was administered by intravenous or intraperitoneal route, blood was drawn in the same manner from 3 animals at 0.08, 0.25, 0.5, 1, 1.5, 2, 3, 4, 6, 8, and 24 hours. Blood drawn from each group was centrifuged and separated into serum, and interleukin-2 analog conjugates and aldesleukin in the blood were quantified by ELISA using a human interleukin-2 antibody. As a result, the interleukin-2 analog conjugates exhibited higher blood exposure levels in normal mice of ICR and C57BL/6 lineage compared to aldesleukin (FIG. 3A and FIG. 3B, Tables 6 and 7). In the dose, AUC, and concentration values of the conjugates shown in the table below, the amount of conjugate (mg, ng) was expressed based on only the weight occupied by the interleukin-2 analog region among the entire conjugate.

TABLE 6
Pharmacokinetics of Interleukin-2 Analog Conjugates in ICR Mice

| | | Interleukin-2 | Interleukin-2 |
|---|---|---|---|
| | | analog | analog |
| | Aldesleukin | conjugate 41 | conjugate 52 |
| Administration dose | 0.8 mg/kg | 0.5 mg/kg | 0.5 mg/kg |
| and route | (intravenous | (subcutaneous | (subcutaneous |
| | injection) | injection) | injection) |
| AUC (ng/mL × hr) | 2,463.8 | 43,511.2 | 82,579.4 |
| C₀ or Cₘₐₓ (ng/mL) | 20,183.2 | 1,887.9 | 2,370.5 |
| T_{1/2} (hr) | 1.4 | 8.5 | 9.0 |

TABLE 7
Pharmacokinetics of Interleukin-2 Analog Conjugates in C57BL/6 Mice

| | | Interleukin-2 | Interleukin-2 | Interleukin-2 | Interleukin-2 |
|---|---|---|---|---|---|
| | | analog | analog | analog | analog |
| | Aldesleukin | conjugate 21 | conjugate 86 | conjugate 104 | conjugate 105 |
| Administration dose | 3.0 mg/kg | 0.5 mg/kg | 0.5 mg/kg | 0.5 mg/kg | 0.5 mg/kg |
| and route | (intraperitoneal | (subcutaneous | (subcutaneous | (subcutaneous | (subcutaneous |
| | injection) | injection) | injection) | injection) | injection) |
| AUC (ng/mL × hr) | 1,458.2 | 375,794.2 | 136,373.5 | 186,068.4 | 107,487.6 |
| C₀ or Cₘₐₓ (ng/mL) | 1,018.6 | 13,319.0 | 7,371.1 | 9,288.3 | 6302.1 |

As can be seen from the above results, the interleukin-2 analog conjugates according to the present invention exhibited a higher blood distribution compared to aldesleukin through intravenous or intraperitoneal injection, despite being administered by the subcutaneous route. These results suggest that when the drug is administered, a larger amount of the interleukin-2 analog conjugate is exposed to the blood even when a smaller amount is administered compared to human recombinant interleukin-2, and that the convenience of administration can be secured.

### Example 10: Evaluation of Anticancer Efficacy of Interleukin-2 Analog Long-Acting Conjugates

In order to investigate the therapeutic efficacy of the interleukin-2 analog conjugates, tumor size and individual mortality were evaluated after each administration of interleukin-2 analog conjugate 21, interleukin-2 analog conjugate 41 and the interleukin-2 analog 52, and aldesleukin as a control material into CT26 colon tumor syngeneic mouse model.

Specifically, CT26 cells (ATCC) cultured in a cell culture flask were subcutaneously injected into the legs of BALB/c mice, and after a few days, when tumors were observed with the naked eye, 7 mice were assigned to each group so that the tumors were similar in size. To each group, 0.5 mg/kg of an interleukin-2 analog conjugate 21, interleukin-2 analog conjugate 41, and interleukin-2 analog conjugate 52 were repeatedly administered by subcutaneous route once a week for a total of two times. Herein, the dosage of the long-acting conjugate represents the weight of only the interleukin-2 analog region among the corresponding conjugate. In the aldesleukin administration group, 3.0 mg/kg of proleukin (Novartis) was administered once a day by the intraperitoneal route for 5 consecutive days, and after a 2-day rest, additional administration was performed for 5 days according to the same schedule.

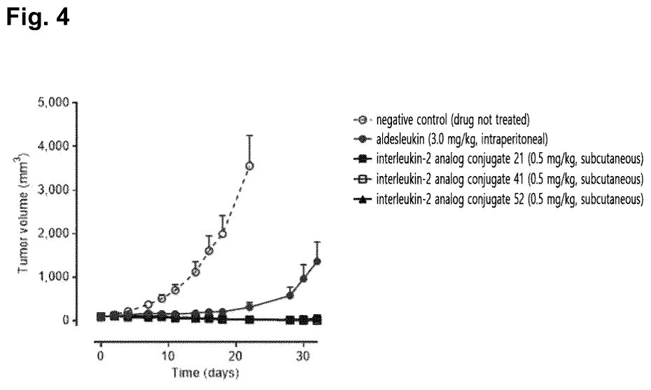

Body weight and individual death were observed for 32 days from the first administration date, and based on the results, excellent anticancer efficacy was confirmed in all the interleukin-2 analog conjugate administration groups. In particular, complete remission was observed in all individuals in the group administered with interleukin-2 analog conjugate 41. In contrast, in the group administered with aldesleukin, 2 out of 7 mice died, and only 2 mice showed complete remission. It is interpreted that the interleukin-2 analog conjugate according to the present invention strongly binds to the human interleukin-2 receptor beta subunit by altering the amino acid sequence, thereby exhibiting excellent anticancer effect (FIG. 4). In addition, the long-acting conjugate structure to which the human immunoglobulin Fc region is linked enables high blood exposure to increase the dosing interval, thereby suggesting that it is possible to provide administration convenience to patients and medical staff by converting the conventional injection therapy to a subcutaneous route.

### Example 11: Evaluation of Memory T Cell Responses According to Repeated Exposure of Tumors

Following the anticancer efficacy evaluation of Example 10, the response of memory T cells according to repeated exposure of tumors was evaluated. Specifically, for the individuals whose complete remission was confirmed after the anticancer efficacy evaluation was completed, CT26 cells were administered subcutaneously in the same manner as the first exposure, on 42 and 70 days after the first drug administration, and subjected to re-exposure to observe the tumor growth. At 4 and 7 days after the second re-exposure, the number of memory T cells from the mouse spleen was analyzed using a FACS Canto II (Becton Dickinson) device.

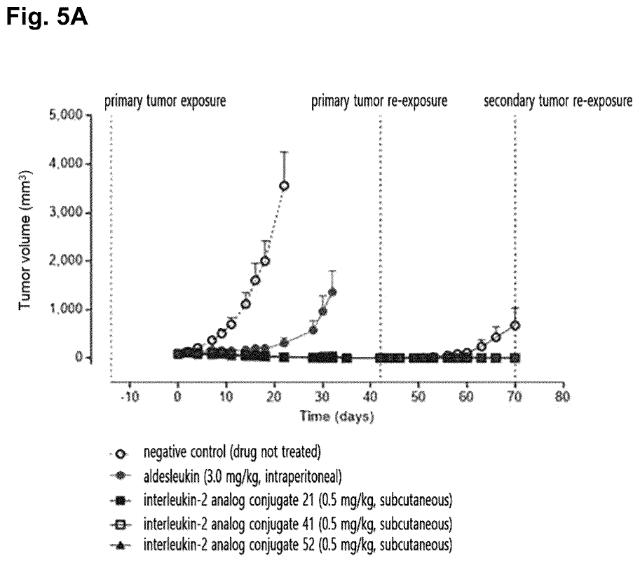

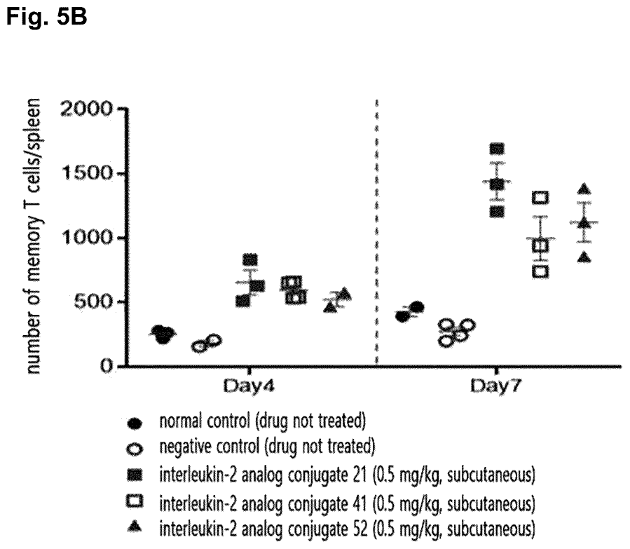

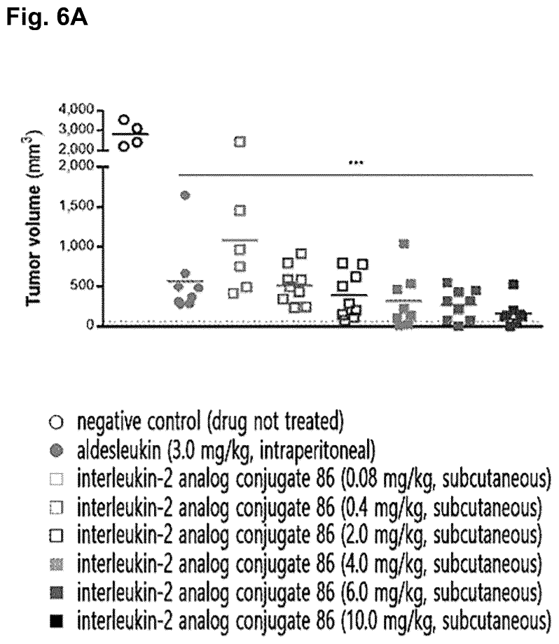

As a result, in all drug administration groups except for the negative control group, tumor growth was suppressed according to re-exposure, and a significant increase in memory T cells was observed in the spleen (FIGS. 5A and 5B). In conclusion, the interleukin-2 analog conjugates according to the present invention induced a memory immune response in the CT26 colon tumor syngeneic mouse model with only a single agent, and thus strong anticancer efficacy was maintained not only in the first tumor exposure but also in the re-exposure of the tumor.

### Example 12: Evaluation of Anticancer Efficacy of Interleukin-2 Analog Long-Acting Conjugate in Malignant Melanoma

In order to evaluate the anticancer efficacy of the interleukin-2 analog conjugate according to the present invention, interleukin-2 analog conjugate 86 and aldesleukin as a control material were each administered to B16F10 melanoma tumor syngeneic mouse model to evaluate the tumor size and individual survival rate.

Specifically, B16F10 cells (ATCC) cultured in a cell culture flask were subcutaneously injected into the thigh of C57BL/6 mice, and when tumors were observed with the naked eye a few days later, 9 mice were assigned to each group so that the tumors were similar in size. Among the long-acting conjugates, based on the weight of the interleukin-2 analog region, 0.08 mg/kg to 10 mg/kg of interleukin-2 analog conjugate 86 was each administered subcutaneously, once a week, for a total of 4 repeated times. In addition, in the aldesleukin administration group as a control group, 3.0 mg/kg of proleukin (Novartis) was administered once a day by intraperitoneal route for 5 consecutive days, followed by a 2-day rest period, and the administration was repeatedly performed for a total of 4 times.

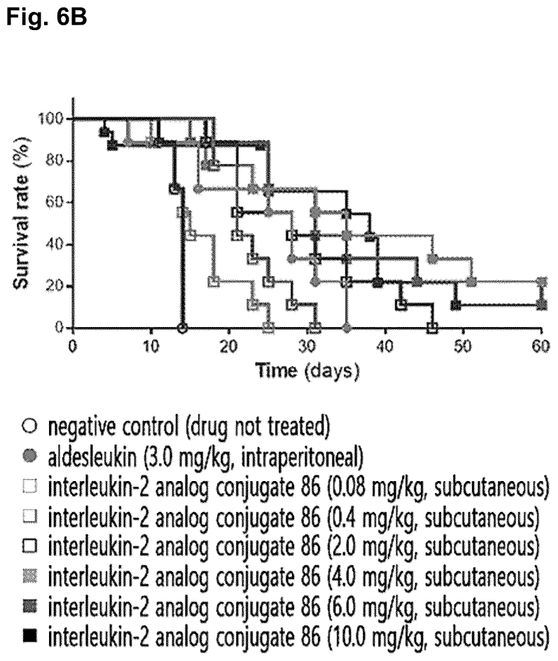

On day 15 from the first dosing day, the tumor size of each group (FIG. 6A) and the survival rate of each individual for 60 days (FIG. 6B) were observed. As a result, it was confirmed that the group administered with interleukin-2 analog conjugate 86 of the present invention had a smaller tumor size and improved individual survival rate compared to the group administered with aldesleukin, and the dose-dependent anticancer efficacy of the long-acting conjugate of the present invention was confirmed. In particular, in the group administered with interleukin-2 analog conjugate 86 at 4.0 mg/kg or more per week, complete remission with complete tumor removal was observed in some individuals. In contrast, in the group in which aldesleukin was administered at 3.0 mg/kg consecutively for 5 days, no complete remission was found in any individual.

This means that the interleukin-2 analog conjugates of the present invention were designed to have a characteristic of binding to the beta subunit of the human interleukin-2 receptor, which is relatively strong compared to aldesleukin, and thus can exhibit excellent anticancer effect. In addition, it effectively controls excessive immune response through binding to the alpha subunit of the human interleukin-2 receptor, which does not cause undesired side effects, so that no side effects were observed to the extent that the drug administration was discontinued even in the high-dosing group of 10 mg/kg or more based on the weight of the interleukin-2 analog region in the long-acting conjugates.

The experimental results described above show that the long-acting conjugate including the interleukin-2 analog in which an amino acid mutation was introduced into native interleukin-2 according to the present invention has altered binding affinity for the interleukin-2 alpha receptors and binding affinity for the interleukin-2 beta receptors, thereby suggesting that it can be used for the development of various drugs using the conjugate, particularly the development of anticancer drugs.

Based on the above description, it will be understood by those skilled in the art that the present disclosure may be implemented in a different specific form without changing the technical spirit or essential characteristics thereof. In this regard, it should be understood that the above embodiment is not limitative, but illustrative in all aspects. The scope of the disclosure is defined by the appended claims rather than by the description preceding them, and therefore all changes and modifications that fall within metes and bounds of the claims, or equivalents of such metes and bounds, are therefore intended to be embraced by the claims.

1. A long-acting conjugate comprising an interleukin-2 analog, represented by Chemical Formula 1 below: 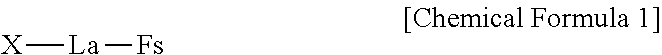
wherein, X is an interleukin-2 analog having an increased binding affinity for interleukin-2 beta receptors compared to aldesleukin;
L is a polyethylene glycol linker;
a is 0 or a natural number, with the proviso that when a is 2 or more, each L is independent of each other;
F is an immunoglobulin Fc region in the form of a dimer; and
- indicates a covalent linkage between X and L and between L and F;
wherein the interleukin-2 analog comprises a sequence in which one or more amino acids corresponding to positions 1, 12, 18, 19, 20, 22, 32, 35, 38, 42, 43, 45, 48, 49, 61, 68, 69, 74, 76, 80, 81, 82, 84, 85, 86, 87, 88, 89, 91, 92, 94, 95, 96, 125, 126, and 133 in native interleukin-2 are modified.

2. The long-acting conjugate of claim 1, wherein the interleukin-2 analog has an altered binding affinity for interleukin-2 alpha receptors compared to aldesleukin.

3. The long-acting conjugate of claim 1, wherein one or more amino acids are added to the amino acid corresponding to position 133; or wherein the interleukin-2 analog is one in which the amino acid at position 1 is removed and the amino acid at position 125 is substituted with a different amino acid in native interleukin-2 analog.

4. The long-acting conjugate of claim 3, wherein the interleukin-2 analog further comprises 1 to 10 amino acid substitutions.

5. The long-acting conjugate of claim 4, wherein the interleukin-2 analog comprises substitutions in which one or more amino acids at positions 18, 19, 20, 22, 38, 42, 43, 45, 61, 68, 69, 74, 80, 81, 84, 85, 86, 88, 89, 91, 92, 94, and 96 are further substituted with different amino acids; or wherein the interleukin-2 analog comprises substitutions in which one or more amino acids at positions 18, 22, 38, 42, 61, 68, 80, 81, 85, 86 and 92 are further substituted with different amino acids.

6. The long-acting conjugate of claim 1, wherein the interleukin-2 analog is any one of the following analogs:
(a) an interleukin-2 analog in which the amino acid at position 1 is removed and the amino acids at positions 125 and 32 are substituted with different amino acids in native interleukin-2;
(b) an interleukin-2 analog in which the amino acid at position 1 is removed and the amino acids at positions 125 and 35 are substituted with different amino acids in native interleukin-2;
(c) an interleukin-2 analog in which the amino acid at position 1 is removed and the amino acids at positions 125 and 38 are substituted with different amino acids in native interleukin-2;
(d) an interleukin-2 analog in which the amino acid at position 1 is removed and the amino acids at positions 125 and 42 are substituted with different amino acids in native interleukin-2;
(e) an interleukin-2 analog in which the amino acid at position 1 is removed and the amino acids at positions 125 and 43 are substituted with different amino acids in native interleukin-2;
(f) an interleukin-2 analog in which the amino acid at position 1 is removed and the amino acids at positions 125 and 48 are substituted with different amino acids in native interleukin-2;
(g) an interleukin-2 analog in which the amino acid at position 1 is removed and the amino acids at positions 125 and 49 are substituted with different amino acids in native interleukin-2;
(h) an interleukin-2 analog in which the amino acid at position 1 is removed and the amino acids at positions 125 and 76 are substituted with different amino acids in native interleukin-2;
(i) an interleukin-2 analog in which the amino acid at position 1 is removed and the amino acids at positions 125, 38, 42, 81, 92, 94, and 96 are substituted with different amino acids in native interleukin-2;
(j) an interleukin-2 analog in which the amino acid at position 1 is removed and the amino acids at positions 125 and 87 are substituted with different amino acids in native interleukin-2;
(k) an interleukin-2 analog in which the amino acid at position 1 is removed and the amino acids at positions 125, 38, and 42 are substituted with different amino acids in native interleukin-2;
(l) an interleukin-2 analog in which the amino acid at position 1 is removed and the amino acids at positions 125, 38, and 80 are substituted with different amino acids in native interleukin-2;
(m) an interleukin-2 analog in which the amino acid at position 1 is removed and the amino acids at positions 125, 38, and 84 are substituted with different amino acids in native interleukin-2;
(n) an interleukin-2 analog in which the amino acid at position 1 is removed and the amino acids at positions 125, 19, 38, and 42 are substituted with different amino acids in native interleukin-2;
(o) an interleukin-2 analog in which the amino acid at position 1 is removed and the amino acids at positions 125, 12, 38, and 42 are substituted with different amino acids in native interleukin-2;
(p) an interleukin-2 analog in which the amino acid at position 1 is removed and the amino acids at positions 125, 38, 42, and 61 are substituted with different amino acids in native interleukin-2;
(q) an interleukin-2 analog in which the amino acid at position 1 is removed and the amino acids at positions 125, 38, 42, and 84 are substituted with different amino acids in native interleukin-2;
(r) an interleukin-2 analog in which the amino acid at position 1 is removed and the amino acids at positions 125, 38, 42, and 88 are substituted with different amino acids in native interleukin-2;
(s) an interleukin-2 analog in which the amino acid at position 1 is removed and the amino acids at positions 125, 38, 42, and 89 are substituted with different amino acids in native interleukin-2;
(t) an interleukin-2 analog in which the amino acid at position 1 is removed and the amino acids at positions 125, 38, 42, and 91 are substituted with different amino acids in native interleukin-2;
(u) an interleukin-2 analog in which the amino acid at position 1 is removed and the amino acids at positions 125, 38, 42, and 94 are substituted with different amino acids in native interleukin-2;
(v) an interleukin-2 analog in which the amino acid at position 1 is removed and the amino acids at positions 125, 38, 42, and 126 are substituted with different amino acids in native interleukin-2;
(w) an interleukin-2 analog in which the amino acid at position 1 is removed and the amino acids at positions 125, 38, 80, and 84 are substituted with different amino acids in native interleukin-2;
(x) an interleukin-2 analog in which the amino acid at position 1 is removed and the amino acids at positions 125, 38, 94, and 96 are substituted with different amino acids in native interleukin-2;
(y) an interleukin-2 analog in which the amino acid at position 1 is removed and the amino acids at positions 125, 38, 81, and 92 are substituted with different amino acids in native interleukin-2;
(z) an interleukin-2 analog in which the amino acid at position 1 is removed and the amino acids at positions 125, 61, 81, and 92 are substituted with different amino acids in native interleukin-2;
(aa) an interleukin-2 analog in which the amino acid at position 1 is removed and the amino acids at positions 125, 38, 42, 81, and 92 are substituted with different amino acids in native interleukin-2;
(ab) an interleukin-2 analog in which the amino acid at position 1 is removed and the amino acids at positions 125, 38, 80, 81, and 92 are substituted with different amino acids in native interleukin-2;
(ac) an interleukin-2 analog in which the amino acid at position 1 is removed and the amino acids at positions 125, 38, 81, 84, and 92 are substituted with different amino acids in native interleukin-2;
(ad) an interleukin-2 analog in which the amino acid at position 1 is removed and the amino acids at positions 125, 20, 38, 42, 81, and 92 are substituted with different amino acids in native interleukin-2;
(ae) an interleukin-2 analog in which the amino acid at position 1 is removed and the amino acids at positions 125, 38, 42, 80, 81, and 92 are substituted with different amino acids in native interleukin-2;
(af) an interleukin-2 analog in which the amino acid at position 1 is removed and the amino acids at positions 125, 38, 42, 74, 81, and 92 are substituted with different amino acids in native interleukin-2;
(ag) an interleukin-2 analog in which the amino acid at position 1 is removed and the amino acids at positions 125, 38, 42, 81, 84, and 92 are substituted with different amino acids in native interleukin-2;
(ah) an interleukin-2 analog in which the amino acid at position 1 is removed and the amino acids at positions 125, 38, 42, 81, 88, and 92 are substituted with different amino acids in native interleukin-2;
(ai) an interleukin-2 analog in which the amino acid at position 1 is removed and the amino acids at positions 125, 38, 42, 85, 86, and 92 are substituted with different amino acids in native interleukin-2;
(aj) an interleukin-2 analog in which the amino acid at position 1 is removed and the amino acids at positions 125, 38, 80, 81, 85, and 92 are substituted with different amino acids in native interleukin-2;
(ak) an interleukin-2 analog in which the amino acid at position 1 is removed and the amino acids at positions 125, 38, 80, 81, 86, and 92 are substituted with different amino acids in native interleukin-2;
(al) an interleukin-2 analog in which the amino acid at position 1 is removed and the amino acids at positions 125, 80, 81, 85, 86, and 92 are substituted with different amino acids in native interleukin-2;
(am) an interleukin-2 analog in which the amino acid at position 1 is removed and the amino acids at positions 125, 38, 42, 45, 74, 81, and 92 are substituted with different amino acids in native interleukin-2;
(an) an interleukin-2 analog in which the amino acid at position 1 is removed and the amino acids at positions 125, 38, 42, 74, 80, 81, and 92 are substituted with different amino acids in native interleukin-2;
(ao) an interleukin-2 analog in which the amino acid at position 1 is removed and the amino acids at positions 125, 38, 42, 80, 81, 84, and 92 are substituted with different amino acids in native interleukin-2;
(ap) an interleukin-2 analog in which the amino acid at position 1 is removed and the amino acids at positions 125, 38, 42, 45, 80, 85, 86, and 92 are substituted with different amino acids in native interleukin-2;
(aq) an interleukin-2 analog in which the amino acid at position 1 is removed and the amino acids at positions 125, 38, 80, 81, 85, 86, and 92 are substituted with different amino acids in native interleukin-2;
(ar) an interleukin-2 analog in which the amino acid at position 1 is removed and the amino acids at positions 125, 42, 80, 81, 85, 86, and 92 are substituted with different amino acids in native interleukin-2;
(as) an interleukin-2 analog in which the amino acid at position 1 is removed and the amino acids at positions 125, 61, 80, 81, 85, 86, and 92 are substituted with different amino acids in native interleukin-2;
(at) an interleukin-2 analog in which the amino acid at position 1 is removed and the amino acids at positions 125, 69, 80, 81, 85, 86, and 92 are substituted with different amino acids in native interleukin-2;
(au) an interleukin-2 analog in which the amino acid at position 1 is removed and the amino acids at positions 125, 80, 81, 85, 86, 91, and 92 are substituted with different amino acids in native interleukin-2;
(av) an interleukin-2 analog in which the amino acid at position 1 is removed and the amino acids at positions 125, 18, 22, 80, 81, 85, 86, and 92 are substituted with different amino acids in native interleukin-2;
(aw) an interleukin-2 analog in which the amino acid at position 1 is removed and the amino acids at positions 125, 38, 42, 80, 81, 85, 86, and 92 are substituted with different amino acids in native interleukin-2;
(ax) an interleukin-2 analog in which the amino acid at position 1 is removed and the amino acids at positions 125, 38, 42, 45, 74, 80, 81, and 92 are substituted with different amino acids in native interleukin-2;
(ay) an interleukin-2 analog in which the amino acid at position 1 is removed and the amino acids at positions 125, 38, 68, 80, 81, 85, 86, and 92 are substituted with different amino acids in native interleukin-2;
(az) an interleukin-2 analog in which the amino acid at position 1 is removed and the amino acids at positions 125, 69, 74, 80, 81, 85, 86, and 92 are substituted with different amino acids in native interleukin-2;
(ba) an interleukin-2 analog in which the amino acid at position 1 is removed and the amino acids at positions 125, 80, 81, 84, 85, 86, 91, and 92 are substituted with different amino acids in native interleukin-2;
(bb) an interleukin-2 analog in which the amino acid at position 1 is removed and the amino acids at positions 125, 80, 81, 85, 86, 92, 94, and 96 are substituted with different amino acids in native interleukin-2;
(bc) an interleukin-2 analog in which the amino acid at position 1 is removed and the amino acids at positions 125, 18, 19, 22, 80, 81, 85, 86, and 92 are substituted with different amino acids in native interleukin-2;
(bd) an interleukin-2 analog in which the amino acid at position 1 is removed and the amino acids at positions 125, 18, 22, 38, 80, 81, 85, 86, and 92 are substituted with different amino acids in native interleukin-2;
(be) an interleukin-2 analog in which the amino acid at position 1 is removed and the amino acids at positions 125, 18, 22, 61, 80, 81, 85, 86, and 92 are substituted with different amino acids in native interleukin-2;
(bf) an interleukin-2 analog in which the amino acid at position 1 is removed and the amino acids at positions 125, 18, 22, 68, 80, 81, 85, 86, and 92 are substituted with different amino acids in native interleukin-2;
(bg) an interleukin-2 analog in which the amino acid at position 1 is removed and the amino acids at positions 125, 35, 38, 42, 80, 81, 85, 86, and 92 are substituted with different amino acids in native interleukin-2;
(bh) an interleukin-2 analog in which the amino acid at position 1 is removed and the amino acids at positions 125, 38, 42, 45, 80, 81, 85, 86, and 92 are substituted with different amino acids in native interleukin-2;
(bi) an interleukin-2 analog in which the amino acid at position 1 is removed and the amino acids at positions 125, 38, 42, 80, 81, 85, 86, 91, and 92 are substituted with different amino acids in native interleukin-2;
(bj) an interleukin-2 analog in which the amino acid at position 1 is removed and the amino acids at positions 125, 38, 42, 80, 81, 85, 86, 92, and 95 are substituted with different amino acids in native interleukin-2;
(bk) an interleukin-2 analog in which the amino acid at position 1 is removed and the amino acids at positions 125, 35, 38, 42, 74, 80, 81, 85, 86, and 92 are substituted with different amino acids in native interleukin-2;
(bl) an interleukin-2 analog in which the amino acid at position 1 is removed and the amino acids at positions 125, 38, 42, 43, 61, 80, 81, 85, 86, and 92 are substituted with different amino acids in native interleukin-2;
(bm) an interleukin-2 analog in which the amino acid at position 1 is removed and the amino acids at positions 125, 38, 42, 80, 81, 85, 86, 91, 92, and 95 are substituted with different amino acids in native interleukin-2;
(bn) an interleukin-2 analog in which the amino acid at position 1 is removed and the amino acids at positions 125, 35, 38, 42, 74, 80, 81, 82, 85, 86, and 92 are substituted with different amino acids in native interleukin-2;
(bo) an interleukin-2 analog in which the amino acid at position 1 is removed and the amino acids at positions 125, 80, 81, 86, and 92 are substituted with different amino acids in native interleukin-2; and
(bp) an interleukin-2 analog in which the amino acid at position 1 is removed and the amino acids at positions 125, 80, 81, 85, and 86 are substituted with different amino acids in native interleukin-2.

7. The long-acting conjugate of claim 6, wherein the amino acid substitution is any one selected from the group consisting of the following amino acid substitutions:
(a) a substitution in which the amino acid at position 12 is substituted with valine or phenylalanine;
(b) a substitution in which the amino acid at position 18 is substituted with arginine;
(c) a substitution in which the amino acid at position 19 is substituted with tyrosine, valine, phenylalanine, or arginine;
(d) a substitution in which the amino acid at position 20 is substituted with valine or phenylalanine;
(e) a substitution in which the amino acid at position 22 is substituted with glutamic acid;
(f) a substitution in which the amino acid at position 32 is substituted with cysteine;
(g) a substitution in which the amino acid at position 35 is substituted with cysteine or glutamic acid;
(h) a substitution in which the amino acid at position 38 is substituted with alanine or aspartic acid;
(i) a substitution in which the amino acid at position 42 is substituted with lysine, alanine, or tryptophan;
(j) a substitution in which the amino acid at position 43 is substituted with cysteine, glutamic acid, or glutamine;
(k) a substitution in which the amino acid at position 45 is substituted with alanine;
(l) a substitution in which the amino acid at position 48 is substituted with cysteine;
(m) a substitution in which the amino acid at position 49 is substituted with cysteine;
(n) a substitution in which the amino acid at position 61 is substituted with glutamine, arginine, or aspartic acid;
(o) a substitution in which the amino acid at position 68 is substituted with aspartic acid or glutamine;
(p) a substitution in which the amino acid at position 69 is substituted with glycine;
(q) a substitution in which the amino acid at position 74 is substituted with histidine or alanine;
(r) a substitution in which the amino acid at position 76 is substituted with cysteine;
(s) a substitution in which the amino acid at position 80 is substituted with phenylalanine, tyrosine, valine, aspartic acid, or tryptophan;
(t) a substitution in which the amino acid at position 81 is substituted with aspartic acid, glutamic acid, or asparagine;
(u) a substitution in which the amino acid at position 82 is substituted with glycine or valine;
(v) a substitution in which the amino acid at position 84 is substituted with glutamic acid, valine, or phenylalanine;
(w) a substitution in which the amino acid at position 85 is substituted with valine, alanine, glycine, tryptophan, tyrosine, threonine, isoleucine, glutamic acid, or phenylalanine;
(x) a substitution in which the amino acid at position 86 is substituted with valine, alanine, glycine, or leucine;
(y) a substitution in which the amino acid at position 87 is substituted with cysteine;
(z) a substitution in which the amino acid at position 88 is substituted with glutamine, valine, or phenylalanine;
(aa) a substitution in which the amino acid at position 89 is substituted with phenylalanine;
(ab) a substitution in which the amino acid at position 91 is substituted with threonine, phenylalanine, or glutamic acid;
(ac) a substitution in which the amino acid at position 92 is substituted with phenylalanine, leucine, tyrosine, or tryptophan;
(ad) a substitution in which the amino acid at position 94 is substituted with phenyl alanine or valine;
(ae) a substitution in which the amino acid at position 95 is substituted with aspartic acid;
(af) a substitution in which the amino acid at position 96 is substituted with phenylalanine, valine, or isoleucine;
(ag) a substitution in which the amino acid at position 125 is substituted with serine; and
(ah) a substitution in which the amino acid at position 126 is substituted with threonine.

8. The long-acting conjugate of claim 6, wherein the interleukin-2 analog is any one of the following analogs:
(a) an interleukin-2 analog in which the amino acid at position 1 is removed and the amino acids at positions 125, 38, 42, 80, 81 and 92 are substituted with different amino acids in native interleukin-2 analog;
(b) an interleukin-2 analog in which the amino acid at position 1 is removed and the amino acids at positions 125, 80, 81, 85, 86, and 92 are substituted with different amino acids in native interleukin-2 analog;
(c) an interleukin-2 analog in which the amino acid at position 1 is removed and the amino acids at positions 125, 18, 22, 80, 81, 85, 86, and 92 are substituted with different amino acids in native interleukin-2 analog;
(d) an interleukin-2 analog in which the amino acid at position 1 is removed and the amino acids at positions 125, 38, 42, 80, 81, 85, 86, and 92 are substituted with different amino acids in native interleukin-2 analog;
(e) an interleukin-2 analog in which the amino acid at position 1 is removed and the amino acids at positions 125, 18, 22, 61, 80, 81, 85, 86, and 92 are substituted with different amino acids in native interleukin-2 analog; and
(f) an interleukin-2 analog in which the amino acid at position 1 is removed and the amino acids at positions 125, 18, 22, 68, 80, 81, 85, 86, and 92 are substituted with different amino acids in native interleukin-2 analog.

9. The long-acting conjugate of claim 8, wherein the amino acid substitution is any one selected from the group consisting of the following amino acid substitutions:
(a) a substitution in which the amino acid at position 18 is substituted with arginine;
(b) a substitution in which the amino acid at position 22 is substituted with glutamic acid;
(c) a substitution in which the amino acid at position 38 is substituted with alanine;
(d) a substitution in which the amino acid at position 42 is substituted with lysine;
(e) a substitution in which the amino acid at position 61 is substituted with aspartic acid;
(f) a substitution in which the amino acid at position 68 is substituted with aspartic acid;
(g) a substitution in which the amino acid at position 80 is substituted with phenylalanine;
(h) a substitution in which the amino acid at position 81 is substituted with glutamic acid;
(i) a substitution in which the amino acid at position 85 is substituted with valine;
(j) a substitution in which the amino acid at position 86 is substituted with valine;
(k) a substitution in which the amino acid at position 92 is substituted with phenylalanine; and
(l) a substitution in which the amino acid at position 125 is substituted with serine.

10. The long-acting conjugate of claim 1, wherein the immunoglobulin Fc region is those derived from IgG, IgA, IgD, IgE, IgM, or a combination thereof, or a hybrid thereof; wherein the immunoglobulin Fc region is an IgG4 Fc region; wherein the immunoglobulin Fc region is non-glycosylated; wherein the L contains ethylene glycol repeating units, and the formula weight of the ethylene glycol repeating unit moiety is in the range of 1 kDa to 100 kDa; or wherein the long-acting conjugate is a long-acting conjugate in which one molecule of X is covalently linked to one of the Fc regions of the dimeric immunoglobulin Fc region via the polyethylene glycol linker.

11. A long-acting conjugate of an interleukin-2 analog, represented by Chemical Formula 2 below: 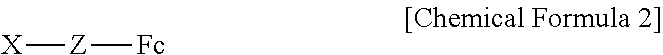
wherein X is an interleukin-2 analog comprising any one of polypeptide sequences selected from the amino acid sequence of SEQ ID NOS: 3 to 106;
Z is a polyethylene glycol linker having a molecular weight of 2 kDa to 30 kDa;
Fc is an immunoglobulin Fc region in the form of a dimer; and
- indicates a covalent linkage between X and Z and between Z and Fc;
wherein the long-acting conjugate is a long-acting conjugate in which one end of Z is covalently linked only to one polypeptide chain in the dimeric Fc region, and one molecule of X is covalently linked to the opposite end of Z.

12. The long-acting conjugate of claim 11, wherein the interleukin-2 analog comprises any one of sequences selected from the group consisting of amino acid sequences of SEQ ID NOS: 10, 13, 14, 15, 16, 17, 20, 21, 22, 32, 35, 36, 42, 53, 54, 56, 58, 59, 60, 62, 71, 72, 74, 75, 76, 77, 78, 85, 87, 89, 91, 92, 93, 94, 95, 98, 99, 100, 101, 103, 104, 105, and 106; wherein the interleukin-2 analog comprises any one of sequences selected from the group consisting of amino acid sequences of SEQ ID NOS: 10, 13, 14, 16, 17, 20, 21, 22, 32, 35, 36, 42, 53, 54, 87, 89, 91, 92, 93, 94, 98, 99, 100, 101, 103, 104, and 105; or wherein the interleukin-2 analog comprises any one of sequences selected from the group consisting of amino acid sequences of SEQ ID NOS: 22, 42, 53, 87, 105, and 106.

13. The long-acting conjugate of claim 11, wherein the interleukin-2 analog further comprises one or more amino acids at the C-terminus thereof.

14. A long-acting conjugate of an interleukin-2 analog, represented by Chemical Formula 3 below: 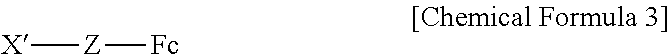
wherein X′ is an interleukin-2 analog comprising an amino acid sequence represented by General Formula 1 below:
[General Formula 1]
(General Formula 1, SEQ ID NO: 212)
X1-P-T-S-S-S-T-K-K-T-Q-L-Q-L-E-H-L-X18-X19-D-L-
X22-M-I-L-N-G-I-N-N-Y-K-N-P-K-L-T-X38-M-L-T-X42-
X43-F-X45-M-P-K-K-A-T-E-L-K-H-L-Q-C-L-E-X61-E-L-K-
P-L-E-X68-V-L-N-L-A-X74-S-K-N-F-H-X80-X81-P-R-X84-
X85-X86-S-N-I-N-X91-X92-V-X94-E-X96-K-G-S-E-T-T-F-
M-C-E-Y-A-D-E-T-A-T-I-V-E-F-L-N-R-W-I-T-F-S-Q-S-I-
I-S-T-L-T
in General Formula 1 above,
X1 is a deletion;
X18 is leucine (L) or arginine (R);
X19 is leucine (L) or tyrosine (Y);
X22 is glutamic acid (E) or glutamine (Q);
X38 is alanine (A), aspartic acid (D), or arginine (R);
X42 is alanine (A), phenylalanine (F), lysine (K), or tryptophan (W);
X43 is glutamic acid (E), lysine (K), or glutamine (Q);
X45 is alanine (A) or tyrosine (Y);
X61 is aspartic acid (D), glutamic acid (E), glutamine (Q), or arginine (R);
X68 is aspartic acid (D) or glutamic acid (E);
X74 is histidine (H) or glutamine (Q);
X80 is phenylalanine (F), leucine (L), valine (V), or tyrosine (Y);
X81 is aspartic acid (D), glutamic acid (E), or arginine (R);
X84 is aspartic acid (D) or glutamic acid (E);
X85 is alanine (A), glutamic acid (E), glycine (G), leucine (L), valine (V), tryptophan (W), or tyrosine (Y);
X86 is alanine (A), glycine (G), isoleucine (I), or valine (V);
X91 is threonine (T) or valine (V);
X92 is phenylalanine (F), isoleucine (I), or tyrosine (Y);
X94 is phenylalanine (F) or leucine (L);
X96 is phenylalanine (F) or leucine (L);
Z is a polyethylene glycol linker having a molecular weight of 2 kDa to 30 kDa;
Fc is an immunoglobulin Fc region in the form of a dimer; and
- indicates a covalent linkage between X′ and Z and between Z and Fc;
wherein the long-acting conjugate is a long-acting conjugate in which one end of Z is covalently linked only to one polypeptide chain in the dimeric Fc region, and one molecule of X′ is covalently linked to the opposite end of Z.

15. The long-acting conjugate of claim 14,
in General Formula 1 above,
X43 is lysine (K);
X45 is tyrosine (Y);
X61 is aspartic acid (D), glutamic acid (E), or glutamine (Q);
X68 is glutamic acid (E);
X74 is glutamine (Q);
X80 is phenylalanine (F) or leucine (L);
X85 is leucine (L), valine (V), or tyrosine (Y);
X86 is isoleucine (I) or valine (V); and
X92 is phenylalanine (F) or isoleucine (I).

16. The long-acting conjugate of claim 14, wherein the interleukin-2 analog further comprises one or more amino acids at the C-terminus thereof.

17. A long-acting conjugate of an interleukin-2 analog, represented by Chemical Formula 4 below: 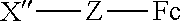
wherein X″ is an interleukin-2 analog comprising an amino acid sequence represented by General Formula 2 below:
[General Formula 2]
(General Formula 2, SEQ ID NO: 213)
X1-P-T-S-S-S-T-K-K-T-Q-L-Q-L-E-H-L-X18-L-D-L-X22-
M-I-L-N-G-I-N-N-Y-K-N-P-K-L-T-X38-M-L-T-X42-K-F-Y-
M-P-K-K-A-T-E-L-K-H-L-Q-C-L-E-X61-E-L-K-P-L-E-X68-
V-L-N-L-A-Q-S-K-N-F-H-F-X81-P-R-D-X85-X86-S-N-I-N-
V-F-V-L-E-L-K-G-S-E-T-T-F-M-C-E-Y-A-D-E-T-A-T-I-V-
E-F-L-N-R-W-I-T-F-S-Q-S-I-I-S-T-L-T
X1 is a deletion;
X18 is leucine (L) or arginine (R);
X22 is glutamic acid (E) or glutamine (Q);
X38 is alanine (A) or arginine (R);
X42 is phenylalanine (F) or lysine (K);
X61 is aspartic acid (D) or glutamic acid (E);
X68 is aspartic acid (D) or glutamic acid (E);
X81 is aspartic acid (D) or glutamic acid (E);
X85 is leucine (L) or valine (V);
X86 is isoleucine (I) or valine (V);
Z is a polyethylene glycol linker having a molecular weight of 2 kDa to 30 kDa;
Fc is an immunoglobulin Fc region in the form of a dimer; and
- indicates a covalent linkage between X″ and Z and between Z and Fc;
wherein the long-acting conjugate is a long-acting conjugate in which one end of Z is covalently linked only to one polypeptide chain in the dimeric Fc region, and one molecule of X″ is covalently linked to the opposite end of Z.

18. The long-acting conjugate of claim 17, wherein the interleukin-2 analog comprises any one of sequences selected from the group consisting of amino acid sequences of SEQ ID NOS: 22, 42, 53, 87, 105, and 106.

19. A pharmaceutical composition, comprising a pharmaceutically effective amount of the long-acting conjugate of claim 1 and a pharmaceutically acceptable excipient.

20. A pharmaceutical composition, comprising a pharmaceutically effective amount of the long-acting conjugate of claim 11 and a pharmaceutically acceptable excipient.


SEQUENCE LISTING (439 sequences)

SEQ ID NO: 1 - protein, 133 aa, Homo sapiens [sequence omitted]

SEQ ID NO: 2 - protein, 132 aa, synthetic construct
  REGION 1..132: note = IL-2 analog 1
PTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 3 - protein, 132 aa, synthetic construct
  REGION 1..132: note = IL-2 analog 2
PTSSSTKKTQLQLEHLLLDLQMILNGINNYKNPKLTRMLTFKFYMPKKATELKHLQCLEEELKPLEEVLNLAQSKNFHLRPRDLICNINVIVLELKGSETTFMCEYADETATIVEFLNRWITFSQSIISTLT

SEQ ID NO: 4 - protein, 132 aa, synthetic construct
  REGION 1..132: note = IL-2 analog 3
PTSSSTKKTQLQLEHLLLDLQMILNGINNYCNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 5 - protein, 132 aa, synthetic construct
  REGION 1..132: note = IL-2 analog 4
PTSSSTKKTQLQLEHLLLDLQMILNGINNYKNPCLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 6 - protein, 132 aa, synthetic construct
  REGION 1..132: note = IL-2 analog 5
PTSSSTKKTQLQLEHLLLDLQMILNGINNYKNPKLTRMLTFCFYMPKKATELKHLQCLEEELKPLEEVLNLAQSKNFHLRPRDLISNINVIVLELKGSETTFMCEYADETATIVEFLNRWITFSQSIISTLT

SEQ ID NO: 7 - protein, 132 aa, synthetic construct
  REGION 1..132: note = IL-2 analog 6
PTSSSTKKTQLQLEHLLLDLQMILNGINNYKNPKLTRMLTFKFYMPCKATELKHLQCLEEELKPLEEVLNLAQSKNFHLRPRDLISNINVIVLELKGSETTFMCEYADETATIVEFLNRWITFSQSIISTLT

SEQ ID NO: 8 - protein, 132 aa, synthetic construct
  REGION 1..132: note = IL-2 analog 7
PTSSSTKKTQLQLEHLLLDLQMILNGINNYKNPKLTRMLTFKFYMPKCATELKHLQCLEEELKPLEEVLNLAQSKNFHLRPRDLISNINVIVLELKGSETTFMCEYADETATIVEFLNRWITFSQSIISTLT

SEQ ID NO: 9 - protein, 132 aa, synthetic construct
  REGION 1..132: note = IL-2 analog 9
PTSSSTKKTQLQLEHLLLDLQMILNGINNYKNPKLTRMLTFKFYMPKKATELKHLQCLEEELKPLEEVLNLAQSCNFHLRPRDLISNINVIVLELKGSETTFMCEYADETATIVEFLNRWITFSQSIISTLT

SEQ ID NO: 10 - protein, 132 aa, synthetic construct
  REGION 1..132: note = IL-2 analog 10
PTSSSTKKTQLQLEHLLLDLQMILNGINNYKNPKLTAMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 11 - protein, 132 aa, synthetic construct
  REGION 1..132: note = IL-2 analog 10
PTSSSTKKTQLQLEHLLLDLQMILNGINNYKNPKLTRMLTKKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 12 - protein, 132 aa, synthetic construct
  REGION 1..132: note = IL-2 analog 11
PTSSSTKKTQLQLEHLLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 13 - protein, 132 aa, synthetic construct
  REGION 1..132: note = IL-2 analog 12
PTSSSTKKTQLQLEHLLLDLQMILNGINNYKNPKLTAMLTKKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 14 - protein, 132 aa, synthetic construct
  REGION 1..132: note = IL-2 analog 13
PTSSSTKKTQLQLEHLLLDLQMILNGINNYKNPKLTAMLTAKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 15 - protein, 132 aa, synthetic construct
  REGION 1..132: note = IL-2 analog 14
PTSSSTKKTQLQLEHLLYDLQMILNGINNYKNPKLTAMLTKKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 16 - protein, 132 aa, synthetic construct
  REGION 1..132: note = IL-2 analog 15
PTSSSTKKTQLQLEHLLLDLQMILNGINNYKNPKLTAMLTKKFYMPKKATELKHLQCLEEELKPLEEVLNLAQSKNFHLRPRELISNINVIVLELKGSETTFMCEYADETATIVEFLNRWITFSQSIISTLT

SEQ ID NO: 17 - protein, 132 aa, synthetic construct
  REGION 1..132: note = IL-2 analog 16
PTSSSTKKTQLQLEHLLLDLQMILNGINNYKNPKLTAMLTKKFYMPKKATELKHLQCLEEELKPLEEVLNLAQSKNFHLRPRDLISQINVIVLELKGSETTFMCEYADETATIVEFLNRWITFSQSIISTLT

SEQ ID NO: 18 - protein, 132 aa, synthetic construct
  REGION 1..132: note = IL-2 analog 17
PTSSSTKKTQLQLEHLLLDLQMILNGINNYKNPKLTAMLTKKFYMPKKATELKHLQCLEEELKPLEEVLNLAQSKNFHLRPRDLISNINTIVLELKGSETTFMCEYADETATIVEFLNRWITFSQSIISTLT

SEQ ID NO: 19 - protein, 132 aa, synthetic construct
  REGION 1..132: note = IL-2 analog 18
PTSSSTKKTQLQLEHLLLDLQMILNGINNYKNPKLTAMLTKKFYMPKKATELKHLQCLEQELKPLEEVLNLAQSKNFHLRPRDLISNINVIVLELKGSETTFMCEYADETATIVEFLNRWITFSQSIISTLT

SEQ ID NO: 20 - protein, 132 aa, synthetic construct
  REGION 1..132: note = IL-2 analog 19
PTSSSTKKTQLQLEHLLLDLQMILNGINNYKNPKLTAMLTKKFYMPKKATELKHLQCLEEELKPLEEVLNLAQSKNFHLDPRDLISNINVFVLELKGSETTFMCEYADETATIVEFLNRWITFSQSIISTLT

SEQ ID NO: 21 - protein, 132 aa, synthetic construct
  REGION 1..132: note = IL-2 analog 20
PTSSSTKKTQLQLEHLLLDLQMILNGINNYKNPKLTAMLTKKFYMPKKATELKHLQCLEEELKPLEEVLNLAQSKNFHLRPRDVVSNINVFVLELKGSETTFMCEYADETATIVEFLNRWITFSQSIISTLT

SEQ ID NO: 22 - protein, 132 aa, synthetic construct
  REGION 1..132: note = IL-2 analog 21
PTSSSTKKTQLQLEHLLLDLQMILNGINNYKNPKLTAMLTKKFYMPKKATELKHLQCLEEELKPLEEVLNLAQSKNFHFDPRDVVSNINVFVLELKGSETTFMCEYADETATIVEFLNRWITFSQSIISTLT

SEQ ID NO: 23 - protein, 132 aa, synthetic construct
  REGION 1..132: note = IL-2 analog 22
PTSSSTKKTQVQLEHLLLDLQMILNGINNYKNPKLTAMLTKKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 24 - protein, 132 aa, synthetic construct
  REGION 1..132: note = IL-2 analog 23
PTSSSTKKTQFQLEHLLLDLQMILNGINNYKNPKLTAMLTKKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 25 - protein, 132 aa, synthetic construct
  REGION 1..132: note = IL-2 analog 24
PTSSSTKKTQLQLEHLLVDLQMILNGINNYKNPKLTAMLTKKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 26 - protein, 132 aa, synthetic construct
  REGION 1..132: note = IL-2 analog 25
PTSSSTKKTQLQLEHLLFDLQMILNGINNYKNPKLTAMLTKKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 27 - protein, 132 aa, synthetic construct
  REGION 1..132: note = IL-2 analog 26
PTSSSTKKTQLQLEHLLLDLQMILNGINNYKNPKLTAMLTKKFYMPKKATELKHLQCLEEELKPLEEVLNLAQSKNFHLRPRDLISNFNVIVLELKGSETTFMCEYADETATIVEFLNRWITFSQSIISTLT

SEQ ID NO: 28 - protein, 132 aa, synthetic construct
  REGION 1..132: note = IL-2 analog 27
PTSSSTKKTQLQLEHLLLDLQMILNGINNYKNPKLTAMLTKKFYMPKKATELKHLQCLEEELKPLEEVLNLAQSKNFHLRPRDLISNINFIVLELKGSETTFMCEYADETATIVEFLNRWITFSQSIISTLT

SEQ ID NO: 29 - protein, 132 aa, synthetic construct
  REGION 1..132: note = IL-2 analog 28
PTSSSTKKTQLQLEHLLLDLQMILNGINNYKNPKLTAMLTKKFYMPKKATELKHLQCLEEELKPLEEVLNLAQSKNFHLRPRDLISNINVIVVELKGSETTFMCEYADETATIVEFLNRWITFSQSIISTLT

SEQ ID NO: 30 - protein, 132 aa, synthetic construct
  REGION 1..132: note = IL-2 analog 29
PTSSSTKKTQLQLEHLLLDLQMILNGINNYKNPKLTAMLTKKFYMPKKATELKHLQCLEEELKPLEEVLNLAQSKNFHLRPRDLISNINVIVLELKGSETTFMCEYADETATIVEFLNRWITFSTSIISTLT

SEQ ID NO: 31 - protein, 132 aa, synthetic construct
  REGION 1..132: note = IL-2 analog 30
PTSSSTKKTQLQLEHLLLDLQMILNGINNYKNPKLTAMLTFKFYMPKKATELKHLQCLEEELKPLEEVLNLAQSKNFHLDPRDLISNINVFVLELKGSETTFMCEYADETATIVEFLNRWITFSQSIISTLT

SEQ ID NO: 32 - protein, 132 aa, synthetic construct
  REGION 1..132: note = IL-2 analog 31
PTSSSTKKTQLQLEHLLLDLQMILNGINNYKNPKLTAMLTFKFYMPKKATELKHLQCLEEELKPLEEVLNLAQSKNFHLRPRELISNINVIVLELKGSETTFMCEYADETATIVEFLNRWITFSQSIISTLT

SEQ ID NO: 33 - protein, 132 aa, synthetic construct
  REGION 1..132: note = IL-2 analog 32
PTSSSTKKTQLQLEHLLLDLQMILNGINNYKNPKLTAMLTFKFYMPKKATELKHLQCLEEELKPLEEVLNLAQSKNFHLDPRELISNINVFVLELKGSETTFMCEYADETATIVEFLNRWITFSQSIISTLT

SEQ ID NO: 34 - protein, 132 aa, synthetic construct
  REGION 1..132: note = IL-2 analog 33
PTSSSTKKTQLQLEHLLLDLQMILNGINNYKNPKLTAMLTFKFYMPKKATELKHLQCLEEELKPLEEVLNLAQSKNFHFRPRDLISNINVIVLELKGSETTFMCEYADETATIVEFLNRWITFSQSIISTLT

SEQ ID NO: 35 - protein, 132 aa, synthetic construct
  REGION 1..132: note = IL-2 analog 34
PTSSSTKKTQLQLEHLLLDLQMILNGINNYKNPKLTAMLTFKFYMPKKATELKHLQCLEEELKPLEEVLNLAQSKNFHFRPRELISNINVIVLELKGSETTFMCEYADETATIVEFLNRWITFSQSIISTLT

SEQ ID NO: 36 - protein, 132 aa, synthetic construct
  REGION 1..132: note = IL-2 analog 35
PTSSSTKKTQLQLEHLLLDLQMILNGINNYKNPKLTAMLTFKFYMPKKATELKHLQCLEEELKPLEEVLNLAQSKNFHLRPRDLISNINVIVFEFKGSETTFMCEYADETATIVEFLNRWITFSQSIISTLT

SEQ ID NO: 37 - protein, 132 aa, synthetic construct
  REGION 1..132: note = IL-2 analog 36
PTSSSTKKTQLQLEHLLLDLQMILNGINNYKNPKLTAMLTFKFYMPKKATELKHLQCLEEELKPLEEVLNLAQSKNFHLRPRDLISNINVIVFEVKGSETTFMCEYADETATIVEFLNRWITFSQSIISTLT

SEQ ID NO: 38 - protein, 132 aa, synthetic construct
  REGION 1..132: note = IL-2 analog 37
PTSSSTKKTQLQLEHLLLDLQMILNGINNYKNPKLTAMLTFKFYMPKKATELKHLQCLEEELKPLEEVLNLAQSKNFHLRPRDLISNINVIVFEIKGSETTFMCEYADETATIVEFLNRWITFSQSIISTLT

SEQ ID NO: 39 - protein, 132 aa, synthetic construct
  REGION 1..132: note = IL-2 analog 38
PTSSSTKKTQLQLEHLLLDLQMILNGINNYKNPKLTAMLTKKFYMPKKATELKHLQCLEEELKPLEEVLNLAQSKNFHLDPRDLISNINVFVFEFKGSETTFMCEYADETATIVEFLNRWITFSQSIISTLT

SEQ ID NO: 40 - protein, 132 aa, synthetic construct
  REGION 1..132: note = IL-2 analog 39
PTSSSTKKTQLQLEHLLLDLQMILNGINNYKNPKLTAMLTKKFYMPKKATELKHLQCLEEELKPLEEVLNLAQSKNFHLDPRDLISNINVFVFEVKGSETTFMCEYADETATIVEFLNRWITFSQSIISTLT

SEQ ID NO: 41 - protein, 132 aa, synthetic construct
  REGION 1..132: note = IL-2 analog 40
PTSSSTKKTQLQLEHLLLDLQMILNGINNYKNPKLTAMLTKKFYMPKKATELKHLQCLEEELKPLEEVLNLAQSKNFHLDPRDLISNINVFVFEIKGSETTFMCEYADETATIVEFLNRWITFSQSIISTLT

SEQ ID NO: 42 - protein, 132 aa, synthetic construct
  REGION 1..132: note = IL-2 analog 41
PTSSSTKKTQLQLEHLLLDLQMILNGINNYKNPKLTRMLTFKFYMPKKATELKHLQCLEEELKPLEEVLNLAQSKNFHFDPRDVVSNINVFVLELKGSETTFMCEYADETATIVEFLNRWITFSQSIISTLT

SEQ ID NO: 43 - protein, 132 aa, synthetic construct
  REGION 1..132: note = IL-2 analog 42
PTSSSTKKTQLQLEHLLLDLQMILNGINNYKNPKLTAMLTKKFYMPKKATELKHLQCLEEELKPLEEVLNLAQSKNFHLEPRDLISNINVFVLELKGSETTFMCEYADETATIVEFLNRWITFSQSIISTLT

SEQ ID NO: 44 - protein, 132 aa, synthetic construct
  REGION 1..132: note = IL-2 analog 43
PTSSSTKKTQLQLEHLLLDLQMILNGINNYKNPKLTAMLTKKFYMPKKATELKHLQCLEEELKPLEEVLNLAQSKNFHLDPRDLISNINVLVLELKGSETTFMCEYADETATIVEFLNRWITFSQSIISTLT

SEQ ID NO: 45 - protein, 132 aa, synthetic construct
  REGION 1..132: note = IL-2 analog 44
PTSSSTKKTQLQLEHLLLDLQMILNGINNYKNPKLTAMLTKKFYMPKKATELKHLQCLEEELKPLEEVLNLAQSKNFHLDPRVLISNINVFVLELKGSETTFMCEYADETATIVEFLNRWITFSQSIISTLT

SEQ ID NO: 46 - protein, 132 aa, synthetic construct
  REGION 1..132: note = IL-2 analog 45
PTSSSTKKTQLQLEHLLLDLQMILNGINNYKNPKLTAMLTKKFYMPKKATELKHLQCLEEELKPLEEVLNLAQSKNFHLDPRFLISNINVFVLELKGSETTFMCEYADETATIVEFLNRWITFSQSIISTLT

SEQ ID NO: 47 - protein, 132 aa, synthetic construct
  REGION 1..132: note = IL-2 analog 46
PTSSSTKKTQLQLEHLLLVLQMILNGINNYKNPKLTAMLTKKFYMPKKATELKHLQCLEEELKPLEEVLNLAQSKNFHLDPRDLISNINVFVLELKGSETTFMCEYADETATIVEFLNRWITFSQSIISTLT

SEQ ID NO: 48 - protein, 132 aa, synthetic construct
  REGION 1..132: note = IL-2 analog 47
PTSSSTKKTQLQLEHLLLFLQMILNGINNYKNPKLTAMLTKKFYMPKKATELKHLQCLEEELKPLEEVLNLAQSKNFHLDPRDLISNINVFVLELKGSETTFMCEYADETATIVEFLNRWITFSQSIISTLT

SEQ ID NO: 49 - protein, 132 aa, synthetic construct
  REGION 1..132: note = IL-2 analog 48
PTSSSTKKTQLQLEHLLLDLQMILNGINNYKNPKLTAMLTKKFYMPKKATELKHLQCLEEELKPLEEVLNLAQSKNFHLDPRDLISVINVFVLELKGSETTFMCEYADETATIVEFLNRWITFSQSIISTLT

SEQ ID NO: 50 - protein, 132 aa, synthetic construct
  REGION 1..132: note = IL-2 analog 49
PTSSSTKKTQLQLEHLLLDLQMILNGINNYKNPKLTAMLTKKFYMPKKATELKHLQCLEEELKPLEEVLNLAQSKNFHLDPRDLISFINVFVLELKGSETTFMCEYADETATIVEFLNRWITFSQSIISTLT

SEQ ID NO: 51 - protein, 132 aa, synthetic construct
  REGION 1..132: note = IL-2 analog 50
PTSSSTKKTQLQLEHLLLDLQMILNGINNYKNPKLTRMLTKKFYMPKKATELKHLQCLEEELKPLEEVLNLAQSKNFHFDPRDVVSNINVFVLELKGSETTFMCEYADETATIVEFLNRWITFSQSIISTLT

SEQ ID NO: 52 - protein, 132 aa, synthetic construct
  REGION 1..132: note = IL-2 analog 51
PTSSSTKKTQLQLEHLLLDLQMILNGINNYKNPKLTRMLTFKFYMPKKATELKHLQCLEQELKPLEEVLNLAQSKNFHLDPRDLISNINVFVLELKGSETTFMCEYADETATIVEFLNRWITFSQSIISTLT

SEQ ID NO: 53 - protein, 132 aa, synthetic construct
  REGION 1..132: note = IL-2 analog 52
PTSSSTKKTQLQLEHLLLDLQMILNGINNYKNPKLTAMLTKKFYMPKKATELKHLQCLEEELKPLEEVLNLAQSKNFHFDPRDLISNINVFVLELKGSETTFMCEYADETATIVEFLNRWITFSQSIISTLT

SEQ ID NO: 54 - protein, 132 aa, synthetic construct
  REGION 1..132: note = IL-2 analog 53
PTSSSTKKTQLQLEHLLLDLQMILNGINNYKNPKLTAMLTKKFYMPKKATELKHLQCLEEELKPLEEVLNLAQSKNFHFDPRELISNINVFVLELKGSETTFMCEYADETATIVEFLNRWITFSQSIISTLT

SEQ ID NO: 55 - protein, 132 aa, synthetic construct
  REGION 1..132: note = IL-2 analog 54
PTSSSTKKTQLQLEHLLLDLQMILNGINNYKNPKLTAMLTKKFYMPKKATELKHLQCLEEELKPLEEVLNLAHSKNFHLDPRDLISNINVFVLELKGSETTFMCEYADETATIVEFLNRWITFSQSIISTLT

SEQ ID NO: 56 - protein, 132 aa, synthetic construct
  REGION 1..132: note = IL-2 analog 55
PTSSSTKKTQLQLEHLLLDLQMILNGINNYKNPKLTAMLTKKFYMPKKATELKHLQCLEEELKPLEEVLNLAHSKNFHFDPRDLISNINVFVLELKGSETTFMCEYADETATIVEFLNRWITFSQSIISTLT

SEQ ID NO: 57 - protein, 132 aa, synthetic construct
  REGION 1..132: note = IL-2 analog 56
PTSSSTKKTQLQLEHLLLDLQMILNGINNYKNPKLTAMLTKKFAMPKKATELKHLQCLEEELKPLEEVLNLAHSKNFHLDPRDLISNINVFVLELKGSETTFMCEYADETATIVEFLNRWITFSQSIISTLT

SEQ ID NO: 58 - protein, 132 aa, synthetic construct
  REGION 1..132: note = IL-2 analog 57
PTSSSTKKTQLQLEHLLLDLQMILNGINNYKNPKLTAMLTKKFAMPKKATELKHLQCLEEELKPLEEVLNLAHSKNFHFDPRDLISNINVFVLELKGSETTFMCEYADETATIVEFLNRWITFSQSIISTLT

SEQ ID NO: 59 - protein, 132 aa, synthetic construct
  REGION 1..132: note = IL-2 analog 61
PTSSSTKKTQLQLEHLLLDLQMILNGINNYKNPKLTAMLTKKFYMPKKATELKHLQCLEEELKPLEEVLNLAQSKNFHFDPRDAASNINVFVLELKGSETTFMCEYADETATIVEFLNRWITFSQSIISTLT

SEQ ID NO: 60 - protein, 132 aa, synthetic construct
  REGION 1..132: note = IL-2 analog 59
PTSSSTKKTQLQLEHLLLDLQMILNGINNYKNPKLTAMLTKKFYMPKKATELKHLQCLEEELKPLEEVLNLAQSKNFHFDPRDAASNINVYVLELKGSETTFMCEYADETATIVEFLNRWITFSQSIISTLT

SEQ ID NO: 61 - protein, 132 aa, synthetic construct
  REGION 1..132: note = IL-2 analog 60
PTSSSTKKTQLQLEHLLLDLQMILNGINNYKNPKLTAMLTKKFAMPKKATELKHLQCLEEELKPLEEVLNLAQSKNFHYRPRDAASNINVYVLELKGSETTFMCEYADETATIVEFLNRWITFSQSIISTLT

SEQ ID NO: 62 - protein, 132 aa, synthetic construct
  REGION 1..132: note = IL-2 analog 61
PTSSSTKKTQLQLEHLLLDLQMILNGINNYKNPKLTAMLTKKFYMPKKATELKHLQCLEEELKPLEEVLNLAQSKNFHYDPRDGVSNINVYVLELKGSETTFMCEYADETATIVEFLNRWITFSQSIISTLT

SEQ ID NO: 63 - protein, 132 aa, synthetic construct
  REGION 1..132: note = IL-2 analog 62
PTSSSTKKTQLQLEHLLLDLQMILNGINNYKNPKLTAMLTKKFYMPKKATELKHLQCLEEELKPLEEVLNLAQSKNFHWEPRDGASNINVFVLELKGSETTFMCEYADETATIVEFLNRWITFSQSIISTLT

SEQ ID NO: 64 - protein, 132 aa, synthetic construct
  REGION 1..132: note = IL-2 analog 63
PTSSSTKKTQLQLEHLLLDLQMILNGINNYKNPKLTAMLTKKFYMPKKATELKHLQCLEEELKPLEEVLNLAQSKNFHDEPRDTGSNINVFVLELKGSETTFMCEYADETATIVEFLNRWITFSQSIISTLT

SEQ ID NO: 65 - protein, 132 aa, synthetic construct
  REGION 1..132: note = IL-2 analog 64
PTSSSTKKTQLQLEHLLLDLQMILNGINNYKNPKLTAMLTKKFYMPKKATELKHLQCLEEELKPLEEVLNLAQSKNFHYNPRDVVSNINVYVLELKGSETTFMCEYADETATIVEFLNRWITFSQSIISTLT

SEQ ID NO: 66 - protein, 132 aa, synthetic construct
  REGION 1..132: note = IL-2 analog 65
PTSSSTKKTQLQLEHLLLDLQMILNGINNYKNPKLTAMLTKKFYMPKKATELKHLQCLEEELKPLEEVLNLAQSKNFHYEPRDVVSNINVFVLELKGSETTFMCEYADETATIVEFLNRWITFSQSIISTLT

SEQ ID NO: 67 - protein, 132 aa, synthetic construct
  REGION 1..132: note = IL-2 analog 66
PTSSSTKKTQLQLEHLLLDLQMILNGINNYKNPKLTAMLTKKFYMPKKATELKHLQCLEEELKPLEEVLNLAQSKNFHFEPRDFVSNINVFVLELKGSETTFMCEYADETATIVEFLNRWITFSQSIISTLT

SEQ ID NO: 68 - protein, 132 aa, synthetic construct
  REGION 1..132: note = IL-2 analog 67
PTSSSTKKTQLQLEHLLLDLQMILNGINNYKNPKLTAMLTKKFYMPKKATELKHLQCLEEELKPLEEVLNLAQSKNFHYDPRDFVSNINVWVLDLKGSETTFMCEYADETATIVEFLNRWITFSQSIISTLT

SEQ ID NO: 69 - protein, 132 aa, synthetic construct
  REGION 1..132: note = IL-2 analog 68
PTSSSTKKTQLQLEHLLLDLQMILNGINNYKNPKLTAMLTKKFYMPKKATELKHLQCLEEELKPLEEVLNLAQSKNFHFEPRDIVSNINEFVLELKGSETTFMCEYADETATIVEFLNRWITFSQSIISTLT

SEQ ID NO: 70 - protein, 132 aa, synthetic construct
  REGION 1..132: note = IL-2 analog 69
PTSSSTKKTQLQLEHLLLDLQMILNGINNYKNPKLTAMLTKKFYMPKKATELKHLQCLEEELKPLEEVLNLAQSKNFHYEPRDFLSNINEWVLDLKGSETTFMCEYADETATIVEFLNRWITFSQSIISTLT

SEQ ID NO: 71 - protein, 132 aa, synthetic construct
  REGION 1..132: note = IL-2 analog 70
PTSSSTKKTQLQLEHLLLDLQMILNGINNYKNPKLTAMLTKKFYMPKKATELKHLQCLEEELKPLEEVLNLAQSKNFHYDPRDVVSNINVFVLELKGSETTFMCEYADETATIVEFLNRWITFSQSIISTLT

SEQ ID NO: 72 - protein, 132 aa, synthetic construct
  REGION 1..132: note = IL-2 analog 71
PTSSSTKKTQLQLEHLLLDLQMILNGINNYKNPKLTAMLTKKFYMPKKATELKHLQCLEEELKPLEEVLNLAQSKNFHFEPRDVVSNINVFVLELKGSETTFMCEYADETATIVEFLNRWITFSQSIISTLT

SEQ ID NO: 73 - protein, 132 aa, synthetic construct
  REGION 1..132: note = IL-2 analog 72
PTSSSTKKTQLQLEHLLLDLQMILNGINNYKNPKLTAMLTKKFYMPKKATELKHLQCLEEELKPLEEVLNLAQSKNFHFDPRDVGSNINVFVLELKGSETTFMCEYADETATIVEFLNRWITFSQSIISTLT

SEQ ID NO: 74 - protein, 132 aa, synthetic construct
  REGION 1..132: note = IL-2 analog 73
PTSSSTKKTQLQLEHLLLDLQMILNGINNYKNPKLTAMLTKKFYMPKKATELKHLQCLEEELKPLEEVLNLAQSKNFHFDPRDWVSNINVFVLELKGSETTFMCEYADETATIVEFLNRWITFSQSIISTLT

SEQ ID NO: 75 - protein, 132 aa, synthetic construct
  REGION 1..132: note = IL-2 analog 74
PTSSSTKKTQLQLEHLLLDLQMILNGINNYKNPKLTDMLTKKFYMPKKATELKHLQCLEEELKPLEEVLNLAQSKNFHYDPRDVVSNINVFVLELKGSETTFMCEYADETATIVEFLNRWITFSQSIISTLT

SEQ ID NO: 76 - protein, 132 aa, synthetic construct
  REGION 1..132: note = IL-2 analog 75
PTSSSTKKTQLQLEHLLLDLQMILNGINNYKNPKLTAMLTKKFAMPKKATELKHLQCLEEELKPLEEVLNLAQSKNFHFEPRDVVSNINVFVLELKGSETTFMCEYADETATIVEFLNRWITFSQSIISTLT

SEQ ID NO: 77 - protein, 132 aa, synthetic construct
  REGION 1..132: note = IL-2 analog 76
PTSSSTKKTQLQLEHLLLDLQMILNGINNYKNPKLTAMLTKQFYMPKKATELKHLQCLERELKPLEEVLNLAQSKNFHFDPRDVGSNINVFVLELKGSETTFMCEYADETATIVEFLNRWITFSQSIISTLT

SEQ ID NO: 78 - protein, 132 aa, synthetic construct
  REGION 1..132: note = IL-2 analog 77
PTSSSTKKTQLQLEHLLLDLQMILNGINNYKNPKLTAMLTKEFYMPKKATELKHLQCLERELKPLEEVLNLAQSKNFHFDPRDWVSNINVFVLELKGSETTFMCEYADETATIVEFLNRWITFSQSIISTLT

SEQ ID NO: 79 - protein, 132 aa, synthetic construct
  REGION 1..132: note = IL-2 analog 78
PTSSSTKKTQLQLEHLLLDLQMILNGINNYKNPELTAMLTKKFYMPKKATELKHLQCLEEELKPLEEVLNLAQSKNFHFEPRDVVSNINVFVLELKGSETTFMCEYADETATIVEFLNRWITFSQSIISTLT

SEQ ID NO: 80 - protein, 132 aa, synthetic construct
  REGION 1..132: note = IL-2 analog 79
PTSSSTKKTQLQLEHLLLDLQMILNGINNYKNPELTAMLTKKFYMPKKATELKHLQCLEEELKPLEEVLNLAHSKNFHFEPRDVVSNINVFVLELKGSETTFMCEYADETATIVEFLNRWITFSQSIISTLT

SEQ ID NO: 81 - protein, 132 aa, synthetic construct
  REGION 1..132: note = IL-2 analog 80
PTSSSTKKTQLQLEHLLLDLQMILNGINNYKNPELTAMLTKKFYMPKKATELKHLQCLEEELKPLEEVLNLAHSKNFHFEGRDVVSNINVFVLELKGSETTFMCEYADETATIVEFLNRWITFSQSIISTLT

SEQ ID NO: 82 - protein, 132 aa, synthetic construct
  REGION 1..132: note = IL-2 analog 81
PTSSSTKKTQLQLEHLLLDLQMILNGINNYKNPELTAMLTKKFYMPKKATELKHLQCLEEELKPLEEVLNLAHSKNFHFEVRDVVSNINVFVLELKGSETTFMCEYADETATIVEFLNRWITFSQSIISTLT

SEQ ID NO: 83 - protein, 132 aa, synthetic construct
  REGION 1..132: note = IL-2 analog 82
PTSSSTKKTQLQLEHLRLDLEMILNGINNYKNPKLTRMLTFKFYMPKKATELKHLQCLEEELKPLEEVLNLAQSKNFHFDPRDEVSNINVFVLELKGSETTFMCEYADETATIVEFLNRWITFSQSIISTLT

SEQ ID NO: 84 - protein, 132 aa, synthetic construct
  REGION 1..132: note = IL-2 analog 83
PTSSSTKKTQLQLEHLRRDLEMILNGINNYKNPKLTRMLTFKFYMPKKATELKHLQCLEEELKPLEEVLNLAQSKNFHFDPRDEVSNINVFVLELKGSETTFMCEYADETATIVEFLNRWITFSQSIISTLT

SEQ ID NO: 85 - protein, 132 aa, synthetic construct
  REGION 1..132: note = IL-2 analog 84
PTSSSTKKTQLQLEHLRLDLEMILNGINNYKNPKLTRMLTFKFYMPKKATELKHLQCLEEELKPLEEVLNLAQSKNFHVDPRDEVSNINVFVLELKGSETTFMCEYADETATIVEFLNRWITFSQSIISTLT

SEQ ID NO: 86 - protein, 132 aa, synthetic construct
  REGION 1..132: note = IL-2 analog 85
PTSSSTKKTQLQLEHLLLDLQMILNGINNYKNPKLTRMLTFKFYMPKKATELKHLQCLEEELKPLEEVLNLAQSKNFHFEPRDVVSNINVFVLELKGSETTFMCEYADETATIVEFLNRWITFSQSIISTLT

SEQ ID NO: 87 - protein, 132 aa, synthetic construct
  REGION 1..132: note = IL-2 analog 86
PTSSSTKKTQLQLEHLRLDLEMILNGINNYKNPKLTRMLTFKFYMPKKATELKHLQCLEEELKPLEEVLNLAQSKNFHFEPRDVVSNINVFVLELKGSETTFMCEYADETATIVEFLNRWITFSQSIISTLT

SEQ ID NO: 88 - protein, 132 aa, synthetic construct
  REGION 1..132: note = IL-2 analog 87
PTSSSTKKTQLQLEHLRRDLEMILNGINNYKNPKLTRMLTFKFYMPKKATELKHLQCLEEELKPLEEVLNLAQSKNFHFEPRDVVSNINVFVLELKGSETTFMCEYADETATIVEFLNRWITFSQSIISTLT

SEQ ID NO: 89 - protein, 132 aa, synthetic construct
  REGION 1..132: note = IL-2 analog 88
PTSSSTKKTQLQLEHLLLDLQMILNGINNYKNPKLTRMLTFKFYMPKKATELKHLQCLEDELKPLEEVLNLAQSKNFHFEPRDVVSNINVFVLELKGSETTFMCEYADETATIVEFLNRWITFSQSIISTLT

SEQ ID NO: 90 - protein, 132 aa, synthetic construct
  REGION 1..132: note = IL-2 analog 89
PTSSSTKKTQLQLEHLLLDLQMILNGINNYKNPKLTAMLTFKFYMPKKATELKHLQCLEEELKPLEQVLNLAQSKNFHFEPRDVVSNINVFVLELKGSETTFMCEYADETATIVEFLNRWITFSQSIISTLT

SEQ ID NO: 91 - protein, 132 aa, synthetic construct
  REGION 1..132: note = IL-2 analog 90
PTSSSTKKTQLQLEHLLLDLQMILNGINNYKNPKLTRMLTWKFYMPKKATELKHLQCLEEELKPLEEVLNLAQSKNFHFEPRDVVSNINVFVLELKGSETTFMCEYADETATIVEFLNRWITFSQSIISTLT

SEQ ID NO: 92 - protein, 132 aa, synthetic construct
  REGION 1..132: note = IL-2 analog 91
PTSSSTKKTQLQLEHLLLDLQMILNGINNYKNPKLTRMLTFKFYMPKKATELKHLQCLEQELKPLEEVLNLAQSKNFHFEPRDVVSNINVFVLELKGSETTFMCEYADETATIVEFLNRWITFSQSIISTLT

SEQ ID NO: 93 - protein, 132 aa, synthetic construct
  REGION 1..132: note = IL-2 analog 92
PTSSSTKKTQLQLEHLLLDLQMILNGINNYKNPKLTRMLTFKFYMPKKATELKHLQCLEEELKPLEEVLNLAQSKNFHFEPRDVVSNINTFVLELKGSETTFMCEYADETATIVEFLNRWITFSQSIISTLT

SEQ ID NO: 94 - protein, 132 aa, synthetic construct
  REGION 1..132: note = IL-2 analog 93
PTSSSTKKTQLQLEHLLLDLQMILNGINNYKNPKLTRMLTFKFYMPKKATELKHLQCLEEELKPLEEVLNLAQSKNFHFEPREVVSNINTFVLELKGSETTFMCEYADETATIVEFLNRWITFSQSIISTLT

SEQ ID NO: 95 - protein, 132 aa, synthetic construct
  REGION 1..132: note = IL-2 analog 94
PTSSSTKKTQLQLEHLLLDLQMILNGINNYKNPKLTRMLTFKFYMPKKATELKHLQCLEEELKPLEEVLNLAQSKNFHFEPRDVVSNINVFVFEFKGSETTFMCEYADETATIVEFLNRWITFSQSIISTLT

SEQ ID NO: 96 - protein, 132 aa, synthetic construct
  REGION 1..132: note = IL-2 analog 95
PTSSSTKKTQLQLEHLLLDLQMILNGINNYKNPKLTRMLTFKFYMPKKATELKHLQCLEEELKPLEEGLNLAQSKNFHFEPRDVVSNINVFVLELKGSETTFMCEYADETATIVEFLNRWITFSQSIISTLT

SEQ ID NO: 97 - protein, 132 aa, synthetic construct
  REGION 1..132: note = IL-2 analog 96
PTSSSTKKTQLQLEHLLLDLQMILNGINNYKNPKLTRMLTFKFYMPKKATELKHLQCLEEELKPLEEGLNLAASKNFHFEPRDVVSNINVFVLELKGSETTFMCEYADETATIVEFLNRWITFSQSIISTLT

SEQ ID NO: 98 - protein, 132 aa, synthetic construct
  REGION 1..132: note = IL-2 analog 97
PTSSSTKKTQLQLEHLLLDLQMILNGINNYKNPKLTAMLTFKFYMPKKATELKHLQCLEEELKPLEEVLNLAQSKNFHFDPRDLISNINVFVLELKGSETTFMCEYADETATIVEFLNRWITFSQSIISTLT

SEQ ID NO: 99 - protein, 132 aa, synthetic construct
  REGION 1..132: note = IL-2 analog 98
PTSSSTKKTQLQLEHLLLDLQMILNGINNYKNPKLTAMLTFKFYMPKKATELKHLQCLEEELKPLEEVLNLAQSKNFHFEPRDVISNINVFVLELKGSETTFMCEYADETATIVEFLNRWITFSQSIISTLT

SEQ ID NO: 100 - protein, 132 aa, synthetic construct
  REGION 1..132: note = IL-2 analog 99
PTSSSTKKTQLQLEHLLLDLQMILNGINNYKNPKLTAMLTFKFYMPKKATELKHLQCLEEELKPLEEVLNLAQSKNFHFEPRDLVSNINVFVLELKGSETTFMCEYADETATIVEFLNRWITFSQSIISTLT

SEQ ID NO: 101 - protein, 132 aa, synthetic construct
  REGION 1..132: note = IL-2 analog 100
PTSSSTKKTQLQLEHLLLDLQMILNGINNYKNPKLTRMLTFKFYMPKKATELKHLQCLEEELKPLEEVLNLAQSKNFHFEPRDYVSNINVFVLELKGSETTFMCEYADETATIVEFLNRWITFSQSIISTLT

SEQ ID NO: 102 - protein, 132 aa, synthetic construct
  REGION 1..132: note = IL-2 analog 101
PTSSSTKKTQLQLEHLLLDLQMILNGINNYKNPKLTRMLTFKFYMPKKATELKHLQCLEEELKPLEEVLNLAQSKNFHFEPRDLASNINVFVLELKGSETTFMCEYADETATIVEFLNRWITFSQSIISTLT

SEQ ID NO: 103 - protein, 132 aa, synthetic construct
  REGION 1..132: note = IL-2 analog 102
PTSSSTKKTQLQLEHLLLDLQMILNGINNYKNPKLTRMLTFKFYMPKKATELKHLQCLEEELKPLEEVLNLAQSKNFHFEPRDVVSNINVIVLELKGSETTFMCEYADETATIVEFLNRWITFSQSIISTLT

SEQ ID NO: 104 - protein, 132 aa, synthetic construct
  REGION 1..132: note = IL-2 analog 103
PTSSSTKKTQLQLEHLRLDLEMILNGINNYKNPKLTAMLTFKFYMPKKATELKHLQCLEEELKPLEEVLNLAQSKNFHFEPRDVVSNINVFVLELKGSETTFMCEYADETATIVEFLNRWITFSQSIISTLT

SEQ ID NO: 105 - protein, 132 aa, synthetic construct
  REGION 1..132: note = IL-2 analog 104
PTSSSTKKTQLQLEHLRLDLEMILNGINNYKNPKLTRMLTFKFYMPKKATELKHLQCLEDELKPLEEVLNLAQSKNFHFEPRDVVSNINVFVLELKGSETTFMCEYADETATIVEFLNRWITFSQSIISTLT

SEQ ID NO: 106 - protein, 132 aa, synthetic construct
  REGION 1..132: note = IL-2 analog 105
PTSSSTKKTQLQLEHLRLDLEMILNGINNYKNPKLTRMLTFKFYMPKKATELKHLQCLEEELKPLEDVLNLAQSKNFHFEPRDVVSNINVFVLELKGSETTFMCEYADETATIVEFLNRWITFSQSIISTLT

SEQ ID NO: 107 - DNA, 399 bp, synthetic construct
  misc_feature 1..399: note = IL-2 analog 1
  source 1..399: mol_type = other DNA
cctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattacctttagtcaaagcatcatctcaacactgacttga

SEQ ID NO: 108 - DNA, 399 bp, synthetic construct
  misc_feature 1..399: note = IL-2 analog 2
  source 1..399: mol_type = other DNA
cctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatctgcaatatcaacgtaatagttctggaactaaagggatctgaaacaacattcatgtgtgaatatgctgatgagacagcaaccattgtagaatttctgaacagatggattacctttagtcaaagcatcatctcaacactgacttga

SEQ ID NO: 109 - DNA, 399 bp, synthetic construct
  misc_feature 1..399: note = IL-2 analog 3
  source 1..399: mol_type = other DNA
cctacttcaagttctacaaagaaaacacagctacaactggagcatttactgctggatttacagatgattttgaatggaattaataattactgtaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattacctttagtcaaagcatcatctcaacactgacttga

SEQ ID NO: 110 - DNA, 399 bp, synthetic construct
  misc_feature 1..399: note = IL-2 analog 4
  source 1..399: mol_type = other DNA
cctacttcaagttctacaaagaaaacacagctacaactggagcatttactgctggatttacagatgattttgaatggaattaataattacaagaatccctgtctcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattacctttagtcaaagcatcatctcaacactgacttga

SEQ ID NO: 111 - DNA, 399 bp, synthetic construct
  misc_feature 1..399: note = IL-2 analog 5
  source 1..399: mol_type = other DNA
cctacttcaagttctacaaagaaaacacagctacaactggagcatttactgctggatttacagatgattttgaatggaattaataattacaagaatcccaaactcaccaggatgctcacattttgtttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattacctttagtcaaagcatcatctcaacactgacttga

SEQ ID NO: 112 - DNA, 399 bp, synthetic construct
  misc_feature 1..399: note = IL-2 analog 6
  source 1..399: mol_type = other DNA
cctacttcaagttctacaaagaaaacacagctacaactggagcatttactgctggatttacagatgattttgaatggaattaataattacaagaatcccaaactcaccaggatgctcacatttaagttttacatgccctgtaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattacctttagtcaaagcatcatctcaacactgacttga

SEQ ID NO: 113 - DNA, 399 bp, synthetic construct
  misc_feature 1..399: note = IL-2 analog 7
  source 1..399: mol_type = other DNA
cctacttcaagttctacaaagaaaacacagctacaactggagcatttactgctggatttacagatgattttgaatggaattaataattacaagaatcccaaactcaccaggatgctcacatttaagttttacatgcccaagtgtgccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattacctttagtcaaagcatcatctcaacactgacttga

SEQ ID NO: 114 - DNA, 399 bp, synthetic construct
  misc_feature 1..399: note = IL-2 analog 8
  source 1..399: mol_type = other DNA
cctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagctgtaactttcacttaagacccagggacttaatcagcaatatcaacgtaatagttctggaactaaagggatctgaaacaacattcatgtgtgaatatgctgatgagacagcaaccattgtagaatttctgaacagatggattacctttagtcaaagcatcatctcaacactgacttga

SEQ ID NO: 115 - DNA, 399 bp, synthetic construct
  misc_feature 1..399: note = IL-2 analog 9
  source 1..399: mol_type = other DNA
cctacttcaagttctacaaagaaaacacagctacaactggagcatttactgctggatttacagatgattttgaatggaattaataattacaagaatcccaaactcaccgcgatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattacctttagtcaaagcatcatctcaacactgacttga

SEQ ID NO: 116 - DNA, 399 bp, synthetic construct
  misc_feature 1..399: note = IL-2 analog 10
  source 1..399: mol_type = other DNA
cctacttcaagttctacaaagaaaacacagctacaactggagcatttactgctggatttacagatgattttgaatggaattaataattacaagaatcccaaactcaccaggatgctcacaaagaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattacctttagtcaaagcatcatctcaacactgacttga

SEQ ID NO: 117 - DNA, 399 bp, synthetic construct
  misc_feature 1..399: note = IL-2 analog 11
  source 1..399: mol_type = other DNA
cctacttcaagttctacaaagaaaacacagctacaactggagcatttactgctggatttacagatgattttgaatggaattaataattacaagaatcccaaactcaccaggatgctcacagctaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattacctttagtcaaagcatcatctcaacactgacttga

SEQ ID NO: 118 - DNA, 399 bp, synthetic construct
  misc_feature 1..399: note = IL-2 analog 12
  source 1..399: mol_type = other DNA
cctacttcaagttctacaaagaaaacacagctacaactggagcatttactgctggatttacagatgattttgaatggaattaataattacaagaatcccaaactcaccgcgatgctcacaaagaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattacctttagtcaaagcatcatctcaacactgacttga

SEQ ID NO: 119 - DNA, 399 bp, synthetic construct
  misc_feature 1..399: note = IL-2 analog 13
  source 1..399: mol_type = other DNA
cctacttcaagttctacaaagaaaacacagctacaactggagcatttactgctggatttacagatgattttgaatggaattaataattacaagaatcccaaactcaccgcgatgctcacagctaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattacctttagtcaaagcatcatctcaacactgacttga

SEQ ID NO: 120 - DNA, 399 bp, synthetic construct
  misc_feature 1..399: note = IL-2 analog 14
  source 1..399: mol_type = other DNA
cctacttcaagttctacaaagaaaacacagctacaactggagcatttactgtacgatttacagatgattttgaatggaattaataattacaagaatcccaaactcaccgcgatgctcacaaagaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattacctttagtcaaagcatcatctcaacactgacttga

SEQ ID NO: 121 - DNA, 399 bp, synthetic construct
  misc_feature 1..399: note = IL-2 analog 15
  source 1..399: mol_type = other DNA
cctacttcaagttctacaaagaaaacacagctacaactggagcatttactgctggatttacagatgattttgaatggaattaataattacaagaatcccaaactcaccgcgatgctcacaaagaagttttacatgcccaagaaggccacagaactgaaacatcttcagtgtctagaagaagaactcaaacctctggaggaagtgctaaatttagctcaaagcaaaaactttcacttaagacccagggagttaatcagcaatatcaacgtaatagttctggaactaaagggatctgaaacaacattcatgtgtgaatatgctgatgagacagcaaccattgtagaatttctgaacagatggattacctttagtcaaagcatcatctcaacactgacttga

SEQ ID NO: 122 - DNA, 399 bp, synthetic construct
  misc_feature 1..399: note = IL-2 analog 16
  source 1..399: mol_type = other DNA
cctacttcaagttctacaaagaaaacacagctacaactggagcatttactgctggatttacagatgattttgaatggaattaataattacaagaatcccaaactcaccgcgatgctcacaaagaagttttacatgcccaagaaggccacagaactgaaacatcttcagtgtctagaagaagaactcaaacctctggaggaagtgctaaatttagctcaaagcaaaaactttcacttaagacccagggacttaatcagccaaatcaacgtaatagttctggaactaaagggatctgaaacaacattcatgtgtgaatatgctgatgagacagcaaccattgtagaatttctgaacagatggattacctttagtcaaagcatcatctcaacactgacttga

SEQ ID NO: 123 - DNA, 399 bp, synthetic construct
  misc_feature 1..399: note = IL-2 analog 17
  source 1..399: mol_type = other DNA
cctacttcaagttctacaaagaaaacacagctacaactggagcatttactgctggatttacagatgattttgaatggaattaataattacaagaatcccaaactcaccgcgatgctcacaaagaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacacaatagttctggaactaaagggatctgaaacaacattcatgtgtgaatatgctgatgagacagcaaccattgtagaatttctgaacagatggattacctttagtcaaagcatcatctcaacactgacttga

SEQ ID NO: 124 - DNA, 399 bp, synthetic construct
  misc_feature 1..399: note = IL-2 analog 18
  source 1..399: mol_type = other DNA
cctacttcaagttctacaaagaaaacacagctacaactggagcatttactgctggatttacagatgattttgaatggaattaataattacaagaatcccaaactcaccgcgatgctcacaaagaagttttacatgcccaagaaggccacagaactgaaacatcttcagtgtctagaacaggaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattacctttagtcaaagcatcatctcaacactgacttga

SEQ ID NO: 125 - DNA, 399 bp, synthetic construct
  misc_feature 1..399: note = IL-2 analog 19
  source 1..399: mol_type = other DNA
cctacttcaagttctacaaagaaaacacagctacaactggagcatttactgctggatttacagatgattttgaatggaattaataattacaagaatcccaaactcaccgcgatgctcacaaagaagttttacatgcccaagaaggccacagaactgaaacatcttcagtgtctagaagaagaactcaaacctctggaggaagtgctaaatttagctcaaagcaaaaactttcacttagaccccagggacttaatcagcaatatcaacgtatttgttctggaactaaagggatctgaaacaacattcatgtgtgaatatgctgatgagacagcaaccattgtagaatttctgaacagatggattacctttagtcaaagcatcatctcaacactgacttga

SEQ ID NO: 126 - DNA, 399 bp, synthetic construct
  misc_feature 1..399: note = IL-2 analog 20
  source 1..399: mol_type = other DNA
cctacttcaagttctacaaagaaaacacagctacaactggagcatttactgctggatttacagatgattttgaatggaattaataattacaagaatcccaaactcaccgcgatgctcacaaagaagttttacatgcccaagaaggccacagaactgaaacatcttcagtgtctagaagaagaactcaaacctctggaggaagtgctaaatttagctcaaagcaaaaactttcacttaagacccagggacgtagtcagcaatatcaacgtatttgttctggaactaaagggatctgaaacaacattcatgtgtgaatatgctgatgagacagcaaccattgtagaatttctgaacagatggattacctttagtcaaagcatcatctcaacactgacttga

SEQ ID NO: 127 - DNA, 399 bp, synthetic construct
  misc_feature 1..399: note = IL-2 analog 21
  source 1..399: mol_type = other DNA
cctacttcaagttctacaaagaaaacacagctacaactggagcatttactgctggatttacagatgattttgaatggaattaataattacaagaatcccaaactcaccgcgatgctcacaaagaagttttacatgcccaagaaggccacagaactgaaacatcttcagtgtctagaagaagaactcaaacctctggaggaagtgctaaatttagctcaaagcaaaaactttcactttgaccccagggacgtagtcagcaatatcaacgtatttgttctggaactaaagggatctgaaacaacattcatgtgtgaatatgctgatgagacagcaaccattgtagaatttctgaacagatggattacctttagtcaaagcatcatctcaacactgacttga

SEQ ID NO: 128 - DNA, 399 bp, synthetic construct
  misc_feature 1..399: note = IL-2 analog 22
  source 1..399: mol_type = other DNA
cctacttcaagttctacaaagaaaacacaggtacaactggagcatttactgctggatttacagatgattttgaatggaattaataattacaagaatcccaaactcaccgcgatgctcacaaagaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattacctttagtcaaagcatcatctcaacactgacttga

SEQ ID NO: 129 - DNA, 399 bp, synthetic construct
  misc_feature 1..399: note = IL-2 analog 23
  source 1..399: mol_type = other DNA
cctacttcaagttctacaaagaaaacacagtttcaactggagcatttactgctggatttacagatgattttgaatggaattaataattacaagaatcccaaactcaccgcgatgctcacaaagaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattacctttagtcaaagcatcatctcaacactgacttga

SEQ ID NO: 130 - DNA, 399 bp, synthetic construct
  misc_feature 1..399: note = IL-2 analog 24
  source 1..399: mol_type = other DNA
cctacttcaagttctacaaagaaaacacagctacaactggagcatttactggtggatttacagatgattttgaatggaattaataattacaagaatcccaaactcaccgcgatgctcacaaagaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattacctttagtcaaagcatcatctcaacactgacttga

SEQ ID NO: 131 - DNA, 399 bp, synthetic construct
  misc_feature 1..399: note = IL-2 analog 25
  source 1..399: mol_type = other DNA
cctacttcaagttctacaaagaaaacacagctacaactggagcatttactgttcgatttacagatgattttgaatggaattaataattacaagaatcccaaactcaccgcgatgctcacaaagaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattacctttagtcaaagcatcatctcaacactgacttga

SEQ ID NO: 132 - DNA, 399 bp, synthetic construct
  misc_feature 1..399: note = IL-2 analog 26
  source 1..399: mol_type = other DNA
cctacttcaagttctacaaagaaaacacagctacaactggagcatttactgctggatttacagatgattttgaatggaattaataattacaagaatcccaaactcaccgcgatgctcacaaagaagttttacatgcccaagaaggccacagaactgaaacatcttcagtgtctagaagaagaactcaaacctctggaggaagtgctaaatttagctcaaagcaaaaactttcacttaagacccagggacttaatcagcaatttcaacgtaatagttctggaactaaagggatctgaaacaacattcatgtgtgaatatgctgatgagacagcaaccattgtagaatttctgaacagatggattacctttagtcaaagcatcatctcaacactgacttga

SEQ ID NO: 133 - DNA, 399 bp, synthetic construct
  misc_feature 1..399: note = IL-2 analog 27
  source 1..399: mol_type = other DNA
cctacttcaagttctacaaagaaaacacagctacaactggagcatttactgctggatttacagatgattttgaatggaattaataattacaagaatcccaaactcaccgcgatgctcacaaagaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaactttatagttctggaactaaagggatctgaaacaacattcatgtgtgaatatgctgatgagacagcaaccattgtagaatttctgaacagatggattacctttagtcaaagcatcatctcaacactgacttga

SEQ ID NO: 134 - DNA, 399 bp, synthetic construct
  misc_feature 1..399: note = IL-2 analog 28
  source 1..399: mol_type = other DNA
cctacttcaagttctacaaagaaaacacagctacaactggagcatttactgctggatttacagatgattttgaatggaattaataattacaagaatcccaaactcaccgcgatgctcacaaagaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttgtggaactaaagggatctgaaacaacattcatgtgtgaatatgctgatgagacagcaaccattgtagaatttctgaacagatggattacctttagtcaaagcatcatctcaacactgacttga

SEQ ID NO: 135 - DNA, 399 bp, synthetic construct
  misc_feature 1..399: note = IL-2 analog 29
  source 1..399: mol_type = other DNA
cctacttcaagttctacaaagaaaacacagctacaactggagcatttactgctggatttacagatgattttgaatggaattaataattacaagaatcccaaactcaccgcgatgctcacaaagaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattacctttagtacaagcatcatctcaacactgacttga

SEQ ID NO: 136 - DNA, 399 bp, synthetic construct
  misc_feature 1..399: note = IL-2 analog 30
  source 1..399: mol_type = other DNA
cctacttcaagttctacaaagaaaacacagctacaactggagcatttactgctggatttacagatgattttgaatggaattaataattacaagaatcccaaactcaccgcgatgctcacatttaagttttacatgcccaagaaggccacagaactgaaacatcttcagtgtctagaagaagaactcaaacctctggaggaagtgctaaatttagctcaaagcaaaaactttcacttagaccccagggacttaatcagcaatatcaacgtatttgttctggaactaaagggatctgaaacaacattcatgtgtgaatatgctgatgagacagcaaccattgtagaatttctgaacagatggattacctttagtcaaagcatcatctcaacactgacttga

SEQ ID NO: 137 - DNA, 399 bp, synthetic construct
  misc_feature 1..399: note = IL-2 analog 31
  source 1..399: mol_type = other DNA
cctacttcaagttctacaaagaaaacacagctacaactggagcatttactgctggatttacagatgattttgaatggaattaataattacaagaatcccaaactcaccgcgatgctcacatttaagttttacatgcccaagaaggccacagaactgaaacatcttcagtgtctagaagaagaactcaaacctctggaggaagtgctaaatttagctcaaagcaaaaactttcacttaagacccagggagttaatcagcaatatcaacgtaatagttctggaactaaagggatctgaaacaacattcatgtgtgaatatgctgatgagacagcaaccattgtagaatttctgaacagatggattacctttagtcaaagcatcatctcaacactgacttga

SEQ ID NO: 138 - DNA, 399 bp, synthetic construct
  misc_feature 1..399: note = IL-2 analog 32
  source 1..399: mol_type = other DNA
cctacttcaagttctacaaagaaaacacagctacaactggagcatttactgctggatttacagatgattttgaatggaattaataattacaagaatcccaaactcaccgcgatgctcacatttaagttttacatgcccaagaaggccacagaactgaaacatcttcagtgtctagaagaagaactcaaacctctggaggaagtgctaaatttagctcaaagcaaaaactttcacttagaccccagggagttaatcagcaatatcaacgtatttgttctggaactaaagggatctgaaacaacattcatgtgtgaatatgctgatgagacagcaaccattgtagaatttctgaacagatggattacctttagtcaaagcatcatctcaacactgacttga

SEQ ID NO: 139 - DNA, 399 bp, synthetic construct
  misc_feature 1..399: note = IL-2 analog 33
  source 1..399: mol_type = other DNA
cctacttcaagttctacaaagaaaacacagctacaactggagcatttactgctggatttacagatgattttgaatggaattaataattacaagaatcccaaactcaccgcgatgctcacatttaagttttacatgcccaagaaggccacagaactgaaacatcttcagtgtctagaagaagaactcaaacctctggaggaagtgctaaatttagctcaaagcaaaaactttcactttagacccagggacttaatcagcaatatcaacgtaatagttctggaactaaagggatctgaaacaacattcatgtgtgaatatgctgatgagacagcaaccattgtagaatttctgaacagatggattacctttagtcaaagcatcatctcaacactgacttga

SEQ ID NO: 140 - DNA, 399 bp, synthetic construct
  misc_feature 1..399: note = IL-2 analog 34
  source 1..399: mol_type = other DNA
cctacttcaagttctacaaagaaaacacagctacaactggagcatttactgctggatttacagatgattttgaatggaattaataattacaagaatcccaaactcaccgcgatgctcacatttaagttttacatgcccaagaaggccacagaactgaaacatcttcagtgtctagaagaagaactcaaacctctggaggaagtgctaaatttagctcaaagcaaaaactttcactttagacccagggagttaatcagcaatatcaacgtaatagttctggaactaaagggatctgaaacaacattcatgtgtgaatatgctgatgagacagcaaccattgtagaatttctgaacagatggattacctttagtcaaagcatcatctcaacactgacttga

SEQ ID NO: 141 - DNA, 399 bp, synthetic construct
  misc_feature 1..399: note = IL-2 analog 35
  source 1..399: mol_type = other DNA
cctacttcaagttctacaaagaaaacacagctacaactggagcatttactgctggatttacagatgattttgaatggaattaataattacaagaatcccaaactcaccgcgatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttttcgaatttaagggatctgaaacaacattcatgtgtgaatatgctgatgagacagcaaccattgtagaatttctgaacagatggattacctttagtcaaagcatcatctcaacactgacttga

SEQ ID NO: 142 - DNA, 399 bp, synthetic construct
  misc_feature 1..399: note = IL-2 analog 36
  source 1..399: mol_type = other DNA
cctacttcaagttctacaaagaaaacacagctacaactggagcatttactgctggatttacagatgattttgaatggaattaataattacaagaatcccaaactcaccgcgatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttttcgaagtaaagggatctgaaacaacattcatgtgtgaatatgctgatgagacagcaaccattgtagaatttctgaacagatggattacctttagtcaaagcatcatctcaacactgacttga

SEQ ID NO: 143 - DNA, 399 bp, synthetic construct
  misc_feature 1..399: note = IL-2 analog 37
  source 1..399: mol_type = other DNA
cctacttcaagttctacaaagaaaacacagctacaactggagcatttactgctggatttacagatgattttgaatggaattaataattacaagaatcccaaactcaccgcgatgctcacaaagaagttttacatgcccaagaaggccacagaactgaaacatcttcagtgtctagaagaagaactcaaacctctggaggaagtgctaaatttagctcaaagcaaaaactttcacttagaccccagggacttaatcagcaatatcaacgtattagttctggaactaaagggatctgaaacaacattcatgtgtgaatatgctgatgagacagcaaccattgtagaatttctgaacagatggattacctttagtcaaagcatcatctcaacactgacttga

SEQ ID NO: 144 - DNA, 399 bp, synthetic construct
  misc_feature 1..399: note = IL-2 analog 38
  source 1..399: mol_type = other DNA
cctacttcaagttctacaaagaaaacacagctacaactggagcatttactgctggatttacagatgattttgaatggaattaataattacaagaatcccaaactcaccgcgatgctcacaaagaagttttacatgcccaagaaggccacagaactgaaacatcttcagtgtctagaagaagaactcaaacctctggaggaagtgctaaatttagctcaaagcaaaaactttcacttagaccccagggacttaatcagcaatatcaacgtatttgttttcgaatttaagggatctgaaacaacattcatgtgtgaatatgctgatgagacagcaaccattgtagaatttctgaacagatggattacctttagtcaaagcatcatctcaacactgacttga

SEQ ID NO: 145 - DNA, 399 bp, synthetic construct
  misc_feature 1..399: note = IL-2 analog 39
  source 1..399: mol_type = other DNA
cctacttcaagttctacaaagaaaacacagctacaactggagcatttactgctggatttacagatgattttgaatggaattaataattacaagaatcccaaactcaccgcgatgctcacaaagaagttttacatgcccaagaaggccacagaactgaaacatcttcagtgtctagaagaagaactcaaacctctggaggaagtgctaaatttagctcaaagcaaaaactttcacttagaccccagggacttaatcagcaatatcaacgtatttgttttcgaagtaaagggatctgaaacaacattcatgtgtgaatatgctgatgagacagcaaccattgtagaatttctgaacagatggattacctttagtcaaagcatcatctcaacactgacttga

SEQ ID NO: 146 - DNA, 399 bp, synthetic construct
  misc_feature 1..399: note = IL-2 analog 40
  source 1..399: mol_type = other DNA
cctacttcaagttctacaaagaaaacacagctacaactggagcatttactgctggatttacagatgattttgaatggaattaataattacaagaatcccaaactcaccgcgatgctcacaaagaagttttacatgcccaagaaggccacagaactgaaacatcttcagtgtctagaagaagaactcaaacctctggaggaagtgctaaatttagctcaaagcaaaaactttcacttagaccccagggacttaatcagcaatatcaacgtatttgttttcgaaattaagggatctgaaacaacattcatgtgtgaatatgctgatgagacagcaaccattgtagaatttctgaacagatggattacctttagtcaaagcatcatctcaacactgacttga

SEQ ID NO: 147 - DNA, 399 bp, synthetic construct
  misc_feature 1..399: note = IL-2 analog 41
  source 1..399: mol_type = other DNA
cctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcactttgaccccagggacgtagtcagcaatatcaacgtatttgttctggaactaaagggatctgaaacaacattcatgtgtgaatatgctgatgagacagcaaccattgtagaatttctgaacagatggattacctttagtcaaagcatcatctcaacactgacttga

SEQ ID NO: 148 - DNA, 399 bp, synthetic construct
  misc_feature 1..399: note = IL-2 analog 42
  source 1..399: mol_type = other DNA
cctacttcaagttctacaaagaaaacacagctacaactggagcatttactgctggatttacagatgattttgaatggaattaataattacaagaatcccaaactcaccgcgatgctcacaaagaagttttacatgcccaagaaggccacagaactgaaacatcttcagtgtctagaagaagaactcaaacctctggaggaagtgctaaatttagctcaaagcaaaaactttcacttagaacccagggacttaatcagcaatatcaacgtatttgttctggaactaaagggatctgaaacaacattcatgtgtgaatatgctgatgagacagcaaccattgtagaatttctgaacagatggattacctttagtcaaagcatcatctcaacactgacttga

SEQ ID NO: 149 - DNA, 399 bp, synthetic construct; identical to SEQ ID NO: 143
  misc_feature 1..399: note = IL-2 analog 43
  source 1..399: mol_type = other DNA

SEQ ID NO: 150 - DNA, 399 bp, synthetic construct
  misc_feature 1..399: note = IL-2 analog 44
  source 1..399: mol_type = other DNA
cctacttcaagttctacaaagaaaacacagctacaactggagcatttactgctggatttacagatgattttgaatggaattaataattacaagaatcccaaactcaccgcgatgctcacaaagaagttttacatgcccaagaaggccacagaactgaaacatcttcagtgtctagaagaagaactcaaacctctggaggaagtgctaaatttagctcaaagcaaaaactttcacttagaccccagggtcttaatcagcaatatcaacgtatttgttctggaactaaagggatctgaaacaacattcatgtgtgaatatgctgatgagacagcaaccattgtagaatttctgaacagatggattacctttagtcaaagcatcatctcaacactgacttga

SEQ ID NO: 151 - DNA, 399 bp, synthetic construct
  misc_feature 1..399: note = IL-2 analog 45
  source 1..399: mol_type = other DNA
cctacttcaagttctacaaagaaaacacagctacaactggagcatttactgctggatttacagatgattttgaatggaattaataattacaagaatcccaaactcaccgcgatgctcacaaagaagttttacatgcccaagaaggccacagaactgaaacatcttcagtgtctagaagaagaactcaaacctctggaggaagtgctaaatttagctcaaagcaaaaactttcacttagaccccaggttcttaatcagcaatatcaacgtatttgttctggaactaaagggatctgaaacaacattcatgtgtgaatatgctgatgagacagcaaccattgtagaatttctgaacagatggattacctttagtcaaagcatcatctcaacactgacttga

SEQ ID NO: 152 - DNA, 399 bp, synthetic construct
  misc_feature 1..399: note = IL-2 analog 46
  source 1..399: mol_type = other DNA
cctacttcaagttctacaaagaaaacacagctacaactggagcatttactgctggttttacagatgattttgaatggaattaataattacaagaatcccaaactcaccgcgatgctcacaaagaagttttacatgcccaagaaggccacagaactgaaacatcttcagtgtctagaagaagaactcaaacctctggaggaagtgctaaatttagctcaaagcaaaaactttcacttagaccccagggacttaatcagcaatatcaacgtatttgttctggaactaaagggatctgaaacaacattcatgtgtgaatatgctgatgagacagcaaccattgtagaatttctgaacagatggattacctttagtcaaagcatcatctcaacactgacttga

SEQ ID NO: 153 - DNA, 399 bp, synthetic construct
  misc_feature 1..399: note = IL-2 analog 47
  source 1..399: mol_type = other DNA
cctacttcaagttctacaaagaaaacacagctacaactggagcatttactgctgtttttacagatgattttgaatggaattaataattacaagaatcccaaactcaccgcgatgctcacaaagaagttttacatgcccaagaaggccacagaactgaaacatcttcagtgtctagaagaagaactcaaacctctggaggaagtgctaaatttagctcaaagcaaaaactttcacttagaccccagggacttaatcagcaatatcaacgtatttgttctggaactaaagggatctgaaacaacattcatgtgtgaatatgctgatgagacagcaaccattgtagaatttctgaacagatggattacctttagtcaaagcatcatctcaacactgacttga

SEQ ID NO: 154 - DNA, 399 bp, synthetic construct
  misc_feature 1..399: note = IL-2 analog 48
  source 1..399: mol_type = other DNA
cctacttcaagttctacaaagaaaacacagctacaactggagcatttactgctggatttacagatgattttgaatggaattaataattacaagaatcccaaactcaccgcgatgctcacaaagaagttttacatgcccaagaaggccacagaactgaaacatcttcagtgtctagaagaagaactcaaacctctggaggaagtgctaaatttagctcaaagcaaaaactttcacttagaccccagggacttaatcagcgttatcaacgtatttgttctggaactaaagggatctgaaacaacattcatgtgtgaatatgctgatgagacagcaaccattgtagaatttctgaacagatggattacctttagtcaaagcatcatctcaacactgacttga

SEQ ID NO: 155 - DNA, 399 bp, synthetic construct
  misc_feature 1..399: note = IL-2 analog 49
  source 1..399: mol_type = other DNA
cctacttcaagttctacaaagaaaacacagctacaactggagcatttactgctggatttacagatgattttgaatggaattaataattacaagaatcccaaactcaccgcgatgctcacaaagaagttttacatgcccaagaaggccacagaactgaaacatcttcagtgtctagaagaagaactcaaacctctggaggaagtgctaaatttagctcaaagcaaaaactttcacttagaccccagggacttaatcagctttatcaacgtatttgttctggaactaaagggatctgaaacaacattcatgtgtgaatatgctgatgagacagcaaccattgtagaatttctgaacagatggattacctttagtcaaagcatcatctcaacactgacttga

SEQ ID NO: 156 - DNA, 399 bp, synthetic construct
  misc_feature 1..399: note = IL-2 analog 50
  source 1..399: mol_type = other DNA
cctacttcaagttctacaaagaaaacacagctacaactggagcatttactgctggatttacagatgattttgaatggaattaataattacaagaatcccaaactcaccaggatgctcacaaagaagttttacatgcccaagaaggccacagaactgaaacatcttcagtgtctagaagaagaactcaaacctctggaggaagtgctaaatttagctcaaagcaaaaactttcactttgaccccagggacgtagtcagcaatatcaacgtatttgttctggaactaaagggatctgaaacaacattcatgtgtgaatatgctgatgagacagcaaccattgtagaatttctgaacagatggattacctttagtcaaagcatcatctcaacactgacttga

SEQ ID NO: 157 - DNA, 399 bp, synthetic construct
  misc_feature 1..399: note = IL-2 analog 51
  source 1..399: mol_type = other DNA
cctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaacaggaactcaaacctctggaggaagtgctaaatttagctcaaagcaaaaactttcacttagaccccagggacttaatcagcaatatcaacgtatttgttctggaactaaagggatctgaaacaacattcatgtgtgaatatgctgatgagacagcaaccattgtagaatttctgaacagatggattacctttagtcaaagcatcatctcaacactgacttga

SEQ ID NO: 158 - DNA, 399 bp, synthetic construct
  misc_feature 1..399: note = IL-2 analog 52
  source 1..399: mol_type = other DNA
cctacttcaagttctacaaagaaaacacagctacaactggagcatttactgctggatttacagatgattttgaatggaattaataattacaagaatcccaaactcaccgcgatgctcacaaagaagttttacatgcccaagaaggccacagaactgaaacatcttcagtgtctagaagaagaactcaaacctctggaggaagtgctaaatttagctcaaagcaaaaactttcactttgaccccagggacttaatcagcaatatcaacgtatttgttctggaactaaagggatctgaaacaacattcatgtgtgaatatgctgatgagacagcaaccattgtagaatttctgaacagatggattacctttagtcaaagcatcatctcaacactgacttga

SEQ ID NO: 159 - DNA, 399 bp, synthetic construct
  misc_feature 1..399: note = IL-2 analog 53
  source 1..399: mol_type = other DNA
cctacttcaagttctacaaagaaaacacagctacaactggagcatttactgctggatttacagatgattttgaatggaattaataattacaagaatcccaaactcaccgcgatgctcacaaagaagttttacatgcccaagaaggccacagaactgaaacatcttcagtgtctagaagaagaactcaaacctctggaggaagtgctaaatttagctcaaagcaaaaactttcactttgaccccagggagttaatcagcaatatcaacgtatttgttctggaactaaagggatctgaaacaacattcatgtgtgaatatgctgatgagacagcaaccattgtagaatttctgaacagatggattacctttagtcaaagcatcatctcaacactgacttga

SEQ ID NO: 160 - DNA, 399 bp, synthetic construct
  misc_feature 1..399: note = IL-2 analog 54
  source 1..399: mol_type = other DNA
cctacttcaagttctacaaagaaaacacagctacaactggagcatttactgctggatttacagatgattttgaatggaattaataattacaagaatcccaaactcaccgcgatgctcacaaagaagttttacatgcccaagaaggccacagaactgaaacatcttcagtgtctagaagaagaactcaaacctctggaggaagtgctaaatttagctcacagcaaaaactttcacttagaccccagggacttaatcagcaatatcaacgtatttgttctggaactaaagggatctgaaacaacattcatgtgtgaatatgctgatgagacagcaaccattgtagaatttctgaacagatggattacctttagtcaaagcatcatctcaacactgacttga

SEQ ID NO: 161 - DNA, 399 bp, synthetic construct
  misc_feature 1..399: note = IL-2 analog 55
  source 1..399: mol_type = other DNA
cctacttcaagttctacaaagaaaacacagctacaactggagcatttactgctggatttacagatgattttgaatggaattaataattacaagaatcccaaactcaccgcgatgctcacaaagaagttttacatgcccaagaaggccacagaactgaaacatcttcagtgtctagaagaagaactcaaacctctggaggaagtgctaaatttagctcacagcaaaaactttcactttgaccccagggacttaatcagcaatatcaacgtatttgttctggaactaaagggatctgaaacaacattcatgtgtgaatatgctgatgagacagcaaccattgtagaatttctgaacagatggattacctttagtcaaagcatcatctcaacactgacttga

SEQ ID NO: 162 - DNA, 399 bp, synthetic construct
  misc_feature 1..399: note = IL-2 analog 56
  source 1..399: mol_type = other DNA
cctacttcaagttctacaaagaaaacacagctacaactggagcatttactgctggatttacagatgattttgaatggaattaataattacaagaatcccaaactcaccgcgatgctcacaaagaagtttgccatgcccaagaaggccacagaactgaaacatcttcagtgtctagaagaagaactcaaacctctggaggaagtgctaaatttagctcacagcaaaaactttcacttagaccccagggacttaatcagcaatatcaacgtatttgttctggaactaaagggatctgaaacaacattcatgtgtgaatatgctgatgagacagcaaccattgtagaatttctgaacagatggattacctttagtcaaagcatcatctcaacactgacttga

SEQ ID NO: 163 - DNA, 399 bp, synthetic construct
  misc_feature 1..399: note = IL-2 analog 57
  source 1..399: mol_type = other DNA
cctacttcaagttctacaaagaaaacacagctacaactggagcatttactgctggatttacagatgattttgaatggaattaataattacaagaatcccaaactcaccgcgatgctcacaaagaagtttgccatgcccaagaaggccacagaactgaaacatcttcagtgtctagaagaagaactcaaacctctggaggaagtgctaaatttagctcacagcaaaaactttcactttgaccccagggacttaatcagcaatatcaacgtatttgttctggaactaaagggatctgaaacaacattcatgtgtgaatatgctgatgagacagcaaccattgtagaatttctgaacagatggattacctttagtcaaagcatcatctcaacactgacttga

SEQ ID NO: 164 - DNA, 399 bp, synthetic construct
  misc_feature 1..399: note = IL-2 analog 58
  source 1..399: mol_type = other DNA
cctacttcaagttctacaaagaaaacacagctacaactggagcatttactgctggatttacagatgattttgaatggaattaataattacaagaatcccaaactcaccgcgatgctcacaaagaagttttacatgcccaagaaggccacagaactgaaacatcttcagtgtctagaagaagaactcaaacctctggaggaagtgctaaatttagctcaaagcaaaaactttcactttgaccccagggacgctgccagcaatatcaacgtatttgttctggaactaaagggatctgaaacaacattcatgtgtgaatatgctgatgagacagcaaccattgtagaatttctgaacagatggattacctttagtcaaagcatcatctcaacactgacttga

SEQ ID NO: 165 - DNA, 399 bp, synthetic construct
  misc_feature 1..399: note = IL-2 analog 59
  source 1..399: mol_type = other DNA
cctacttcaagttctacaaagaaaacacagctacaactggagcatttactgctggatttacagatgattttgaatggaattaataattacaagaatcccaaactcaccgcgatgctcacaaagaagttttacatgcccaagaaggccacagaactgaaacatcttcagtgtctagaagaagaactcaaacctctggaggaagtgctaaatttagctcaaagcaaaaactttcactttgaccccagggacgctgccagcaatatcaacgtatatgttctggaactaaagggatctgaaacaacattcatgtgtgaatatgctgatgagacagcaaccattgtagaatttctgaacagatggattacctttagtcaaagcatcatctcaacactgacttga

SEQ ID NO: 166 - DNA, 399 bp, synthetic construct
  misc_feature 1..399: note = IL-2 analog 60
  source 1..399: mol_type = other DNA
cctacttcaagttctacaaagaaaacacagctacaactggagcatttactgctggatttacagatgattttgaatggaattaataattacaagaatcccaaactcaccgcgatgctcacaaagaagtttgccatgcccaagaaggccacagaactgaaacatcttcagtgtctagaagaagaactcaaacctctggaggaagtgctaaatttagctcaaagcaaaaactttcactatagacccagggacgctgccagcaatatcaacgtatatgttctggaactaaagggatctgaaacaacattcatgtgtgaatatgctgatgagacagcaaccattgtagaatttctgaacagatggattacctttagtcaaagcatcatctcaacactgacttga

SEQ ID NO: 167 - DNA, 399 bp, synthetic construct
  misc_feature 1..399: note = IL-2 analog 61
  source 1..399: mol_type = other DNA
cctacttcaagttctacaaagaaaacacagctacaactggagcatttactgctggatttacagatgattttgaatggaattaataattacaagaatcccaaactcaccgcgatgctcacaaagaagttttacatgcccaagaaggccacagaactgaaacatcttcagtgtctagaagaagaactcaaacctctggaggaagtgctaaatttagctcaaagcaaaaactttcactatgaccccagggacggtgttagcaatatcaacgtatatgttctggaactaaagggatctgaaacaacattcatgtgtgaatatgctgatgagacagcaaccattgtagaatttctgaacagatggattacctttagtcaaagcatcatctcaacactgacttga

SEQ ID NO: 168 - DNA, 399 bp, synthetic construct
  misc_feature 1..399: note = IL-2 analog 62
  source 1..399: mol_type = other DNA
cctacttcaagttctacaaagaaaacacagctacaactggagcatttactgctggatttacagatgattttgaatggaattaataattacaagaatcccaaactcaccgcgatgctcacaaagaagttttacatgcccaagaaggccacagaactgaaacatcttcagtgtctagaagaagaactcaaacctctggaggaagtgctaaatttagctcaaagcaaaaactttcactgggaacccagggacggagccagcaatatcaacgtatttgttctggaactaaagggatctgaaacaacattcatgtgtgaatatgctgatgagacagcaaccattgtagaatttctgaacagatggattacctttagtcaaagcatcatctcaacactgacttga

SEQ ID NO: 169 - DNA, 399 bp, synthetic construct
  misc_feature 1..399: note = IL-2 analog 63
  source 1..399: mol_type = other DNA
cctacttcaagttctacaaagaaaacacagctacaactggagcatttactgctggatttacagatgattttgaatggaattaataattacaagaatcccaaactcaccgcgatgctcacaaagaagttttacatgcccaagaaggccacagaactgaaacatcttcagtgtctagaagaagaactcaaacctctggaggaagtgctaaatttagctcaaagcaaaaactttcacgacgaacccagggacacaggcagcaatatcaacgtatttgttctggaactaaagggatctgaaacaacattcatgtgtgaatatgctgatgagacagcaaccattgtagaatttctgaacagatggattacctttagtcaaagcatcatctcaacactgacttga

SEQ ID NO: 170 - DNA, 399 bp, synthetic construct
  misc_feature 1..399: note = IL-2 analog 64
  source 1..399: mol_type = other DNA
cctacttcaagttctacaaagaaaacacagctacaactggagcatttactgctggatttacagatgattttgaatggaattaataattacaagaatcccaaactcaccgcgatgctcacaaagaagttttacatgcccaagaaggccacagaactgaaacatcttcagtgtctagaagaagaactcaaacctctggaggaagtgctaaatttagctcaaagcaaaaactttcactacaaccccagggacgtagtcagcaatatcaacgtatttgttctggaactaaagggatctgaaacaacattcatgtgtgaatatgctgatgagacagcaaccattgtagaatttctgaacagatggattacctttagtcaaagcatcatctcaacactgacttga

SEQ ID NO: 171 - DNA, 399 bp, synthetic construct
  misc_feature 1..399: note = IL-2 analog 65
  source 1..399: mol_type = other DNA
cctacttcaagttctacaaagaaaacacagctacaactggagcatttactgctggatttacagatgattttgaatggaattaataattacaagaatcccaaactcaccgcgatgctcacaaagaagttttacatgcccaagaaggccacagaactgaaacatcttcagtgtctagaagaagaactcaaacctctggaggaagtgctaaatttagctcaaagcaaaaactttcactacgaacccagggacgtagtcagcaatatcaacgtatttgttctggaactaaagggatctgaaacaacattcatgtgtgaatatgctgatgagacagcaaccattgtagaatttctgaacagatggattacctttagtcaaagcatcatctcaacactgacttga

SEQ ID NO: 172 - DNA, 399 bp, synthetic construct
  misc_feature 1..399: note = IL-2 analog 66
  source 1..399: mol_type = other DNA
cctacttcaagttctacaaagaaaacacagctacaactggagcatttactgctggatttacagatgattttgaatggaattaataattacaagaatcccaaactcaccgcgatgctcacaaagaagttttacatgcccaagaaggccacagaactgaaacatcttcagtgtctagaagaagaactcaaacctctggaggaagtgctaaatttagctcaaagcaaaaactttcacttcgaacccagggacttcgtcagcaatatcaacgtatttgttctggaactaaagggatctgaaacaacattcatgtgtgaatatgctgatgagacagcaaccattgtagaatttctgaacagatggattacctttagtcaaagcatcatctcaacactgacttga

SEQ ID NO: 173 - DNA, 399 bp, synthetic construct
  misc_feature 1..399: note = IL-2 analog 67
  source 1..399: mol_type = other DNA
cctacttcaagttctacaaagaaaacacagctacaactggagcatttactgctggatttacagatgattttgaatggaattaataattacaagaatcccaaactcaccgcgatgctcacaaagaagttttacatgcccaagaaggccacagaactgaaacatcttcagtgtctagaagaagaactcaaacctctggaggaagtgctaaatttagctcaaagcaaaaactttcactatgaccccagggacttcgtcagcaatatcaacgtatgggttctggacctaaagggatctgaaacaacattcatgtgtgaatatgctgatgagacagcaaccattgtagaatttctgaacagatggattacctttagtcaaagcatcatctcaacactgacttga

SEQ ID NO: 174 - DNA, 399 bp, synthetic construct
  misc_feature 1..399: note = IL-2 analog 68
  source 1..399: mol_type = other DNA
cctacttcaagttctacaaagaaaacacagctacaactggagcatttactgctggatttacagatgattttgaatggaattaataattacaagaatcccaaactcaccgcgatgctcacaaagaagttttacatgcccaagaaggccacagaactgaaacatcttcagtgtctagaagaagaactcaaacctctggaggaagtgctaaatttagctcaaagcaaaaactttcacttcgaacccagggacatagtcagcaatatcaacgaattcgttctggaactaaagggatctgaaacaacattcatgtgtgaatatgctgatgagacagcaaccattgtagaatttctgaacagatggattacctttagtcaaagcatcatctcaacactgacttga

SEQ ID NO: 175 - DNA, 399 bp, synthetic construct
  misc_feature 1..399: note = IL-2 analog 69
  source 1..399: mol_type = other DNA
cctacttcaagttctacaaagaaaacacagctacaactggagcatttactgctggatttacagatgattttgaatggaattaataattacaagaatcccaaactcaccgcgatgctcacaaagaagttttacatgcccaagaaggccacagaactgaaacatcttcagtgtctagaagaagaactcaaacctctggaggaagtgctaaatttagctcaaagcaaaaactttcactacgaacccagggacttcctcagcaatatcaacgaatgggttctggacctaaagggatctgaaacaacattcatgtgtgaatatgctgatgagacagcaaccattgtagaatttctgaacagatggattacctttagtcaaagcatcatctcaacactgacttga

SEQ ID NO: 176 - DNA, 399 bp, synthetic construct
  misc_feature 1..399: note = IL-2 analog 70
  source 1..399: mol_type = other DNA
cctacttcaagttctacaaagaaaacacagctacaactggagcatttactgctggatttacagatgattttgaatggaattaataattacaagaatcccaaactcaccgcgatgctcacaaagaagttttacatgcccaagaaggccacagaactgaaacatcttcagtgtctagaagaagaactcaaacctctggaggaagtgctaaatttagctcaaagcaaaaactttcactatgaccccagggacgtagtcagcaatatcaacgtatttgttctggaactaaagggatctgaaacaacattcatgtgtgaatatgctgatgagacagcaaccattgtagaatttctgaacagatggattacctttagtcaaagcatcatctcaacactgacttga

SEQ ID NO: 177 - DNA, 399 bp, synthetic construct
  misc_feature 1..399: note = IL-2 analog 71
  source 1..399: mol_type = other DNA
cctacttcaagttctacaaagaaaacacagctacaactggagcatttactgctggatttacagatgattttgaatggaattaataattacaagaatcccaaactcaccgcgatgctcacaaagaagttttacatgcccaagaaggccacagaactgaaacatcttcagtgtctagaagaagaactcaaacctctggaggaagtgctaaatttagctcaaagcaaaaactttcacttcgaacccagggacgtagtcagcaatatcaacgtatttgttctggaactaaagggatctgaaacaacattcatgtgtgaatatgctgatgagacagcaaccattgtagaatttctgaacagatggattacctttagtcaaagcatcatctcaacactgacttga

SEQ ID NO: 178 - DNA, 399 bp, synthetic construct
  misc_feature 1..399: note = IL-2 analog 72
  source 1..399: mol_type = other DNA
cctacttcaagttctacaaagaaaacacagctacaactggagcatttactgctggatttacagatgattttgaatggaattaataattacaagaatcccaaactcaccgcgatgctcacaaagaagttttacatgcccaagaaggccacagaactgaaacatcttcagtgtctagaagaagaactcaaacctctggaggaagtgctaaatttagctcaaagcaaaaactttcactttgaccccagggacgtaggcagcaatatcaacgtatttgttctggaactaaagggatctgaaacaacattcatgtgtgaatatgctgatgagacagcaaccattgtagaatttctgaacagatggattacctttagtcaaagcatcatctcaacactgacttga

SEQ ID NO: 179 - DNA, 399 bp, synthetic construct
  misc_feature 1..399: note = IL-2 analog 73
  source 1..399: mol_type = other DNA
cctacttcaagttctacaaagaaaacacagctacaactggagcatttactgctggatttacagatgattttgaatggaattaataattacaagaatcccaaactcaccgcgatgctcacaaagaagttttacatgcccaagaaggccacagaactgaaacatcttcagtgtctagaagaagaactcaaacctctggaggaagtgctaaatttagctcaaagcaaaaactttcactttgaccccagggactgggtcagcaatatcaacgtatttgttctggaactaaagggatctgaaacaacattcatgtgtgaatatgctgatgagacagcaaccattgtagaatttctgaacagatggattacctttagtcaaagcatcatctcaacactgacttga

SEQ ID NO: 180 - DNA, 399 bp, synthetic construct
  misc_feature 1..399: note = IL-2 analog 74
  source 1..399: mol_type = other DNA
cctacttcaagttctacaaagaaaacacagctacaactggagcatttactgctggatttacagatgattttgaatggaattaataattacaagaatcccaaactcaccgatatgctcacaaagaagttttacatgcccaagaaggccacagaactgaaacatcttcagtgtctagaagaagaactcaaacctctggaggaagtgctaaatttagctcaaagcaaaaactttcactatgaccccagggacgtagtcagcaatatcaacgtatttgttctggaactaaagggatctgaaacaacattcatgtgtgaatatgctgatgagacagcaaccattgtagaatttctgaacagatggattacctttagtcaaagcatcatctcaacactgacttga

SEQ ID NO: 181 - DNA, 399 bp, synthetic construct
  misc_feature 1..399: note = IL-2 analog 75
  source 1..399: mol_type = other DNA
cctacttcaagttctacaaagaaaacacagctacaactggagcatttactgctggatttacagatgattttgaatggaattaataattacaagaatcccaaactcaccgcgatgctcacaaagaagtttgccatgcccaagaaggccacagaactgaaacatcttcagtgtctagaagaagaactcaaacctctggaggaagtgctaaatttagctcaaagcaaaaactttcacttcgaacccagggacgtagtcagcaatatcaacgtatttgttctggaactaaagggatctgaaacaacattcatgtgtgaatatgctgatgagacagcaaccattgtagaatttctgaacagatggattacctttagtcaaagcatcatctcaacactgacttga

SEQ ID NO: 182 - DNA, 399 bp, synthetic construct
  misc_feature 1..399: note = IL-2 analog 76
  source 1..399: mol_type = other DNA
cctacttcaagttctacaaagaaaacacagctacaactggagcatttactgctggatttacagatgattttgaatggaattaataattacaagaatcccaaactcaccgcgatgctcacaaagcagttttacatgcccaagaaggccacagaactgaaacatcttcagtgtctagaacgggaactcaaacctctggaggaagtgctaaatttagctcaaagcaaaaactttcactttgaccccagggacgtaggcagcaatatcaacgtatttgttctggaactaaagggatctgaaacaacattcatgtgtgaatatgctgatgagacagcaaccattgtagaatttctgaacagatggattacctttagtcaaagcatcatctcaacactgacttga

SEQ ID NO: 183 - DNA, 399 bp, synthetic construct
  misc_feature 1..399: note = IL-2 analog 77
  source 1..399: mol_type = other DNA
cctacttcaagttctacaaagaaaacacagctacaactggagcatttactgctggatttacagatgattttgaatggaattaataattacaagaatcccaaactcaccgcgatgctcacaaaggagttttacatgcccaagaaggccacagaactgaaacatcttcagtgtctagaacgggaactcaaacctctggaggaagtgctaaatttagctcaaagcaaaaactttcactttgaccccagggactgggtcagcaatatcaacgtatttgttctggaactaaagggatctgaaacaacattcatgtgtgaatatgctgatgagacagcaaccattgtagaatttctgaacagatggattacctttagtcaaagcatcatctcaacactgacttga

SEQ ID NO: 184 - DNA, 399 bp, synthetic construct
  misc_feature 1..399: note = IL-2 analog 78
  source 1..399: mol_type = other DNA
cctacttcaagttctacaaagaaaacacagctacaactggagcatttactgctggatttacagatgattttgaatggaattaataattacaagaatcccgaactcaccgcgatgctcacaaagaagttttacatgcccaagaaggccacagaactgaaacatcttcagtgtctagaagaagaactcaaacctctggaggaagtgctaaatttagctcaaagcaaaaactttcacttcgaacccagggacgtagtcagcaatatcaacgtatttgttctggaactaaagggatctgaaacaacattcatgtgtgaatatgctgatgagacagcaaccattgtagaatttctgaacagatggattacctttagtcaaagcatcatctcaacactgacttga

SEQ ID NO: 185 - DNA, 399 bp, synthetic construct
  misc_feature 1..399: note = IL-2 analog 79
  source 1..399: mol_type = other DNA
cctacttcaagttctacaaagaaaacacagctacaactggagcatttactgctggatttacagatgattttgaatggaattaataattacaagaatcccgaactcaccgcgatgctcacaaagaagttttacatgcccaagaaggccacagaactgaaacatcttcagtgtctagaagaagaactcaaacctctggaggaagtgctaaatttagctcacagcaaaaactttcacttcgaacccagggacgtagtcagcaatatcaacgtatttgttctggaactaaagggatctgaaacaacattcatgtgtgaatatgctgatgagacagcaaccattgtagaatttctgaacagatggattacctttagtcaaagcatcatctcaacactgacttga

SEQ ID NO: 186 - DNA, 399 bp, synthetic construct
  misc_feature 1..399: note = IL-2 analog 80
  source 1..399: mol_type = other DNA
cctacttcaagttctacaaagaaaacacagctacaactggagcatttactgctggatttacagatgattttgaatggaattaataattacaagaatcccgaactcaccgcgatgctcacaaagaagttttacatgcccaagaaggccacagaactgaaacatcttcagtgtctagaagaagaactcaaacctctggaggaagtgctaaatttagctcacagcaaaaactttcacttcgaaggcagggacgtagtcagcaatatcaacgtatttgttctggaactaaagggatctgaaacaacattcatgtgtgaatatgctgatgagacagcaaccattgtagaatttctgaacagatggattacctttagtcaaagcatcatctcaacactgacttga

SEQ ID NO: 187 - DNA, 399 bp, synthetic construct
  misc_feature 1..399: note = IL-2 analog 81
  source 1..399: mol_type = other DNA
cctacttcaagttctacaaagaaaacacagctacaactggagcatttactgctggatttacagatgattttgaatggaattaataattacaagaatcccgaactcaccgcgatgctcacaaagaagttttacatgcccaagaaggccacagaactgaaacatcttcagtgtctagaagaagaactcaaacctctggaggaagtgctaaatttagctcacagcaaaaactttcacttcgaagtcagggacgtagtcagcaatatcaacgtatttgttctggaactaaagggatctgaaacaacattcatgtgtgaatatgctgatgagacagcaaccattgtagaatttctgaacagatggattacctttagtcaaagcatcatctcaacactgacttga

SEQ ID NO: 188 - DNA, 399 bp, synthetic construct
  misc_feature 1..399: note = IL-2 analog 82
  source 1..399: mol_type = other DNA
cctacttcaagttctacaaagaaaacacagctacaactggagcatttacgtctggatttagaaatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcactttgaccccagggacgaagtgagcaatatcaacgtatttgttctggaactaaagggatctgaaacaacattcatgtgtgaatatgctgatgagacagcaaccattgtagaatttctgaacagatggattacctttagtcaaagcatcatctcaacactgacttga

SEQ ID NO: 189 - DNA, 399 bp, synthetic construct
  misc_feature 1..399: note = IL-2 analog 83
  source 1..399: mol_type = other DNA
cctacttcaagttctacaaagaaaacacagctacaactggagcatttacgtcgtgatttagaaatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcactttgaccccagggacgaagtgagcaatatcaacgtatttgttctggaactaaagggatctgaaacaacattcatgtgtgaatatgctgatgagacagcaaccattgtagaatttctgaacagatggattacctttagtcaaagcatcatctcaacactgacttga

SEQ ID NO: 190 - DNA, 399 bp, synthetic construct
  misc_feature 1..399: note = IL-2 analog 84
  source 1..399: mol_type = other DNA
cctacttcaagttctacaaagaaaacacagctacaactggagcatttacgtctggatttagaaatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacgtggatcccagggacgaagtgagcaatatcaacgtatttgttctggaactaaagggatctgaaacaacattcatgtgtgaatatgctgatgagacagcaaccattgtagaatttctgaacagatggattacctttagtcaaagcatcatctcaacactgacttga

SEQ ID NO: 191 - DNA, 399 bp, synthetic construct
  misc_feature 1..399: note = IL-2 analog 85
  source 1..399: mol_type = other DNA
cctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttcgaacccagggacgtagtcagcaatatcaacgtatttgttctggaactaaagggatctgaaacaacattcatgtgtgaatatgctgatgagacagcaaccattgtagaatttctgaacagatggattacctttagtcaaagcatcatctcaacactgacttga

SEQ ID NO: 192 - DNA, 399 bp, synthetic construct
  misc_feature 1..399: note = IL-2 analog 86
  source 1..399: mol_type = other DNA
cctacttcaagttctacaaagaaaacacagctacaactggagcatttacgtctggatttagaaatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttcgaacccagggacgtagtcagcaatatcaacgtatttgttctggaactaaagggatctgaaacaacattcatgtgtgaatatgctgatgagacagcaaccattgtagaatttctgaacagatggattacctttagtcaaagcatcatctcaacactgacttga

SEQ ID NO: 193 - DNA, 399 bp, synthetic construct
  misc_feature 1..399: note = IL-2 analog 87
  source 1..399: mol_type = other DNA
cctacttcaagttctacaaagaaaacacagctacaactggagcatttacgtcgtgatttagaaatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttcgaacccagggacgtagtcagcaatatcaacgtatttgttctggaactaaagggatctgaaacaacattcatgtgtgaatatgctgatgagacagcaaccattgtagaatttctgaacagatggattacctttagtcaaagcatcatctcaacactgacttga

SEQ ID NO: 194 - DNA, 399 bp, synthetic construct
  misc_feature 1..399: note = IL-2 analog 88
  source 1..399: mol_type = other DNA
cctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagacgaactcaaacctctggaggaagtgctaaatttagctcaaagcaaaaactttcacttcgaacccagggacgtagtcagcaatatcaacgtatttgttctggaactaaagggatctgaaacaacattcatgtgtgaatatgctgatgagacagcaaccattgtagaatttctgaacagatggattacctttagtcaaagcatcatctcaacactgacttga

SEQ ID NO: 195 - DNA, 399 bp, synthetic construct
  misc_feature 1..399: note = IL-2 analog 89
  source 1..399: mol_type = other DNA
cctacttcaagttctacaaagaaaacacagctacaactggagcatttactgctggatttacagatgattttgaatggaattaataattacaagaatcccaaactcaccgcgatgctcacatttaagttttacatgcccaagaaggccacagaactgaaacatcttcagtgtctagaagaagaactcaaacctctggagcaggtgctaaatttagctcaaagcaaaaactttcacttcgaacccagggacgtagtcagcaatatcaacgtatttgttctggaactaaagggatctgaaacaacattcatgtgtgaatatgctgatgagacagcaaccattgtagaatttctgaacagatggattacctttagtcaaagcatcatctcaacactgacttga

SEQ ID NO: 196 - DNA, 399 bp, synthetic construct
  misc_feature 1..399: note = IL-2 analog 90
  source 1..399: mol_type = other DNA
cctacttcaagttctacaaagaaaacacagctacaactggagcatttactgctggatttacagatgattttgaatggaattaataattacaagaatcccaaactcaccaggatgctcacatggaagttttacatgcccaagaaggccacagaactgaaacatcttcagtgtctagaagaagaactcaaacctctggaggaagtgctaaatttagctcaaagcaaaaactttcacttcgaacccagggacgtagtcagcaatatcaacgtatttgttctggaactaaagggatctgaaacaacattcatgtgtgaatatgctgatgagacagcaaccattgtagaatttctgaacagatggattacctttagtcaaagcatcatctcaacactgacttga

SEQ ID NO: 197 - DNA, 399 bp, synthetic construct
  misc_feature 1..399: note = IL-2 analog 91
  source 1..399: mol_type = other DNA
cctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaacaagaactcaaacctctggaggaagtgctaaatttagctcaaagcaaaaactttcacttcgaacccagggacgtagtcagcaatatcaacgtatttgttctggaactaaagggatctgaaacaacattcatgtgtgaatatgctgatgagacagcaaccattgtagaatttctgaacagatggattacctttagtcaaagcatcatctcaacactgacttga

SEQ ID NO: 198 - DNA, 399 bp, synthetic construct
  misc_feature 1..399: note = IL-2 analog 92
  source 1..399: mol_type = other DNA
cctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttcgaacccagggacgtagtcagcaatatcaacacatttgttctggaactaaagggatctgaaacaacattcatgtgtgaatatgctgatgagacagcaaccattgtagaatttctgaacagatggattacctttagtcaaagcatcatctcaacactgacttga

SEQ ID NO: 199 - DNA, 399 bp, synthetic construct
  misc_feature 1..399: note = IL-2 analog 93
  source 1..399: mol_type = other DNA
cctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttcgaacccagggaggtagtcagcaatatcaacacatttgttctggaactaaagggatctgaaacaacattcatgtgtgaatatgctgatgagacagcaaccattgtagaatttctgaacagatggattacctttagtcaaagcatcatctcaacactgacttga

SEQ ID NO: 200 - DNA, 399 bp, synthetic construct
  misc_feature 1..399: note = IL-2 analog 94
  source 1..399: mol_type = other DNA
cctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttcgaacccagggacgtagtcagcaatatcaacgtatttgttttcgaattcaagggatctgaaacaacattcatgtgtgaatatgctgatgagacagcaaccattgtagaatttctgaacagatggattacctttagtcaaagcatcatctcaacactgacttga

SEQ ID NO: 201 - DNA, 399 bp, synthetic construct
  misc_feature 1..399: note = IL-2 analog 95
  source 1..399: mol_type = other DNA
cctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagggctaaatttagctcaaagcaaaaactttcacttcgaacccagggacgtagtcagcaatatcaacgtatttgttctggaactaaagggatctgaaacaacattcatgtgtgaatatgctgatgagacagcaaccattgtagaatttctgaacagatggattacctttagtcaaagcatcatctcaacactgacttga

SEQ ID NO: 202 - DNA, 399 bp, synthetic construct
  misc_feature 1..399: note = IL-2 analog 96
  source 1..399: mol_type = other DNA
cctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagggctaaatttagctgcaagcaaaaactttcacttcgaacccagggacgtagtcagcaatatcaacgtatttgttctggaactaaagggatctgaaacaacattcatgtgtgaatatgctgatgagacagcaaccattgtagaatttctgaacagatggattacctttagtcaaagcatcatctcaacactgacttga

SEQ ID NO: 203 - DNA, 399 bp, synthetic construct
  misc_feature 1..399: note = IL-2 analog 97
  source 1..399: mol_type = other DNA
cctacttcaagttctacaaagaaaacacagctacaactggagcatttactgctggatttacagatgattttgaatggaattaataattacaagaatcccaaactcaccgcgatgctcacatttaagttttacatgcccaagaaggccacagaactgaaacatcttcagtgtctagaagaagaactcaaacctctggaggaagtgctaaatttagctcaaagcaaaaactttcactttgaccccagggacttaatcagcaatatcaacgtatttgttctggaactaaagggatctgaaacaacattcatgtgtgaatatgctgatgagacagcaaccattgtagaatttctgaacagatggattacctttagtcaaagcatcatctcaacactgacttga

SEQ ID NO: 204 - DNA, 399 bp, synthetic construct
  misc_feature 1..399: note = IL-2 analog 98
  source 1..399: mol_type = other DNA
cctacttcaagttctacaaagaaaacacagctacaactggagcatttactgctggatttacagatgattttgaatggaattaataattacaagaatcccaaactcaccgcgatgctcacatttaagttttacatgcccaagaaggccacagaactgaaacatcttcagtgtctagaagaagaactcaaacctctggaggaagtgctaaatttagctcaaagcaaaaactttcactttgagcccagggacgtaatcagcaatatcaacgtatttgttctggaactaaagggatctgaaacaacattcatgtgtgaatatgctgatgagacagcaaccattgtagaatttctgaacagatggattacctttagtcaaagcatcatctcaacactgacttga

SEQ ID NO: 205 - DNA, 399 bp, synthetic construct
  misc_feature 1..399: note = IL-2 analog 99
  source 1..399: mol_type = other DNA
cctacttcaagttctacaaagaaaacacagctacaactggagcatttactgctggatttacagatgattttgaatggaattaataattacaagaatcccaaactcaccgcgatgctcacatttaagttttacatgcccaagaaggccacagaactgaaacatcttcagtgtctagaagaagaactcaaacctctggaggaagtgctaaatttagctcaaagcaaaaactttcactttgagcccagggacttagtcagcaatatcaacgtatttgttctggaactaaagggatctgaaacaacattcatgtgtgaatatgctgatgagacagcaaccattgtagaatttctgaacagatggattacctttagtcaaagcatcatctcaacactgacttga

SEQ ID NO: 206 - DNA, 399 bp, synthetic construct
  misc_feature 1..399: note = IL-2 analog 100
  source 1..399: mol_type = other DNA
cctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttcgaacccagggactacgtcagcaatatcaacgtatttgttctggaactaaagggatctgaaacaacattcatgtgtgaatatgctgatgagacagcaaccattgtagaatttctgaacagatggattacctttagtcaaagcatcatctcaacactgacttga

SEQ ID NO: 207 - DNA, 399 bp, synthetic construct
  misc_feature 1..399: note = IL-2 analog 101
  source 1..399: mol_type = other DNA
cctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttcgaacccagggacctggccagcaatatcaacgtatttgttctggaactaaagggatctgaaacaacattcatgtgtgaatatgctgatgagacagcaaccattgtagaatttctgaacagatggattacctttagtcaaagcatcatctcaacactgacttga

SEQ ID NO: 208 - DNA, 399 bp, synthetic construct
  misc_feature 1..399: note = IL-2 analog 102
  source 1..399: mol_type = other DNA
cctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttcgaacccagggacgtagtcagcaatatcaacgtaattgttctggaactaaagggatctgaaacaacattcatgtgtgaatatgctgatgagacagcaaccattgtagaatttctgaacagatggattacctttagtcaaagcatcatctcaacactgacttga

SEQ ID NO: 209 - DNA, 399 bp, synthetic construct
  misc_feature 1..399: note = IL-2 analog 103
  source 1..399: mol_type = other DNA
cctacttcaagttctacaaagaaaacacagctacaactggagcatttacgtctggatttagaaatgattttgaatggaattaataattacaagaatcccaaactcaccgcgatgctcacatttaagttttacatgcccaagaaggccacagaactgaaacatcttcagtgtctagaagaagaactcaaacctctggaggaagtgctaaatttagctcaaagcaaaaactttcacttcgaacccagggacgtagtcagcaatatcaacgtatttgttctggaactaaagggatctgaaacaacattcatgtgtgaatatgctgatgagacagcaaccattgtagaatttctgaacagatggattacctttagtcaaagcatcatctcaacactgacttga

SEQ ID NO: 210 - DNA, 399 bp, synthetic construct
  misc_feature 1..399: note = IL-2 analog 104
  source 1..399: mol_type = other DNA
cctacttcaagttctacaaagaaaacacagctacaactggagcatttacgtctggatttagaaatgattttgaatggaattaataattacaagaatcccaaactcaccaggatgctcacatttaagttttacatgcccaagaaggccacagaactgaaacatcttcagtgtctagaagacgaactcaaacctctggaggaagtgctaaatttagctcaaagcaaaaactttcacttcgaacccagggacgtagtcagcaatatcaacgtatttgttctggaactaaagggatctgaaacaacattcatgtgtgaatatgctgatgagacagcaaccattgtagaatttctgaacagatggattacctttagtcaaagcatcatctcaacactgacttga

SEQ ID NO: 211 - DNA, 399 bp, synthetic construct
  misc_feature 1..399: note = IL-2 analog 105
  source 1..399: mol_type = other DNA
cctacttcaagttctacaaagaaaacacagctacaactggagcatttacgtctggatttagaaatgattttgaatggaattaataattacaagaatcccaaactcaccaggatgctcacatttaagttttacatgcccaagaaggccacagaactgaaacatcttcagtgtctagaagaagaactcaaacctctggaggacgtgctaaatttagctcaaagcaaaaactttcacttcgaacccagggacgtagtcagcaatatcaacgtatttgttctggaactaaagggatctgaaacaacattcatgtgtgaatatgctgatgagacagcaaccattgtagaatttctgaacagatggattacctttagtcaaagcatcatctcaacactgacttga

SEQ ID NO: 212 - protein, 133 aa, synthetic construct
  REGION 1..133: note = General Formula 1
  SITE 1: note = Xaa is a deletion
  VARIANT 18: note = Xaa is leucine (L) or arginine (R)
  VARIANT 19: note = Xaa is leucine (L) or tyrosine (Y)
  VARIANT 22: note = Xaa is glutamic acid (E) or glutamine (Q)
  VARIANT 38: note = Xaa is alanine (A), aspartic acid (D), or arginine (R)
  VARIANT 42: note = Xaa is alanine (A), phenylalanine (F), lysine (K), or tryptophan (W)
  VARIANT 43: note = Xaa is glutamic acid (E), lysine (K), or glutamine (Q)
  VARIANT 45: note = Xaa is alanine (A) or tyrosine (Y)
  VARIANT 61: note = Xaa is aspartic acid (D), glutamic acid (E), glutamine (Q), or arginine (R)
  VARIANT 68: note = Xaa is aspartic acid (D) or glutamic acid (E)
  VARIANT 74: note = Xaa is histidine (H) or glutamine (Q)
  VARIANT 80: note = Xaa is phenylalanine (F), leucine (L), valine (V), or tyrosine (Y)
  VARIANT 81: note = Xaa is aspartic acid (D), glutamic acid (E), or arginine (R)
  VARIANT 84: note = Xaa is aspartic acid (D) or glutamic acid (E)
  VARIANT 85: note = Xaa is alanine (A), glutamic acid (E), glycine (G), leucine (L), valine (V), tryptophan (W), or tyrosine (Y)
  VARIANT 86: note = Xaa is alanine (A), glycine (G), isoleucine (I), or valine (V)
  VARIANT 91: note = Xaa is threonine (T) or valine (V)
  VARIANT 92: note = Xaa is phenylalanine (F), isoleucine (I), or tyrosine (Y)
  VARIANT 94: note = Xaa is phenylalanine (F) or leucine (L)
  VARIANT 96: note = Xaa is phenylalanine (F) or leucine (L)
XPTSSSTKKTQLQLEHLXXDLXMILNGINNYKNPKLTXMLTXXFXMPKKATELKHLQCLEXELKPLEXVLNLAXSKNFHXXPRXXXSNINXXVXEXKGSETTFMCEYADETATIVEFLNRWITFSQSIISTLT

SEQ ID NO: 213 - protein, 133 aa, synthetic construct
  REGION 1..133: note = General Formula 2
  SITE 1: note = Xaa is a deletion
  VARIANT 18: note = Xaa is leucine (L) or arginine (R)
  VARIANT 22: note = Xaa is glutamic acid (E) or glutamine (Q)
  VARIANT 38: note = Xaa is alanine (A) or arginine (R)
  VARIANT 42: note = Xaa is phenylalanine (F) or lysine (K)
  VARIANT 61: note = Xaa is aspartic acid (D) or glutamic acid (E)
  VARIANT 68: note = Xaa is aspartic acid (D) or glutamic acid (E)
  VARIANT 81: note = Xaa is aspartic acid (D) or glutamic acid (E)
  VARIANT 85: note = Xaa is leucine (L) or valine (V)
  VARIANT 86: note = Xaa is isoleucine (I) or valine (V)
XPTSSSTKKTQLQLEHLXLDLXMILNGINNYKNPKLTXMLTXKFYMPKKATELKHLQCLEXELKPLEXVLNLAQSKNFHFXPRDXXSNINVFVLELKGSETTFMCEYADETATIVEFLNRWITFSQSIISTLT

SEQ ID NO: 214 - DNA, 33 bp, synthetic construct
  misc_feature 1..33: note = primer #1 (IL2_L12F_F)
  source 1..33: mol_type = other DNA
caaagaaaacacagtttcaactggagcatttac

SEQ ID NO: 215 - DNA, 33 bp, synthetic construct
  misc_feature 1..33: note = primer #2 (IL2_L12F_R)
  source 1..33: mol_type = other DNA
gtaaatgctccagttgaaactgtgttttctttg

SEQ ID NO: 216 - DNA, 33 bp, synthetic construct
  misc_feature 1..33: note = primer #3 (IL2_L12V_F)
  source 1..33: mol_type = other DNA
caaagaaaacacaggtacaactggagcatttac

SEQ ID NO: 217 - DNA, 33 bp, synthetic construct
  misc_feature 1..33: note = primer #4 (IL2_L12V_R)
  source 1..33: mol_type = other DNA
gtaaatgctccagttgtacctgtgttttctttg

SEQ ID NO: 218 - DNA, 39 bp, synthetic construct
  misc_feature 1..39: note = primer #5 (IL2_L18R_L19R_Q22E_F)
  source 1..39: mol_type = other DNA
ctggagcatttacgtcgtgatttagaaatgattttgaat

SEQ ID NO: 219 - DNA, 39 bp, synthetic construct
  misc_feature 1..39: note = primer #6 (IL2_L18R_L19R_Q22E_R)
  source 1..39: mol_type = other DNA
attcaaaatcatttctaaatcacgacgtaaatgctccag

SEQ ID NO: 220 - DNA, 39 bp, synthetic construct
  misc_feature 1..39: note = primer #7 (IL2_L18R_Q22E_F)
  source 1..39: mol_type = other DNA
ctggagcatttacgtctggatttagaaatgattttgaat

SEQ ID NO: 221 - DNA, 39 bp, synthetic construct
  misc_feature 1..39: note = primer #8 (IL2_L18R_Q22E_R)
  source 1..39: mol_type = other DNA
attcaaaatcatttctaaatccagacgtaaatgctccag

SEQ ID NO: 222 - DNA, 33 bp, synthetic construct
  misc_feature 1..33: note = primer #9 (IL2_L19F_F)
  source 1..33: mol_type = other DNA
gagcatttactgttcgatttacagatgattttg

SEQ ID NO: 223 - DNA, 33 bp, synthetic construct
  misc_feature 1..33: note = primer #10 (IL2_L19F_R)
  source 1..33: mol_type = other DNA
caaaatcatctgtaaatcgaacagtaaatgctc

SEQ ID NO: 224 - DNA, 33 bp, synthetic construct
  misc_feature 1..33: note = primer #11 (IL2_L19V_F)
  source 1..33: mol_type = other DNA
gagcatttactggtggatttacagatgattttg

SEQ ID NO: 225 - DNA, 33 bp, synthetic construct
  misc_feature 1..33: note = primer #12 (IL2_L19V_R)
  source 1..33: mol_type = other DNA
caaaatcatctgtaaatccaccagtaaatgctc

SEQ ID NO: 226 - DNA, 33 bp, synthetic construct
  misc_feature 1..33: note = primer #13 (IL2_L19Y_F)
  source 1..33: mol_type = other DNA
caactggagcatttactgtacgatttacagatg

SEQ ID NO: 227 - DNA, 33 bp, synthetic construct
  misc_feature 1..33: note = primer #14 (IL2_L19Y_R)
  source 1..33: mol_type = other DNA
catctgtaaatcgtacagtaaatgctccagttg

SEQ ID NO: 228 - DNA, 34 bp, synthetic construct
  misc_feature 1..34: note = primer #15 (IL2_D20F_F)
  source 1..34: mol_type = other DNA
ggagcatttactgctgtttttacagatgattttg

SEQ ID NO: 229 - DNA, 34 bp, synthetic construct
  misc_feature 1..34: note = primer #16 (IL2_D20F_R)
  source 1..34: mol_type = other DNA
caaaatcatctgtaaaaacagcagtaaatgctcc

SEQ ID NO: 230 - DNA, 33 bp, synthetic construct
  misc_feature 1..33: note = primer #17 (IL2_D20V_F)
  source 1..33: mol_type = other DNA
gagcatttactgctggttttacagatgattttg

SEQ ID NO: 231 - DNA, 33 bp, synthetic construct
  misc_feature 1..33: note = primer #18 (IL2_D20V_R)
  source 1..33: mol_type = other DNA
caaaatcatctgtaaaaccagcagtaaatgctc

SEQ ID NO: 232 - DNA, 33 bp, synthetic construct
  misc_feature 1..33: note = primer #19 (IL2_K32C_F)
  source 1..33: mol_type = other DNA
ggaattaataattactgtaatcccaaactcacc

SEQ ID NO: 233 - DNA, 33 bp, synthetic construct
  misc_feature 1..33: note = primer #20 (IL2_K32C_R)
  source 1..33: mol_type = other DNA
ggtgagtttgggattacagtaattattaattcc

SEQ ID NO: 234 - DNA, 33 bp, synthetic construct
  misc_feature 1..33: note = primer #21 (IL2_K35C_F)
  source 1..33: mol_type = other DNA
aattacaagaatccctgtctcaccaggatgctc

SEQ ID NO: 235 - DNA, 33 bp, synthetic construct
  misc_feature 1..33: note = primer #22 (IL2_K35C_R)
  source 1..33: mol_type = other DNA
gagcatcctggtgagacagggattcttgtaatt

SEQ ID NO: 236 - DNA, 33 bp, synthetic construct
  misc_feature 1..33: note = primer #23 (IL2_K35E_F)
  source 1..33: mol_type = other DNA
taattacaagaatcccgaactcaccgcgatgct

SEQ ID NO: 237 - DNA, 33 bp, synthetic construct
  misc_feature 1..33: note = primer #24 (IL2_K35E_R)
  source 1..33: mol_type = other DNA
agcatcgcggtgagttcgggattcttgtaatta

SEQ ID NO: 238 - DNA, 25 bp, synthetic construct
  misc_feature 1..25: note = primer #25 (IL2_R38A_F)
  source 1..25: mol_type = other DNA
ccaaactcaccgcgatgctcacatt

SEQ ID NO: 239 - DNA, 25 bp, synthetic construct
  misc_feature 1..25: note = primer #26 (IL2_R38A_R)
  source 1..25: mol_type = other DNA
aatgtgagcatcgcggtgagtttgg

SEQ ID NO: 240 - DNA, 33 bp, synthetic construct
  misc_feature 1..33: note = primer #27 (IL2_R38A_F42F_F)
  source 1..33: mol_type = other DNA
caccgcgatgctcacatttaagttttacatgcc

SEQ ID NO: 241 - DNA, 33 bp, synthetic construct
  misc_feature 1..33: note = primer #28 (IL2_R38A_F42F_R)
  source 1..33: mol_type = other DNA
ggcatgtaaaacttaaatgtgagcatcgcggtg

SEQ ID NO: 242 - DNA, 25 bp, synthetic construct
  misc_feature 1..25: note = primer #29 (IL2_A38R_F)
  source 1..25: mol_type = other DNA
ccaaactcaccaggatgctcacatt

SEQ ID NO: 243 - DNA, 25 bp, synthetic construct
  misc_feature 1..25: note = primer #30 (IL2_A38R_R)
  source 1..25: mol_type = other DNA
aatgtgagcatcctggtgagtttgg

SEQ ID NO: 244 - DNA, 30 bp, synthetic construct
  misc_feature 1..30: note = primer #31 (IL2_R38D_F)
  source 1..30: mol_type = other DNA
aagaatcccaaactcaccgatatgctcaca

SEQ ID NO: 245 - DNA, 30 bp, synthetic construct
  misc_feature 1..30: note = primer #32 (IL2_R38D_R)
  source 1..30: mol_type = other DNA
tgtgagcatatcggtgagtttgggattctt

SEQ ID NO: 246 - DNA, 30 bp, synthetic construct
  misc_feature 1..30: note = primer #33 (IL2_F42A_F)
  source 1..30: mol_type = other DNA
ccaggatgctcacagctaagttttacatgc

SEQ ID NO: 247 - DNA, 30 bp, synthetic construct
  misc_feature 1..30: note = primer #34 (IL2_F42A_R)
  source 1..30: mol_type = other DNA
gcatgtaaaacttagctgtgagcatcctgg

SEQ ID NO: 248 - DNA, 30 bp, synthetic construct
  misc_feature 1..30: note = primer #35 (IL2_F42K_F)
  source 1..30: mol_type = other DNA
ccaggatgctcacaaagaagttttacatgc

SEQ ID NO: 249 - DNA, 30 bp, synthetic construct
  misc_feature 1..30: note = primer #36 (IL2_F42K_R)
  source 1..30: mol_type = other DNA
gcatgtaaaacttctttgtgagcatcctgg

SEQ ID NO: 250 - DNA, 30 bp, synthetic construct
  misc_feature 1..30: note = primer #37 (IL2_F42K_K43E_F)
  source 1..30: mol_type = other DNA
atgctcacaaaggagttttacatgcccaag

SEQ ID NO: 251 - DNA, 30 bp, synthetic construct
  misc_feature 1..30: note = primer #38 (IL2_F42K_K43E_R)
  source 1..30: mol_type = other DNA
cttgggcatgtaaaactcctttgtgagcat

SEQ ID NO: 252 - DNA, 30 bp, synthetic construct
  misc_feature 1..30: note = primer #39 (IL2_F42K_K43Q_F)
  source 1..30: mol_type = other DNA
atgctcacaaagcagttttacatgcccaag

SEQ ID NO: 253 - DNA, 30 bp, synthetic construct
  misc_feature 1..30: note = primer #40 (IL2_F42K_K43Q_R)
  source 1..30: mol_type = other DNA
cttgggcatgtaaaactgctttgtgagcat

SEQ ID NO: 254 - DNA, 30 bp, synthetic construct
  misc_feature 1..30: note = primer #41 (IL2_K42F_F)
  source 1..30: mol_type = other DNA
ccaggatgctcacatttaagttttacatgc

SEQ ID NO: 255 - DNA, 30 bp, synthetic construct
  misc_feature 1..30: note = primer #42 (IL2_K42F_R)
  source 1..30: mol_type = other DNA
gcatgtaaaacttaaatgtgagcatcctgg

SEQ ID NO: 256 - DNA, 39 bp, synthetic construct
  misc_feature 1..39: note = primer #43 (IL2_K42F_F_long)
  source 1..39: mol_type = other DNA
ctcaccaggatgctcacatttaagttttacatgcccaag

SEQ ID NO: 257 - DNA, 39 bp, synthetic construct
  misc_feature 1..39: note = primer #44 (IL2_K42F_R_long)
  source 1..39: mol_type = other DNA
cttgggcatgtaaaacttaaatgtgagcatcctggtgag

SEQ ID NO: 258 - DNA, 33 bp, synthetic construct
  misc_feature 1..33: note = primer #45 (IL2_F42W_F)
  source 1..33: mol_type = other DNA
accaggatgctcacatggaagttttacatgccc

SEQ ID NO: 259 - DNA, 33 bp, synthetic construct
  misc_feature 1..33: note = primer #46 (IL2_F42W_R)
  source 1..33: mol_type = other DNA
gggcatgtaaaacttccatgtgagcatcctggt

SEQ ID NO: 260 - DNA, 33 bp, synthetic construct
  misc_feature 1..33: note = primer #47 (IL2_K43C_F)
  source 1..33: mol_type = other DNA
aggatgctcacattttgtttttacatgcccaag

SEQ ID NO: 261 - DNA, 33 bp, synthetic construct
  misc_feature 1..33: note = primer #48 (IL2_K43C_R)
  source 1..33: mol_type = other DNA
cttgggcatgtaaaaacaaaatgtgagcatcct

SEQ ID NO: 262 - DNA, 19 bp, synthetic construct
  misc_feature 1..19: note = primer #49 (IL2_Y45A_F)
  source 1..19: mol_type = other DNA
gaagtttgccatgcccaag

SEQ ID NO: 263 - DNA, 19 bp, synthetic construct
  misc_feature 1..19: note = primer #50 (IL2_Y45A_R)
  source 1..19: mol_type = other DNA
cttgggcatggcaaacttc

SEQ ID NO: 264 - DNA, 34 bp, synthetic construct
  misc_feature 1..34: note = primer #51 (IL2_Y45A_F_long)
  source 1..34: mol_type = other DNA
gctcacaaagaagtttgccatgcccaagaaggcc

SEQ ID NO: 265 - DNA, 34 bp, synthetic construct
  misc_feature 1..34: note = primer #52 (IL2_Y45A_R_long)
  source 1..34: mol_type = other DNA
ggccttcttgggcatggcaaacttctttgtgagc

SEQ ID NO: 266 - DNA, 33 bp, synthetic construct
  misc_feature 1..33: note = primer #53 (IL2_K48C_F)
  source 1..33: mol_type = other DNA
aagttttacatgccctgtaaggccacagaactg

SEQ ID NO: 267 - DNA, 33 bp, synthetic construct
  misc_feature 1..33: note = primer #54 (IL2_K48C_R)
  source 1..33: mol_type = other DNA
cagttctgtggccttacagggcatgtaaaactt

SEQ ID NO: 268 - DNA, 33 bp, synthetic construct
  misc_feature 1..33: note = primer #55 (IL2_K49C_F)
  source 1..33: mol_type = other DNA
ttttacatgcccaagtgtgccacagaactgaaa

SEQ ID NO: 269 - DNA, 33 bp, synthetic construct
  misc_feature 1..33: note = primer #56 (IL2_K49C_R)
  source 1..33: mol_type = other DNA
tttcagttctgtggcacacttgggcatgtaaaa

SEQ ID NO: 270 - DNA, 33 bp, synthetic construct
  misc_feature 1..33: note = primer #57 (IL2_E61D_F)
  source 1..33: mol_type = other DNA
cttcagtgtctagaagacgaactcaaacctctg

SEQ ID NO: 271 - DNA, 33 bp, synthetic construct
  misc_feature 1..33: note = primer #58 (IL2_E61D_R)
  source 1..33: mol_type = other DNA
cagaggtttgagttcgtcttctagacactgaag

SEQ ID NO: 272 - DNA, 33 bp, synthetic construct
  misc_feature 1..33: note = primer #59 (IL2_E61Q_F)
  source 1..33: mol_type = other DNA
cttcagtgtctagaacaagaactcaaacctctg

SEQ ID NO: 273 - DNA, 33 bp, synthetic construct
  misc_feature 1..33: note = primer #60 (IL2_E61Q_R)
  source 1..33: mol_type = other DNA
cagaggtttgagttcttgttctagacactgaag

SEQ ID NO: 274 - DNA, 30 bp, synthetic construct
  misc_feature 1..30: note = primer #61 (IL2_E61R_F)
  source 1..30: mol_type = other DNA
cttcagtgtctagaacgggaactcaaacct

SEQ ID NO: 275 - DNA, 30 bp, synthetic construct
  misc_feature 1..30: note = primer #62 (IL2_E61R_R)
  source 1..30: mol_type = other DNA
aggtttgagttcccgttctagacactgaag

SEQ ID NO: 276 - DNA, 33 bp, synthetic construct
  misc_feature 1..33: note = primer #63 (IL2_E68D_F)
  source 1..33: mol_type = other DNA
ctcaaacctctggaggacgtgctaaatttagct

SEQ ID NO: 277 - DNA, 33 bp, synthetic construct
  misc_feature 1..33: note = primer #64 (IL2_E68D_R)
  source 1..33: mol_type = other DNA
agctaaatttagcacgtcctccagaggtttgag

SEQ ID NO: 278 - DNA, 33 bp, synthetic construct
  misc_feature 1..33: note = primer #65 (IL2_E68Q_F)
  source 1..33: mol_type = other DNA
ctcaaacctctggagcaggtgctaaatttagct

SEQ ID NO: 279 - DNA, 33 bp, synthetic construct
  misc_feature 1..33: note = primer #66 (IL2_E68Q_R)
  source 1..33: mol_type = other DNA
agctaaatttagcacctgctccagaggtttgag

SEQ ID NO: 280 - DNA, 32 bp, synthetic construct
  misc_feature 1..32: note = primer #67 (IL2_V69G_F)
  source 1..32: mol_type = other DNA
caaacctctggaggaagggctaaatttagctc

SEQ ID NO: 281 - DNA, 32 bp, synthetic construct
  misc_feature 1..32: note = primer #68 (IL2_V69G_R)
  source 1..32: mol_type = other DNA
gagctaaatttagcccttcctccagaggtttg

SEQ ID NO: 282 - DNA, 40 bp, synthetic construct
  misc_feature 1..40: note = primer #69 (IL2_V69G_Q74A_F)
  source 1..40: mol_type = other DNA
ctggaggaagggctaaatttagctgcaagcaaaaactttc

SEQ ID NO: 283 - DNA, 40 bp, synthetic construct
  misc_feature 1..40: note = primer #70 (IL2_V69G_Q74A_R)
  source 1..40: mol_type = other DNA
gaaagtttttgcttgcagctaaatttagcccttcctccag

SEQ ID NO: 284 - DNA, 24 bp, synthetic construct
  misc_feature 1..24: note = primer #71 (IL2_Q74H_F)
  source 1..24: mol_type = other DNA
ctaaatttagctcacagcaaaaac

SEQ ID NO: 285 - DNA, 24 bp, synthetic construct
  misc_feature 1..24: note = primer #72 (IL2_Q74H_R)
  source 1..24: mol_type = other DNA
gtttttgctgtgagctaaatttag

SEQ ID NO: 286 - DNA, 33 bp, synthetic construct
  misc_feature 1..33: note = primer #73 (IL2_K76C_F)
  source 1..33: mol_type = other DNA
aatttagctcaaagctgtaactttcacttaaga

SEQ ID NO: 287 - DNA, 33 bp, synthetic construct
  misc_feature 1..33: note = primer #74 (IL2_K76C_R)
  source 1..33: mol_type = other DNA
tcttaagtgaaagttacagctttgagctaaatt

SEQ ID NO: 288 - DNA, 32 bp, synthetic construct
  misc_feature 1..32: note = primer #75 (IL2_L80D_R81E_F)
  source 1..32: mol_type = other DNA
aaagcaaaaactttcacgacgaacccagggac

SEQ ID NO: 289 - DNA, 32 bp, synthetic construct
  misc_feature 1..32: note = primer #76 (IL2_L80D_R81E_R)
  source 1..32: mol_type = other DNA
gtccctgggttcgtcgtgaaagtttttgcttt

SEQ ID NO: 290 - DNA, 29 bp, synthetic construct
  misc_feature 1..29: note = primer #77 (IL2_L80F_F)
  source 1..29: mol_type = other DNA
gcaaaaactttcactttagacccagggac

SEQ ID NO: 291 - DNA, 29 bp, synthetic construct
  misc_feature 1..29: note = primer #78 (IL2_L80F_R)
  source 1..29: mol_type = other DNA
gtccctgggtctaaagtgaaagtttttgc

SEQ ID NO: 292 - DNA, 29 bp, synthetic construct
  misc_feature 1..29: note = primer #79 (IL2_L80F_R81D_F)
  source 1..29: mol_type = other DNA
gcaaaaactttcactttgaccccagggac

SEQ ID NO: 293 - DNA, 29 bp, synthetic construct
  misc_feature 1..29: note = primer #80 (IL2_L80F_R81D_R)
  source 1..29: mol_type = other DNA
gtccctggggtcaaagtgaaagtttttgc

SEQ ID NO: 294 - DNA, 32 bp, synthetic construct
  misc_feature 1..32: note = primer #81 (IL2_L80F_R81E_F)
  source 1..32: mol_type = other DNA
aaagcaaaaactttcacttcgaacccagggac

SEQ ID NO: 295 - DNA, 32 bp, synthetic construct
  misc_feature 1..32: note = primer #82 (IL2_L80F_R81E_R)
  source 1..32: mol_type = other DNA
gtccctgggttcgaagtgaaagtttttgcttt

SEQ ID NO: 296 - DNA, 32 bp, synthetic construct
  misc_feature 1..32: note = primer #83 (IL2_L80V_R81D_F)
  source 1..32: mol_type = other DNA
aaagcaaaaactttcacgtggatcccagggac

SEQ ID NO: 297 - DNA, 32 bp, synthetic construct
  misc_feature 1..32: note = primer #84 (IL2_L80V_R81D_R)
  source 1..32: mol_type = other DNA
gtccctgggatccacgtgaaagtttttgcttt

SEQ ID NO: 298 - DNA, 32 bp, synthetic construct
  misc_feature 1..32: note = primer #85 (IL2_L80W_R81E_F)
  source 1..32: mol_type = other DNA
aaagcaaaaactttcactgggaacccagggac

SEQ ID NO: 299 - DNA, 32 bp, synthetic construct
  misc_feature 1..32: note = primer #86 (IL2_L80W_R81E_R)
  source 1..32: mol_type = other DNA
gtccctgggttcccagtgaaagtttttgcttt

SEQ ID NO: 300 - DNA, 29 bp, synthetic construct
  misc_feature 1..29: note = primer #87 (IL2_L80Y_R81D_F)
  source 1..29: mol_type = other DNA
gcaaaaactttcactatgaccccagggac

SEQ ID NO: 301 - DNA, 29 bp, synthetic construct
  misc_feature 1..29: note = primer #88 (IL2_L80Y_R81D_R)
  source 1..29: mol_type = other DNA
gtccctggggtcatagtgaaagtttttgc

SEQ ID NO: 302 - DNA, 32 bp, synthetic construct
  misc_feature 1..32: note = primer #89 (IL2_L80Y_R81E_F)
  source 1..32: mol_type = other DNA
aaagcaaaaactttcactacgaacccagggac

SEQ ID NO: 303 - DNA, 32 bp, synthetic construct
  misc_feature 1..32: note = primer #90 (IL2_L80Y_R81E_R)
  source 1..32: mol_type = other DNA
gtccctgggttcgtagtgaaagtttttgcttt

SEQ ID NO: 304 - DNA, 32 bp, synthetic construct
  misc_feature 1..32: note = primer #91 (IL2_L80Y_R81N_F)
  source 1..32: mol_type = other DNA
aaagcaaaaactttcactacaaccccagggac

SEQ ID NO: 305 - DNA, 32 bp, synthetic construct
  misc_feature 1..32: note = primer #92 (IL2_L80Y_R81N_R)
  source 1..32: mol_type = other DNA
gtccctggggttgtagtgaaagtttttgcttt

SEQ ID NO: 306 - DNA, 29 bp, synthetic construct
  misc_feature 1..29: note = primer #93 (IL2_L80Y_R81R_F)
  source 1..29: mol_type = other DNA
gcaaaaactttcactatagacccagggac

SEQ ID NO: 307 - DNA, 29 bp, synthetic construct
  misc_feature 1..29: note = primer #94 (IL2_L80Y_R81R_R)
  source 1..29: mol_type = other DNA
gtccctgggtctatagtgaaagtttttgc

SEQ ID NO: 308 - DNA, 31 bp, synthetic construct
  misc_feature 1..31: note = primer #95 (IL2_R81D_D84E_F)
  source 1..31: mol_type = other DNA
ctttcacttagaccccagggagttaatcagc

SEQ ID NO: 309 - DNA, 31 bp, synthetic construct
  misc_feature 1..31: note = primer #96 (IL2_R81D_D84E_R)
  source 1..31: mol_type = other DNA
gctgattaactccctggggtctaagtgaaag

SEQ ID NO: 310 - DNA, 34 bp, synthetic construct
  misc_feature 1..34: note = primer #97 (IL2_R81D_F)
  source 1..34: mol_type = other DNA
caaaaactttcacttagaccccagggacttaatc

SEQ ID NO: 311 - DNA, 34 bp, synthetic construct
  misc_feature 1..34: note = primer #98 (IL2_R81D_R)
  source 1..34: mol_type = other DNA
gattaagtccctggggtctaagtgaaagtttttg

SEQ ID NO: 312 - DNA, 51 bp, synthetic construct
  misc_feature 1..51: note = primer #99 (IL2_R81D_F_long)
  source 1..51: mol_type = other DNA
ctcaaagcaaaaactttcacttagaccccagggacttaatcagcaaatatc

SEQ ID NO: 313 - DNA, 51 bp, synthetic construct
  misc_feature 1..51: note = primer #100 (IL2_R81D_R_long)
  source 1..51: mol_type = other DNA
gatatttgctgattaagtccctggggtctaagtgaaagtttttgctttgag

SEQ ID NO: 314 - DNA, 43 bp, synthetic construct
  misc_feature 1..43: note = primer #101 (IL2_R81E_D84E_L85V_I86V_V91T_I92F_F)
  source 1..43: mol_type = other DNA
gaacccagggaggtagtcagcaatatcaacacatttgttctgg

SEQ ID NO: 315 - DNA, 43 bp, synthetic construct
  misc_feature 1..43: note = primer #102 (IL2_R81E_D84E_L85V_I86V_V91T_I92F_R)
  source 1..43: mol_type = other DNA
ccagaacaaatgtgttgatattgctgactacctccctgggttc

SEQ ID NO: 316 - DNA, 31 bp, synthetic construct
  misc_feature 1..31: note = primer #103 (IL2_R81E_F)
  source 1..31: mol_type = other DNA
aaactttcacttagaacccagggacttaatc

SEQ ID NO: 317 - DNA, 31 bp, synthetic construct
  misc_feature 1..31: note = primer #104 (IL2_R81E_R)
  source 1..31: mol_type = other DNA
gattaagtccctgggttctaagtgaaagttt

SEQ ID NO: 318 - DNA, 33 bp, synthetic construct
  misc_feature 1..33: note = primer #105 (IL2_P82G_F)
  source 1..33: mol_type = other DNA
aactttcacttcgaaggcagggacgtagtcagc

SEQ ID NO: 319 - DNA, 33 bp, synthetic construct
  misc_feature 1..33: note = primer #106 (IL2_P82G_R)
  source 1..33: mol_type = other DNA
gctgactacgtccctgccttcgaagtgaaagtt

SEQ ID NO: 320 - DNA, 33 bp, synthetic construct
  misc_feature 1..33: note = primer #107 (IL2_P82V_F)
  source 1..33: mol_type = other DNA
aactttcacttcgaagtcagggacgtagtcagc

SEQ ID NO: 321 - DNA, 33 bp, synthetic construct
  misc_feature 1..33: note = primer #108 (IL2_P82V_R)
  source 1..33: mol_type = other DNA
gctgactacgtccctgacttcgaagtgaaagtt

SEQ ID NO: 322 - DNA, 34 bp, synthetic construct
  misc_feature 1..34: note = primer #109 (IL2_D84E_F)
  source 1..34: mol_type = other DNA
cttaagacccagggagttaatcagcaatatcaac

SEQ ID NO: 323 - DNA, 34 bp, synthetic construct
  misc_feature 1..34: note = primer #110 (IL2_D84E_R)
  source 1..34: mol_type = other DNA
gttgatattgctgattaactccctgggtcttaag

SEQ ID NO: 324 - DNA, 32 bp, synthetic construct
  misc_feature 1..32: note = primer #111 (IL2_D84F_F)
  source 1..32: mol_type = other DNA
cacttagaccccaggttcttaatcagcaatat

SEQ ID NO: 325 - DNA, 32 bp, synthetic construct
  misc_feature 1..32: note = primer #112 (IL2_D84F_R)
  source 1..32: mol_type = other DNA
atattgctgattaagaacctggggtctaagtg

SEQ ID NO: 326 - DNA, 31 bp, synthetic construct
  misc_feature 1..31: note = primer #113 (IL2_D84V_F)
  source 1..31: mol_type = other DNA
acttagaccccagggtcttaatcagcaatat

SEQ ID NO: 327 - DNA, 31 bp, synthetic construct
  misc_feature 1..31: note = primer #114 (IL2_D84V_R)
  source 1..31: mol_type = other DNA
atattgctgattaagaccctggggtctaagt

SEQ ID NO: 328 - DNA, 35 bp, synthetic construct
  misc_feature 1..35: note = primer #115 (IL2_L85A_I86A_F)
  source 1..35: mol_type = other DNA
ctttgaccccagggacgctgccagcaatatcaacg

SEQ ID NO: 329 - DNA, 35 bp, synthetic construct
  misc_feature 1..35: note = primer #116 (IL2_L85A_I86A_R)
  source 1..35: mol_type = other DNA
cgttgatattgctggcagcgtccctggggtcaaag

SEQ ID NO: 330 - DNA, 30 bp, synthetic construct
  misc_feature 1..30: note = primer #117 (IL2_L85E_I86V_F)
  source 1..30: mol_type = other DNA
cccagggacgaagtgagcaatatcaacgta

SEQ ID NO: 331 - DNA, 30 bp, synthetic construct
  misc_feature 1..30: note = primer #118 (IL2_L85E_I86V_R)
  source 1..30: mol_type = other DNA
tacgttgatattgctcacttcgtccctggg

SEQ ID NO: 332 - DNA, 27 bp, synthetic construct
  misc_feature 1..27: note = primer #119 (IL2_L85F_I86L_F)
  source 1..27: mol_type = other DNA
cccagggacttcctcagcaatatcaac

SEQ ID NO: 333 - DNA, 27 bp, synthetic construct
  misc_feature 1..27: note = primer #120 (IL2_L85F_I86L_R)
  source 1..27: mol_type = other DNA
gttgatattgctgaggaagtccctggg

SEQ ID NO: 334 - DNA, 27 bp, synthetic construct
  misc_feature 1..27: note = primer #121 (IL2_L85F_I86V_F)
  source 1..27: mol_type = other DNA
cccagggacttcgtcagcaatatcaac

SEQ ID NO: 335 - DNA, 27 bp, synthetic construct
  misc_feature 1..27: note = primer #122 (IL2_L85F_I86V_R)
  source 1..27: mol_type = other DNA
gttgatattgctgacgaagtccctggg

SEQ ID NO: 336 - DNA, 27 bp, synthetic construct
  misc_feature 1..27: note = primer #123 (IL2_L85G_I86A_F)
  source 1..27: mol_type = other DNA
cccagggacggagccagcaatatcaac

SEQ ID NO: 337 - DNA, 27 bp, synthetic construct
  misc_feature 1..27: note = primer #124 (IL2_L85G_I86A_R)
  source 1..27: mol_type = other DNA
gttgatattgctggctccgtccctggg

SEQ ID NO: 338 - DNA, 35 bp, synthetic construct
  misc_feature 1..35: note = primer #125 (IL2_L85G_I86V_F)
  source 1..35: mol_type = other DNA
ctttgaccccagggacggtgttagcaatatcaacg

SEQ ID NO: 339 - DNA, 35 bp, synthetic construct
  misc_feature 1..35: note = primer #126 (IL2_L85G_I86V_R)
  source 1..35: mol_type = other DNA
cgttgatattgctaacccagtccctggggtcaaag

SEQ ID NO: 340 - DNA, 27 bp, synthetic construct
  misc_feature 1..27: note = primer #127 (IL2_L85I_I86V_F)
  source 1..27: mol_type = other DNA
cccagggacatagtcagcaatatcaac

SEQ ID NO: 341 - DNA, 27 bp, synthetic construct
  misc_feature 1..27: note = primer #128 (IL2_L85I_I86V_R)
  source 1..27: mol_type = other DNA
gttgatattgctgactatgtccctggg

SEQ ID NO: 342 - DNA, 34 bp, synthetic construct
  misc_feature 1..34: note = primer #129 (IL2_L85L_I86A_F)
  source 1..34: mol_type = other DNA
cttcgaacccagggacctggccagcaatatcaac

SEQ ID NO: 343 - DNA, 34 bp, synthetic construct
  misc_feature 1..34: note = primer #130 (IL2_L85L_I86A_R)
  source 1..34: mol_type = other DNA
gttgatattgctggccaggtccctgggttcgaag

SEQ ID NO: 344 - DNA, 34 bp, synthetic construct
  misc_feature 1..34: note = primer #131 (IL2_L85L_I86V_F)
  source 1..34: mol_type = other DNA
ctttgagcccagggacttagtcagcaatatcaac

SEQ ID NO: 345 - DNA, 34 bp, synthetic construct
  misc_feature 1..34: note = primer #132 (IL2_L85L_I86V_R)
  source 1..34: mol_type = other DNA
gttgatattgctgactaagtccctgggctcaaag

SEQ ID NO: 346 - DNA, 27 bp, synthetic construct
  misc_feature 1..27: note = primer #133 (IL2_L85T_I86G_F)
  source 1..27: mol_type = other DNA
cccagggacacaggcagcaatatcaac

SEQ ID NO: 347 - DNA, 27 bp, synthetic construct
  misc_feature 1..27: note = primer #134 (IL2_L85T_I86G_R)
  source 1..27: mol_type = other DNA
gttgatattgctgcctgtgtccctggg

SEQ ID NO: 348 - DNA, 29 bp, synthetic construct
  misc_feature 1..29: note = primer #135 (IL2_L85V_I86G_F)
  source 1..29: mol_type = other DNA
cccagggacgtaggcagcaatatcaacgt

SEQ ID NO: 349 - DNA, 29 bp, synthetic construct
  misc_feature 1..29: note = primer #136 (IL2_L85V_I86G_R)
  source 1..29: mol_type = other DNA
acgttgatattgctgcctacgtccctggg

SEQ ID NO: 350 - DNA, 31 bp, synthetic construct
  misc_feature 1..31: note = primer #137 (IL2_L85V_I86I_codon_F)
  source 1..31: mol_type = other DNA
gaacccagggacgtaatcagcaatatcaacg

SEQ ID NO: 351 - DNA, 31 bp, synthetic construct
  misc_feature 1..31: note = primer #138 (IL2_L85V_I86I_codon_R)
  source 1..31: mol_type = other DNA
cgttgatattgctgattacgtccctgggttc

SEQ ID NO: 352 - DNA, 30 bp, synthetic construct
  misc_feature 1..30: note = primer #139 (IL2_L85V_I86V_F)
  source 1..30: mol_type = other DNA
gacccagggacgtagtcagcaatatcaacg

SEQ ID NO: 353 - DNA, 30 bp, synthetic construct
  misc_feature 1..30: note = primer #140 (IL2_L85V_I86V_R)
  source 1..30: mol_type = other DNA
cgttgatattgctgactacgtccctgggtc

SEQ ID NO: 354 - DNA, 29 bp, synthetic construct
  misc_feature 1..29: note = primer #141 (IL2_L85W_I86V_F)
  source 1..29: mol_type = other DNA
cccagggactgggtcagcaatatcaacgt

SEQ ID NO: 355 - DNA, 29 bp, synthetic construct
  misc_feature 1..29: note = primer #142 (IL2_L85W_I86V_R)
  source 1..29: mol_type = other DNA
acgttgatattgctgacccagtccctggg

SEQ ID NO: 356 - DNA, 30 bp, synthetic construct
  misc_feature 1..30: note = primer #143 (IL2_L85W_I86V_Long_F)
  source 1..30: mol_type = other DNA
tttgaccccagggactgggtcagcaatatc

SEQ ID NO: 357 - DNA, 30 bp, synthetic construct
  misc_feature 1..30: note = primer #144 (IL2_L85W_I86V_Long_R)
  source 1..30: mol_type = other DNA
gatattgctgacccagtccctggggtcaaa

SEQ ID NO: 358 - DNA, 34 bp, synthetic construct
  misc_feature 1..34: note = primer #145 (IL2_L85Y_I86V_F)
  source 1..34: mol_type = other DNA
cttcgaacccagggactacgtcagcaatatcaac

SEQ ID NO: 359 - DNA, 34 bp, synthetic construct
  misc_feature 1..34: note = primer #146 (IL2_L85Y_I86V_R)
  source 1..34: mol_type = other DNA
gttgatattgctgacgtagtccctgggttcgaag

SEQ ID NO: 360 - DNA, 31 bp, synthetic construct
  misc_feature 1..31: note = primer #147 (IL2_I86V_V91T_I92F_F)
  source 1..31: mol_type = other DNA
gtcagcaatatcaacacatttgttctggaac

SEQ ID NO: 361 - DNA, 31 bp, synthetic construct
  misc_feature 1..31: note = primer #148 (IL2_I86V_V91T_I92F_R)
  source 1..31: mol_type = other DNA
gttccagaacaaatgtgttgatattgctgac

SEQ ID NO: 362 - DNA, 33 bp, synthetic construct
  misc_feature 1..33: note = primer #149 (IL2_S87C_F)
  source 1..33: mol_type = other DNA
cccagggacttaatctgcaatatcaacgtaata

SEQ ID NO: 363 - DNA, 33 bp, synthetic construct
  misc_feature 1..33: note = primer #150 (IL2_S87C_R)
  source 1..33: mol_type = other DNA
tattacgttgatattgcagattaagtccctggg

SEQ ID NO: 364 - DNA, 34 bp, synthetic construct
  misc_feature 1..34: note = primer #151 (IL2_N88F_F)
  source 1..34: mol_type = other DNA
cagggacttaatcagctttatcaacgtatttgtt

SEQ ID NO: 365 - DNA, 34 bp, synthetic construct
  misc_feature 1..34: note = primer #152 (IL2_N88F_R)
  source 1..34: mol_type = other DNA
aacaaatacgttgataaagctgattaagtccctg

SEQ ID NO: 366 - DNA, 33 bp, synthetic construct
  misc_feature 1..33: note = primer #153 (IL2_N88Q_F)
  source 1..33: mol_type = other DNA
gggacttaatcagccaaatcaacgtaatagttc

SEQ ID NO: 367 - DNA, 33 bp, synthetic construct
  misc_feature 1..33: note = primer #154 (IL2_N88Q_R)
  source 1..33: mol_type = other DNA
gaactattacgttgatttggctgattaagtccc

SEQ ID NO: 368 - DNA, 34 bp, synthetic construct
  misc_feature 1..34: note = primer #155 (IL2_N88V_F)
  source 1..34: mol_type = other DNA
cagggacttaatcagcgttatcaacgtatttgtt

SEQ ID NO: 369 - DNA, 34 bp, synthetic construct
  misc_feature 1..34: note = primer #156 (IL2_N88V_R)
  source 1..34: mol_type = other DNA
aacaaatacgttgataacgctgattaagtccctg

SEQ ID NO: 370 - DNA, 33 bp, synthetic construct
  misc_feature 1..33: note = primer #157 (IL2_I89F_F)
  source 1..33: mol_type = other DNA
gacttaatcagcaatttcaacgtaatagttctg

SEQ ID NO: 371 - DNA, 33 bp, synthetic construct
  misc_feature 1..33: note = primer #158 (IL2_I89F_R)
  source 1..33: mol_type = other DNA
cagaactattacgttgaaattgctgattaagtc

SEQ ID NO: 372 - DNA, 33 bp, synthetic construct
  misc_feature 1..33: note = primer #159 (IL2_V91E_I92F_F)
  source 1..33: mol_type = other DNA
agcaatatcaacgaattcgttctggaactaaag

SEQ ID NO: 373 - DNA, 33 bp, synthetic construct
  misc_feature 1..33: note = primer #160 (IL2_V91E_I92F_R)
  source 1..33: mol_type = other DNA
ctttagttccagaacgaattcgttgatattgct

SEQ ID NO: 374 - DNA, 33 bp, synthetic construct
  misc_feature 1..33: note = primer #161 (IL2_V91E_I92W_F)
  source 1..33: mol_type = other DNA
agcaatatcaacgaatgggttctggaactaaag

SEQ ID NO: 375 - DNA, 33 bp, synthetic construct
  misc_feature 1..33: note = primer #162 (IL2_V91E_I92W_R)
  source 1..33: mol_type = other DNA
ctttagttccagaacccattcgttgatattgct

SEQ ID NO: 376 - DNA, 34 bp, synthetic construct
  misc_feature 1..34: note = primer #163 (IL2_V91F_F)
  source 1..34: mol_type = other DNA
cagcaatatcaactttatagttctggaactaaag

SEQ ID NO: 377 - DNA, 34 bp, synthetic construct
  misc_feature 1..34: note = primer #164 (IL2_V91F_R)
  source 1..34: mol_type = other DNA
ctttagttccagaactataaagttgatattgctg

SEQ ID NO: 378 - DNA, 34 bp, synthetic construct
  misc_feature 1..34: note = primer #165 (IL2_V91T_F)
  source 1..34: mol_type = other DNA
cagcaatatcaacacaatagttctggaactaaag

SEQ ID NO: 379 - DNA, 34 bp, synthetic construct
  misc_feature 1..34: note = primer #166 (IL2_V91T_R)
  source 1..34: mol_type = other DNA
ctttagttccagaactattgtgttgatattgctg

SEQ ID NO: 380 - DNA, 32 bp, synthetic construct
  misc_feature 1..32: note = primer #167 (IL2_I92F_F)
  source 1..32: mol_type = other DNA
gcaatatcaacgtatttgttctggaactaaag

SEQ ID NO: 381 - DNA, 32 bp, synthetic construct
  misc_feature 1..32: note = primer #168 (IL2_I92F_R)
  source 1..32: mol_type = other DNA
ctttagttccagaacaaatacgttgatattgc

SEQ ID NO: 382 - DNA, 49 bp, synthetic construct
  misc_feature 1..49: note = primer #169 (IL2_I92F_F_long)
  source 1..49: mol_type = other DNA
gacttaatcagcaatatcaacgtatttgttctggaactaaagggatctg

SEQ ID NO: 383 - DNA, 49 bp, synthetic construct
  misc_feature 1..49: note = primer #170 (IL2_I92F_R_long)
  source 1..49: mol_type = other DNA
cagatccctttagttccagaacaaatacgttgatattgctgattaagtc

SEQ ID NO: 384 - DNA, 45 bp, synthetic construct
  misc_feature 1..45: note = primer #171 (IL2_I92F_F_long2)
  source 1..45: mol_type = other DNA
cttaatcagcaatatcaacgtatttgttctggaactaaagggatc

SEQ ID NO: 385 - DNA, 45 bp, synthetic construct
  misc_feature 1..45: note = primer #172 (IL2_I92F_R_long2)
  source 1..45: mol_type = other DNA
gatccctttagttccagaacaaatacgttgatattgctgattaag

SEQ ID NO: 386 - DNA, 32 bp, synthetic construct
  misc_feature 1..32: note = primer #173 (IL2_I92F_L94F_L96F_F)
  source 1..32: mol_type = other DNA
caacgtatttgttttcgaattcaagggatctg

SEQ ID NO: 387 - DNA, 32 bp, synthetic construct
  misc_feature 1..32: note = primer #174 (IL2_I92F_L94F_L96F_R)
  source 1..32: mol_type = other DNA
cagatcccttgaattcgaaaacaaatacgttg

SEQ ID NO: 388 - DNA, 39 bp, synthetic construct
  misc_feature 1..39: note = primer #175 (IL2_I92F_L94F_L96F_F)
  source 1..39: mol_type = other DNA
gcaatatcaacgtatttgttttcgaatttaagggatctg

SEQ ID NO: 389 - DNA, 39 bp, synthetic construct
  misc_feature 1..39: note = primer #176 (IL2_I92F_L94F_L96F_R)
  source 1..39: mol_type = other DNA
cagatcccttaaattcgaaaacaaatacgttgatattgc

SEQ ID NO: 390 - DNA, 39 bp, synthetic construct
  misc_feature 1..39: note = primer #177 (IL2_I92F_L94F_L96I_F)
  source 1..39: mol_type = other DNA
gcaatatcaacgtatttgttttcgaaattaagggatctg

SEQ ID NO: 391 - DNA, 39 bp, synthetic construct
  misc_feature 1..39: note = primer #178 (IL2_I92F_L94F_L96I_R)
  source 1..39: mol_type = other DNA
cagatcccttaatttcgaaaacaaatacgttgatattgc

SEQ ID NO: 392 - DNA, 39 bp, synthetic construct
  misc_feature 1..39: note = primer #179 (IL2_I92F_L94F_L96V_F)
  source 1..39: mol_type = other DNA
gcaatatcaacgtatttgttttcgaagtaaagggatctg

SEQ ID NO: 393 - DNA, 39 bp, synthetic construct
  misc_feature 1..39: note = primer #180 (IL2_I92F_L94F_L96V_R)
  source 1..39: mol_type = other DNA
cagatccctttacttcgaaaacaaatacgttgatattgc

SEQ ID NO: 394 - DNA, 33 bp, synthetic construct
  misc_feature 1..33: note = primer #181 (IL2_I92I_F)
  source 1..33: mol_type = other DNA
gcaatatcaacgtaattgttctggaactaaagg

SEQ ID NO: 395 - DNA, 33 bp, synthetic construct
  misc_feature 1..33: note = primer #182 (IL2_I92I_R)
  source 1..33: mol_type = other DNA
cctttagttccagaacaattacgttgatattgc

SEQ ID NO: 396 - DNA, 33 bp, synthetic construct
  misc_feature 1..33: note = primer #183 (IL2_I92L_F)
  source 1..33: mol_type = other DNA
gcaatatcaacgtattagttctggaactaaagg

SEQ ID NO: 397 - DNA, 33 bp, synthetic construct
  misc_feature 1..33: note = primer #184 (IL2_I92L_R)
  source 1..33: mol_type = other DNA
cctttagttccagaactaatacgttgatattgc

SEQ ID NO: 398 - DNA, 33 bp, synthetic construct
  misc_feature 1..33: note = primer #185 (IL2_I92W_F)
  source 1..33: mol_type = other DNA
agcaatatcaacgtatgggttctggaactaaag

SEQ ID NO: 399 - DNA, 33 bp, synthetic construct
  misc_feature 1..33: note = primer #186 (IL2_I92W_R)
  source 1..33: mol_type = other DNA
ctttagttccagaacccatacgttgatattgct

SEQ ID NO: 400 - DNA, 32 bp, synthetic construct
  misc_feature 1..32: note = primer #187 (IL2_I92Y_F)
  source 1..32: mol_type = other DNA
gcaatatcaacgtatatgttctggaactaaag

SEQ ID NO: 401 - DNA, 32 bp, synthetic construct
  misc_feature 1..32: note = primer #188 (IL2_I92Y_R)
  source 1..32: mol_type = other DNA
ctttagttccagaacatatacgttgatattgc

SEQ ID NO: 402 - DNA, 33 bp, synthetic construct
  misc_feature 1..33: note = primer #189 (IL2_L94F_L96F_F)
  source 1..33: mol_type = other DNA
cgtaatagttttcgaatttaagggatctgaaac

SEQ ID NO: 403 - DNA, 33 bp, synthetic construct
  misc_feature 1..33: note = primer #190 (IL2_L94F_L96F_R)
  source 1..33: mol_type = other DNA
gtttcagatcccttaaattcgaaaactattacg

SEQ ID NO: 404 - DNA, 33 bp, synthetic construct
  misc_feature 1..33: note = primer #191 (IL2_L94F_L96I_F)
  source 1..33: mol_type = other DNA
cgtaatagttttcgaaattaagggatctgaaac

SEQ ID NO: 405 - DNA, 33 bp, synthetic construct
  misc_feature 1..33: note = primer #192 (IL2_L94F_L96I_R)
  source 1..33: mol_type = other DNA
gtttcagatcccttaatttcgaaaactattacg

SEQ ID NO: 406 - DNA, 33 bp, synthetic construct
  misc_feature 1..33: note = primer #193 (IL2_L94F_L96V_F)
  source 1..33: mol_type = other DNA
cgtaatagttttcgaagtaaagggatctgaaac

SEQ ID NO: 407 - DNA, 33 bp, synthetic construct
  misc_feature 1..33: note = primer #194 (IL2_L94F_L96V_R)
  source 1..33: mol_type = other DNA
gtttcagatccctttacttcgaaaactattacg

SEQ ID NO: 408 - DNA, 30 bp, synthetic construct
  misc_feature 1..30: note = primer #195 (IL2_E95D_F)
  source 1..30: mol_type = other DNA
gttctggacctaaagggatctgaaacaaca

SEQ ID NO: 409 - DNA, 30 bp, synthetic construct
  misc_feature 1..30: note = primer #196 (IL2_E95D_R)
  source 1..30: mol_type = other DNA
tgttgtttcagatccctttaggtccagaac

SEQ ID NO: 410 - DNA, 33 bp, synthetic construct
  misc_feature 1..33: note = primer #197 (IL2_C125S_F)
  source 1..33: mol_type = other DNA
agatggattacctttagtcaaagcatcatctca

SEQ ID NO: 411 - DNA, 33 bp, synthetic construct
  misc_feature 1..33: note = primer #198 (IL2_C125S_R)
  source 1..33: mol_type = other DNA
tgagatgatgctttgactaaaggtaatccatct

SEQ ID NO: 412 - DNA, 34 bp, synthetic construct
  misc_feature 1..34: note = primer #199 (IL2_Q126T_F)
  source 1..34: mol_type = other DNA
gatggattacctttagtacaagcatcatctcaac

SEQ ID NO: 413 - DNA, 34 bp, synthetic construct
  misc_feature 1..34: note = primer #200 (IL2_Q126T_R)
  source 1..34: mol_type = other DNA
gttgagatgatgcttgtactaaaggtaatccatc

SEQ ID NO: 414 - DNA, 33 bp, synthetic construct
  misc_feature 1..33: note = primer #201 (IL2_desA1_F)
  source 1..33: mol_type = other DNA
taagaatatacatatgcctacttcaagttctac

SEQ ID NO: 415 - DNA, 33 bp, synthetic construct
  misc_feature 1..33: note = primer #202 (IL2_desA1_R)
  source 1..33: mol_type = other DNA
gtagaacttgaagtaggcatatgtatattctta

SEQ ID NO: 416 - DNA, 34 bp, synthetic construct
  misc_feature 1..34: note = primer #203 (IL2_NdeI_desA1_N-term)
  source 1..34: mol_type = other DNA
cgccatatgcctacttcaagttctacaaagaaaa

SEQ ID NO: 417 - DNA, 34 bp, synthetic construct
  misc_feature 1..34: note = primer #204 (IL2_BHI_C-term)
  source 1..34: mol_type = other DNA
cgggatcctcaagtcagtgttgagatgatgcttt

SEQ ID NO: 418 - protein, 12 aa, Homo sapiens
ESKYGPPCPSCP

SEQ ID NO: 419 - protein, 11 aa, synthetic construct
  REGION 1..11: note = Variant of hinge region
ESKYGPPPSCP

SEQ ID NO: 420 - protein, 11 aa, synthetic construct
  REGION 1..11: note = Variant of hinge region
ESKYGPPCPSP

SEQ ID NO: 421 - protein, 10 aa, synthetic construct
  REGION 1..10: note = Variant of hinge region
ESKYGPPCPS

SEQ ID NO: 422 - protein, 10 aa, synthetic construct
  REGION 1..10: note = Variant of hinge region
ESKYGPPCPP

SEQ ID NO: 423 - protein, 8 aa, synthetic construct
  REGION 1..8: note = Variant of hinge region
KYGPPCPS

SEQ ID NO: 424 - protein, 8 aa, synthetic construct
  REGION 1..8: note = Variant of hinge region
ESKYGPPC

SEQ ID NO: 425 - protein, 7 aa, synthetic construct
  REGION 1..7: note = Variant of hinge region
EKYGPPC

SEQ ID NO: 426 - protein, 6 aa, synthetic construct
  REGION 1..6: note = Variant of hinge region
ESPSCP

SEQ ID NO: 427 - protein, 5 aa, synthetic construct
  REGION 1..5: note = Variant of hinge region
EPSCP

SEQ ID NO: 428 - protein, 4 aa, synthetic construct
  REGION 1..4: note = Variant of hinge region
PSCP

SEQ ID NO: 429 - protein, 10 aa, synthetic construct
  REGION 1..10: note = Variant of hinge region
ESKYGPPSCP

SEQ ID NO: 430 - protein, 9 aa, synthetic construct
  REGION 1..9: note = Variant of hinge region
KYGPPPSCP

SEQ ID NO: 431 - protein, 9 aa, synthetic construct
  REGION 1..9: note = Variant of hinge region
ESKYGPSCP

SEQ ID NO: 432 - protein, 8 aa, synthetic construct
  REGION 1..8: note = Variant of hinge region
ESKYGPPC

SEQ ID NO: 433 - protein, 7 aa, synthetic construct
  REGION 1..7: note = Variant of hinge region
KYGPPCP

SEQ ID NO: 434 - protein, 7 aa, synthetic construct
  REGION 1..7: note = Variant of hinge region
ESKPSCP

SEQ ID NO: 435 - protein, 6 aa, synthetic construct
  REGION 1..6: note = Variant of hinge region
ESPSCP

SEQ ID NO: 436 - protein, 4 aa, synthetic construct
  REGION 1..4: note = Variant of hinge region
EPSC

SEQ ID NO: 437 - skipped

SEQ ID NO: 438 - protein, 221 aa, Homo sapiens
PSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 439 - protein, 442 aa, Homo sapiens
  REGION 1..442: note = MISC_FEATURE - Amino acids at position 1 to 442 form Human Immunoglobulin G4 Fc Fragment (homodimer)
  REGION 1..221: note = MISC_FEATURE - Amino acids at position 1 to 221 form one monomer
  REGION 222..442: note = MISC_FEATURE - Amino acids at position 222 to 442 form one monomer
  DISULFID 3..224: note = Amino acids at position 3 and position 224 form an inter-disulfide bond
  DISULFID 35..95: note = Amino acids at position 35 and position 95 form an intra-chain disulfide bond
  DISULFID 141..199: note = Amino acids at position 141 and position 199 form an intra-chain disulfide bond
  DISULFID 256..316: note = Amino acids at position 256 and position 316 form an intra-chain disulfide bond
  DISULFID 362..420: note = Amino acids at position 362 and position 420 form an intra-chain disulfide bond
PSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK